(19) United States
(12) Patent Application Publication
(10) Pub. No.: US 2009/0276918 A1
(43) Pub. Date: Nov. 5, 2009

(54) PLANTS HAVING ENHANCED YIELD-RELATED TRAITS AND A METHOD FOR MAKING THE SAME

(75) Inventors: Yang Do Choi, Seoul (KR); Ju-Kon Kim, Seoul (KR); Baek Hie Nahm, Seoul (KR); Jin Seo Jeong, Seoul (KR); Se-Jun Oh, Seoul (KR); Chang-d'eok Han, Seoul (KR); Sung Han Park, Seoul (KR); Pil Joong Chung, Seoul (KR)

Correspondence Address: CONNOLLY BOVE LODGE & HUTZ, LLP, P O BOX 2207, WILMINGTON, DE, 19899, US

(73) Assignees: CROPDESIGN N.V., Zwijnaarde (BE); CROP FUNCTIONAL GENOMICS CENTER, Seoul (KR)

(21) Appl. No.: 12/304,581

(22) PCT Filed: Jun. 15, 2007
(86) PCT No.: PCT/EP2007/005292

§ 371 Date: Dec. 12, 2008

Related U.S. Application Data
(60) Provisional application No. 60/816,447, filed on Jun. 26, 2006.
(60) Provisional application No. 60/816,444, filed on Jun. 26, 2006.
(60) Provisional application No. 60/889,957, filed on Feb. 15, 2007.
(60) Provisional application No. 60/891,045, filed on Feb. 22, 2007.
(60) Provisional application No. 60/891,047, filed on Feb. 22, 2007.

(30) Foreign Application Priority Data
Jun. 15, 2006 (EP) 06447081.8
Jun. 15, 2006 (EP) 06447082.6
Jan. 19, 2007 (EP) 07100862.7
Jan. 31, 2007 (EP) 07101534.1
Jan. 31, 2007 (EP) 07101535.8

(51) Int. Cl. C12N 15/82

(52) U.S. Cl. 800/278

(57) ABSTRACT

The present invention concerns a method for enhancing yield-related traits in plants by modulating expression of a particular type of NAC transcription factor in plants. The particular type of NAC transcription factor is one having an amino acid sequence, which when used in the construction of a NAC phylogenetic tree, tends to cluster with the group of NACs comprising the amino acid sequence represented by SEQ ID NO: 2, SEQ ID NO: 51, SEQ ID NO: 53, SEQ ID NO: 55, SEQ ID NO: 57 or SEQ ID NO: 59 rather than with any other NAC group. The present invention also concerns plants having modulated expression of a nucleic acid encoding such a NAC transcription factor, which plants have enhanced yield-related relative to control plants. The present invention further concerns a method for enhancing yield-related traits in plants by modulating expression in a plant of a nucleic acid encoding an AP2-2 polypeptide. The present invention also concerns plants having modulated expression of a nucleic acid encoding an AP2-2 polypeptide, which plants have enhanced yield-related traits relative to control plants. The present invention further concerns a method for enhancing yield-related traits in plants by modulating expression in a plant of a nucleic acid encoding an APETELA2-70-like (AP2-70-like) polypeptide. The present invention also concerns plants having modulated expression of a nucleic acid encoding an AP2-70-like polypeptide, which plants have enhanced yield-related traits relative to control plants. The invention also provides hitherto unknown NAC, AP2-2 and AP2-70-like-encoding nucleic acids, and constructs comprising the same, useful in performing the methods of the invention. The invention also provides constructs useful in the methods of the invention.

The present invention relates generally to the field of molecular biology and concerns a method for enhancing various economically important yield-related traits in plants. More specifically, the present invention concerns methods for enhancing yield-related traits in plants by modulating expression in a plant of a nucleic acid encoding a particular type of NAC transcription factor. The present invention also concerns plants having modulated expression of a nucleic acid encoding a NAC transcription factor, which plants have enhanced yield-related traits relative to control plants.

In addition, the present invention concerns a method for enhancing yield-related traits in plants by modulating expression in a plant of a nucleic acid encoding a Apetala 2-2 (AP2-2) polypeptide. The present invention also concerns plants having modulated expression of a nucleic acid encoding an AP2-2 polypeptide, which plants have enhanced yield-related traits relative to control plants. The present invention also concerns a method for enhancing yield-related traits in plants by modulating expression in a plant of a nucleic acid encoding an APETALA2-70-like (AP2-70-like) polypeptide. The present invention also concerns plants having modulated expression of a nucleic acid encoding an AP2-70-like polypeptide, which plants have enhanced yield-related traits relative to control plants. The invention also provides hitherto unknown AP2-70-like-encoding nucleic acids, and constructs comprising the same, useful in performing the methods of the invention.

The invention also provides constructs useful in the methods of the invention.

The ever-increasing world population and the dwindling supply of arable land available for agriculture fuels research towards increasing the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic and horticultural traits.

A trait of particular economic interest is increased yield. Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance and early vigour may also be important factors in determining yield. Optimizing the abovementioned factors may therefore contribute to increasing crop yield.

Seed yield is a particularly important trait, since the seeds of many plants are important for human and animal nutrition. Crops such as, corn, rice, wheat, canola and soybean account for over half the total human caloric intake, whether through direct consumption of the seeds themselves or through consumption of meat products raised on processed seeds. They are also a source of sugars, oils and many kinds of metabolites used in industrial processes. Seeds contain an embryo (the source of new shoots and roots) and an endosperm (the source of nutrients for embryo growth during germination and during early growth of seedlings). The development of a seed involves many genes, and requires the transfer of metabolites from the roots, leaves and stems into the growing seed. The endosperm, in particular, assimilates the metabolic precursors of carbohydrates, oils and proteins and synthesizes them into storage macromolecules to fill out the grain.

A further trait of economic importance for many crops is early vigour. Improving early vigour is an important objective of modern rice breeding programs in both temperate and tropical rice cultivars. Long roots are important for proper soil anchorage in water-seeded rice. Where rice is sown directly into flooded fields, and where plants must emerge rapidly through water, longer shoots are associated with vigour. Where drill-seeding is practiced, longer mesocotyls and coleoptiles are important for good seedling emergence. Early vigour may also result from increased plant fitness due to, for example, the plants being better adapted to their environment (i.e. being more able to cope with various abiotic or biotic stress factors). Plants having early vigour also show better establishment of the crop (with the crop growing in a more uniform manner, i.e. with the majority of plants reaching the various stages of development at substantially the same time), and show better growth and often better yield.

A further important trait is improved abiotic stress tolerance. Abiotic stress is a primary cause of crop loss worldwide, reducing average yields for most major crop plants by more than 50% (Wang et al., Planta (2003) 218: 1-14). Abiotic stresses may be caused by drought, salinity, extremes of temperature, chemical toxicity and oxidative stress. The ability to improve plant tolerance to abiotic stress would be of great economic advantage to farmers worldwide and would allow for the cultivation of crops during adverse conditions and in territories where cultivation of crops may not otherwise be possible.

The ability to engineer abiotic stress tolerance or early vigour into plants would be of great importance in agriculture, as would the ability to increase plant seed yield, whether through seed number, seed biomass, seed development, seed filling, or any other seed-related trait. Aside form the many applications in agriculture including in the production of ornamental plants, arboriculture, horticulture and forestry, increasing yield would also have many non-agricultural uses, such as in the production of algae for use in bioreactors (for the biotechnological production of substances such as pharmaceuticals, antibodies or vaccines or for the bioconversion of organic waste) and other such areas.

Transcription factors are usually defined as proteins that show sequence-specific DNA binding and that are capable of activating and/or repressing transcription. The Arabidopsis genome codes for at least 1533 transcriptional regulators, which account for ˜5.9% of its estimated total number of genes. About 45% of these transcription factors are reported to be from families specific to plants (Riechmann et al., 2000 (Science Vol. 290, 2105-2109)).

The present invention concerns the use of a particular type of NAC transcription factor for enhancing yield-related traits in plants.

NAC is an acronym derived from the names of the three genes first described as containing a NAC domain, namely NAM (no apical meristem), ATAF1,2 and CUC2 (cup-shaped cotyledon). NAC proteins appear to be widespread in plants, with the genome of Arabidopsis thaliana estimated to contain at least a hundred NAC-encoding genes, but without any examples having been found to date in other eukaryotes (Riechmann et al., 2000).

The NAC protein family comprises a variety of plant proteins that are identifiable by the presence of a highly conserved N-terminal NAC domain, accompanied by diverse C-terminal domains. The DNA-binding ability of NAC proteins is generally localized to the NAC domain, with the C-terminal regions constituting transcriptional activation domains. Several NAC genes have been found to be hormone inducible. NAC domains have also been implicated in interactions with other proteins, such as viral proteins and RING finger proteins. NAC proteins have also been implicated in transcriptional control in a variety of plant processes, including in the development of the shoot apical meristem and floral organs, and in the formation of lateral roots. NAC proteins have also been implicated in responses to stress and viral infections, Ernst et al., 2004 (EMBO Reports 5, 3, 297-303).

U.S. Pat. No. 6,844,486 describes a member of the NAC family, NACI, isolated from Arabidopsis thaliana. NACI was reported to be involved in the regulation of cotyledon and lateral root development. Overexpression of the nacI gene was reported to give larger plants with larger roots and more lateral roots than wild-type plants.

Surprisingly, it has now been found that modulating expression in a plant of a nucleic acid encoding a particular type of NAC transcription factor gives plants having enhanced yield-related traits relative to control plants. The particular class of NAC transcription factor suitable for enhancing yield-related traits in plants is described in detail below.

The present invention provides a method for enhancing yield-related traits in plants relative to control plants, comprising modulating expression in a plant of a nucleic acid encoding a particular type of NAC transcription factor.

In addition, the present invention concerns the use of an Apetala type transcription factor, AP2-2, for enhancing yield-related traits in plants.

AP2 (APETALA2) and EREBPs (ethylene-responsive element binding proteins, or ERF, ethylene response factors) are the prototypic members of a family of transcription factors unique to plants, whose distinguishing characteristic is that they contain the so-called AP2 DNA-binding domain. AP2/EREBP genes form a large multigene family (the AP2/ERF superfamily), and they play a variety of roles throughout the plant life cycle: from being key regulators of several developmental processes, like floral organ identity determination or control of leaf epidermal cell identity, to forming part of the mechanisms used by plants to respond to various types of biotic and environmental stress. Within the AP2/ERF superfamily, 3 large families are discriminated: the AP2 family with two AP2/ERF domains, the ERF family with a single AP2/ERF domain and the RAV family comprising a B3-type DNA binding domain. Nakano et al. (Plant Physiology 140, 411-432, 2006) studied the ERF gene family in Arabidopsis and rice, and divided the Arabidopsis ERF gene family into 12 groups (designated Group I to X, and a Group VI-like and Group Xb-like), whereas in the case of rice 15 groups were discriminated. The Arabidopsis Group VII proteins are characterised by a conserved N-terminal motif, referred to as Conserved Motif VII-1 (CMVII-1). In rice, Group VII comprises more proteins than the Arabidopsis Group VII, and though many conserved motifs are in common between the rice and Arabidopsis Group VII, a separate rice Group VIIb was created for a sequence lacking this typical CMVII-1 motif. Functionally, members of Group VII are described to be involved in osmotic stress and disease responses (for example in WO 2003007699). Ectopic overexpression of tomato JERF3 in tobacco increased the salt tolerance of the transgenics (Wang et al., Plant Molecular Biology 58, 183-192, 2004), and pepper transcription factor CaPF1 overexpression resulted in increased osmotic tolerance in pine (Tang et al, Plant Cell Rep. 26, 115-124, 2007), but also increased pathogen resistance in Arabidopsis (Yi et al., Plant Physiol. 136, 2862-2874, 2004). A similar observation was made for barley HvRAF (Jung et al., Planta Epub 26 Aug. 2006). Furthermore, a Group VII type ERF protein was used in a process for the production of Methionine (EP2005003297).

Surprisingly, it has now been found that modulating expression in a plant of a nucleic acid encoding a Group VII ERF protein (hereafter named AP2-2 polypeptide) gives plants having enhanced yield-related traits relative to control plants. These enhanced yield-related traits were not the result of increased stress resistance.

The ERF (Ethylene Response Factor) family is a large gene family of transcription factors and is part of the AP2/ERF superfamily, which also contains the AP2 and RAV families. The AP2/ERF superfamily is defined by the AP2/ERF domain, which consists of about 60 to 70 amino acids and is involved in DNA binding. These three families have been defined as follows. The AP2 family of proteins contain two repeated AP2/ERF domains, the ERF family of proteins contain a single AP2/ERF domain, and the RAV family proteins contain a B3 domain, which is a DNA-binding domain conserved in other plant-specific transcription factors, including VP1/ABI3, in addition to the single AP2/ERF domain. The ERF family is sometimes further divided into two major subfamilies, the ERF subfamily and the CBF/DREB subfamily. 147 genes have been identified in the AP2/ERF superfamily in the Arabidopsis genome, including 122 genes in the ERF family. The AP2 domain was first identified as a repeated motif within the Arabidopsis (Arabidopsis thaliana) AP2 protein, which is involved in flower development. Nakano et al., 2006 (Plant Physiol. Vol. 140, pp. 411-432).

Nakano et al., 2006 further report that genes in the AP2 family have been shown to participate in the regulation of developmental processes, e.g. flower development, spikelet meristem determinacy, leaf epidermal cell identity, and embryo development. Several proteins in the ERF family have been identified and implicated in many diverse functions in cellular processes, such as hormonal signal transduction, response to biotic and abiotic stresses, and regulation of metabolism, and in developmental processes in various plant species.

Surprisingly, it has now been found that modulating expression in a plant of a nucleic acid encoding an AP2-70-like polypeptide gives plants having enhanced yield-related traits relative to control plants.

AP2-70-like polypeptides described herein as being useful in enhancing yield-related traits in plants are classified in Group Ib (A-6) according to the classification system of Nakano et al., 2006. Nakano reports that DBF1 from maize (Zea mays)—a member of Group Ib—has been shown to activate the drought-responsive element 2 (DRE2)-dependent transcription of ABA-responsive rab17 in transiently transformed maize callus. In another Group Ib member, Medicago truncatula WXP1, overexpression of the same was reported to activate wax production in transgenic alfalfa (Medicago sativa).

### DEFINITIONS

### Polypeptide(s)/Protein(s)

The terms “polypeptide” and “protein” are used interchangeably herein and refer to amino acids in a polymeric form of any length, linked together by peptide bonds.

### Polynucleotide(s)/Nucleic Acid(s)/Nucleic Acid Sequence(s)/Nucleotide Sequence(s)

The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

### Control Plant(s)

The choice of suitable control plants is a routine part of an experimental setup and may include corresponding wild type plants or corresponding plants without the gene of interest. The control plant is typically of the same plant species or even of the same variety as the plant to be assessed. The control plant may also be a nullizygote of the plant to be assessed. Nullizygotes are individuals missing the transgene by segregation. A “control plant” as used herein refers not only to whole plants, but also to plant parts, including seeds and seed parts.

### Homologue(s)

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A deletion refers to removal of one or more amino acids from a protein.

An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag-100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues, but may be clustered depending upon functional constraints placed upon the polypeptide; insertions will usually be of the order of about 1 to 10 amino acid residues. The amino acid substitutions are preferably conservative amino acid substitutions. Conservative substitution tables are well known in the art (see for example Creighton (1984) Proteins. W.H. Freeman and Company (Eds) and Table 1 below).

TABLE 1
Examples of conserved amino acid substitutions

| | Conservative |
|---|---|
| Residue | Substitutions |
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

### Derivatives

“Derivatives” include peptides, oligopeptides, polypeptides which may, compared to the amino acid sequence of the naturally-occurring form of the protein, such as the protein of interest, comprise substitutions of amino acids with non-naturally occurring amino acid residues, or additions of non-naturally occurring amino acid residues. “Derivatives” of a protein also encompass peptides, oligopeptides, polypeptides which comprise naturally occurring altered (glycosylated, acylated, prenylated, phosphorylated, myristoylated, sulphated etc.) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein. Furthermore, “derivatives” also include fusions of the naturally-occurring form of the protein with tagging peptides such as FLAG, HIS6 or thioredoxin (for a review of tagging peptides, see Terpe, Appl. Microbiol. Biotechnol. 60, 523-533, 2003).

### Orthologue(s)/Paralogue(s)

Orthologues and paralogues encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene.

### Domain

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

### Motif/Consensus Sequence/Signature

The term “motif” or “consensus sequence” or “signature” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

### Hybridisation

The term “hybridisation” as defined herein is a process wherein substantially homologous complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore medium stringency hybridisation conditions may sometimes be needed to identify such nucleic acid molecules.

The Tₘ is the temperature under defined ionic strength and pH, at which 50% of the target sequence hybridises to a perfectly matched probe. The Tₘ is dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tₘ decreases about 1° C. per % base mismatch. The Tₘ may be calculated using the following equations, depending on the types of hybrids:

1) DNA-DNA hybrids (Meinkoth and Wahl, Anal. Biochem., 138: 267-284, 1984):

Tₘ=81.5° C.+16.6×log₁₀[Na⁺]^{a}+0.41x%[G/C^{b}]−500x[L^{c}]⁻¹−0.61x% formamide

2) DNA-RNA or RNA-RNA hybrids:

Tₘ=79.8+18.5(log₁₀[Na⁺]^{a})+0.58(% G/C^{b})+11.8(% G/C^{b})²−820/L^{c}

3) oligo-DNA or oligo-RNA^{d} hybrids:

For <20 nucleotides: Tₘ=2(Iₙ)

For 20-35 nucleotides: Tₘ=22+1.46(Iₙ)

^{a} or for other monovalent cation, but only accurate in the 0.01-0.4 M range.
^{b} only accurate for % GC in the 30% to 75% range.
^{c} L=length of duplex in base pairs.
^{d} oligo, oligonucleotide; Iₙ=effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-homologous probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5× Denhardt's reagent, 0.5-1.0% SDS, 100 μg/ml denatured, fragmented salmon sperm DNA, 0.5% sodium pyrophosphate.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

### Splice Variant

The term “splice variant” as used herein encompasses variants of a nucleic acid sequence in which selected introns and/or exons have been excised, replaced, displaced or added, or in which introns have been shortened or lengthened. Such variants will be ones in which the biological activity of the protein is substantially retained; this may be achieved by selectively retaining functional segments of the protein. Such splice variants may be found in nature or may be manmade. Methods for predicting and isolating such splice variants are well known in the art (see for example Foissac and Schiex (2005) BMC Bioinformatics 6: 25).

### Allelic Variant

Alleles or allelic variants are alternative forms of a given gene, located at the same chromosomal position. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

### Gene Shuffling/Directed Evolution

Gene shuffling or directed evolution consists of iterations of DNA shuffling followed by appropriate screening and/or selection to generate variants of nucleic acids or portions thereof encoding proteins having a modified biological activity (Castle et al., (2004) Science 304(5674): 1151-4; U.S. Pat. Nos. 5,811,238 and 6,395,547).

### Regulatory Element/Control Sequence/Promoter

The terms “regulatory element”, “control sequence” and “promoter” are all used interchangeably herein and are to be taken in a broad context to refer to regulatory nucleic acid sequences capable of effecting expression of the sequences to which they are ligated. The term “promoter” typically refers to a nucleic acid control sequence located upstream from the transcriptional start of a gene and which is involved in recognising and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ.

A “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described above, be linked operably to or comprise a suitable promoter which expresses the gene at the right point in time and with the required spatial expression pattern.

For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes include for example beta-glucuronidase or beta-galactosidase. The promoter activity is assayed by measuring the enzymatic activity of the beta-glucuronidase or beta-galactosidase. The promoter strength and/or expression pattern may then be compared to that of a reference promoter (such as the one used in the methods of the present invention). Alternatively, promoter strength may be assayed by quantifying mRNA levels or by comparing mRNA levels of the nucleic acid used in the methods of the present invention, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6: 986-994). Generally by “weak promoter” is intended a promoter that drives expression of a coding sequence at a low level. By “low level” is intended at levels of about 1/10,000 transcripts to about 1/100,000 transcripts, to about 1/500,0000 transcripts per cell. Conversely, a “strong promoter” drives expression of a coding sequence at high level, or at about 1/10 transcripts to about 1/100 transcripts to about 1/1000 transcripts per cell.

### Operably Linked

The term “operably linked” as used herein refers to a functional linkage between the promoter sequence and the gene of interest, such that the promoter sequence is able to initiate transcription of the gene of interest.

### Constitutive Promoter

A “constitutive promoter” refers to a promoter that is transcriptionally active during most, but not necessarily all, phases of growth and development and under most environmental conditions, in at least one cell, tissue or organ. Table 2a below gives examples of constitutive promoters.

TABLE 2a
Examples of constitutive promoters

| Gene Source | Reference |
|---|---|
| Actin | McElroy et al, Plant Cell, 2: 163-171, 1990 |
| HMGP | WO 2004/070039 |
| CAMV 35S | Odell et al, Nature, 313: 810-812, 1985 |
| CaMV 19S | Nilsson et al., Physiol. Plant. 100: 456-462, 1997 |
| GOS2 | de Pater et al, Plant J Nov; 2(6): 837-44, 1992, |
| | WO 2004/065596 |
| Ubiquitin | Christensen et al, Plant Mol. Biol. 18: 675-689, 1992 |
| Rice cyclophilin | Buchholz et al, Plant Mol Biol. 25(5): 837-43, 1994 |
| Maize H3 histone | Lepetit et al, Mol. Gen. Genet. 231: 276-285, 1992 |
| Alfalfa H3 | Wu et al. Plant Mol. Biol. 11: 641-649, 1988 |
| histone | |
| Actin 2 | An et al, Plant J. 10(1); 107-121, 1996 |
| 34S FMV | Sanger et al., Plant. Mol. Biol., 14, 1990: 433-443 |
| Rubisco small | U.S. Pat. No. 4,962,028 |
| subunit | |
| OCS | Leisner (1988) Proc Natl Acad Sci USA 85(5): 2553 |
| SAD1 | Jain et al., Crop Science, 39 (6), 1999: 1696 |
| SAD2 | Jain et al., Crop Science, 39 (6), 1999: 1696 |
| nos | Shaw et al. (1984) Nucleic Acids Res. 12(20): |
| | 7831-7846 |
| V-ATPase | WO 01/14572 |
| Super promoter | WO 95/14098 |
| G-box proteins | WO 94/12015 |

### Ubiquitous Promoter

A ubiquitous promoter is active in substantially all tissues or cells of an organism.

### Developmentally-Regulated Promoter

A developmentally-regulated promoter is active during certain developmental stages or in parts of the plant that undergo developmental changes.

### Inducible Promoter

An inducible promoter has induced or increased transcription initiation in response to a chemical (for a review see Gatz 1997, Annu. Rev. Plant Physiol. Plant Mol. Biol., 48:89-108), environmental or physical stimulus, or may be “stress-inducible”, i.e. activated when a plant is exposed to various stress conditions, or a “pathogen-inducible” i.e. activated when a plant is exposed to exposure to various pathogens.

### Organ-specific/Tissue-Specific Promoter

An organ-specific or tissue-specific promoter is one that is capable of preferentially initiating transcription in certain organs or tissues, such as the leaves, roots, seed tissue etc. For example, a “root-specific promoter” is a promoter that is transcriptionally active predominantly in plant roots, substantially to the exclusion of any other parts of a plant, whilst still allowing for any leaky expression in these other plant parts. Promoters able to initiate transcription in certain cells only are referred to herein as “cell-specific”.

Examples of root-specific promoters are listed in Table 2b below:

TABLE 2b
Examples of root-specific promoters

| Gene Source | Reference |
|---|---|
| RCc3 | Plant Mol Biol. 1995 Jan; 27(2): 237-48 |
| Arabidopsis phosphate | Kovama et al., 2005 |
| transporter PHT1 | |
| Medicago phosphate | Xiao et al., 2006 |
| transporter | |
| Arabidopsis Pyk10 | Nitz et al. (2001) Plant Sci 161(2): 337-346 |

A seed-specific promoter is transcriptionally active predominantly in seed tissue, but not necessarily exclusively in seed tissue (in cases of leaky expression). The seed-specific promoter may be active during seed development and/or during germination.

A green tissue-specific promoter as defined herein is a promoter that is transcriptionally active predominantly in green tissue, substantially to the exclusion of any other parts of a plant, whilst still allowing for any leaky expression in these other plant parts.

Examples of green tissue-specific promoters which may be used to perform the methods of the invention are shown in Table 2c below.

TABLE 2c
Examples of green tissue-specific promoters

| Gene | Expression | Reference |
|---|---|---|
| Maize Orthophosphate dikinase | Leaf specific | Fukavama et al., 2001 |
| Maize Phosphoenolpyruvate | Leaf specific | Kausch et al., 2001 |
| carboxylase | | |
| Rice Phosphoenolpyruvate | Leaf specific | Liu et al., 2003 |
| carboxylase | | |
| Rice small subunit Rubisco | Leaf specific | Nomura et al., 2000 |
| rice beta expansin EXBP9 | Shoot specific | WO 2004/070039 |
| Pigeonpea small subunit Rubisco | Leaf specific | Panguluri et al., 2005 |
| Pea RBCS3A | Leaf specific | |

Another example of a tissue-specific promoter is a meristem-specific promoter, which is transcriptionally active predominantly in meristematic tissue, substantially to the exclusion of any other parts of a plant, whilst still allowing for any leaky expression in these other plant parts.

### Terminator

The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

### Modulation

The term “modulation” means in relation to expression or gene expression, a process in which the expression level is changed by said gene expression in comparison to the control plant, preferably the expression level is increased. The original, unmodulated expression may be of any kind of expression of a structural RNA (rRNA, tRNA) or mRNA with subsequent translation. The term “modulating the activity” shall mean any change of the expression of the inventive nucleic acid sequences or encoded proteins, which leads to increased yield and/or increased growth of the plants.

### Expression

The term “expression” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product.

### Increased Expression/Overexpression

The term “increased expression” or “overexpression” as used herein means any form of expression that is additional to the original wild-type expression level.

Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell biol. 8: 4395-4405; Callis et al. (1987) Genes Dev 1:1183-1200). Such intron enhancement of gene expression is typically greatest when placed near the 5′ end of the transcription unit. Use of the maize introns Adh1-S intron 1, 2, and 6, the Bronze-1 intron are known in the art. For general information see: The Maize Handbook, Chapter 116, Freeling and Walbot, Eds., Springer, N.Y. (1994).

Optionally, one or more terminator sequences may be used in the construct introduced into a plant. Additional regulatory elements may include transcriptional as well as translational enhancers. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

### Endogenous Gene

Reference herein to an “endogenous” gene not only refers to the gene in question as found in a plant in its natural form (i.e., without there being any human intervention), but also refers to that same gene (or a substantially homologous nucleic acid/gene) in an isolated form subsequently (re)introduced into a plant (a transgene). For example, a transgenic plant containing such a transgene may encounter a substantial reduction of the transgene expression and/or substantial reduction of expression of the endogenous gene. The isolated gene may be isolated from an organism or may be manmade, for example by chemical synthesis.

### Decreased Expression

Reference herein to “decreased expression” or “reduction or substantial elimination” of expression is taken to mean a decrease in endogenous gene expression and/or polypeptide levels and/or polypeptide activity relative to control plants. The reduction or substantial elimination is in increasing order of preference at least 10%, 20%, 30%, 40% or 50%, 60%, 70%, 80%, 85%, 90%, or 95%, 96%, 97%, 98%, 99% or more reduced compared to that of control plants. Reduction or substantial elimination of expression may be achieved using routine tools and techniques known in the art.

### Selectable Marker (Gene)/Reporter Gene

“Selectable marker”, “selectable marker gene” or “reporter gene” includes any gene that confers a phenotype on a cell in which it is expressed to facilitate the identification and/or selection of cells that are transfected or transformed with a nucleic acid construct of the invention. These marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to Basta®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilisation of xylose, or antinutritive markers such as the resistance to 2-deoxyglucose). Expression of visual marker genes results in the formation of colour (for example β-glucuronidase, GUS or β-galactosidase with its coloured substrates, for example X-Gal), luminescence (such as the luciferin/luciferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die).

Since the marker genes, particularly genes for resistance to antibiotics and herbicides, are no longer required or are undesired in the transgenic host cell once the nucleic acids have been introduced successfully, the process according to the invention for introducing the nucleic acids advantageously employs techniques which enable the removal or excision of these marker genes. One such a method is what is known as co-transformation. The co-transformation method employs two vectors simultaneously for the transformation, one vector bearing the nucleic acid according to the invention and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e. the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases the marker gene must be eliminated by performing crosses. In microbiology, techniques were developed which make possible, or facilitate, the detection of such events. A further advantageous method relies on what is known as recombination systems; whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the plant genome of the nucleic acid sequences according to the invention is possible. Naturally, these methods can also be applied to microorganisms such as yeast, fungi or bacteria.

### Transgenic/Transgene/Recombinant

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either

(a) the nucleic acid sequences encoding proteins useful in the methods of the invention, or
(b) genetic control sequence(s) which is operably linked with the nucleic acid sequence according to the invention, for example a promoter, or
(c) a) and b)
are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic plants are mentioned herein.

### Transformation

The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen Genet 202: 179-185); DNA or RNA-coated particle bombardment (Klein T M et al., (1987) Nature 327: 70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Höfgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, K A and Marks M D (1987). Mol Gen Genet 208:274-289; Feldmann K (1992). In: C Koncz, N-H Chua and J Shell, eds, Methods in Arabidopsis Research. Word Scientific, Singapore, pp. 274-289]. Alternative methods are based on the repeated removal of the inflorescences and incubation of the excision site in the center of the rosette with transformed agrobacteria, whereby transformed seeds can likewise be obtained at a later point in time (Chang (1994). Plant J. 5: 551-558; Katavic (1994). Mol Gen Genet, 245: 363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). C R Acad Sci Paris Life Sci, 316: 1194-1199], while in the case of the “floral dip” method the developing floral tissue is incubated briefly with a surfactant-treated agrobacterial suspension [Clough, S J and Bent A F (1998) The Plant J. 16, 735-743]. A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol Biol. 2001 Sep. 21; 312 (3):425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22(2), 225-229).

### T-DNA Activation Tagginq

T-DNA activation tagging (Hayashi et al. Science (1992) 1350-1353), involves insertion of T-DNA, usually containing a promoter (may also be a translation enhancer or an intron), in the genomic region of the gene of interest or 10 kb up- or downstream of the coding region of a gene in a configuration such that the promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to modified expression of genes near the inserted T-DNA. The resulting transgenic plants show dominant phenotypes due to modified expression of genes close to the introduced promoter.

### TILLING

The term “TILLING” is an abbreviation of “Targeted Induced Local Lesions In Genomes” and refers to a mutagenesis technology useful to generate and/or identify nucleic acids encoding proteins with modified expression and/or activity. TILLING also allows selection of plants carrying such mutant variants. These mutant variants may exhibit modified expression, either in strength or in location or in timing (if the mutations affect the promoter for example). These mutant variants may exhibit higher activity than that exhibited by the gene in its natural form. TILLING combines high-density mutagenesis with high-throughput screening methods. The steps typically followed in TILLING are: (a) EMS mutagenesis (Redei G P and Koncz C (1992) In Methods in Arabidopsis Research, Koncz C, Chua N H, Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et al., (2000) Nat Biotechnol 18: 455-457; reviewed by Stemple (2004) Nat Rev Genet 5(2): 145-50).

### Homologous Recombination

Homologous recombination allows introduction in a genome of a selected nucleic acid at a defined selected position. Homologous recombination is a standard technology used routinely in biological sciences for lower organisms such as yeast or the moss Physcomitrella. Methods for performing homologous recombination in plants have been described not only for model plants (Offring a et al. (1990) EMBO J 9(10): 3077-84) but also for crop plants, for example rice (Terada et al. (2002) Nat Biotech 20(10): 1030-4; Iida and Terada (2004) Curr Opin Biotech 15(2): 132-8).

### Yield

The term “yield” in general means a measurable produce of economic value, typically related to a specified crop, to an area, and to a period of time. Individual plant parts directly contribute to yield based on their number, size and/or weight, or the actual yield is the yield per acre for a crop and year, which is determined by dividing total production (includes both harvested and appraised production) by planted acres. The term “yield” of a plant may relate to vegetative biomass (root and/or shoot biomass), to reproductive organs, and/or to propagules (such as seeds) of that plant.

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

### Early Vigour

“Early vigour” refers to active healthy well-balanced growth especially during early stages of plant growth, and may result from increased plant fitness due to, for example, the plants being better adapted to their environment (i.e. optimizing the use of energy resources and partitioning between shoot and root). Plants having early vigour also show increased seedling survival and a better establishment of the crop, which often results in highly uniform fields (with the crop growing in uniform manner, i.e. with the majority of plants reaching the various stages of development at substantially the same time), and often better and higher yield. Therefore, early vigour may be determined by measuring various factors, such as thousand kernel weight, percentage germination, percentage emergence, seedling growth, seedling height, root length, root and shoot biomass and many more.

### Increase/Improve/Enhance

The terms “increase”, “improve” or “enhance” are interchangeable and shall mean in the sense of the application at least a 3%, 4%, 5%, 6%, 7%, 8%, 9% or 10%, preferably at least 15% or 20%, more preferably 25%, 30%, 35% or 40% more yield and/or growth in comparison to control plants as defined herein.

### Greenness Index

The “greenness index” as used herein is calculated from digital images of plants. For each pixel belonging to the plant object on the image, the ratio of the green value versus the red value (in the RGB model for encoding color) is calculated. The greenness index is expressed as the percentage of pixels for which the green-to-red ratio exceeds a given threshold. Under normal growth conditions, under salt stress growth conditions, and under reduced nutrient availability growth conditions, the greenness index of plants is measured in the last imaging before flowering. In contrast, under drought stress growth conditions, the greenness index of plants is measured in the first imaging after drought.

### Seed Yield

Increased seed yield may manifest itself as one or more of the following: a) an increase in seed biomass (total seed weight) which may be on an individual seed basis and/or per plant and/or per hectare or acre; b) increased number of flowers per plant; c) increased number of (filled) seeds; d) increased seed filling rate (which is expressed as the ratio between the number of filled seeds divided by the total number of seeds; e) increased harvest index, which is expressed as a ratio of the yield of harvestable parts, such as seeds, divided by the total biomass; and f) increased thousand kernel weight (TKW), which is extrapolated from the number of filled seeds counted and their total weight. An increased TKW may result from an increased seed size and/or seed weight, and may also result from an increase in embryo and/or endosperm size.

An increase in seed yield may also be manifested as an increase in seed size and/or seed volume. Furthermore, an increase in seed yield may also manifest itself as an increase in seed area and/or seed length and/or seed width and/or seed perimeter. Increased yield may also result in modified architecture, or may occur because of modified architecture.

### Increased Growth Rate

Since the transgenic plants according to the present invention have increased yield, it is likely that these plants exhibit an increased growth rate (during at least part of their life cycle), relative to the growth rate of corresponding wild type plants at a corresponding stage in their life cycle. Besides the increased yield capacity, an increased efficiency of nutrient uptake may also contribute to the increase in yield. It is observed that the plants according to the present invention show a higher efficiency in nutrient uptake. Increased efficiency of nutrient uptake allows better growth of the plant, whether the plant is under stress or non-stress conditions.

The increased growth rate may be specific to one or more parts of a plant (including seeds), or may be throughout substantially the whole plant. A plant having an increased growth rate may even exhibit early flowering. The increase in growth rate may take place at one or more stages in the life cycle of a plant or during substantially the whole plant life cycle. Increased growth rate during the early stages in the life cycle of a plant may reflect enhanced vigour (increased seedling vigor at emergence). The increase in growth rate may alter the harvest cycle of a plant allowing plants to be sown later and/or harvested sooner than would otherwise be possible. If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of rice plants followed by, for example, the sowing and optional harvesting of soy bean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi and insects.

In particular, the methods of the present invention may be performed under non-stress conditions to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress may cause denaturing of functional and structural proteins. As a consequence, these diverse environmental stresses often activate similar cell signaling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location.

### Plant

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Abelmoschus spp., Agave sisalana, Agropyron spp., Agrostis stolonifera, Allium spp., Amaranthus spp., Ammophila arenaria, Ananas comosus, Annona spp., Apium graveolens, Artocarpus spp., Asparagus officinalis, Avena spp. (e.g. Avena sativa, Avena fatua, Avena byzantina, Avena fatua var. sativa, Avena hybrida), Averrhoa carambola Baikiaea pluriuga, Betula spp., Bruguiera gymnorrhiza, Burkea africana, Butea frondosa, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Calliandra spp, Camellia sinensis, Canna indica, Capsicum spp., Cassia spp., Centroema pubescens, Chaenomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Cadaba farinosa, Camellia sinensis, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cynara spp., Dalbergia monetaria, Davallia divaricata, Desmodium spp., Dicksonia squarosa, Diheteropogon amplectens, Dioclea spp, Dolichos spp., Dorycnium rectum, Daucus carota, Dimocarpus longan, Dioscorea spp., Diospyros spp., Echinochloa pyramidalis, Ehrartia spp., Eleusine coracana, Eragrestis spp., Erythrina spp., Eucalyptus spp., Euclea schimperi, Eulalia villosa, Elaeis (e.g. Elaeis guineensis, Elaeis oleifera), Erianthus sp., Eriobotrya japonica, Eugenia uniflora, Fagopyrum spp., Feijoa sellowiana, Fragaria spp., Flemingia spp, Freycinetia banksii, Fagus spp., Festuca arundinacea, Ficus carica, Fortunella spp., Fragaria spp., Geranium thunbergii, Ginkgo biloba, Glycine spp. (e.g. Glycine max, Soja hispida or Soja max), Glycine javanica, Gliricidia spp, Gossypium hirsutum, Grevillea spp., Guibourtia coleosperma, Gossypium hirsutum, Helianthus spp. (e.g. Helianthus annuus), Hemerocallis fulva, Hedysarum spp., Hemarthia altissima, Heteropogon contortus, Hibiscus spp., Hordeum spp. (e.g. Hordeum vulgare), Hyparrhenia rufa, Hypericum erectum, Hyperthelia dissoluta, Indigo incarnata, Iris spp., Ipomoea batatas, Juglans spp., Leptarrhena pyrolifolia, Lespediza spp., Lettuca spp., Leucaena leucocephala, Loudetia simplex, Lotonus bainesi, Lotus spp., Lactuca sativa, Lathyrus spp., Lens culinaris, Linum usitatissimum, Litchi chinensis, Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Macrotyloma axillare, Malus spp., Malus spp., Malpighia emarginata, Mammea americana, Mangifera indica, Manihot spp., Manihot esculenta, Medicago sativa, Metasequoia glyptostroboides, Manilkara zapota, Medicago sativa, Melilotus spp., Mentha spp., Miscanthus sinensis, Momordica spp., Morus nigra, Musa spp., Musa sapientum, Nicotianum spp., Onobrychis spp., Ornithopus spp., Peltophorum africanum, Pennisetum spp., Olea spp., Opuntia spp., Oryza spp. (e.g. Oryza sativa, Oryza latifolia), Panicum miliaceum, Panicum virgatum, Passiflora edulis, Pastinaca sativa, Pennisetum sp., Persea spp., Persea gratissima, Petunia spp., Phaseolus spp., Phoenix canariensis, Phormium cookianum, Photinia spp., Picea glauca, Pinus spp., Pisum sativum, Podocarpus totara, Pogonarthria fleckii, Pogonarthria squarrosa, Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prosopis cineraria, Prunus spp., Psidium spp., Punica granatum, Pyrus communis, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Raphanus sativus, Rheum rhabarbarum, Ricinus communis, Rubus spp., Salix spp., Schyzachyrium sanguineum, Sciadopitys verticillata, Sequoia sempervirens, Sequoiadendron giganteum, Saccharum spp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Sporobolus fimbriatus, Stiburus alopecuroides, Stylosanthos humilis, Syzygium spp., Tadehagi spp, Taxodium distichum, Themeda triandra, Trifolium spp., Triticum spp. (e.g. Triticum aestivum, Triticum durum, Triticum turgidum, Triticum hybemum, Triticum macha, Triticum sativum or Triticum vulgare), Tagetes spp., Tamarindus indica, Theobroma cacao, Triticosecale rimpaui, Tsuga heterophylla, Tropaeolum minus, Tropaeolum majus, Vaccinium spp., Vicia spp., Vitis vinifera, Vigna spp., Viola odorata, Vitis spp., Watsonia pyramidata, Zantedeschia aethiopica, Zea mays, Zizania palustris, Ziziphus spp., amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugarcane, sunflower, tomato, squash, tea and algae, amongst others.

### Alignment of Sequences

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (over the whole the sequence) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimise alignment between conserved motifs, as would be apparent to a person skilled in the art. Furthermore, instead of using full-length sequences for the identification of homologues, specific domains may also be used. The sequence identity values, which are indicated below in Example 3 as a percentage were determined over the entire nucleic acid or amino acid sequence, and/or over selected domains or conserved motif(s), using the programs mentioned above using the default parameters.

### Reciprocal Blast Search

Reciprocal blast search typically involves a first BLAST involving BLASTing a query sequence (for example using any of the sequences listed in Table 1, Table 2, Table 14 of Example 9 or Table 18 of Example 18) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is any of SEQ ID NO: 1 or SEQ ID NO: 2, or SEQ ID NO: 50 to SEQ ID NO: 59, or SEQ ID NO: 131 or SEQ ID NO: 132 or SEQ ID NO: 257 or SEQ ID NO: 258, the second BLAST would therefore be against Oryza sativa sequences). The results of the first and second BLASTs are then compared. A paralogue is identified if a high-ranking hit from the first blast is from the same species as from which the query sequence is derived, a BLAST back then ideally results in the query sequence as highest hit; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

### Amplification

A variety of nucleic acid amplification-based methods for genetic and physical mapping may be carried out using the nucleic acids. Examples include allele-specific amplification (Kazazian (1989) J. Lab. Clin. Med 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

### DETAILED DESCRIPTION OF THE INVENTION

The present invention provides a method for enhancing yield-related traits in plants relative to control plants, comprising modulating expression in a plant of a nucleic acid encoding a NAC transcription factor. A preferred method for modulating (preferably, increasing) expression of a nucleic acid encoding a NAC transcription factor is by introducing and expressing in a plant a nucleic acid encoding a particular class of NAC transcription factor as further defined below.

In addition, the present invention provides a method for enhancing yield-related traits in plants relative to control plants, comprising modulating expression in a plant of a nucleic acid encoding an AP2-2 polypeptide. The present invention further provides a method for enhancing yield-related traits in plants relative to control plants, comprising modulating expression in a plant of a nucleic acid encoding an AP2-70-like polypeptide.

With regard to a NAC transcription factor, the nucleic acid to be introduced into a plant (and therefore useful in performing the methods of the invention) is any nucleic acid encoding the type of NAC transcription factor which will now be described. A “NAC transcription factor” as defined herein refers to any amino acid sequence which when used in the construction of a NAC phylogenetic tree, such as the one depicted in FIG. 1, tends to cluster with the group of NACs comprising an amino acid sequence represented by any one of SEQ ID NO: 2, SEQ ID NO: 51, SEQ ID NO: 53, SEQ ID NO: 55, SEQ ID NO: 57 or SEQ ID NO: 59, rather than with any other NAC group.

A person skilled in the art could readily determine whether any amino acid sequence in question falls within the definition of a “NAC transcription factor” using known techniques and software for the making of such a phylogenetic tree, such as a GCG, EBI or CLUSTAL package, using default parameters. The phylogenetic tree of FIG. 1 is taken from Ooka et al., 2003 (DNA Research 10, 239-247, 2003). The method for constructing such a phylogenetic tree is described in Ooka et al., 2003. In the phylogenetic tree of FIG. 1, SEQ ID NO: 2 is found in the group third up from the bottom, labelled “OsNAC7” and SEQ ID NO: 51, SEQ ID NO: 53, SEQ ID NO: 55, SEQ ID NO: 57 and SEQ ID NO: 59 are found in the area starting from (and including) the group labelled on the right hand side of the page as “ONAC022” and ending with (and including) the group labelled on the right hand side of the page as “OsNAC3”. Any sequence clustering within these areas, which comprises the sequences of SEQ ID NO: 2 on the one hand and SEQ ID NO: 51, SEQ ID NO: 53, SEQ ID NO: 55 SEQ ID NO: 57 and SEQ ID NO: 59 on the other hand, would be considered to fall within the aforementioned definition of a NAC transcription factor, and would be considered suitable for use in the methods of the invention. Additionally or alternatively, a “NAC transcription factor” as defined herein is one comprising any one or more of the Motifs described below.

Motif I: KIDLDIIQELD, or a motif having in increasing order of preference at least 50%, 60%, 70%, 80% or 90% sequence identity to the sequence of Motif 1.

Motif I is preferably K/P/RIG I/S/M D/A/E/Q U/I/V D I/V/F I Q/V/R/K E/D U/I/V D.

Motif II: CKYGXGHGGDEQTEW, or a motif having in increasing order of preference at least 50%, 60%, 70%, 80% or 90% sequence identity to the sequence of Motif 11, where ‘X’ is taken to be any amino acid or a gap.

Motif II is preferably C K/R Y/L/I G XXX G/Y/N D/E E Q/R T/N/S EW, where ‘X’ is any amino acid or a gap.

Motif III: GWVVCRAFQKP, or a motif having in increasing order of preference at least 50%, 60%, 70%, 80% or 90% sequence identity to the sequence of Motif III.

Motif III is preferably GWVVCR A/V F X¹ K X², where ‘X¹’ and ‘X²’ may be any amino acid, preferably X¹ is Q/R/K, preferably X² is P/R/K.

Motifs I to III are found in the NACs represented by SEQ ID NO: 2, and are also typically found in NACs clustering (in a phylogenetic tree of NACs) with the group of NACs comprising SEQ ID NO: 2-rather than with any other NAC group.

Motif IV: PVPIIA, or a motif having in increasing order of preference at least 50%, 60%, 70%, 80% or 90% sequence identity to the sequence of Motif IV.

Motif IV is preferably A/P/S/N V/L/I/A P/S/D/V/Q V/I I A/T/G.

Motif V: NGSRPN, or a motif having in increasing order of preference at least 50%, 60%, 70%, 80% or 90% sequence identity to the sequence of Motif V.

Motif V is preferably N G/S S/Q/A/V RP N/S.

Motif VI: CRLYNKK, or a motif having in increasing order of preference at least 50%, 60%, 70%, 80% or 90% sequence identity to the sequence of Motif VI.

Motif VI is preferably C/Y R/K L/I Y/H/F N/K K K/N/C/S/T

Motifs III to VI are typically found in NACs of SEQ ID NO: 51, SEQ ID NO: 53, SEQ ID NO: 55 SEQ ID NO: 57 and SEQ ID NO: 59 and in NACs clustering (in a phylogenetic tree) with the NACs represented by the aforementioned SEQ ID NOs rather than with any other NAC group.

Motif VII: NEWEKMQ, or a motif having in increasing order of preference at least 50%, 60%, 70%, 80% or 90% sequence identity to the sequence of Motif VII.

Motif VII is preferably N E/Q/T WEK M/V Q/R/K

Motif VIII: WGETRTPESE, or a motif having in increasing order of preference at least 50%, 60%, 70%, 80% or 90% sequence identity to the sequence Motif VIII.

Motif VIII is preferably WGE T/A RTPES E/D

Motif IX: VPKKESMDDA, or a motif having in increasing order of preference at least 50%, 60%, 70%, 80% or 90% sequence identity to the sequence of Motif IX.

Motif IX is preferably V/L PK K/E E S/R/A/V M/V/A/Q/R D/E D/E/L A/G/D

Motif X: SYDDIQGMYS, or a motif having in increasing order of preference at least 50%, 60%, 70%, 80% or 90% sequence identity to the sequence of Motif X.

Motif X is preferably S L/Y DD L/I Q G/S L/M/P G/Y S/N.

Motifs VII, VIII, IX and X are typically found in NACs of SEQ ID NO: 51 and in NACs clustering (in a phylogenetic tree) with the NAC of SEQ ID NO: 51 rather than with any other NAC group.

Motif XI: DSMPRLHADSSCSE, or a motif having in increasing order of preference at least 50%, 60%, 70%, 80% or 90% sequence identity to the sequence of Motif XI.

Motif XI is preferably DS M/V/I P R/K L/I/A H T/A/S D/E SS C/G SE.

Motif XI is typically found in NACs of SEQ ID NO: 53 and SEQ ID NO: 55 and in NACs clustering (in a phylogenetic tree) with the NACs represented by the aforementioned SEQ ID NOs rather than with any other NAC group.

Each of motifs I to XI may comprise one or more conservative amino acid substitution at any position. Examples of NAC transcription factors as defined herein, i.e. any amino acid sequence which when used in the construction of a NAC phylogenetic tree, such as the one depicted in FIG. 1, tends to cluster with the group of NACs represented by any one of SEQ ID NO: 2, SEQ ID NO: 51, SEQ ID NO: 53, SEQ ID NO: 55 SEQ ID NO: 57 and SEQ ID NO: 59 rather than with any other NAC group, are as given in Tables 3 and 4 below.

TABLE 3
Examples of NAC transcription factors that cluster (in a phylogenetic tree) with the NAC
represented by SEQ ID NO: 2 rather than with any other NAC group

| | | NCBI Accession | | |
|---|---|---|---|---|
| Nucleic Acid | Amino Acid | Number | Other name (if any) | Source |
| SEQ ID NO: 1 | SEQ ID NO: 2 | XM_479673.1 | NAM2 (also called | Oryza sativa |
| | | | CDS4054 in the | |
| | | | alignment of FIG. 2) | |
| SEQ ID NO: 3 | SEQ ID NO: 4 | AK106313 | | Oryza sativa |
| SEQ ID NO: 5 | SEQ ID NO: 6 | NM_187584.1 | | Oryza sativa |
| SEQ ID NO: 7 | SEQ ID NO: 8 | XM_467007.1 | | Oryza sativa |
| SEQ ID NO: 9 | SEQ ID NO: 10 | XM_473322.1 | | Oryza sativa |
| SEQ ID NO: 11 | SEQ ID NO: 12 | ABE89364 | | Medicago |
| | | | | truncatula |
| SEQ ID NO: 13 | SEQ ID NO: 14 | NM_101098.2 | ANAC007/EMB2749 | Arabidopsis |
| | | | | thaliana |
| SEQ ID NO: 15 | SEQ ID NO: 16 | NM_104947.1 | ANAC026 | Arabidopsis |
| | | | | thaliana |
| SEQ ID NO: 17 | SEQ ID NO: 18 | XM_476289.1 | | Oryza sativa |
| SEQ ID NO: 19 | SEQ ID NO: 20 | NM_127362.1 | ANAC037 | Arabidopsis |
| | | | | thaliana |
| SEQ ID NO: 21 | SEQ ID NO: 22 | NM_119783.2 | ANAC076 | Arabidopsis |
| | | | | thaliana |
| SEQ ID NO: 23 | SEQ ID NO: 24 | NM_187584.1 | | Oryza sativa |
| SEQ ID NO: 25 | SEQ ID NO: 26 | NM_125632.1 | ANAC101 | Arabidopsis |
| | | | | thaliana |
| SEQ ID NO: 27 | SEQ ID NO: 28 | NM_126028.1 | ANAC105 | Arabidopsis |
| | | | | thaliana |
| SEQ ID NO: 29 | SEQ ID NO: 30 | AK071064.1 | | Oryza sativa |
| SEQ ID NO: 31 | SEQ ID NO: 32 | NM_105851.1 | ANAC030 | Arabidopsis |
| | | | | thaliana |
| SEQ ID NO: 33 | SEQ ID NO: 34 | NM_197511.1 | | Oryza sativa |
| SEQ ID NO: 35 | SEQ ID NO: 36 | AB217775.1 | | Zinnia elegans |
| SEQ ID NO: 37 | SEQ ID NO: 38 | AJ833965.1 | | Zea mays |

TABLE 4
Examples of NAC transcription factors that cluster (in a phylogenetic tree) with the NAC
represented by any one of SEQ ID NO: 51, SEQ ID NO: 53, SEQ ID NO: 55
SEQ ID NO: 57 and SEQ ID NO: 59 rather than with any other NAC group

| SEQ ID NO: 50 | SEQ ID NO: 51 | XM_470088.1 | NAC1 | Oryza sativa |
|---|---|---|---|---|
| | | | (also known as | |
| | | | CDS4035 in the | |
| | | | alignment of FIG. 2) | |
| SEQ ID NO: 52 | SEQ ID NO: 53 | AB028185.1 | NAC4 | Oryza sativa |
| | | BAA89800.1 | (known as OsNAC6 | |
| | | | in alignment of FIG. 2) | |
| SEQ ID NO: 54 | SEQ ID NO: 55 | AB028184.1 | NAC6 | Oryza sativa |
| | | BAA89799.1 | (known as OsNAC5 | |
| | | | in alignment of FIG. 2) | |
| SEQ ID NO: 56 | SEQ ID NO: 57 | AK107330.1 | NAC7 | Oryza sativa |
| | | | (known as ONAC24 | |
| | | | in alignment of FIG. 2) | |
| SEQ ID NO: 58 | SEQ ID NO: 59 | AK069257.1 | NAC3 | Oryza sativa |
| | | | (known as CDS4034 | |
| | | | in alignment of FIG. 2) | |
| SEQ ID NO: 60 | SEQ ID NO: 61 | AY625683.1 | NAC2-like | Triticum aestivum |
| | | AAU08786.1 | TaNAC2 | |
| SEQ ID NO: 62 | SEQ ID NO: 63 | AY103772.1 | PCO133091 | Zea mays |
| SEQ ID NO: 64 | SEQ ID NO: 65 | AJ010829.1 | GRAB1 | Triticum aestivum |
| | | CAA09371.1 | TACAA09371.1 | |
| SEQ ID NO: 66 | SEQ ID NO: 67 | NM_186659.2 | NAC3-like | Oryza sativa |
| | | NP_911548.1 | RiceNP_911548.1 | |
| SEQ ID NO: 68 | SEQ ID NO: 69 | DQ267442.1 | NAC1-like | Elaeis guineensis |
| | | ABB72844.1 | elaeis | |
| SEQ ID NO: 70 | SEQ ID NO: 71 | AB028183.1 | NAC4-like | Oryza sativa |
| | | BAA89798.1 | OsNAC4 | |
| SEQ ID NO: 72 | SEQ ID NO: 73 | DQ028770.1 | NAC2-like | Glycine max |
| | | AAY46122.1 | GmNAC2AAY46122.1 | |
| SEQ ID NO: 74 | SEQ ID NO: 75 | AY498713.1 | NAC-like | Lycopersicon |
| | | AAR88435.1 | tomatoNAC | esculentum |
| SEQ ID NO: 76 | SEQ ID NO: 77 | AY245883.1 | NAC5-8 | Brassica napus |
| | | AAP35052.1 | BrassicaNAC5- | |
| | | | 8”NAC-domain | |
| SEQ ID NO: 78 | SEQ ID NO: 79 | XM_475238.1 | NAC48 | Oryza sativa |
| | | XP_475238.1 | AK068392”putative | |
| SEQ ID NO: 80 | SEQ ID NO: 81 | AJ401151.1 | NAC | Solanum |
| | | CAC42087.1 | solanum”putative | tuberosum |
| SEQ ID NO: 82 | SEQ ID NO: 83 | NM_147856.2 | ATAF2 | Arabidopsis |
| | | NP_680161.1 | AT5G08790” = ”ATAF | thaliana |
| | | | 2”NP_680161.1” | |
| SEQ ID NO: 84 | SEQ ID NO: 85 | AF509873.1 | NH10 | Petunia × hybrida |
| | | AAM34773.1 | PetuniaNH10 = ”nam- | |
| | | | like | |
| SEQ ID NO: 86 | SEQ ID NO: 87 | AB218789 | NAC | Prunus mume |
| | | BAE48667.1 | PrunusPm74”NAC | |
| SEQ ID NO: 88 | SEQ ID NO: 89 | AY245885.1 | NAC18 | Brassica napus |
| | | AAP35054.1 | BnNAC18”NAC- | |
| | | | domain | |
| SEQ ID NO: 90 | SEQ ID NO: 91 | NM_125774.3 | ANAC102 | Arabidopsis |
| | | NP_201184.2 | | thaliana |
| SEQ ID NO: 92 | SEQ ID NO: 93 | AY714222.1 | NAC | Capsicum annuum |
| | | AAW48094.1 | CaNAC1 | |
| SEQ ID NO: 94 | SEQ ID NO: 95 | NM_100054.2 | ATAF1 | Arabidopsis |
| | | NP_171677 | | thaliana |
| SEQ ID NO: 96 | SEQ ID NO: 97 | AY573802.1 | NAC-NOR | Lycopersicon |
| | | AAU43922.1 | LeNAC- | esculentum |
| | | | NOR”transcription | |
| SEQ ID NO: 98 | SEQ ID NO: 99 | NM_118875.2 | RD26 | Arabidopsis |
| | | NP_567773.1 | Arabidopsis | thaliana |
| SEQ ID NO: | SEQ ID NO: | DQ022843.1 | NAC69-3 | Triticum aestivum |
| 100 | 101 | AAY44098.1 | NAC69-3”TaNAC69- | |
| | | | 3””NAC | |
| SEQ ID NO: | SEQ ID NO: | A625682.1 | NAC69-1 | Triticum aestivum |
| 102 | 103 | AAU08785.1 | NAC69- | |
| | | | 1”TaNAC69””NAC | |
| SEQ ID NO: | SEQ ID NO: | AY742218.1 | NAC23 | Saccharaum |
| 104 | 105 | AAW62955.1 | Saccharum | officinarum |
| SEQ ID NO: | SEQ ID NO: | AK063406.1 | NAC10 | Oryza sativa |
| 106 | 107 | | ONAC10AK063406 | |
| SEQ ID NO: | SEQ ID NO: | AC145753.1 | NAM | Medicago |
| 108 | 109 | | medicagoNAClike | truncatula |

The invention is illustrated by transforming plants with the Oryza sativa sequence represented by SEQ ID NO: 1, encoding the polypeptide sequence of SEQ ID NO: 2, and with the Oryza sativa sequence represented by SEQ ID NO: 50, encoding the polypeptide sequence of SEQ ID NO: 51, and with the Oryza sativa sequence represented by SEQ ID NO: 52, encoding the polypeptide sequence of SEQ ID NO: 53, and with the Oryza sativa sequence represented by SEQ ID NO: 54, encoding the polypeptide of SEQ ID NO: 55, and with the Oryza sativa sequence represented by SEQ ID NO: 56, encoding the polypeptide of SEQ ID NO: 57, and with the Oryza sativa sequence represented by SEQ ID NO: 58, encoding the polypeptide of SEQ ID NO: 59, however performance of the invention is not restricted to these sequences. The methods of the invention may be performed using any nucleic acid encoding a NAC transcription factor as defined herein, such as any of the nucleic acid sequences given in Tables 3 and 4.

The NAC amino acid sequences given in Table 3 may be considered to be orthologues and paralogues of the NAC represented by SEQ ID NO: 2. The NAC amino acid sequences given in Table 4 may be considered to be orthologues and paralogues of the NAC represented by any one of SEQ ID NO: 51, SEQ ID NO: 53, SEQ ID NO: 55 SEQ ID NO: 57 and SEQ ID NO: 59.

Orthologues and paralogues may easily be found by performing a so-called reciprocal blast search as described in the “definitions” section herein. Where the query sequence is any of SEQ ID NO: 1 or SEQ ID NO: 2, or SEQ ID NO: 50 to SEQ ID NO: 59, the second BLAST would therefore be against Oryza sequences.

Table 3 gives examples of orthologues and paralogues of the NAC represented by SEQ ID NO 2. Table 4 gives examples of orthologues and paralogues of the NAC represented by SEQ ID NO: 51, SEQ ID NO: 53, SEQ ID NO: 55 SEQ ID NO: 57 and SEQ ID NO: 59. Further orthologues and paralogues may readily be identified using the BLAST procedure described above and by following the procedure given in the Examples section.

The NAC proteins are identifiable by the presence of a highly conserved N-terminal NAC domain (shown in FIG. 5), accompanied by diverse C-terminal domains.

Specialist databases also exist for the identification of domains. The NAC domain in a NAC transcription factor may be identified as explained in the “definitions” section.

NAC domains may also be identified using routine techniques, such as by sequence alignment as explained in the “definitions” section herein.

The invention also provides hitherto unknown NAC-encoding nucleic acids and NAC polypeptides.

According to a further embodiment of the present invention, there is therefore provided an isolated nucleic acid molecule comprising:

(i) a nucleic acid represented by one of SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, or SEQ ID NO: 363;
(ii) the complement of a nucleic acid represented by one of SEQ ID NO: 347, SEQ ID NO: 349,

SEQ ID NO: 351, SEQ ID NO: 353, SEQ ID NO: 355, SEQ ID NO: 357, SEQ ID NO: 359, SEQ ID NO: 361, or SEQ ID NO: 363;

(iii) a nucleic acid encoding a NAC polypeptide having, in increasing order of preference, at least 50%, 55%, 60%, 65%, 70%, 75%, 80%, 85%, 90%, 95%, 96%, 97%, 98%, 99% or more sequence identity to the amino acid sequence represented by one of SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, or SEQ ID NO: 364.

According to a further embodiment of the present invention, there is also provided an isolated polypeptide comprising:

(i) an amino acid sequence represented by one of SEQ ID NO: SEQ ID NO: 348, SEQ ID NO: 350,

SEQ ID NO: 352, SEQ ID NO: 354, SEQ ID NO: 356, SEQ ID NO: 358, SEQ ID NO: 360, SEQ ID NO: 362, or SEQ ID NO: 364;

(ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequence represented by one of SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, or SEQ ID NO: 364;
(iii) derivatives of any of the amino acid sequences given in (i) or (ii) above.

Nucleic acids encoding NAC transcription factors defined herein need not be full-length nucleic acids, since performance of the methods of the invention does not rely on the use of full length nucleic acid sequences. Examples of nucleic acids suitable for use in performing the methods of the invention include the nucleic acid sequences given in Tables 3 and 4, but are not limited to those sequences. Nucleic acid variants may also be useful in practicing the methods of the invention. Examples of such nucleic acid variants include portions of nucleic acids encoding a NAC transcription factor as defined herein, splice variants of nucleic acids encoding a NAC transcription factor as defined herein, allelic variants of nucleic acids encoding a NAC transcription factor as defined herein and variants of nucleic acids encoding a NAC transcription factor as defined herein that are obtained by gene shuffling. The terms portion, splice variant, allelic variant and gene shuffling will now be described.

According to the present invention, there is provided a method for enhancing yield-related traits in plants, comprising introducing and expressing in a plant a portion of any one of the nucleic acid sequences given in Tables 3 and 4, or a portion of a nucleic acid encoding an orthologue, paralogue or homologue of any of the amino acid sequences given in Tables 3 and 4.

Portions useful in the methods of the invention, encode a polypeptide falling within the definition of a NAC transcription factor as defined herein and having substantially the same biological activity as the NAC transcription factor represented by any of the amino acid sequences given in Tables 3 and 4. The portion is typically at least 600 consecutive nucleotides in length, preferably at least 700 consecutive nucleotides in length, more preferably at least 800 consecutive nucleotides in length and most preferably at least 900 consecutive nucleotides in length, the consecutive nucleotides being of any one of the nucleic acid sequences given in Tables 3 and 4. Preferably, the portion is a portion of any one of the nucleic acids given in Tables 3 and 4. Most preferably the portion is a portion of the nucleic acid of SEQ ID NO: 1, SEQ ID NO: 50, SEQ ID NO: 52, SEQ ID NO: 54, SEQ ID NO: 56 or SEQ ID NO: 58. Preferably, the portion encodes an amino acid sequence comprising any one or more of Motifs I to XI as defined herein.

A portion of a nucleic acid encoding a NAC transcription factor as defined herein may be prepared, for example, by making one or more deletions to the nucleic acid in question. The portions may be used in isolated form or they may be fused to other coding (or non coding) sequences in order to, for example, produce a protein that combines several activities. When fused to other coding sequences, the resultant polypeptide produced upon translation may be bigger than that predicted for the NAC transcription factor portion.

Another nucleic acid variant useful in the methods of the invention is a nucleic acid capable of hybridising, under reduced stringency conditions, preferably under stringent conditions, with a nucleic acid encoding a NAC transcription factor as defined herein, or with a portion as defined herein.

Hybridising sequences useful in the methods of the invention, encode a polypeptide having a NAC domain (as described above) and having substantially the same biological activity as the NAC transcription factor represented by any of the amino acid sequences given in Tables 3 and 4. The hybridising sequence is typically at least 600 consecutive nucleotides in length, preferably at least 700 consecutive nucleotides in length, more preferably at least 800 consecutive nucleotides in length and most preferably at least 900 consecutive nucleotides in length. Preferably, the hybridising sequence is one that is capable of hybridising to any of the nucleic acids given in Table 3 and 4, or to a portion of any of these sequences, a portion being as defined above. Most preferably, the hybridising sequence is capable of hybridising to a nucleic acid as represented by SEQ ID NO: 1, SEQ ID NO: 50, SEQ ID NO: 52, SEQ ID NO: 54, SEQ ID NO: 56 or SEQ ID NO: 58 capable of hybridising to a portion of any of the aforementioned sequences. Preferably, the sequence capable of hybridizing encodes a polypeptide comprising any one or more of Motifs I to XI as defined herein.

According to the present invention, there is provided a method for enhancing yield-related traits in plants, comprising introducing and expressing in a plant a nucleic acid capable of hybridizing to any one of the nucleic acids given in Tables 3 and 4, or comprising introducing and expressing in a plant a nucleic acid capable of hybridising to a nucleic acid encoding an orthologue, paralogue or homologue of any of the nucleic acid sequences given in Tables 3 and 4.

Another nucleic acid variant useful in the methods of the invention is a splice variant encoding a NAC transcription factor as defined hereinabove.

According to the present invention, there is provided a method for enhancing yield-related traits in plants, comprising introducing and expressing in a plant a splice variant of any one of the nucleic acid sequences given in Tables 3 and 4, or a splice variant of a nucleic acid encoding an orthologue, paralogue or homologue of any of the amino acid sequences given in Tables 3 and 4.

Preferred splice variants are splice variants of a nucleic acid represented by any of SEQ ID NO: 1, SEQ ID NO: 50, SEQ ID NO: 52, SEQ ID NO: 54, SEQ ID NO: 56 or SEQ ID NO: 58, or splice variants encoding orthologues or paralogues of any of the amino acid sequences given in Tables 3 and 4. Preferably, the splice variants encode a polypeptide comprising any one or more of Motifs I to XI as defined herein.

Another nucleic acid variant useful in performing the methods of the invention is an allelic variant of a nucleic acid encoding a NAC transcription factor as defined hereinabove.

According to the present invention, there is provided a method for enhancing yield-related traits in plants, comprising introducing and expressing in a plant an allelic variant of any one of the nucleic acids given in Tables 3 and 4, or comprising introducing and expressing in a plant an allelic variant of a nucleic acid encoding an orthologue, paralogue or homologue of any of the amino acid sequences given in Tables 3 and 4.

Preferably, the allelic variant is an allelic variant of SEQ ID NO: 1, SEQ ID NO: 50, SEQ ID NO: 52, SEQ ID NO: 54, SEQ ID NO: 56 or SEQ ID NO: 58, or an allelic variant of a nucleic acid encoding an orthologue or paralogue of any of the aforementioned SEQ ID NOs. Preferably, the allelic variant encodes a polypeptide comprising any one or more of Motifs I to XI as defined herein.

A further nucleic acid variant useful in the methods of the invention is a nucleic acid variant obtained by gene shuffling. Gene shuffling or directed evolution may also be used to generate variants of nucleic acids encoding NAC transcription factors as defined above.

According to the present invention, there is provided a method for enhancing yield-related traits in plants, comprising introducing and expressing in a plant a variant of any one of the nucleic acid sequences given in Tables 3 and 4, or comprising introducing and expressing in a plant a variant of a nucleic acid encoding an orthologue, paralogue or homologue of any of the amino acid sequences given in Tables 3 and 4, which variant nucleic acid is obtained by gene shuffling. Preferably, the variants obtained by gene shuffling encode a polypeptide comprising any one or more of Motifs I to XI as defined herein.

Furthermore, nucleic acid variants may also be obtained by site-directed mutagenesis. Several methods are available to achieve site-directed mutagenesis, the most common being PCR based methods (current protocols in molecular biology. Wiley Eds).

Also useful in the methods of the invention are nucleic acids encoding homologues of any one of the amino acid sequences given in Tables 3 and 4.

Also useful in the methods of the invention are nucleic acids encoding derivatives of any one of the amino acids given in Table 3 or orthologues or paralogues of any of the aforementioned SEQ ID NOs. Preferred derivatives are derivatives of the proteins represented by SEQ ID NO: 2, SEQ ID NO: 51, SEQ ID NO: 53, SEQ ID NO: 55, SEQ ID NO: 57 or SEQ ID NO: 59.

Furthermore, NAC transcription factors (at least in their native form) typically have DNA-binding activity and an activation domain. A person skilled in the art may easily determine the presence of an activation domain and DNA-binding activity using routine tools and techniques.

Nucleic acids encoding NAC transcription factors may be derived from any natural or artificial source. The nucleic acid may be modified from its native form in composition and/or genomic environment through deliberate human manipulation. Preferably the NAC transcription factor-encoding nucleic acid is from a plant, further preferably from a monocot, more preferably from the Poaceae family, most preferably the nucleic acid is from Oryza sativa.

Any reference herein to a NAC transcription factor is therefore taken to mean a NAC transcription factor as defined above. Any nucleic acid encoding such a NAC transcription factor is suitable for use in performing the methods of the invention.

The present invention also encompasses plants or parts thereof (including seeds) obtainable by the methods according to the present invention. The plants or parts thereof comprise a nucleic acid transgene encoding a NAC transcription factor as defined above.

The invention also provides genetic constructs and vectors to facilitate introduction and/or expression of the nucleic acid sequences useful in the methods according to the invention, in a plant.

Therefore, there is provided a gene construct comprising:
i. Any nucleic acid encoding a NAC transcription factor as defined hereinabove;
ii. One or more control sequences operably linked to the nucleic acid of (i).

Constructs useful in the methods according to the present invention may be constructed using recombinant DNA technology well known to persons skilled in the art. The gene constructs may be inserted into vectors, which may be commercially available, suitable for transforming into plants and suitable for expression of the gene of interest in the transformed cells. The invention therefore provides use of a gene construct as defined hereinabove in the methods of the invention.

Plants are transformed with a vector comprising the sequence of interest (i.e., a nucleic acid encoding a NAC transcription factor). The skilled artisan is well aware of the genetic elements that must be present in the vector in order to successfully transform, select and propagate host cells containing the sequence of interest. The sequence of interest is operably linked to one or more control sequences (at least to a promoter).

According to one preferred feature of the invention, the nucleic acid encoding a NAC transcription factor is operably linked to a constitutive promoter. A constitutive promoter is transcriptionally active during most but not necessarily all phases of its growth and development and is substantially ubiquitously expressed. The constitutive promoter is preferably a GOS2 promoter, more preferably the constitutive promoter is a rice GOS2 promoter, further preferably the constitutive promoter is represented by a nucleic acid sequence substantially similar to SEQ ID NO: 39 or SEQ ID NO: 339, most preferably the constitutive promoter is as represented by SEQ ID NO: 39 or SEQ ID NO: 339.

It should be clear that the applicability of the present invention is not restricted to the NAC transcription factor-encoding nucleic acid represented by SEQ ID NO: 1, SEQ ID NO: 50, SEQ ID NO: 52, SEQ ID NO: 54, SEQ ID NO: 56 or SEQ ID NO: 58, nor is the applicability of the invention restricted to expression of a such a NAC transcription factor-encoding nucleic acid when driven by a GOS2 promoter. Examples of other constitutive promoters which may also be used to perform the methods of the invention are shown in Table 2a in the “definitions” section herein.

According to another preferred feature of the invention, the nucleic acid encoding a NAC-type transcription factor is operably linked to a young green tissue-specific promoter. A young green tissue-specific promoter as defined herein is a promoter that is transcriptionally active predominantly in young green tissue, substantially to the exclusion of any other parts of a plant, whilst still allowing for any leaky expression in these other plant parts. The young green tissue-specific promoter is preferably a protochlorophylid reductase promoter, more preferably the protochlorophylid reductase promoter represented by a nucleic acid sequence substantially similar to SEQ ID NO: 40, most preferably the promoter is as represented by SEQ ID NO: 40.

It should be clear that the applicability of the present invention is not restricted to the NAC transcription factor-encoding nucleic acid represented by SEQ ID NO: 1, nor is the applicability of the invention restricted to expression of a such a NAC transcription factor-encoding nucleic acid when driven by a protochlorophylid reductase promoter.

Examples of other young green tissue-specific promoters which may also be used to perform the methods of the invention are shown in Table 2c above.

According to another preferred feature of the invention, the nucleic acid encoding a NAC-type transcription factor is operably linked to a root-specific promoter (internal reference pro0110). A root-specific promoter as defined herein is a promoter that is transcriptionally active predominantly in plant roots, substantially to the exclusion of any other parts of a plant, whilst still allowing for any leaky expression in these other plant parts. The root-specific promoter is preferably an RCc3 promoter (Plant Mol Biol. 1995 January; 27(2):237-48), more preferably the RCc3 promoter is from rice, further preferably the RCc3 promoter is represented by a nucleic acid sequence substantially similar to SEQ ID NO: 110, most preferably the promoter is as represented by SEQ ID NO: 110.

It should be clear that the applicability of the present invention is not restricted to the NAC transcription factor-encoding nucleic acid represented by SEQ ID NO: 50, SEQ ID NO: 52, SEQ ID NO: 54, SEQ ID NO: 56 or SEQ ID NO: 58, nor is the applicability of the invention restricted to expression of a such a NAC transcription factor-encoding nucleic acid when driven by an RCc3 promoter.

Examples of other root-specific promoters which may also be used to perform the methods of the invention are shown in Table 2b above.

Optionally, one or more terminator sequences may be used in the construct introduced into a plant. Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1. The genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

The invention also provides a method for the production of transgenic plants having enhanced yield-related traits relative to control plants, comprising introduction and expression in a plant of any nucleic acid encoding a NAC transcription factor as defined hereinabove.

More specifically, the present invention provides a method for the production of transgenic plants having enhanced yield-related traits, which method comprises:
i. introducing and expressing a nucleic acid encoding a NAC transcription factor (as defined herein) in a plant cell; and
ii. cultivating the plant cell under conditions promoting plant growth and development.

The nucleic acid may be introduced directly into a plant cell or into the plant itself (including introduction into a tissue, organ or any other part of a plant). According to a preferred feature of the present invention, the nucleic acid is preferably introduced into a plant by transformation.

The genetically modified plant cells can be regenerated via all methods with which the skilled worker is familiar. Suitable methods can be found in the abovementioned publications by S. D. Kung and R. Wu, Potrykus or Höfgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding a NAC transcription factor as defined hereinabove. Preferred host cells according to the invention are plant cells.

The invention also extends to harvestable parts of a plant such as, but not limited to seeds, leaves, fruits, flowers, stems, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art. Examples of methods for increasing expression are given in the “definitions” section herein.

As mentioned above, a preferred method for modulating (preferably, increasing) expression of a nucleic acid encoding a NAC transcription factor is by introducing and expressing in a plant a nucleic acid encoding a NAC transcription factor; however the effects of performing the method, i.e. enhancing yield-related traits may also be achieved using other well known techniques, such as T-DNA activation, TILLING, homologous recombination or directed evolution. A description of some of these techniques is given in the “definitions” section herein.

According to a preferred feature of the present invention, performance of the methods of the invention gives plants having an increased growth rate relative to control plants, particularly during the early stages of plant development (typically three weeks post germination in the case of rice and maize, but this will vary from species to species) leading to early vigour. Therefore, according to the present invention, there is provided a method for increasing the growth rate of plants, which method comprises modulating expression, preferably increasing expression, in a plant of a nucleic acid encoding a particular type of NAC transcription factor, as defined herein. The present invention also provides a method for obtaining plants having early vigour relative to control plants, which method comprises modulating, preferably increasing, expression in a plant of a nucleic acid encoding a NAC transcription factor as defined herein.

Early vigour may also result from or be manifested as increased plant fitness relative to control plants due to, for example, the plants being better adapted to their environment (i.e. being more able to cope with various abiotic or biotic stress factors). Plants having early vigour also show better establishment of the crop (with the crop growing in uniform manner, i.e. with the majority of plants reaching the various stages of development at substantially the same time), and show better growth and often better yield. Early vigour may be determined by measuring various factors, such as seedling growth rate, thousand kernel weight, percentage germination, percentage emergence, seedling height, root length and shoot biomass and many more.

Performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises increasing expression in a plant of a nucleic acid encoding a NAC transcription factor.

Performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises increasing expression in a plant of a nucleic acid encoding a POI polypeptide. Nutrient deficiency may result from a lack of nutrients such as nitrogen, phosphates and other phosphorous-containing compounds, potassium, calcium, cadmium, magnesium, manganese, iron and boron, amongst others.

In the case of use in the methods of the invention of nucleic acids encoding NAC4 transcription factors (for example, SEQ ID NO: 52) and NAC4 transcription factors themselves (for example SEQ ID NO: 53), an increase in yield and/or seed yield and/or growth rate was found to occur in plants grown under substantially non-stress conditions and under conditions of stress compared to control plants.

A “NAC4 transcription factor” as defined herein refers to any amino acid sequence which when used in the construction of a NAC phylogenetic tree, such as the one depicted in FIG. 1, tends to cluster with the group of NACs comprising an amino acid sequence represented by SEQ ID NO: 53 rather than with any other NAC group.

The NAC4 transcription factor may also comprise Motif XI: DSMPRLHADSSCSE, or a motif having in increasing order of preference at least 50%, 60%, 70%, 80% or 90% sequence identity to the sequence of Motif XI.

Motif XI is preferably DS M/V/I P R/K L/I/A H T/A/S D/E SS C/G SE.

Motif XI is typically found in NACs of SEQ ID NO: 53 and SEQ ID NO: 55 and in NACs clustering (in a phylogenetic tree) with the NACs represented by the aforementioned SEQ ID NOs rather than with any other NAC group. Motif XI may comprise one or more conservative amino acid substitution at any position.

Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the typical stresses to which a plant may be exposed. These stresses may be the everyday biotic and/or abiotic (environmental) stresses to which a plant is exposed. Typical abiotic or environmental stresses include temperature stresses caused by atypical hot or cold/freezing temperatures; salt stress; water stress (drought or excess water), anaerobic stress, chemical toxicity and oxidative stress. The abiotic stress may be an osmotic stress caused by a water stress (particularly due to drought), salt stress, oxidative stress or an ionic stress. Chemicals may also cause abiotic stresses (for example too high or too low concentrations of minerals or nutrients). Biotic stresses are typically those stresses caused by pathogens, such as bacteria, viruses, fungi and insects. The term “non-stress conditions” as used herein are those environmental conditions that do not significantly go beyond the everyday climatic and other abiotic conditions that plants may encounter, and which allow optimal growth of the plant. Persons skilled in the art are aware of normal soil conditions and climatic conditions for a given geographic location.

Abiotic stress leads to a series of morphological, physiological, biochemical and molecular changes that adversely affect plant growth and productivity (Wang et al. (Planta (2003) 218: 1-14). Drought, salinity, extreme temperatures and oxidative stress are known to be interconnected and may induce growth and cellular damage through similar mechanisms. For example, drought and/or salinisation are manifested primarily as osmotic stress, resulting in the disruption of homeostasis and ion distribution in the cell. Oxidative stress, which frequently accompanies high or low temperature, salinity or drought stress may cause denaturation of functional and structural proteins. As a consequence, these diverse environmental stresses often activate similar cell signaling pathways and cellular responses, such as the production of stress proteins, up-regulation of anti-oxidants, accumulation of compatible solutes and growth arrest.

Since diverse environmental stresses activate similar pathways, the exemplification of the present invention with drought stress (in the case of use of nucleic acids encoding NAC4 transcription factors, and NAC4 transcription factors themselves, in the methods of the invention) should not be seen as a limitation to drought stress, but more as a screen to indicate the involvement of NAC4-encoding nucleic acids and NAC4 transcription factors in abiotic stresses in general. Furthermore, the methods of the present invention may be performed under non-stress conditions or under conditions of mild drought to give plants having enhanced yield-related traits (particularly increased yield and seed yield) relative to control plants.

A particularly high degree of “cross talk” is reported between drought stress and high-salinity stress (Rabbani et al. (2003) Plant Physiol 133: 1755-1767). For example, drought and/or salinisation are manifested primarily as osmotic stress, resulting in the disruption of homeostasis and ion distribution in the cell. Therefore, it would be apparent that nucleic acids encoding NAC4 transcription factors, and NAC4 transcription factors themselves would, along with their usefulness in conferring drought-tolerance in plants, also find use in protecting the plant against various other osmotic stresses. Similarly, it would be apparent that nucleic acids encoding NAC4 transcription factors, and NAC4 transcription factors themselves would, along with their usefulness in conferring salt-tolerance in plants, also find use in protecting the plant against various other abiotic stresses. Furthermore, oxidative stress, which frequently accompanies high or low temperatures, salinity or drought stress, may cause denaturing of functional and structural proteins. As a consequence, these diverse environmental stresses often activate similar cell signaling pathways and cellular responses, such as the production of stress proteins, up-regulation of anti-oxidants, accumulation of compatible solutes and growth arrest. Furthermore, Rabbani et al. (2003, Plant Physiol 133: 1755-1767) report that similar molecular mechanisms of stress tolerance and responses exist between dicots and monocots. The methods of the invention are therefore advantageously applicable to any plant.

The term “abiotic stress” as defined herein is taken to mean any one or more of: water stress (due to drought or excess water), anaerobic stress, salt stress, nutrient stress, temperature stress (due to hot, cold or freezing temperatures), chemical toxicity stress and oxidative stress. According to one aspect of the invention, the abiotic stress is an osmotic stress, selected from water stress, salt stress, oxidative stress and ionic stress. Preferably, the water stress is drought stress. The term salt stress is not restricted to common salt (NaCl), but may be any one or more of: NaCl, KCl, LiCl, MgCl₂, CaCl₂, Phosphorous salts, amongst others. Nutrient stress may be caused by a lack or excess of nutrients such as nitrogen, phosphates and other phosphorous-containing compounds, potassium, calcium, cadmium, magnesium, manganese, iron and boron, amongst others.

Increased tolerance to abiotic stress is manifested by increased plant yield under abiotic stress conditions as defined above. Insofar as the invention concerns the use of NAC4 transcription factors and their encoding nucleic acids, such increased yield may include one or more of the following: increased seed size, increased seed weight, increased aboveground biomass, increased plant height, increased root biomass, increased number of flowers per panicle and increased number of first panicles, each relative to control plants.

According to the present invention, there is provided a method for enhancing yield-related traits in plants grown under abiotic stress conditions relative to control plants, comprising modulating expression in a plant of a nucleic acid encoding a NAC4 transcription factor. According to one aspect of the invention, the abiotic stress is osmotic stress, selected from one or more of the following: water stress, salt stress, oxidative stress, nutrient stress and ionic stress. Preferably, the water stress is drought stress.

The methods of the invention are advantageously applicable to any plant.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, triticale, rye, sorghum and oats. Plants in which early vigour is a particularly desirably trait include rice, maize, wheat, sunflower, sorghum.

The present invention also encompasses use of nucleic acids encoding NAC transcription factors and use of NAC transcription factor polypeptides in enhancing yield-related traits.

Nucleic acids encoding NAC transcription factors or NAC transcription factors themselves, may find use in breeding programmes in which a DNA marker is identified which may be genetically linked to a NAC transcription factor-encoding gene. The nucleic acids/genes, or the NAC transcription factors themselves may be used to define a molecular marker. This DNA or protein marker may then be used in breeding programmes to select plants having enhanced yield as defined hereinabove in the methods of the invention.

Allelic variants of NAC transcription factor-encoding nucleic acids/genes may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acids encoding NAC transcription factors may also be used as probes for genetically and physically mapping the genes that they are a part of, and as markers for traits linked to those genes. Such information may be useful in plant breeding in order to develop lines with desired phenotypes. Such use of NAC transcription factor encoding nucleic acids requires only a nucleic acid sequence of at least 15 nucleotides in length. The NAC transcription factor-encoding nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the NAC transcription factor-encoding nucleic acids. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the NAC transcription factor-encoding nucleic acid in the genetic map previously obtained using this population (Botstein et al. (1980) Am. J. Hum. Genet. 32:314-331).

The production and use of plant gene-derived probes for use in genetic mapping is described in Bematzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favor use of large clones (several kb to several hundred kb; see Laan et al. (1995) Genome Res. 5:13-20), improvements in sensitivity may allow performance of FISH mapping using shorter probes.

The present invention further provides a method for enhancing yield-related traits in plants relative to control plants, comprising modulating expression in a plant of a nucleic acid encoding an AP2-2 polypeptide.

A preferred method for modulating (preferably, increasing) expression of a nucleic acid encoding an AP2-2 polypeptide is by introducing and expressing in a plant a nucleic acid encoding an AP2-2 polypeptide.

The term “AP2-2 polypeptide” as defined herein refers to a transcription factor that classifies as an ERF protein, and more particularly as a member of Group VII of the ERF proteins, according to the definition by Nakano et al. (2006). Group VII of ERF proteins is, besides the presence of a single AP2/ERF domain, which is part of all ERF proteins, also characterised by other motifs, such as CMVII-1 to CMVII-8 (Nakano et al., 2006). These CMVII motifs were delineated based on analysis of Arabidopsis thaliana and Oryza sativa sequences and are part of the Arabidopsis and/or rice sequences (FIG. 2), but may also be present in Group VII ERF proteins of other species.

The AP2-2 polypeptide useful in the methods of the present invention comprises the conserved

sequence Motif XII (SEQ ID NO: 133):
(R/I/V/L/K)(R/K/Q/D/E/A)(I/L)(R/Y/H)G(A/S/R/K/T/D/
G/H/L/N)(K/T/R/N/G)A(K/R/E)(V/L/P/T)NF(P/V)
Preferably, motif XII is
(R/I/K)(R/K/Q/D/A)(I/L)(R/Y/H)G(A/S/R/K/T/D/E/G/H/
L/N)(K/T/R/N/G)A(K/R/E)(V/L/P/T)NF(P/V/A)

Preferably, the AP2-2 polypeptide useful in the methods of the present invention furthermore also comprises one or more of the following motifs:

Motif XIII (SEQ ID NO: 134):
MCGG(A/S)(I/V/L)(I/L)(S/H/A/Y/G/P/E)(D/G/H/Y/E/Q/
N)
Preferably, motif XIII is
MCGG(A/S)I(I/L)(S/H/A/Y)(D/G/H/Y/E)
More preferably motif XIII is
MCGG(A/S)I(I/L)(S/H)(D/G/H/E)
Motif XIV (SEQ /D NO: 135)
(K/N/R/S/H/M/Q/P/A/E)(R/K/H/A/G/E/V/P/M)(E/K/A/R/
S/Q/T/V/G/H/P)(R/K/Q/S/G)(K/G/P/S/R/T/A/N)(T/N/R/
S/A/Y/H/G)(L/Q/H/V/R/K/A/P/G)(Y/F)(R/W/K/L/M)G(I/
V)(R/Q/H)(R/Q/W/K)R(P/K/T)
Preferably, motif XIV is:
(K/N/R/S/H/M)(R/K/H/A/G/E/V/P)(E/K/A/R/S/Q)(R/K/Q/
S)(K/G/P/S/R/T)(T/N/R/S/A/Y/H/G)(L/Q/H/V/R/K/A/P/
G/F/I)(Y/F/L)(R/W/K/L/M/H)G(I/V)(R/Q/H)(R/Q/W/K)R
(P/K/T)
More preferably, motif XIV is:
(K/N/R/S)(R/K/H)(E/K/A/R)(R/K/Q)(K/G/P/S)(T/N/R/S/
A)(L/Q/H/V/R/K)(Y/F)(R/W/K)G(I/V)R(R/Q)RP
Most preferably, motif XIV is:
(K/N)(R/H)KRKNQ(Y/F)RGIRQRP
Motif XV (SEQ ID NO: 136):
SD(Q/T/E/V)(G/S)SNSF(G/D/E/S/N)(C/S)S(D/E)(F/Y/L)
(G/S)(W/Q/L)(E/G/S)(N/E/D)
Preferably, motif XV is:
SD(Q/T)(G/S/A)SNS(F/I)(G/D)(C/S)S(D/E)F(G/S)(W/Q/
L)(E/S)(N/D)
Motif XVI (SEQ /D NO: 137):
(L/I/F/M)W(S/T/N/M)(F/Y/L/I)(D/E/Q/G)(N/D/H/E)(I/
Y/F/S/M/L/V/D/H/N/E/G)
Preferably, motif XVI is
(L/I/M)W(S/T/M)(F/Y/L/I)(D/E/Q/G)(N/D/E)(I/Y/F/S/
M/L/V/D)
More preferably, motif XVI is
(L/I/M)W(S/M)(F/L/I)D(D/E)(I/M/L/V)
Motif XVII (SEQ ID NO 138):
(D/E/S)(F/A/W/D)(E/A)(A/D/L)(D/A/G/E)(F/G/L)(N/E/
R/G/Q/W)(E/G/V/D/R)F(E/K/V/Y/G/D/L/I)(V/R/D/S/N/A/
E)(D/G/T/E/R/A/Y/L/F)
Preferably, motif XVII is
(D/E/S)(F/A/W/D)(E/A)(A/D)(D/A)(F/G)(E/R/Q/W)(E/G/
D/R)F(E/K/Y/G/D/L/I)(V/R/D/S/N/A/E)(D/G/T/E/R)
Further preferably, motif XVII is
(D/E/S)(F/A)(E/A)(A/D)(D/A)F(E/R/Q/W)(E/G/D/R)F(E/
K/Y/G/D)(V/R/D/S/N)(D/G/T/E)
More preferably, motif XVII is
(D/E/S)(F/A)(E/A)(A/D)DF(E/R/Q/W)(E/G)F(E/K/Y)(V/
R/D/S)(D/G/T)
Most preferably, motif XVII is
(D/E)FEADF(E/R/Q)EF(E/K)(V/R/D)(D/G)

These motifs were derived mainly from Arabidopsis and rice sequences, therefore, one or more conserved substitutions are allowed in these motifs for AP2-2 sequences from other plant species.

Furthermore, AP2-2 polypeptides (at least in their native form) may have DNA-binding activity. Tools and techniques for measuring DNA-binding activity are well known in the art.

The terms “domain” and “motif” are defined in the “definitions” section herein. Specialist databases exist for the identification of domains. Examples are given in the “definitions” section herein.

Analysis of the polypeptide sequence of SEQ ID NO: 132 in the SMART database, revealed there to be an AP2 domain (SMART entry SM00380, FIG. 2). This domain is plant specific and is known to play a role in protein-DNA interactions (binds to the GCC-box, essential for the response to ethylene).

The present invention is illustrated by transforming plants with the nucleic acid sequence represented by SEQ ID NO: 131, encoding the polypeptide sequence of SEQ ID NO: 132. However, performance of the invention is not restricted to these sequences; the methods of the invention may advantageously be performed using any AP2-2-encoding nucleic acid or AP2-2 polypeptides as defined herein.

Examples of nucleic acids encoding AP2-2 polypeptides are given in Table 14 of Example 9 herein. Such nucleic acids are useful in performing the methods of the invention. The amino acid sequences given in Table 14 of Example 9 are example sequences of orthologues and paralogues of the AP2-2 polypeptides represented by SEQ ID NO: 132, the terms “orthologues” and “paralogues” being as defined herein. Further orthologues and paralogues may readily be identified by performing a so-called reciprocal blast search.

The invention also provides hitherto unknown AP2-2-encoding nucleic acids and AP2-2 polypeptides.

According to a further embodiment of the present invention, there is therefore provided an isolated nucleic acid molecule comprising:

(i) a nucleic acid represented by one of SEQ ID NO: 341, SEQ ID NO: 343, or SEQ ID NO: 345;
(ii) the complement of a nucleic acid represented by one of SEQ ID NO: 341, SEQ ID NO: 343, or SEQ ID NO: 345;
(iii) a nucleic acid encoding a POI polypeptide having, in increasing order of preference, at least 50%, 55%, 60%, 65%, 70%, 75%, 80%, 85%, 90%, 95%, 96%, 97%, 98%, 99% or more sequence identity to the amino acid sequence represented by one of SEQ ID NO: 342, SEQ ID NO: 344, or SEQ ID NO: 346.

According to a further embodiment of the present invention, there is also provided an isolated polypeptide comprising:

(i) an amino acid sequence represented by one of SEQ ID NO: 342, SEQ ID NO: 344, or SEQ ID NO: 346;
(ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequence represented by one of SEQ ID NO: 342, SEQ ID NO: 344, or SEQ ID NO: 346;
(iii) derivatives of any of the amino acid sequences given in (i) or (ii) above. Nucleic acid variants may also be useful in practicing the methods of the invention. Examples of such variants include nucleic acids encoding homologues and derivatives of any one of the amino acid sequences given in Table 14 of Example 9, the terms “homologue” and “derivative” being as defined herein. Also useful in the methods of the invention are nucleic acids encoding homologues and derivatives of orthologues or paralogues of any one of the amino acid sequences given in Table 14 of Example 9. Homologues and derivatives useful in the methods of the present invention have substantially the same biological and functional activity as the unmodified protein from which they are derived.

Further nucleic acid variants useful in practicing the methods of the invention include portions of nucleic acids encoding AP2-2 polypeptides, nucleic acids hybridising to nucleic acids encoding AP2-2 polypeptides, splice variants of nucleic acids encoding AP2-2 polypeptides, allelic variants of nucleic acids encoding AP2-2 polypeptides and variants of nucleic acids encoding AP2-2 polypeptides obtained by gene shuffling. The terms hybridising sequence, splice variant, allelic variant and gene shuffling are as described herein.

Nucleic acids encoding AP2-2 polypeptides need not be full-length nucleic acids, since performance of the methods of the invention does not rely on the use of full-length nucleic acid sequences. According to the present invention, there is provided a method for enhancing yield-related traits in plants, comprising introducing and expressing in a plant a portion of any one of the nucleic acid sequences given in Table 14 of Example 9, or a portion of a nucleic acid encoding an orthologue, paralogue or homologue of any of the amino acid sequences given in Table 14 of Example 9.

A portion of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode an AP2-2 polypeptide as defined herein, and have substantially the same biological activity as the amino acid sequences given in Table 14 of Example 9. Preferably, the portion is a portion of any one of the nucleic acids given in Table 14 of Example 9, or is a portion of a nucleic acid encoding an orthologue or paralogue of any one of the amino acid sequences given in Table 14 of Example 9. Preferably the portion is, in increasing order of preference at least 600, 800, 900 or 1000 consecutive nucleotides in length, the consecutive nucleotides being of any one of the nucleic acid sequences given in Table 14 of Example 9, or of a nucleic acid encoding an orthologue or paralogue of any one of the amino acid sequences given in Table 14 of Example 9. Most preferably the portion is a portion of the nucleic acid of SEQ ID NO: 131. Preferably, the portion encodes an amino acid sequence comprising (any one or more of the domains or motifs defined herein). Preferably, the portion encodes an amino acid sequence which, when used in the construction of a phylogenetic tree, tends to cluster with the group of AP2-2 polypeptides comprising the amino acid sequence represented by SEQ ID NO: 132 rather than with any other group.

Another nucleic acid variant useful in the methods of the invention is a nucleic acid capable of hybridising, under reduced stringency conditions, preferably under stringent conditions, with a nucleic acid encoding an AP2-2 polypeptide as defined herein, or with a portion as defined herein.

According to the present invention, there is provided a method for enhancing yield-related traits in plants, comprising introducing and expressing in a plant a nucleic acid capable of hybridizing to any one of the nucleic acids given in Table 14 of Example 9, or comprising introducing and expressing in a plant a nucleic acid capable of hybridising to a nucleic acid encoding an orthologue, paralogue or homologue of any of the nucleic acid sequences given in Table 14 of Example 9.

Hybridising sequences useful in the methods of the invention encode an AP2-2 polypeptide as defined herein, and have substantially the same biological activity as the amino acid sequences given in Table 14 of Example 9. Preferably, the hybridising sequence is capable of hybridising to any one of the nucleic acids given in Table 14 of Example 9, or to a portion of any of these sequences, a portion being as defined above, or wherein the hybridising sequence is capable of hybridising to a nucleic acid encoding an orthologue or paralogue of any one of the amino acid sequences given in Table 14 of Example 9. Most preferably, the hybridising sequence is capable of hybridising to a nucleic acid as represented by SEQ ID NO: 131 or to a portion thereof. Preferably, the hybridising sequence encodes an amino acid sequence comprising any one or more of the motifs or domains as defined herein. Preferably, the hybridising sequence encodes an amino acid sequence which, when used in the construction of a phylogenetic tree, tends to cluster with the group of AP2-2 polypeptides comprising the amino acid sequence represented by SEQ ID NO: 132 rather than with any other group.

Another nucleic acid variant useful in the methods of the invention is a splice variant encoding an AP2-2 polypeptide as defined hereinabove, a splice variant being as defined herein.

According to the present invention, there is provided a method for enhancing yield-related traits in plants, comprising introducing and expressing in a plant a splice variant of any one of the nucleic acid sequences given in Table 14 of Example 9, or a splice variant of a nucleic acid encoding an orthologue, paralogue or homologue of any of the amino acid sequences given in Table 14 of Example 9.

Preferred splice variants are splice variants of a nucleic acid represented by SEQ ID NO: 131, or a splice variant of a nucleic acid encoding an orthologue or paralogue of SEQ ID NO: 132. Preferably, the amino acid sequence encoded by the splice variant comprises any one or more of the motifs or domains as defined herein. Preferably, the amino acid sequence encoded by the splice variant, when used in the construction of a phylogenetic tree, tends to cluster with the group of AP2-2 polypeptides comprising the amino acid sequence represented by SEQ ID NO: 132 rather than with any other group.

Another nucleic acid variant useful in performing the methods of the invention is an allelic variant of a nucleic acid encoding an AP2-2 polypeptide as defined hereinabove, an allelic variant being as defined herein.

According to the present invention, there is provided a method for enhancing yield-related traits in plants, comprising introducing and expressing in a plant an allelic variant of any one of the nucleic acids given in Table 14 of Example 9, or comprising introducing and expressing in a plant an allelic variant of a nucleic acid encoding an orthologue, paralogue or homologue of any of the amino acid sequences given in Table 14 of Example 9.

The allelic variants useful in the methods of the present invention have substantially the same biological activity as the AP2-2 polypeptide of SEQ ID NO: 132 and any of the amino acids depicted in Table 14 of Example 9. Allelic variants exist in nature, and encompassed within the methods of the present invention is the use of these natural alleles. Preferably, the allelic variant is an allelic variant of SEQ ID NO: 131 or an allelic variant of a nucleic acid encoding an orthologue or paralogue of SEQ ID NO: 132. Preferably, the amino acid encoded by the allelic variant comprises any one or more of the motifs or domains as defined herein. Preferably, the amino acid sequence encoded by the allelic variant, when used in the construction of a phylogenetic tree, tends to cluster with the group of AP2-2 polypeptides comprising the amino acid sequence represented by SEQ ID NO: 132 rather than with any other group.

Gene shuffling or directed evolution may also be used to generate variants of nucleic acids encoding AP2-2 polypeptides as defined above; the term “gene shuffling” being as defined herein.

According to the present invention, there is provided a method for enhancing yield-related traits in plants, comprising introducing and expressing in a plant a variant of any one of the nucleic acid sequences given in Table 14 of Example 9, or comprising introducing and expressing in a plant a variant of a nucleic acid encoding an orthologue, paralogue or homologue of any of the amino acid sequences given in Table 14 of Example 9, which variant nucleic acid is obtained by gene shuffling.

Preferably, the variant nucleic acid obtained by gene shuffling encodes an amino acid sequence comprising any one or more of the motifs or domains as defined herein. Preferably, the amino acid sequence encoded by the variant nucleic acid obtained by gene shuffling, when used in the construction of a phylogenetic tree, tends to cluster with the group of AP2-2 polypeptides comprising the amino acid sequence represented by SEQ ID NO: 132 rather than with any other group.

Furthermore, nucleic acid variants may also be obtained by site-directed mutagenesis. Several methods are available to achieve site-directed mutagenesis, the most common being PCR based methods (Current Protocols in Molecular Biology. Wiley Eds.).

Nucleic acids encoding AP2-2 polypeptides may be derived from any natural or artificial source. The nucleic acid may be modified from its native form in composition and/or genomic environment through deliberate human manipulation. Preferably the AP2-2 polypeptide-encoding nucleic acid is from a plant, further preferably from a dicotyledonous plant, more preferably from the family Poaceae, more preferably from the genus Oryza, most preferably from Oryza sativa.

Performance of the methods of the invention gives plants having enhanced yield-related traits. In particular, performance of the methods of the invention gives plants having increased yield, especially increased seed yield relative to control plants. The terms “yield” and “seed yield” are described in more detail in the “definitions” section herein. However it should be noted that the term “yield-related traits” does not encompass the metabolite content of plant cells and that the enhanced yield-related traits are not the result of increased stress resistance.

The present invention provides a method for increasing yield, especially seed yield of plants, relative to control plants, which method comprises modulating expression, preferably increasing expression, in a plant of a nucleic acid encoding an AP2-2 polypeptide as defined herein.

According to a preferred feature of the present invention, performance of the methods of the invention gives plants having an increased growth rate relative to control plants. Therefore, according to the present invention, there is provided a method for increasing the growth rate of plants, which method comprises modulating expression, preferably increasing expression, in a plant of a nucleic acid encoding an AP2-2 polypeptide as defined herein.

Performance of the methods of the invention gives plants grown under non-stress conditions or under mild drought conditions increased yield relative to suitable control plants grown under comparable conditions. Therefore, according to the present invention, there is provided a method for increasing yield in plants grown under non-stress conditions, which method comprises increasing expression in a plant of a nucleic acid encoding an AP2-2 polypeptide.

Performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises increasing expression in a plant of a nucleic acid encoding a POI polypeptide. Nutrient deficiency may result from a lack of nutrients such as nitrogen, phosphates and other phosphorous-containing compounds, potassium, calcium, cadmium, magnesium, manganese, iron and boron, amongst others.

The present invention encompasses plants or parts thereof (including seeds) obtainable by the methods according to the present invention. The plants or parts thereof comprise a nucleic acid transgene encoding an AP2-2 polypeptide as defined above.

The invention also provides genetic constructs and vectors to facilitate introduction and/or expression in plants of nucleic acids encoding AP2-2 polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(a) a nucleic acid encoding an AP2-2 polypeptide as defined above;
(b) one or more control sequences capable of driving expression of the nucleic acid sequence of (a); and optionally
(c) a transcription termination sequence.

The term “control sequence” and “termination sequence” are as defined herein. Plants are transformed with a vector comprising any of the nucleic acids described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter may be used to drive expression of the nucleic acid sequence. A constitutive promoter is particularly useful in the methods of the invention, preferably the constitutive promoter is a strong constitutive promoters. It should be clear that the applicability of the present invention is not restricted to the AP2-2 polypeptide-encoding nucleic acid represented by SEQ ID NO: 131, nor is the applicability of the invention restricted to expression of an AP2-2 polypeptide-encoding nucleic acid when driven by a constitutive promoter.

The constitutive promoter is preferably a GOS2 promoter, more preferably the rice GOS2 promoter. See Table 2a in the “definitions” section herein for further examples of constitutive promoters.

Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

The invention also provides a method for the production of transgenic plants having enhanced yield-related traits relative to control plants, comprising introduction and expression in a plant of any nucleic acid encoding an AP2-2 polypeptide as defined hereinabove.

More specifically, the present invention provides a method for the production of transgenic plants having increased yield, which method comprises:
(i) introducing and expressing in a plant or plant cell an AP2-2 polypeptide-encoding nucleic acid; and
(ii) cultivating the plant cell under conditions promoting plant growth and development.

The nucleic acid may be introduced directly into a plant cell or into the plant itself (including introduction into a tissue, organ or any other part of a plant). According to a preferred feature of the present invention, the nucleic acid is preferably introduced into a plant by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Höfgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding an AP2-2 polypeptide as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

The invention also extends to harvestable parts of a plant such as, but not limited to seeds, leaves, fruits, flowers, stems, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art. Examples of methods for increasing expression are given in the “definitions” section.

As mentioned above, a preferred method for modulating (preferably, increasing) expression of a nucleic acid encoding an AP2-2 polypeptide is by introducing and expressing in a plant a nucleic acid encoding an AP2-2 polypeptide; however the effects of performing the method, i.e. enhancing yield-related traits may also be achieved using other well known techniques. A description of some of these techniques will now follow.

The effects of the invention may also be reproduced using the technique of T-DNA activation or TILLING (Targeted Induced Local Lesions In Genomes); for a description of the same see the “definitions” section.

The effects of the invention may also be reproduced using homologous recombination; for a description of the same see the “definitions” section.

The present invention also encompasses use of nucleic acids encoding AP2-2 polypeptides as described herein and use of this AP2-2 polypeptide in enhancing any of the aforementioned yield-related traits in plants.

Nucleic acids encoding AP2-2 polypeptide described herein, or the AP2-2 polypeptides themselves, may find use in breeding programmes in which a DNA marker is identified which may be genetically linked to an AP2-2 polypeptide-encoding gene. The nucleic acids/genes, or the AP2-2 polypeptides themselves may be used to define a molecular marker. This DNA or protein marker may then be used in breeding programmes to select plants having enhanced yield-related traits as defined hereinabove in the methods of the invention.

Allelic variants of an AP2-2 polypeptide-encoding nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acids encoding AP2-2 polypeptides may also be used as probes for genetically and physically mapping the genes that they are a part of, and as markers for traits linked to those genes. Such information may be useful in plant breeding in order to develop lines with desired phenotypes. Such use of AP2-2 polypeptide-encoding nucleic acids requires only a nucleic acid sequence of at least 15 nucleotides in length. The AP2-2 polypeptide-encoding nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the AP2-2-encoding nucleic acids. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the AP2-2 polypeptide-encoding nucleic acid in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples can be found in the “definitions” section herein.

The present invention further provides a method for enhancing yield-related traits in plants relative to control plants, comprising modulating expression in a plant of a nucleic acid encoding an AP2-70-like polypeptide.

The invention also provides hitherto unknown AP2-70-like-encoding nucleic acids and AP2-70-like polypeptides.

According to a further embodiment of the present invention, there is therefore provided an isolated nucleic acid molecule comprising:
(i) a nucleic acid represented by SEQ ID NO: 257;
(ii) the complement of a nucleic acid represented by SEQ ID NO: 257;
(iii) a nucleic acid encoding an AP2-70-like polypeptide having, in increasing order of preference, at least 50%, 55%, 60%, 65%, 70%, 75%, 80%, 85%, 90%, 95%, 96%, 97%, 98%, 99% or more sequence identity to the amino acid sequence represented by SEQ ID NO: 258, and having in increasing order of preference at least 50%, 55%, 60%, 65%, 70%, 75%, 80%, 85%, 90%, 95%, 96%, 97%, 98%, 99% or more sequence identity to SEQ ID NO: 331: PFLMQWLNLLPLPVLDSSSWCPEHFHNSESDALP (which represents the C-terminal region of SEQ ID NO: 258).

According to a further embodiment of the present invention, there is also provided an isolated polypeptide comprising:
(i) an amino acid sequence represented by SEQ ID NO: 258;
(ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequence represented by SEQ ID NO: 258, and having in increasing order of preference at least 50%, 55%, 60%, 65%, 70%, 75%, 80%, 85%, 90%, 95%, 96%, 97%, 98%, 99% or more sequence identity to SEQ ID NO: 331: PFLMQWLNLLPLPVLDSSSWCPEHFHNSESDALP (which represents the C-terminal region of SEQ ID NO: 258);
(iii) derivatives of any of the amino acid sequences given in (i) or (ii) above.

A preferred method for modulating (preferably, increasing) expression of a nucleic acid encoding an AP2-70-like polypeptide is by introducing and expressing in a plant a nucleic acid encoding an AP2-70-like polypeptide.

Any reference hereinafter to a “protein useful in the methods of the invention” is taken to mean an AP2-70-like polypeptide as defined herein. Any reference hereinafter to a “nucleic acid useful in the methods of the invention” is taken to mean a nucleic acid capable of encoding such an AP2-70-like polypeptide. The nucleic acid to be introduced into a plant (and therefore useful in performing the methods of the invention) is any nucleic acid encoding the type of protein which will now be described, hereafter also named “AP2-70-like nucleic acid” or “AP2-70-like gene”.

AP2-70-like polypeptides as defined below fall within Group Ib (A-6) according to the classification of Nakano et al., 2006.

An “AP2-70-like polypeptide” as defined herein refers to any polypeptide comprising the following:
(i) An AP2 DNA-binding domain as represented by SEQ ID NO: 332: YRGVRQRHWGKWVAEIRLPRNRTRLWLGTFDTAEEAALAYDSAAFRLRGESARLNF, or a domain having in increasing order of preference at least 50%, 55%, 60%, 65%, 70%, 75%, 80%, 85%, 90%, 95% or more sequence identity to the domain represented by SEQ ID NO: 332; and
(ii) Having in increasing order of preference at least 50%, 55%, 60%, 65%, 70%, 75%, 80%, 85%, 90%, 95% or more sequence identity to the (Oryza sativa) polypeptide sequence represented by SEQ ID NO: 258; and
(iii) Comprising Motif XVIII represented by SEQ ID NO: 333: RLPXNX¹RXRXWLGT F/Y D/E T/S, where X is any amino acid and X¹ is zero, one or more, and up to 30, gaps; and
(iv) Comprising Motif XIX represented by SEQ ID NO: 334: RG D/E.

In the AP2 DNA-binding domain described above under (i), YRG and LAYD, as highlighted below, are preferred specific boxes for DNA-binding.

SEQ ID NO: 332:
YRGVRQRHWGKWVAEIRLPRNRTRLWLGTFDTAEEAALAYDSAAFRLRGE
SARLNF.

Preferably, the AP2 DNA-binding domain described above under (i) comprises at least residues LPRNRTRLWLGTFDT.

Motif XVIII (SEQ ID NO: 333) is preferably RLP K/R/Q NX¹R T/V/M R L/V WLGT F/Y D/E T/S, where X¹ is zero, one or more, and up to 30, gaps.

More preferably, Motif XVIII (SEQ ID NO: 333) is RLPRNX¹RTRLWLGTFDT, wherein X¹ is zero, one or more, and up to 30, gaps.

AP2-70 polypeptides as defined herein may also comprise one or more of the following motifs:

Motif XX/SEQ ID NO: 335: WDESESFLLHKYPSLEIDWDAILS, or a motif having in increasing order of preference at least 50%, 55%, 60%, 65%, 70%, 75%, 80%, 85%, 90%, 95% or more sequence identity to Motif XX. Adapted from Nakano et al., 2006 (CMI-1).
Motif XXI/SEQ ID NO: 336: GPPLHAAVDAKLHAICH, or a motif having in increasing order of preference at least 50%, 55%, 60%, 65%, 70%, 75%, 80%, 85%, 90%, 95% or more sequence identity to Motif XXI. Adapted from Nakano et al., 2006 (CMI-2).
Motif XXII/SEQ ID NO: 337: GANYLTPAQVLHVQAQLQRLRRP, or a motif having in increasing order of preference at least 50%, 55%, 60%, 65%, 70%, 75%, 80%, 85%, 90%, 95% or more sequence identity to Motif XXII. Adapted from Nakano et al., 2006 (CMI-3).
Motif XXIII/SEQ ID NO: 338: VDSKELMGALAPSMVSFSYPCSEQSASS, or a motif having in increasing order of preference at least 50%, 55%, 60%, 65%, 70%, 75%, 80%, 85%, 90%, 95% or more sequence identity to Motif XXIII. Adapted from Nakano et al., 2006 (CMI-4).

Any of Motifs XVIII and XX to XXIII described above may comprise one, two or three conservative and/or non-conservative change and/or deletion at any position.

The term “domain” and “motif” is defined in the “definitions” section herein. Specialist databases exist for the identification of domains. Examples are given in the “definitions” section herein.

Furthermore, AP2-70-like polypeptides (at least in their native form) typically have DNA-binding activity. Tools and techniques for measuring DNA-binding activity are well known in the art.

The present invention is illustrated by transforming plants with the nucleic acid sequence represented by SEQ ID NO: 257, encoding the polypeptide sequence of SEQ ID NO: 258. However, performance of the invention is not restricted to these sequences; the methods of the invention may advantageously be performed using any AP2-70-like-encoding nucleic acid or AP2-70-like polypeptide as defined herein.

Examples of nucleic acids encoding AP2-70-like polypeptides are given in Table 18 of Example 18 herein. Such nucleic acids are useful in performing the methods of the invention. The amino acid sequences given in Table 18 of Example 18 are example sequences of orthologues and paralogues of the AP2-70-like polypeptide represented by SEQ ID NO: 258, the terms “orthologues” and “paralogues” being as defined herein.

Nucleic acid variants may also be useful in practicing the methods of the invention. Examples of such variants include nucleic acids encoding homologues and derivatives of any one of the amino acid sequences given in Table 18 of Example 18, the terms “homologue” and “derivative” being as defined herein. Also useful in the methods of the invention are nucleic acids encoding homologues and derivatives of orthologues or paralogues of any one of the amino acid sequences given in Table 18 of Example 18. Homologues and derivatives useful in the methods of the present invention have substantially the same biological and functional activity as the unmodified protein from which they are derived.

Further nucleic acid variants useful in practicing the methods of the invention include portions of nucleic acids encoding AP2-70-like polypeptides, nucleic acids hybridising to nucleic acids encoding AP2-70-like polypeptides, splice variants of nucleic acids encoding AP2-70-like polypeptides, allelic variants of nucleic acids encoding AP2-70-like polypeptides and variants of nucleic acids encoding AP2-70-like polypeptides obtained by gene shuffling. The terms hybridising sequence, splice variant, allelic variant and gene shuffling are as described herein.

Nucleic acids encoding AP2-70-like polypeptides need not be full-length nucleic acids, since performance of the methods of the invention does not rely on the use of full-length nucleic acid sequences. According to the present invention, there is provided a method for enhancing yield-related traits in plants, comprising introducing and expressing in a plant a portion of any one of the nucleic acid sequences given in Table 18 of Example 18, or a portion of a nucleic acid encoding an orthologue, paralogue or homologue of any of the amino acid sequences given in Table 18 of Example 18.

A portion of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode an AP2-70-like polypeptide as defined herein, and have substantially the same biological activity as the amino acid sequences given in Table 18 of Example 18. Preferably, the portion is a portion of any one of the nucleic acids given in Table 18 of Example 18, or is a portion of a nucleic acid encoding an orthologue or paralogue of any one of the amino acid sequences given in Table 18 of Example 18. Preferably the portion is, in increasing order of preference at least 600, 650, 700, 750 consecutive nucleotides in length, the consecutive nucleotides being of any one of the nucleic acid sequences given in Table 18 of Example 18, or of a nucleic acid encoding an orthologue or paralogue of any one of the amino acid sequences given in Table 18 of Example 18. Most preferably the portion is a portion of the nucleic acid of SEQ ID NO: 257. Preferably, the portion encodes an amino acid sequence which when used in the construction of a phylogenetic tree, such as the one depicted in FIGS. 3 and 4, tends to cluster with the group of AP2-70-like polypeptides (Group I(A-6), specifically Group Ib: see Nakanao et al., 2006 for classification) comprising the amino acid sequence represented by SEQ ID NO: 258 rather than with any other group.

Another nucleic acid variant useful in the methods of the invention is a nucleic acid capable of hybridising, under reduced stringency conditions, preferably under stringent conditions, with a nucleic acid encoding an AP2-70-like polypeptide as defined herein, or with a portion as defined herein.

According to the present invention, there is provided a method for enhancing yield-related traits in plants, comprising introducing and expressing in a plant a nucleic acid capable of hybridizing to any one of the nucleic acids given in Table 18 of Example 18, or comprising introducing and expressing in a plant a nucleic acid capable of hybridising to a nucleic acid encoding an orthologue, paralogue or homologue of any of the nucleic acid sequences given in Table 18 of Example 18.

Hybridising sequences useful in the methods of the invention encode an AP2-70-like polypeptide as defined herein, and have substantially the same biological activity as the amino acid sequences given in Table 18 of Example 18. Preferably, the hybridising sequence is capable of hybridising to any one of the nucleic acids given in Table 18 of Example 18, or to a portion of any of these sequences, a portion being as defined above, or wherein the hybridising sequence is capable of hybridising to a nucleic acid encoding an orthologue or paralogue of any one of the amino acid sequences given in Table 18 of Example 18. Most preferably, the hybridising sequence is capable of hybridising to a nucleic acid as represented by SEQ ID NO: 257 or to a portion thereof.

Preferably, the hybridising sequence encodes an amino acid sequence which when used in the construction of a phylogenetic tree, such as the one depicted in FIGS. 3 and 4, tends to cluster with the group of AP2-70-like polypeptides (Group I(A-6), specifically Group Ib: see Nakanao et al., 2006 for classification) comprising the amino acid sequence represented by SEQ ID NO: 258 rather than with any other group.

Another nucleic acid variant useful in the methods of the invention is a splice variant encoding an AP2-70-like polypeptide as defined hereinabove, a splice variant being as defined herein.

According to the present invention, there is provided a method for enhancing yield-related traits in plants, comprising introducing and expressing in a plant a splice variant of any one of the nucleic acid sequences given in Table 18 of Example 18, or a splice variant of a nucleic acid encoding an orthologue, paralogue or homologue of any of the amino acid sequences given in Table 18 of Example 18.

Preferred splice variants are splice variants of a nucleic acid represented by SEQ ID NO: 257, or a splice variant of a nucleic acid encoding an orthologue or paralogue of SEQ ID NO: 258. Preferably, the amino acid sequence encoded by the splice variant, when used in the construction of a phylogenetic tree, such as the one depicted in FIGS. 3 and 4, tends to cluster with the group of AP2-70-like polypeptides (Group I(A-6), specifically Group Ib: see Nakanao et al., 2006 for classification) comprising the amino acid sequence represented by SEQ ID NO: 258 rather than with any other group.

Another nucleic acid variant useful in performing the methods of the invention is an allelic variant of a nucleic acid encoding an AP2-70-like polypeptide as defined hereinabove, an allelic variant being as defined herein.

According to the present invention, there is provided a method for enhancing yield-related traits in plants, comprising introducing and expressing in a plant an allelic variant of any one of the nucleic acids given in Table 18 of Example 18, or comprising introducing and expressing in a plant an allelic variant of a nucleic acid encoding an orthologue, paralogue or homologue of any of the amino acid sequences given in Table 18 of Example 18.

The allelic variants useful in the methods of the present invention have substantially the same biological activity as the AP2-70-like polypeptide of SEQ ID NO: 258 and any of the amino acids depicted in Table 18 of Example 18. Allelic variants exist in nature, and encompassed within the methods of the present invention is the use of these natural alleles. Preferably, the allelic variant is an allelic variant of SEQ ID NO: 257 or an allelic variant of a nucleic acid encoding an orthologue or paralogue of SEQ ID NO: 258. Preferably, the amino acid sequence encoded by the allelic variant, when used in the construction of a phylogenetic tree, such as the one depicted in FIGS. 3 and 4, tends to cluster with the AP2-70-like polypeptides (Group I(A-6), specifically Group Ib: see Nakanao et al., 2006 for classification) comprising the amino acid sequence represented by SEQ ID NO: 258 rather than with any other group.

Gene shuffling or directed evolution may also be used to generate variants of nucleic acids encoding AP2-70-like polypeptides as defined above; the term “gene shuffling” being as defined herein.

According to the present invention, there is provided a method for enhancing yield-related traits in plants, comprising introducing and expressing in a plant a variant of any one of the nucleic acid sequences given in Table 18 of Example 18, or comprising introducing and expressing in a plant a variant of a nucleic acid encoding an orthologue, paralogue or homologue of any of the amino acid sequences given in Table 18 of Example 18, which variant nucleic acid is obtained by gene shuffling.

Preferably, the amino acid sequence encoded by the variant nucleic acid obtained by gene shuffling, when used in the construction of a phylogenetic tree such as the one depicted in FIGS. 3 and 4, tends to cluster with the group of AP2-70-like polypeptides (Group I(A-6), specifically Group Ib: see Nakanao et al., 2006 for classification) comprising the amino acid sequence represented by SEQ ID NO: 258 rather than with any other group.

Furthermore, nucleic acid variants may also be obtained by site-directed mutagenesis. Several methods are available to achieve site-directed mutagenesis, the most common being PCR based methods (Current Protocols in Molecular Biology. Wiley Eds.).

Nucleic acids encoding AP2-70-like polypeptides may be derived from any natural or artificial source. The nucleic acid may be modified from its native form in composition and/or genomic environment through deliberate human manipulation. Preferably the AP2-70-like polypeptide-encoding nucleic acid is from a plant, further preferably from a monocotyledonous plant, more preferably from the family poaceae, most preferably the nucleic acid is from Oryza sativa.

Performance of the methods of the invention gives plants having enhanced yield-related traits. In particular performance of the methods of the invention gives plants having increased yield, especially increased seed yield relative to control plants. The terms “yield” and “seed yield” are described in more detail in the “definitions” section herein.

The present invention provides a method for increasing yield, especially seed yield of plants, relative to control plants, which method comprises modulating expression, preferably increasing expression, in a plant of a nucleic acid encoding an AP2-70-like polypeptide as defined herein.

According to a preferred feature of the present invention, performance of the methods of the invention gives plants having an increased growth rate relative to control plants. Therefore, according to the present invention, there is provided a method for increasing the growth rate of plants, which method comprises modulating expression, preferably increasing expression, in a plant of a nucleic acid encoding an AP2-70-like polypeptide as defined herein.

Performance of the methods of the invention gives plants grown under non-stress conditions or under mild drought conditions increased yield relative to suitable control plants grown under comparable conditions. Therefore, according to the present invention, there is provided a method for increasing yield in plants grown under non-stress conditions or under mild drought conditions, which method comprises increasing expression in a plant of a nucleic acid encoding an AP2-70-like polypeptide.

Performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises increasing expression in a plant of a nucleic acid encoding an AP2-70-like polypeptide. Nutrient deficiency may result from a lack or excess of nutrients such as nitrogen, phosphates and other phosphorous-containing compounds, potassium, calcium, cadmium, magnesium, manganese, iron and boron, amongst others.

The present invention encompasses plants or parts thereof (including seeds) obtainable by the methods according to the present invention. The plants or parts thereof comprise a nucleic acid transgene encoding an AP2-70-like polypeptide as defined above.

The invention also provides genetic constructs and vectors to facilitate introduction and/or expression in plants of nucleic acids encoding AP2-70-like polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(d) a nucleic acid encoding an AP2-70-like polypeptide as defined above;
(e) one or more control sequences capable of driving expression of the nucleic acid sequence of (a); and optionally
(f) a transcription termination sequence.

Preferably, the nucleic acid encoding an AP2-70-like polypeptide is:
(i) a nucleic acid represented by SEQ ID NO: 257;
(ii) the complement of a nucleic acid represented by SEQ ID NO: 257;
(iv) a nucleic acid encoding an AP2-70-like polypeptide having, in increasing order of preference, at least 50%, 55%, 60%, 65%, 70%, 75%, 80%, 85%, 90%, 95%, 96%, 97%, 98%, 99% or more sequence identity to the amino acid sequence represented by SEQ ID NO: 258, and having in increasing order of preference at least 50%, 55%, 60%, 65%, 70%, 75%, 80%, 85%, 90%, 95%, 96%, 97%, 98%, 99% or more sequence identity to SEQ ID NO: 331: PFLMQWLNLLPLPVLDSSSWCPEHFHNSESDALP (which represents the C-terminal region of SEQ ID NO: 258).

The term “control sequence” and “termination sequence” are as defined herein.

Plants are transformed with a vector comprising any of the nucleic acids described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence. A constitutive promoter is particularly useful in the methods. See the “Definitions” section herein for definitions of the various promoter types. Also useful in the methods of the invention is a root-specific promoter.

It should be clear that the applicability of the present invention is not restricted to the AP2-70-like polypeptide-encoding nucleic acid represented by SEQ ID NO: 257, nor is the applicability of the invention restricted to expression of an AP2-70-like polypeptide-encoding nucleic acid when driven by a constitutive promoter, or when driven by a root-specific promoter.

The constitutive promoter is preferably a GOS2 promoter, preferably a GOS2 promoter from rice. Further preferably the constitutive promoter is represented by a nucleic acid sequence substantially similar to SEQ ID NO: 39 or SEQ ID NO: 339 most preferably the constitutive promoter is as represented by SEQ ID NO: 39 or SEQ ID NO: 339. See Table 2a in the “Definitions” section herein for further examples of constitutive promoters.

According to another preferred feature of the invention, the nucleic acid encoding an AP2-70-like polypeptide is operably linked to a root-specific promoter. The root-specific promoter is preferably an RCc3 promoter (Plant Mol Biol. 1995 January; 27(2):237-48), more preferably the RCc3 promoter is from rice, further preferably the RCc3 promoter is represented by a nucleic acid sequence substantially similar to SEQ ID NO: 110 or SEQ ID NO: 340, most preferably the promoter is as represented by SEQ ID NO: 110. Examples of other root-specific promoters which may also be used to perform the methods of the invention are shown in Table 2b herein.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein.

The invention also provides a method for the production of transgenic plants having enhanced yield-related traits relative to control plants, comprising introduction and expression in a plant of any nucleic acid encoding an AP2-70-like polypeptide as defined hereinabove.

More specifically, the present invention provides a method for the production of transgenic plants having increased enhanced yield-related traits, particularly increased (seed) yield, which method comprises:
(iii) introducing and expressing in a plant or plant cell an AP2-70-like polypeptide-encoding nucleic acid; and
(iv) cultivating the plant cell under conditions promoting plant growth and development.

The nucleic acid of (i) may be any of the nucleic acids capable of encoding an AP2-70-like polypeptide as defined herein. Preferably, the nucleic acid is:
(i) a nucleic acid represented by SEQ ID NO: 257;
(ii) the complement of a nucleic acid represented by SEQ ID NO: 257;
(iii) a nucleic acid encoding an AP2-70-like polypeptide having, in increasing order of preference, at least 50%, 55%, 60%, 65%, 70%, 75%, 80%, 85%, 90%, 95%, 96%, 97%, 98%, 99% or more sequence identity to the amino acid sequence represented by SEQ ID NO: 258, and having in increasing order of preference at least 50%, 55%, 60%, 65%, 70%, 75%, 80%, 85%, 90%, 95%, 96%, 97%, 98%, 99% or more sequence identity to SEQ ID NO: 331: PFLMQWLNLLPLPVLDSSSWCPEHFHNSESDALP (which represents the C-terminal region of SEQ ID NO: 258).

The nucleic acid may be introduced directly into a plant cell or into the plant itself (including introduction into a tissue, organ or any other part of a plant). According to a preferred feature of the present invention, the nucleic acid is preferably introduced into a plant by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Höfgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding a AP2-70-like polypeptide as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

The invention also extends to harvestable parts of a plant such as, but not limited to seeds, leaves, fruits, flowers, stems, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art. Examples are given in the “definitions” section herein.

As mentioned above, a preferred method for modulating (preferably, increasing) expression of a nucleic acid encoding an AP2-70-like polypeptide is by introducing and expressing in a plant a nucleic acid encoding an AP2-70-like polypeptide; however the effects of performing the method, i.e. enhancing yield-related traits may also be achieved using other well known techniques. A description of some of these techniques can be found in the “definitions” section.

The effects of the invention may also be reproduced using the technique of T-DNA activation or TILLING (Targeted Induced Local Lesions In Genomes); for a description of the same see the “definitions” section.

The effects of the invention may also be reproduced using homologous recombination; for a description of the same see the “definitions” section.

The present invention also encompasses use of nucleic acids encoding AP2-70-like polypeptides as described herein and use of these AP2-70-like polypeptide in enhancing any of the aforementioned yield-related traits in plants.

Nucleic acids encoding AP2-70-like polypeptide described herein, or the AP2-70-like polypeptides themselves, may find use in breeding programmes in which a DNA marker is identified which may be genetically linked to an AP2-70-like polypeptide-encoding gene. The nucleic acids/genes, or the AP2-70-like polypeptides themselves may be used to define a molecular marker. This DNA or protein marker may then be used in breeding programmes to select plants having enhanced yield-related traits as defined hereinabove in the methods of the invention.

Allelic variants of an AP2-70-like polypeptide-encoding nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acids encoding AP2-70-like polypeptides may also be used as probes for genetically and physically mapping the genes that they are a part of, and as markers for traits linked to those genes. Such information may be useful in plant breeding in order to develop lines with desired phenotypes. Such use of AP2-70-like polypeptide-encoding nucleic acids requires only a nucleic acid sequence of at least 15 nucleotides in length. The AP2-70-like polypeptide-encoding nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the AP2-70-like-encoding nucleic acids. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the AP2-70-like polypeptide-encoding nucleic acid in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

The methods according to the present invention result in plants having enhanced yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

### DESCRIPTION OF FIGURES

The present invention will now be described with reference to the following figures in which:

FIG. 1 shows a phylogenetic tree taken from Ooka et al., 2003 (DNA Research 10, 239-247). The dashed box 3^{rd} from the bottom, marked “OsNAC7”, is the group comprising the sequence of SEQ ID NO: 2. The cluster of NACs starting from ONAC022 until OsNAC3, represent the cluster comprising the sequences of SEQ ID NO: 51, SEQ ID NO: 53, SEQ ID NO: 55, SEQ ID NO: 57 and SEQ ID NO: 59. Amino acid sequences clustering in these groups (the amino acid sequences given in Tables 3 and 4, for example) are considered to be useful in the methods of the invention.

FIG. 2 shows the sequence of SEQ ID NO: 132 with (A): Putative nuclear localisation signal, (B): AP2/ERF DNA binding domain, (1): CMVII-1 motif, (3): CMVII-3 motif, (4): CMVII-4 motif, (5): CMVII-5 motif, (6): CMVII-6 motif, (7): CMVII-7 motif, (8): CMVII-8 motif. The CMVII motifs were identified according to Nakano et al. (2006).

FIG. 3 shows a phylogenetic tree taken from Nakano et al. (Plant Physiol. Vol. 140, 2006) in which the group indicated as I(A-6) includes the group of AP2-70-like polypeptides defined herein.

FIG. 4 shows a section of a phylogenetic tree comprising AP2-70-like polypeptide sequences defined herein and expanding the group indicated as Group I(A-6) in Nakano et al., 2006. The phylogenetic tree was constructed using a neighbour-joining clustering algorithm as provided in the AlignX programme from the Vector NTI (Invitrogen).

FIG. 5 shows an alignment of NAC transcription factors as defined hereinabove. The sequences were aligned using AlignX program from Vector NTI suite (InforMax, Bethesda, Md.). Multiple alignment was done with a gap opening penalty of 10 and a gap extension of 0.01. Minor manual editing was also carried out where necessary to better position some conserved regions. The NAC domain and Motifs I to III are indicated.

FIG. 6 shows an alignment of NAC transcription factors as defined hereinabove. The sequences were aligned using AlignX program from Vector NTI suite (InforMax, Bethesda, Md.). Multiple alignment was done with a gap opening penalty of 10 and a gap extension of 0.01. Minor manual editing was also carried out where necessary to better position some conserved regions. The NAC domain and Motifs IV to XI are indicated.

FIG. 7 shows a CLUSTAL W multiple sequence alignment of AP2-2 polypeptides from Arabidopsis and rice. SEQ ID NO: 132 (Os06g09390) is indicated in bold and the conserved regions (motifs XII to XVII, SEQ ID NO: 133 to SEQ ID NO: 138) are underlined.

FIG. 8 shows an alignment of AP2-70-like polypeptides with Motifs XVIII and XIX indicated and with the AP2 DNA-binding domain also indicated.

FIG. 9 shows a binary vector p163, for increased expression in Oryza sativa of an Oryza sativa NAC transcription factor-encoding nucleic acid under the control of a GOS2 promoter (internal reference PRO0129).

FIG. 10 shows a binary vector p164, for increased expression in Oryza saliva of an Oryza saliva NAC1 (SEQ ID NO: 50) transcription factor-encoding nucleic acid under the control of a GOS2 promoter (internal reference PRO0129) for constitutive expression.

FIG. 11 shows a binary vector p165, for increased expression in Oryza saliva of an Oryza saliva NAC4 (SEQ ID NO: 52) transcription factor-encoding nucleic acid under the control of a GOS2 promoter (internal reference PRO0129) for constitutive expression.

FIG. 12 shows a binary vector p166, for increased expression in Oryza saliva of an Oryza saliva NAC4 (SEQ ID NO: 52) transcription factor-encoding nucleic acid under the control of an RCc3 promoter (internal reference PRO0110) for root-specific expression.

FIG. 13 shows a binary vector p167, for increased expression in Oryza sativa of an Oryza sativa NAC transcription factor-encoding nucleic acid under the control of a protochlorophylid reductase promoter (internal reference PRO0123).

FIG. 14 shows a binary vector p167, for increased expression in Oryza saliva of an Oryza sativa NAC6 (SEQ ID NO: 54) transcription factor-encoding nucleic acid under the control of an RCc3 promoter (internal reference PRO0110) for root-specific expression.

FIG. 15 shows a binary vector p168, for increased expression in Oryza saliva of an Oryza saliva NAC7 (SEQ ID NO: 56) transcription factor-encoding nucleic acid under the control of a GOS2 promoter (internal reference PRO0129) for constitutive expression.

FIG. 16 shows a binary vector p169, for increased expression in Oryza saliva of an Oryza saliva NAC3 (SEQ ID NO: 58) transcription factor-encoding nucleic acid under the control of an RCc3 promoter (internal reference PRO0110) for root-specific expression.

FIG. 17 shows a binary vector pGOS2::NAC1, for increased expression in Oryza saliva of an Oryza sativa NAC1 (SEQ ID NO: 50) transcription factor-encoding nucleic acid under the control of a GOS2 promoter for constitutive expression.

FIG. 18 shows a binary vector pGOS2::NAC4, for increased expression in Oryza saliva of an Oryza saliva NAC4 (SEQ ID NO: 52) transcription factor-encoding nucleic acid under the control of a GOS2 promoter for constitutive expression.

FIG. 19 shows a binary vector pRCc3::NAC4, for increased expression in Oryza sativa of an Oryza sativa NAC4 (SEQ ID NO: 52) transcription factor-encoding nucleic acid under the control of an RCc3 promoter for root-specific expression.

FIG. 20 shows a binary vector pRCc3::NAC6, for increased expression in Oryza saliva of an Oryza saliva NAC6 (SEQ ID NO: 54) transcription factor-encoding nucleic acid under the control of an RCc3 promoter for root-specific expression.

FIG. 21 shows a binary vector pGOS2::NAC7, for increased expression in Oryza sativa of an Oryza sativa NAC7 (SEQ ID NO: 56) transcription factor-encoding nucleic acid under the control of a GOS2 promoter for constitutive expression.

FIG. 22 shows a binary vector pRCc3::NAC3, for increased expression in Oryza saliva of an Oryza saliva NAC3 (SEQ ID NO: 58) transcription factor-encoding nucleic acid under the control of an RCc3 promoter for root-specific expression.

FIG. 23 shows the binary vector for increased expression in Oryza sativa of a rice AP2-2 protein-encoding nucleic acid under the control of a GOS2 promoter.

FIG. 24 shows the binary vector for increased expression in Oryza saliva of an Oryza sativa AP2-70-like protein-encoding nucleic acid under the control of a rice RCC3 promoter (pRCC3).

FIG. 25 shows the binary vector for increased expression in Oryza saliva of an Oryza sativa AP2-70-like protein-encoding nucleic acid under the control of a rice GOS2 promoter (pGOS2).

FIGS. 26 to 29 details examples of sequences useful in performing the methods according to the present invention.

### EXAMPLES

The present invention will now be described with reference to the following examples, which are by way of illustration alone. The following examples are not intended to completely define or otherwise limit the scope of the invention.

### A) OsNAM2

### Example 1

### Cloning of OsNAM2 cDNA

Total cellular RNA from 2-week-old rice seedlings was used to clone OsNAM2 cDNA. RNA was extracted using an RNeasy Kit (Qiagen, Germany). OsNAM2 cDNA was 1292 bp long, from 5′ UTR (untranslated region) to the stop codon. Two overlapping cDNAs were joined in the OsNAM2 cDNA clone. A 5′ cDNA of 1025 bp was obtained by 5′ RACE-PCR using an adapter primer (5′-AAGCAGTGGTATCAACGCAGAGTACGCGGG-3′) and the OsNAM2 specific primer (OsNAM2-2) (5′-CTC TCC AGA GGC GGC ATC ATG TCG GA-3′). The 5′ RACE-PCR was performed with the BD SMART™ RACE cDNA Amplification Kit (Clontech, USA). The adapter primer (SMART II A Oligonucleotide) was as provided by the manufacturer. The 3′ cDNA of 620 bp was obtained by PCR with two OsNAM2-specific primers (NAM2-1 (5′-TGA TCG GGA TGA GGA AGA C-3′) and NAM2-3 (5′-GAT CAG TCT CGG TCA TCG ATG-3′)). The first-stranded cDNA as PCR template was synthesized with the Oligotex mRNA kit (Qiagen, Valencia, Calif.). Candidate PCR products were gel-purified, subcloned into the pGEM-T Easy (Promega, USA), and confirmed by sequencing. The vector harboring the 5′ cDNA was cut with HindIII and ligated with a HindIII fragment of the 3′ cDNA vector. The result was a 1292 bp OsNAM2 cDNA.

### Example 2

### Vector Construction

### 2.1 OsNAM2 Under the Control of a GOS2 Promoter

The entry clone was subsequently used in an LR reaction with a destination vector used for Oryza sativa transformation. This vector contains as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 39 or SEQ ID NO: 339) for constitutive expression (internal reference PRO0129) was located upstream of this Gateway cassette.

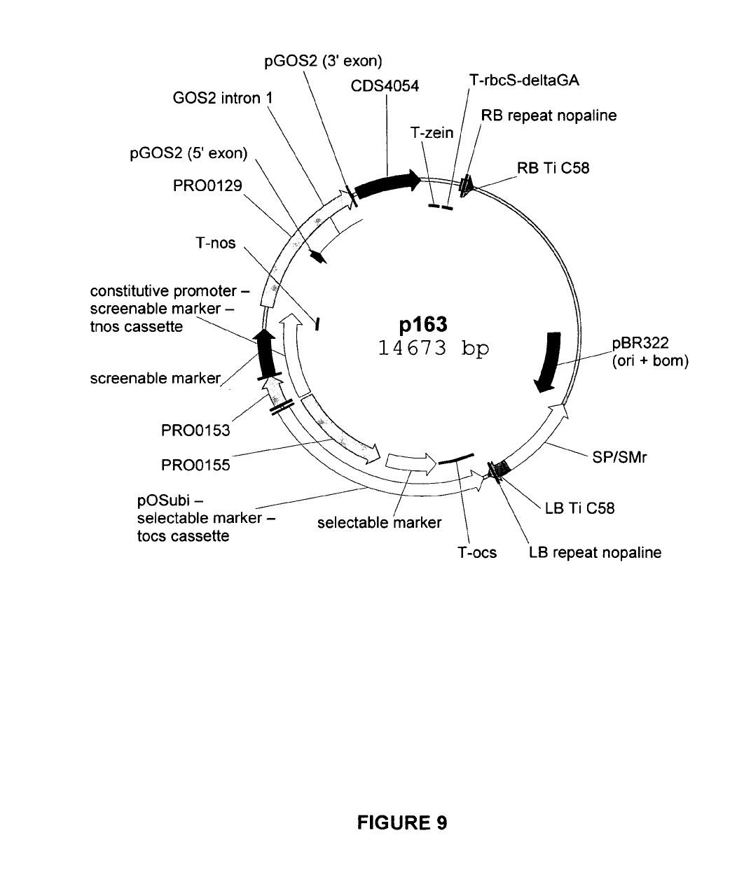

After the LR recombination step, the resulting expression vector p163 (FIG. 9) was transformed into Agrobacterium strain LBA4044 and subsequently to Oryza sativa plants. Transformed rice plants were allowed to grow and were then examined for the parameters described below.

### 2.2 OsNAM2 Under the Control of a Protochlorophylid Reductase Promoter

The entry clone was subsequently used in an LR reaction with a destination vector used for Oryza sativa transformation. This vector contains as functional elements within the T-DNA borders: a plant selectable marker; a screenable marker expression cassette; and a Gateway cassette intended for LR in vivo recombination with the nucleic acid sequence of interest already cloned in the entry clone. A protochlorophylid reductase promoter (SEQ ID NO: 40) for green tissue-specific expression (internal reference PRO0123) was located upstream of this Gateway cassette.

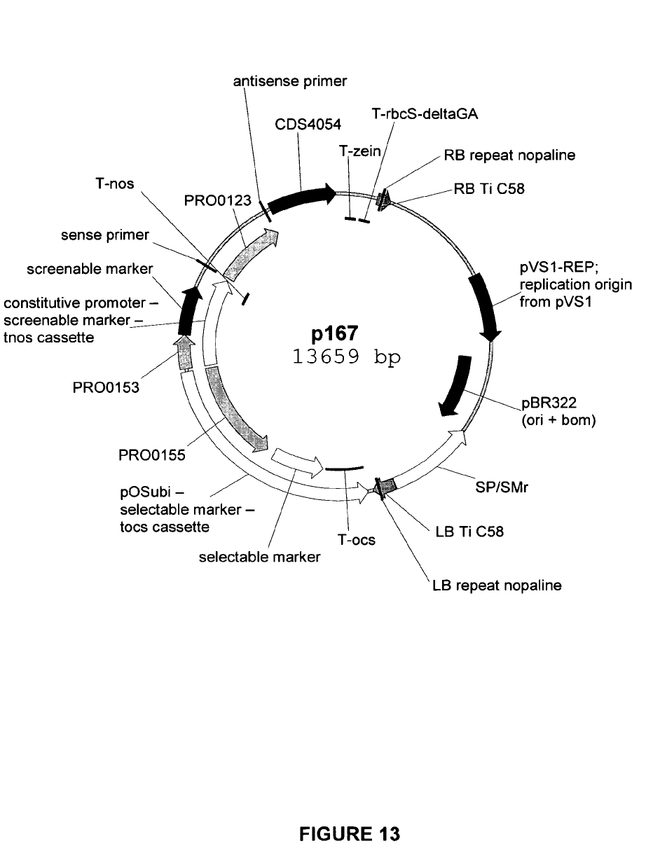

After the LR recombination step, the resulting expression vector p167 (FIG. 13) was transformed into Agrobacterium strain LBA4044 and subsequently to Oryza sativa plants. Transformed rice plants were allowed to grow and were then examined for the parameters described below.

### Example 3

### Evaluation Procedure

### 3.1 Evaluation Setup

Approximately 30 independent T0 rice transformants were generated. The primary transformants were transferred from a tissue culture chamber to a greenhouse for growing and harvest of T1 seed. Seven events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%. From the stage of sowing until the stage of maturity the plants were passed several times through a digital imaging cabinet. At each time point digital images (2048×1536 pixels, 16 million colours) were taken of each plant from at least 6 different angles.

### 3.2 Statistical Analysis: T-Test and F-Test

A two factor ANOVA (analysis of variants) was used as a statistical model for the overall evaluation of plant phenotypic characteristics. An F-test was carried out on all the parameters measured of all the plants of all the events transformed with the gene of the present invention. The F-test was carried out to check for an effect of the gene over all the transformation events and to verify an overall effect of the gene, also known as a global gene effect. The threshold for significance for a true global gene effect was set at a 5% probability level for the F-test. A significant F-test value points to a gene effect, meaning that it is not only the mere presence or position of the gene that is causing the differences in phenotype.

To check for an effect of the genes within an event, i.e., for a line-specific effect, a t-test was performed within each event using data sets from the transgenic plants and the corresponding null plants. “Null plants” or “null segregants” or “nullizygotes” are the plants treated in the same way as the transgenic plant, but from which the transgene has segregated. Null plants can also be described as the homozygous negative transformed plants. The threshold for significance for the t-test is set at 10% probability level. The results for some events can be above or below this threshold. This is based on the hypothesis that a gene might only have an effect in certain positions in the genome, and that the occurrence of this position-dependent effect is not uncommon. This kind of gene effect is also named herein a “line effect of the gene”. The p-value is obtained by comparing the t-value to the t-distribution or alternatively, by comparing the F-value to the F-distribution. The p-value then gives the probability that the null hypothesis (i.e., that there is no effect of the transgene) is correct.

### Example 4

### Evaluation Results

### 4.1 OsNAM2 Under the Control of a GOS2 Promoter

There was an increase in aboveground area, number of panicles, the number of flowers per panicle and the total number of seeds for transgenic plants expressing an OsNAM2 gene under the control of a GOS2 promoter compared to nullizygotes. There was a significant increase in the number of flowers per panicle and the total number of seeds for transgenic plants compared to nullizygotes. There was a >12% increase (average for 4 events) in the number of flowers per panicle for transgenic plants compared to nullizygotes, with a significant p-value from the F-test of <0.0014. Furthermore, there was a 35% increase in the total number of seeds for transgenic plants compared to nullizygotes, with a significant p-value from the F-test of <0.0045.

### 4.2 OsNAM2 Under the Control of a Protochlorophylid Reductase Promoter

There was an increase in aboveground area, number of panicles and the total number of seeds for transgenic plants expressing an OsNAM2 gene under the control of a protochlorophylid reductase promoter compared to nullizygotes. There was a >16% increase (average for 2 events) in the aboveground area for transgenic plants compared to nullizygotes, with a significant p-value from the F-test of <0.0014. There was a >16% increase in the number of panicles for transgenic plants compared to nullizygotes and a >18% increase in the total number of seeds for transgenic plants compared to nullizygotes.

### B) OsNAC1, 3, 4, 6, and 7

### Example 5

### Cloning of OsNAC1, 3, 4, 6, and 7

Oryza sativa genes containing a NAC domain, OsNAC1 (Gene bank accession number AK067690), OsNAC3 (AK069257), OsNAC4 (AK068392), OsNAC6 (AK102475) and OsNAC7 (AK107330), were identified to be stress-inducible in rice by expression profiling conducted with the 60K Rice Whole Genome Microarray (GreenGene Biotech Inc., Korea). This microarray contained 70-mer oligonucleotide probes with sequences corresponding to 58,417 known or predicted ORFs covering the entire rice genome (Oh et al., 2005 Plant Physiology 138: 341-351). The full-length cDNAs of OsNAC1 and OsNAC3 were obtained from rice EST stocks generated by GreenGene Biotech Inc. These cDNAs were inserted into EcoR1 and Xho1 sites of Bluescript SKII (Stratagene). The full-length cDNAs of OsNAC4, OsNAC6 and OsNAC7 were amplified by PCR using as template an Oryza sativa seedling cDNA library (GreenGene Biotech, Korea). After reverse transcription of RNA extracted from 14-day-old seedlings that were treated with 400 mM NaCl for 6 hours, the cDNAs were cloned into Uni-ZAP XR (Stratagene). Average insert size of the library was 1.5 kb and original number of plaques was of the order of 1.106 pfu. Original titre was determined to be 2.109 pfu/ml after first amplification of 2.106 pfu/ml. 0.1 μg of cDNA library was used in each 50 μl PCR mixture. PCR primers that were used for the amplification of the OsNAC genes are listed in Table 7. PCR amplifications were performed using Pfu DNA polymerase in standard conditions. The DNA fragments were amplified and purified also using standard methods. PCR products were ligated into EcoR1site of pBluescript SKII and sequenced.

TABLE 7
List of primers used for isolation of OsNACs.

| | | |
|---|---|---|
| Gene | Forward Primers | Reverse Primers |

| | | |
|---|---|---|
| OsNAC4 | 5′CCAACACTAGTAGGATAAA | 5′ACCACTGGGCTAATTAATT |
| | G3′ | A3′ |
| OsNAC6 | 5′GGTCGACCCACGCGTCCGC | 5′AACTGAAGTACCCGTTCTT |
| | T3′ | A3′ |
| OsNAC7 | 5′ATCCTCCACAAGAGAAACT | 5′AGTACAGTGTAGCACAATA |
| | A3′ | A3′ |

### Example 6

### Expression Vector Construction

### 6.1 GOS2 Constructs

The entry clone was subsequently used in an LR reaction with a destination vector used for Oryza sativa transformation. This vector contains as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 39) for constitutive expression (internal reference PRO0129) was located upstream of this Gateway cassette.

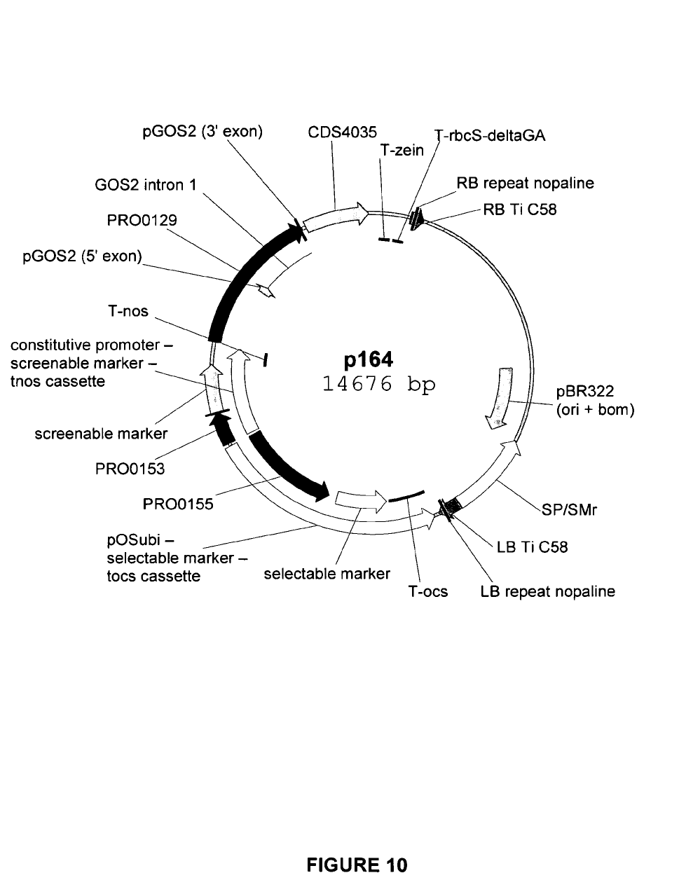

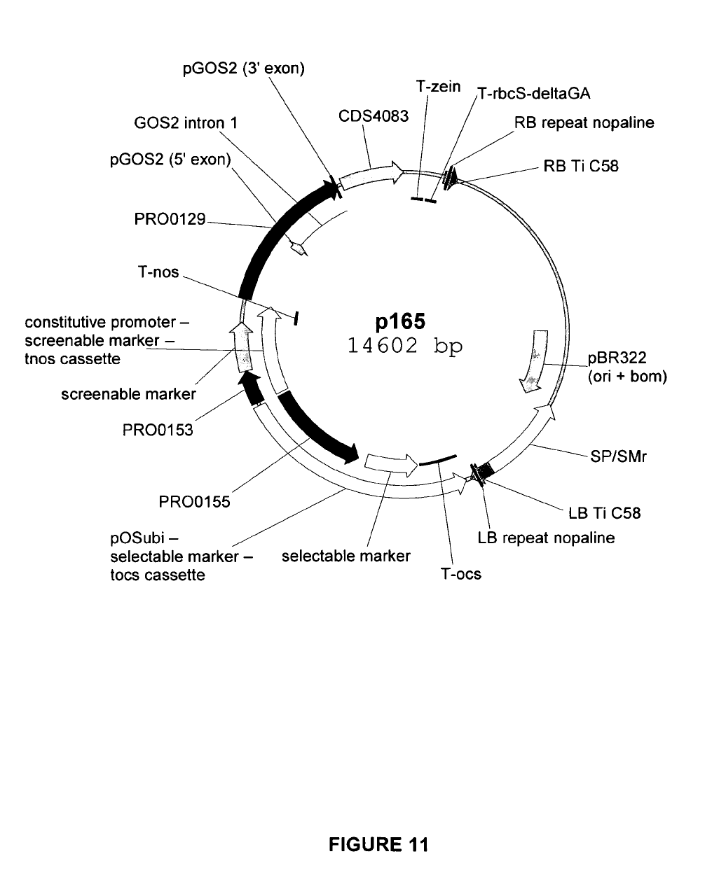

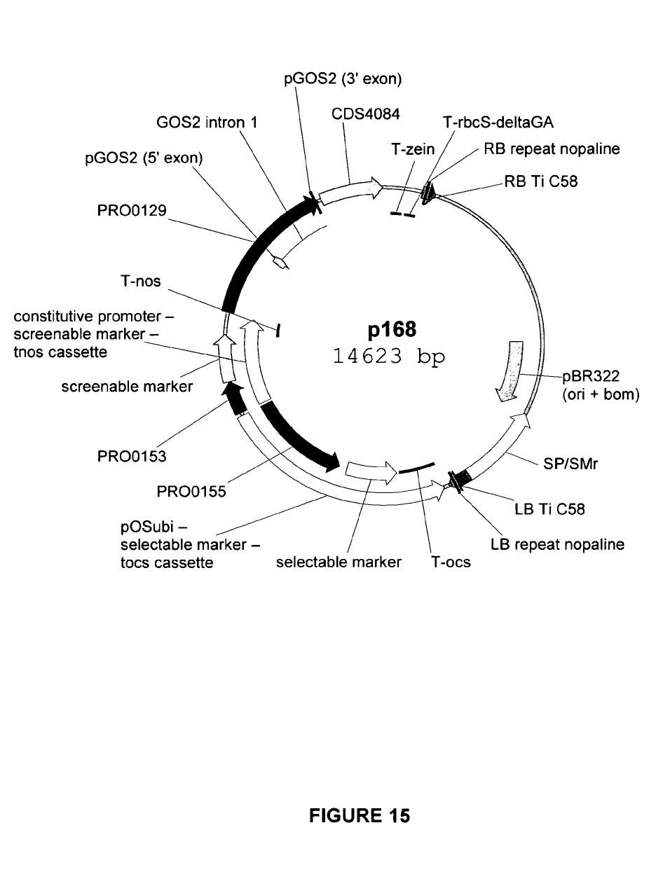

After the LR recombination step, the resulting expression vectors p164 (FIG. 10), p165 (FIG. 11) and p168 (FIG. 15) were each transformed into Agrobacterium strain LBA4044 and subsequently to Oryza sativa plants. Transformed rice plants were allowed to grow and were then examined for the parameters described below.

### 6.2 RCc3 Constructs

The entry clone was subsequently used in an LR reaction with a destination vector used for Oryza sativa transformation. This vector contains as functional elements within the T-DNA borders: a plant selectable marker; a screenable marker expression cassette; and a Gateway cassette intended for LR in vivo recombination with the nucleic acid sequence of interest already cloned in the entry clone. A rice RCc3 promoter (SEQ ID NO: 110) for root-specific expression (internal reference PRO0110) was located upstream of this Gateway cassette.

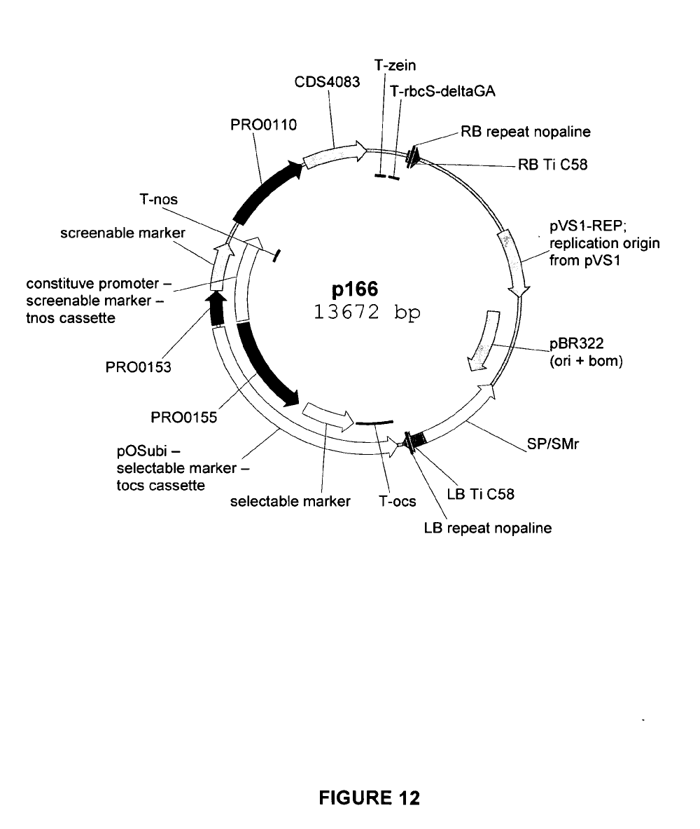

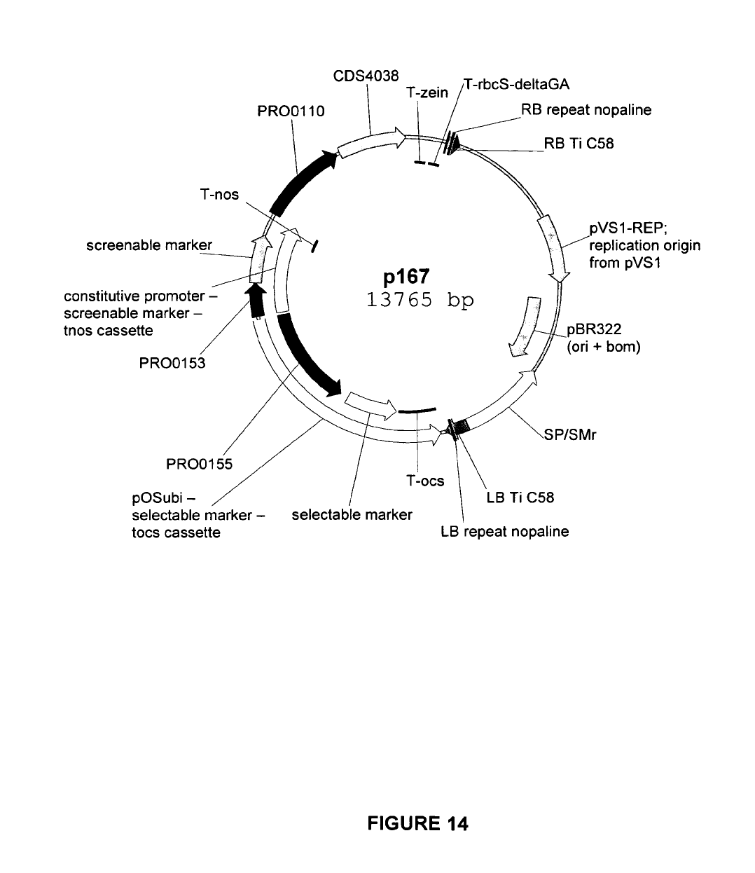

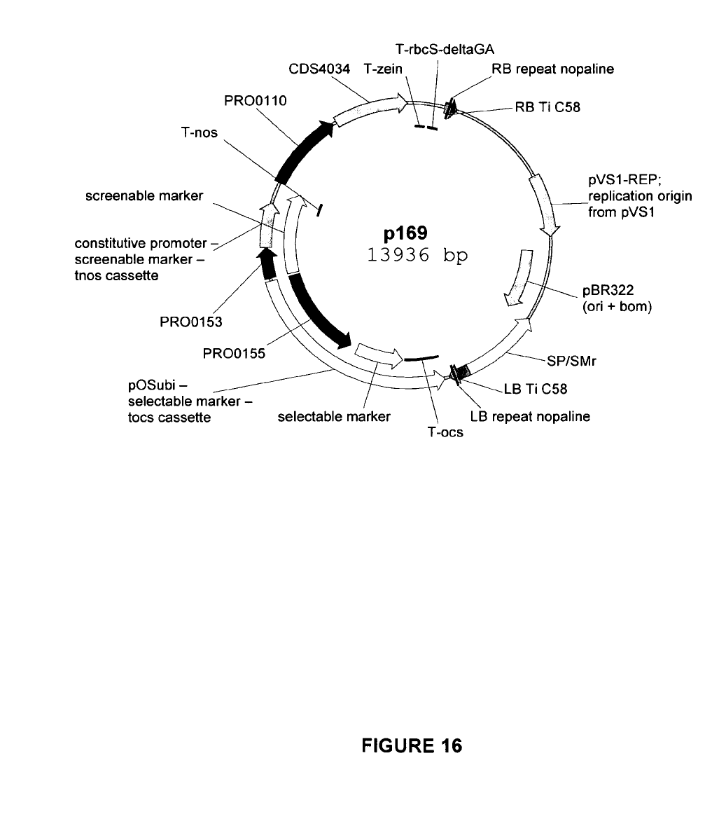

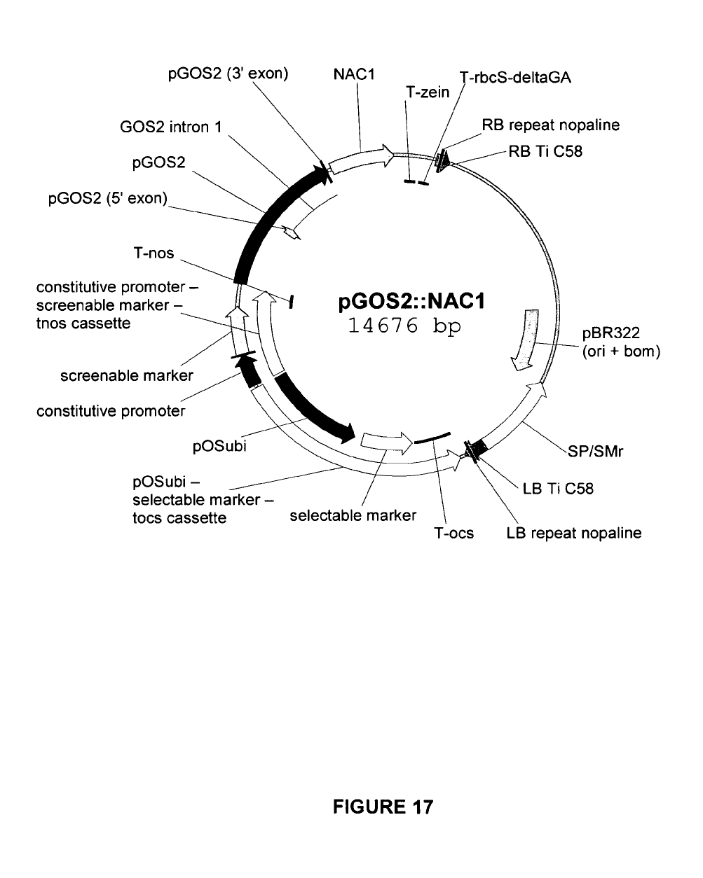

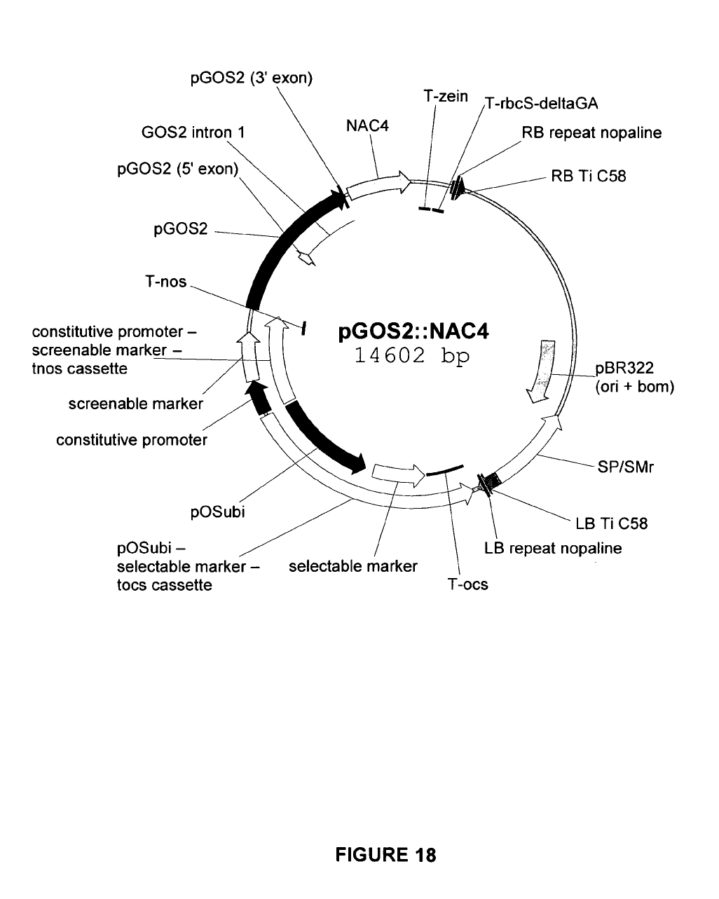

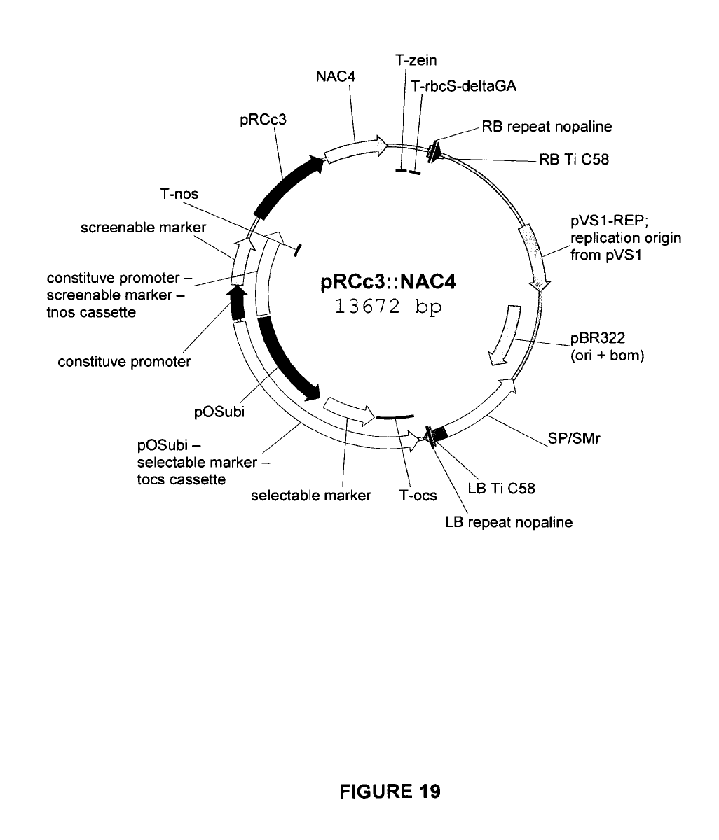

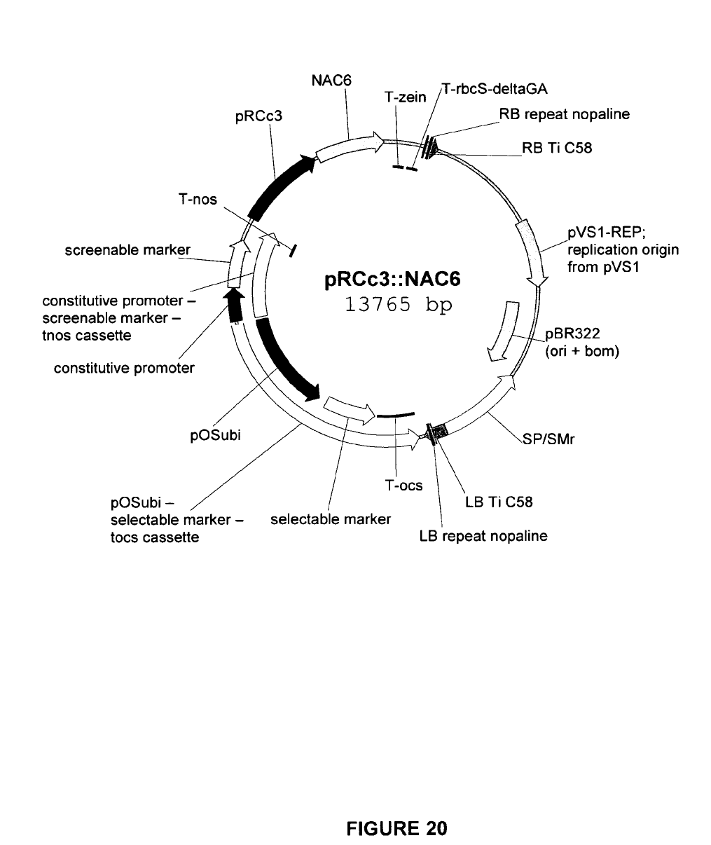

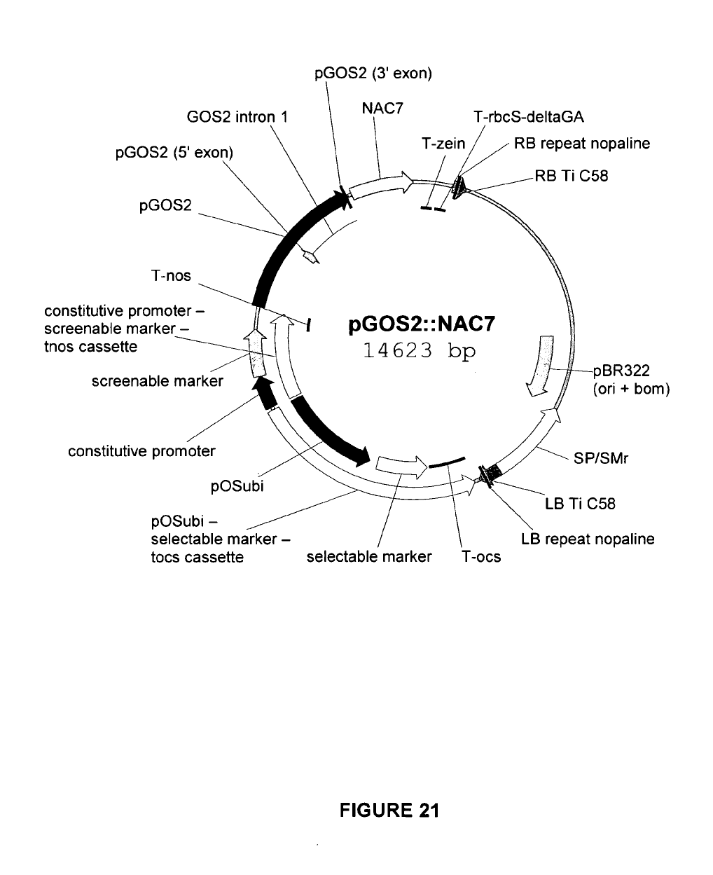

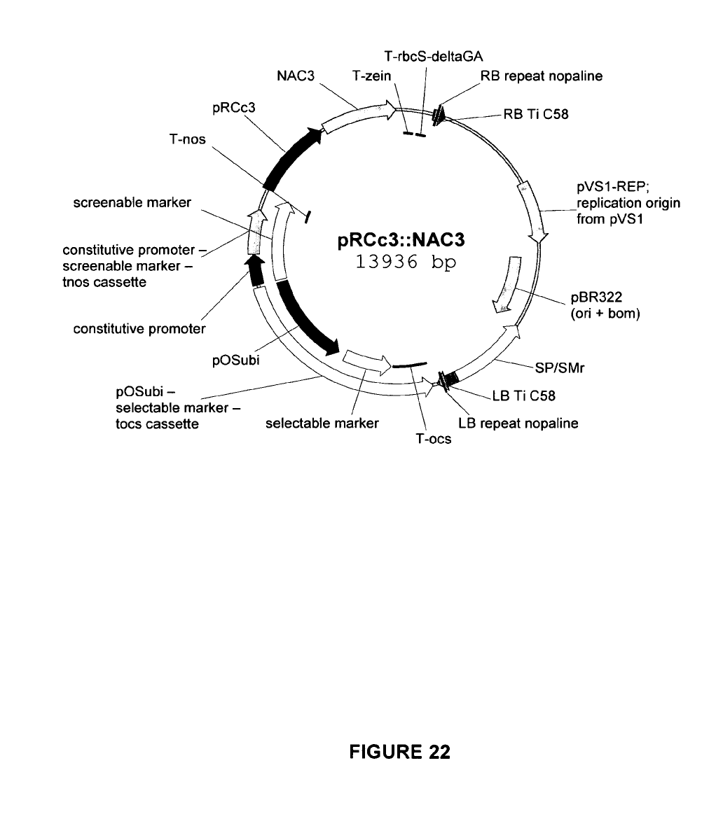

After the LR recombination step, the resulting expression vector p166 (FIG. 12), p167 (FIG. 14) and p169 (FIG. 16) were each transformed into Agrobacterium strain LBA4044 and subsequently to Oryza sativa plants. Transformed rice plants were allowed to grow and were then examined for the parameters described below.

### Example 7

### Evaluation Procedure

### 7.1 Evaluation Setup

Approximately 30 independent T0 rice transformants were generated. The primary transformants were transferred from a tissue culture chamber to a greenhouse for growing and harvest of T1 seed. Seven events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%. From the stage of sowing until the stage of maturity the plants were passed several times through a digital imaging cabinet. At each time point digital images (2048×1536 pixels, 16 million colours) were taken of each plant from at least 6 different angles.

### Drought Screen

Plants from five events (T2 seeds) were grown in potting soil under normal conditions until they approached the heading stage. They were then transferred to a “dry” section where irrigation was withheld. Humidity probes were inserted in randomly chosen pots to monitor the soil water content (SWC). When SWC went below certain thresholds, the plants were automatically re-watered continuously until a normal level was reached again. The plants were then re-transferred again to normal conditions. The rest of the cultivation (plant maturation, seed harvest) was the same as for plants not grown under abiotic stress conditions. A confirmation round was performed consisting of repeating the screen with T2 seeds not harvested from plants of the first drought screen, but from plants grown under normal conditions.

### Parameters Measured

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The Areamax is the above ground area at the time point at which the plant had reached its maximal leafy biomass.

The mature primary panicles were harvested, bagged, barcode-labelled and then dried for three days in the oven at 37° C. The panicles were then threshed and all the seeds collected. The filled husks were separated from the empty ones using an air-blowing device. After separation, both seed lots were then counted using a commercially available counting machine. The empty husks were discarded. The filled husks were weighed on an analytical balance and the cross-sectional area of the seeds was measured using digital imaging. This procedure resulted in the set of the following seed-related parameters:

The flowers-per-panicle estimates the average number of florets per panicle on a plant, derived from the number of total seeds divided by the number of first panicles. The tallest panicle and all the panicles that overlapped with the tallest panicle when aligned vertically, were considered as first panicles and were counted manually. The number of filled seeds was determined by counting the number of filled husks that remained after the separation step. The total seed yield (total seed weight) was measured by weighing all filled husks harvested from a plant. Total seed number per plant was measured by counting the number of husks harvested from a plant and corresponds to the number of florets per plant. Thousand Kernel Weight (TKW) is extrapolated from the number of filled seeds counted and their total weight. Harvest index is defined as the ratio between the total seed weight and the above-ground area (mm²), multiplied by a factor 10⁶. The parameter EmerVigor is an indication of the seedling vigour. It is calculated from the area (in mm²) covered by leafy biomass in the first imaging. The seed fill rate (fillrate) is an indication of the filling of the seeds. It is expressed as a proportion (in %) of the number of filled seeds over the number of florets (nrtotalseed).

These parameters were derived in an automated way from the digital images using image analysis software and were analysed statistically. Individual seed parameters (including width, length, area, weight) were measured using a custom-made device consisting of two main components, a weighing and imaging device, coupled to software for image analysis.

### 7.2 Statistical Analysis: T-Test and F-Test

A two factor ANOVA (analysis of variants) was used as a statistical model for the overall evaluation of plant phenotypic characteristics. An F-test was carried out on all the parameters measured of all the plants of all the events transformed with the gene of the present invention. The F-test was carried out to check for an effect of the gene over all the transformation events and to verify an overall effect of the gene, also known as a global gene effect. The threshold for significance for a true global gene effect was set at a 5% probability level for the F-test. A significant F-test value points to a gene effect, meaning that it is not only the mere presence or position of the gene that is causing the differences in phenotype.

To check for an effect of the genes within an event, i.e., for a line-specific effect, a t-test was performed within each event using data sets from the transgenic plants and the corresponding null plants. “Null plants” or “null segregants” or “nullizygotes” are the plants treated in the same way as the transgenic plant, but from which the transgene has segregated. Null plants can also be described as the homozygous negative transformed plants. The threshold for significance for the t-test is set at 10% probability level. The results for some events can be above or below this threshold. This is based on the hypothesis that a gene might only have an effect in certain positions in the genome, and that the occurrence of this position-dependent effect is not uncommon. This kind of gene effect is also named herein a “line effect of the gene”. The p-value was obtained by comparing the t-value to the t-distribution or alternatively, by comparing the F-value to the F-distribution. The p-value then gives the probability that the null hypothesis (i.e., that there is no effect of the transgene) is correct.

### Example 8

### Evaluation Results

### 8.1 NAC1 Under the Control of a GOS2 Promoter

An increase in the following yield-related parameters was observed for transgenic plants overexpressing NAC1 under the control of a GOS2 promoter. The p-value shown is from the t-test unless otherwise indicated. The percentage difference is for transgenic plants compared to nullizygotes. Although not shown in Table 8, an increase in root biomass was also observed, with the best event giving a 7% increase compared to nullizygotes.

TABLE 8
Results for NAC1 under the control of a GOS2 promoter

| Trait | Positive events | Average difference | p value |
|---|---|---|---|
| Area max | 6 events | 13.70% | <0.077 |
| | | overall +11% | 0 |
| | | | (F test) |
| Emergence vigour | 5 events | 29.60% | <0.225 |
| | | overall +16% | <0.0108 |
| | | | (F test) |
| TKW | 5 events | 5.40% | <0.097 |
| | | overall +3% | 0 |
| | | | (F test) |
| First panicles | 3 events | 20.33% | <0.061 |
| Total number seeds | 2 events | 16 | <0.15 |

### 8.2 NAC4 Under the Control of a GOS2 Promoter

An increase in the following yield-related parameters was observed for transgenic plants overexpressing NAC4 under the control of a GOS2 promoter. The p-value shown is from the t-test unless otherwise indicated. The percentage difference is for transgenic plants compared to nullizygotes. Although not shown in the Table 9a, an increase in seed weight was also observed, with the best event giving a 30% increase compared to nullizygotes. Table 9a shows the results obtained under non-stress conditions and Table 9b shows the results obtained under conditions of stress.

TABLE 9a
Results for NAC4 under the control of a GOS2 promoter

| Trait | Positive events | Average difference | p value |
|---|---|---|---|
| Area max | 3 events | 18.33% | <0.051 |
| | | overall +9% | <0.0023 |
| | | | (F test) |
| Emergence vigor | 3 events | 30.67% | <0.38 |
| Root max | 2 events | 10.50% | <0.088 |
| TKW | 4 events | 6.50% | <0.042 |
| | | overall +4% | 0 |
| | | | (F test) |
| First panicles | 3 events | 15.67% | <0.34 |
| Total number seeds | 3 events | 13.33 | <0.281 |
| | | overall +5% | <0.091 |
| | | | (F test) |

TABLE 9b
Results for NAC4 under the control of a GOS2 promoter (drought screen)

| Trait | Positive events | Average difference | p value |
|---|---|---|---|
| Area max | 2 events | 10.5% | <0.0011 |
| | | overall +8% | 0 |
| | | | (F test) |
| Emergence vigor | 3 events | 42% | <0.0007 |
| TKW | 3 events | 4% | <0.042 |
| | | overall +4.66% | <0.0251 |
| | | | (F test) |
| First panicles | 3 events | 4.33% | <0.091 |
| | | Overall +2% | <0.00275 |
| | | | (F test) |
| Root thin | 2 events | 6% | <0.0901 |

### 8.3 NAC4 Under the Control of an RCc3 Promoter

An increase in the following yield-related parameters was observed for transgenic plants overexpressing NAC4 under the control of an RCc3 promoter. The p-value shown is from the t-test unless otherwise indicated. The percentage difference is for transgenic plants compared to nullizygotes. Although not shown in Table 10a, an increase in thousand kernel weight was also observed, with the best event giving a 4% increase compared to nullizygotes.

TABLE 10a
Results for NAC4 under the control of an RCc3 promoter

| Trait | Positive events | Average difference | p value |
|---|---|---|---|
| Area max | 2 events | 13.00% | <0.0125 |
| | | overall +4% | <0.0313 |
| | | | (F test) |
| Emergence vigour | 2 events | 13.50% | <0.288 |
| | | overall +7% | <0.11 |
| | | | (F test) |
| Root max | 1 event | 9.00% | <0.0597 |
| | | overall +4% | <0.0722 |
| | | | (F test) |
| Total seed yield | 2 events | 16.00% | <0.094 |
| | | overall +6% | <0.0733 |
| | | | (F test) |
| Total number seeds | 2 events | 14.5 | <0.137 |

TABLE 10b
Results for NAC4 under the control of an RCc3 promoter
(drought screen)

| Trait | Positive events | Average difference | p value |
|---|---|---|---|
| Area max | 2 events | 8% | <0.1077 |
| Total seed yield | 2 events | 49% | <0.018 |
| | | Overall +20% | 0.002 |
| | | | (F test) |
| Number filled seeds | 2 events | 50.5% | <0.0138 |
| | | Overall +20% | 0.0017 |
| | | | (F test) |
| Fill rate | 2 events | 46.5% | <0.0418 |
| | | Overall +16% | 0.011 |
| | | | (F test) |
| Harvest Index | 2 events | 47% | <0.0168 |
| | | Overall +19% | 0.0037 |
| Height | 2 events | 6.5% | <0.0103 |
| Number total seeds | 2 events | 26% | <0.0579 |
| | | Overall +4% | 0.1951 |
| | | | (F test |
| Root max | 2 events | 6.5% | <0.1038 |
| Flower per panicle | 2 events | 17.5% | <0.1019 |

### 8.4 NAC6 Under the Control of an RCc3 Promoter

An increase in the following yield-related parameters was observed for transgenic plants overexpressing NAC6 under the control of an RCc3 promoter. The p-value shown is from the t-test unless otherwise indicated. The percentage difference is for transgenic plants compared to nullizygotes. Although not shown in Table 11, an increase in the number of flowers per panicle was also observed, with the best event giving a 20% increase compared to nullizygotes. Furthermore, and also not shown in the table, an increase in the number of panicles was also observed, with the best event giving a 28% increase compared to nullizygotes.

TABLE 11
Results for NAC6 under the control of an RCc3 promoter

| Trait | Positive events | Average difference | p value |
|---|---|---|---|
| Area max | 2 events | 17.50% | <0.0104 |
| Emergence vigor | 2 events | 21.00% | <0.2385 |
| Root max | 3 events | 13.33% | <0.0093 |
| | | overall +5.00% | <0.0673 |
| | | | (F test) |
| rootshoot index | 4 events | 64.00% | <0.1279 |
| | | overall +37% | <0.0005 |
| | | | (F test) |
| Total seed yield | 2 events | 20.00% | <0.2378 |
| Total number seeds | 2 events | 20% | <0.0323 |

### 8.5 NAC7 Under the Control of a GOS2 Promoter

An increase in the following yield-related parameters was observed for transgenic plants overexpressing NAC7 under the control of a GOS2 promoter. The p-value shown is from the t-test unless otherwise indicated. The percentage difference is for transgenic plants compared to nullizygotes.

TABLE 12
Results for NAC7 under the control of a GOS2 promoter

| Trait | Positive events | Average difference | p value |
|---|---|---|---|
| Area max | 3 events | 18.33% | <0.011 |
| | | overall +11% | 0 |
| | | | (F test) |
| Emergence vigor | 3 events | 17.33% | <0.422 |
| Root max | 1 event | 11.00% | <0.0249 |
| | | overall +4% | <0.0370 |
| | | | (F test) |
| Total seed yield | 2 events | 20.50% | <0.08 |
| Flower per panicle | 3 events | 10% | <0.101 |
| First panicles | | 19.00% | <0.0769 |
| Total number seeds | 2 events | 15% | <0.177 |
| | | overall +5% | <0.0751 |
| | | | (F test) |

### 8.6 NAC3 Under the Control of an RCc3 Promoter

An increase in the following yield-related parameters was observed for transgenic plants overexpressing NAC3 under the control of an RCc3 promoter. The p-value shown is from the t-test unless otherwise indicated. The percentage difference is for transgenic plants compared to nullizygotes. Although not shown in Table 13, An increase in aboveground area was observed (with the best event giving a 19% increase), an increase in early vigour (with the best event giving a 20% increase), an increase in root biomass (with the best event giving a 15% increase), an increase in thousand kernel weight, an increase in harvest index (with the best event giving an increase of 26%), an increase in the number of panicles (with the best event giving an increase of 33%).

TABLE 13
Results for NAC3 under the control of an RCc3 promoter

| Trait | Positive events | Average difference | p value |
|---|---|---|---|
| Total seed yield | 2 events | 29.00% | <0.00777 |
| | | overall +6% | <0.00553 |
| | | | (F test) |
| Total number seeds | 1 event | 44% | <0.0001 |
| | | overall +5% | <0.1084 |
| | | | (F test) |

### C) AP2-2 polypeptide

### Example 9

### Identification of Sequences Related to SEQ ID NO: 131 and SEQ ID NO: 132

Sequences (full length cDNA, ESTs or genomic) related to SEQ ID NO: 131 and/or protein sequences related to SEQ ID NO: 132 were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program was used to find regions of local similarity between sequences by comparing nucleic acid or polypeptide sequences to sequence databases and by calculating the statistical significance of matches. The polypeptide encoded by SEQ ID NO: 131 was used for the TBLASTN algorithm, with default settings and the filter to ignore low complexity sequences set off. The output of the analysis was viewed by pairwise comparison, and ranked according to the probability score (E-value), where the score reflects the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search.

Table 14 provides a list of nucleic acid and protein sequences related to the nucleic acid sequence as represented by SEQ ID NO: 131 and the protein sequence represented by SEQ ID NO: 132.

TABLE 14
Nucleic acid sequences encoding AP2-2 polypeptides and
AP2-2 polypeptides.

| | | Nucleic | |
|---|---|---|---|
| | | acid | |
| | | SEQ ID | Poly-peptide |
| Name/identifier | Source organism | NO: | SEQ ID NO: |
| AP2-EREBP | Oryza sativa | 131 | 132 |
| Os07g47790 | Oryza sativa | 139 | 140 |
| AAX13280 | Triticum aestivum | 141 | 142 |
| Os01g21120 | Oryza sativa | 143 | 144 |
| Os03g08470 | Oryza sativa | 145 | 146 |
| CAD56466 | Triticum aestivum | 147 | 148 |
| AT2G47520.1 | Arabidopsis thaliana | 149 | 150 |
| Os02g54160 | Oryza sativa | 151 | 152 |
| Os09g26420 | Oryza sativa | 153 | 154 |
| AAP32468 | Triticum aestivum | 155 | 156 |
| AAP32467 | Triticum aestivum | 157 | 158 |
| AT1G53910.1 | Arabidopsis thaliana | 159 | 160 |
| AAM65746 | Arabidopsis thaliana | 161 | 162 |
| AT3G14230.1 | Arabidopsis thaliana | 163 | 164 |
| AT3G14230.2 | Arabidopsis thaliana | 165 | 166 |
| AT3G14230.3 | Arabidopsis thaliana | 167 | 168 |
| AT3G16770 | Arabidopsis thaliana | 169 | 170 |
| Os07g42510 | Oryza sativa | 171 | 172 |
| Os03g08490 | Oryza sativa | 173 | 174 |
| Os03g08500 | Oryza sativa | 175 | 176 |
| AT1G72360 | Arabidopsis thaliana | 177 | 178 |
| Os03g08460 | Oryza sativa | 179 | 180 |
| Os05g29810 | Oryza sativa | 181 | 182 |
| Os03g22170 | Oryza sativa | 183 | 184 |
| Os10g25170 | Oryza sativa | 185 | 186 |
| Os09g11460 | Oryza sativa | 187 | 188 |
| AAK95687 | Lycopersicon esculentum | 189 | 190 |
| Os09g11480 | Oryza sativa | 191 | 192 |
| AAQ91334 | Lycopersicon esculentum | 193 | 194 |
| AAR87866 | Lycopersicon esculentum | 195 | 196 |
| AT1G80580 | Arabidopsis thaliana | 197 | 198 |
| AAP40022 | Nicotiana tabacum | 199 | 200 |
| CAE54591 | Fagus sylvatica | 201 | 202 |
| CAD21849 | Fagus sylvatica | 203 | 204 |
| ABD65407 | Capsicum annuum | 205 | 206 |
| AAP72289 | Capsicum annuum | 207 | 208 |
| AAS20427 | Capsicum annuum | 209 | 210 |
| AAX07458 | Gossypium hirsutum | 211 | 212 |
| AAT77192 | Gossypium barbadense | 213 | 214 |
| AAX20013 | Gossypium hirsutum | 215 | 216 |
| AAX68526 | Gossypium hirsutum | 217 | 218 |
| AAX68525 | Gossypium hirsutum | 219 | 220 |
| AAT77191 | Gossypium barbadense | 221 | 222 |
| AAV51937 | Gossypium hirsutum | 223 | 224 |
| AAV85777 | Gossypium hirsutum | 225 | 226 |
| AAX07460 | Gossypium hirsutum | 227 | 228 |
| AAX84670 | Manihot esculenta | 229 | 230 |
| AAW33881 | Populus alba × Populus tremula | 231 | 232 |
| BAE71206 | Trifolium pratense | 233 | 234 |
| AAQ10777 | Glycine max | 235 | 236 |
| AAL67489 | Narcissus pseudonarcissus | 237 | 238 |
| CAA05084 | Arabidopsis thaliana | 239 | 240 |
| ABE84970 | Medicago truncatula | 241 | 242 |
| ABE80536 | Medicago truncatula | 243 | 244 |
| AAC29516 | Solanum tuberosum | 245 | 246 |
| BAC56862 | Solanum tuberosum | 247 | 248 |
| AAS01337 | Coffea canephora | 249 | 250 |
| AAZ14085 | Hordeum vulgare | 251 | 252 |
| BAD01556 | Cucumis melo | 253 | 254 |
| BAF43419 | Malus × domestica | 255 | 256 |

### Example 10

### Alignment of AP2-2 polypeptide Sequences

Alignment of polypeptide sequences was performed using the AlignX programme from the Vector NTI (Invitrogen) which is based on the popular Clustal algorithm of progressive alignment (Thompson et al. (1997) Nucleic Acids Res 25:4876-4882; Chenna et al. (2003). Nucleic Acids Res 31:3497-3500). Default values are for the gap open penalty of 10, for the gap extension penalty of 0,1 and the selected weight matrix is Blosum 62 (if polypeptides are aligned). Results in FIG. 7 show that AP2-2 polypeptides share regions of high sequence conservation. Motifs XII to XVII are underlined for Os06g09390 (SEQ ID NO: 132).

### Example 11

### Calculation of Global Percentage Identity Between Polypeptide Sequences Useful in Performing the Methods of the Invention

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum62
First Gap: 12
Extending gap: 2

Results of the software analysis are shown in Table 15 for the global similarity and identity over the full length of the polypeptide sequences (excluding the partial polypeptide sequences). Percentage identity is given above the diagonal and percentage similarity is given below the diagonal.

The percentage identity between the full length polypeptide sequences useful in performing the methods of the invention can be as low as 20% amino acid identity compared to SEQ ID NO: 132. The sequence identity will be higher when specific domains, such as the AP2 domain are compared.

TABLE 15
MatGAT results for global similarity and identity over the full length of the polypeptide
sequences.

| | | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|---|
| 1. | SEQID 132 | | 27.7 | 63.3 | 29.8 | 38.3 | 62.4 | 24 | 59.5 | 40.8 | 61.1 | 58.2 | 37.2 | 37.2 | 37.1 | 37 |
| 2. | SEQID 140 | 37.8 | | 27.2 | 43.9 | 29.7 | 26.6 | 36.4 | 27.5 | 26.5 | 26.2 | 25.7 | 29.1 | 29.1 | 27.4 | 27.7 |
| 3. | SEQID 142 | 76.5 | 35.8 | | 29.9 | 33.1 | 90.8 | 24.5 | 55.9 | 39 | 94.6 | 88.5 | 35.9 | 35.9 | 36.7 | 37.3 |
| 4. | SEQID 144 | 37.6 | 55.6 | 35.5 | | 31.1 | 30.3 | 41.1 | 27.6 | 25 | 28.7 | 29.2 | 27.7 | 27.9 | 27.2 | 27.5 |
| 5. | SEQID 146 | 50.6 | 37.7 | 48.2 | 39.5 | | 32 | 24.9 | 36.1 | 33.2 | 30.7 | 30.2 | 34.3 | 34.3 | 32.1 | 32.4 |
| 6. | SEQID 148 | 74.9 | 35.2 | 94.4 | 35.2 | 47 | | 25.2 | 56.5 | 38.6 | 87.4 | 88 | 36.2 | 36.2 | 37.6 | 37.9 |
| 7. | SEQID 150 | 31.8 | 48.1 | 32.1 | 52.7 | 34.1 | 32.1 | | 23.6 | 22.2 | 23.9 | 24.7 | 26.3 | 26.3 | 24.8 | 25.3 |
| 8. | SEQID 152 | 74.8 | 35.1 | 69.9 | 34.2 | 47.1 | 69 | 31.2 | | 42.7 | 53.5 | 53.6 | 39.9 | 39.9 | 36.1 | 36.3 |
| 9. | SEQID 154 | 54 | 33.6 | 51.5 | 32.6 | 45.7 | 51 | 28 | 56.1 | | 37.3 | 36.1 | 36.8 | 36.8 | 35 | 35.3 |
| 10. | SEQID 156 | 73.2 | 33.8 | 96.1 | 34.6 | 46.5 | 91.3 | 31.5 | 67.1 | 50 | | 85.1 | 34.8 | 35.1 | 35.9 | 36.3 |
| 11. | SEQID 158 | 70.7 | 34.6 | 89.9 | 36.1 | 45.8 | 90 | 34 | 66.6 | 48 | 86.8 | | 32.5 | 32.5 | 34 | 34.4 |
| 12. | SEQID 160 | 53.3 | 38.5 | 54.2 | 35.8 | 48 | 52.5 | 33 | 53.7 | 50 | 53.6 | 49.2 | | 99.4 | 62.2 | 62.8 |
| 13. | SEQID 162 | 53.3 | 38.5 | 54.2 | 35.8 | 48 | 52.5 | 33 | 53.7 | 50 | 53.6 | 49.2 | 100 | | 62.2 | 62.8 |
| 14. | SEQID 164 | 56.7 | 34.3 | 54.4 | 33.5 | 47.2 | 54.6 | 29.6 | 55.4 | 49.7 | 54.1 | 50.1 | 70.7 | 71 | | 98.9 |
| 15. | SEQID 166 | 57.1 | 34.7 | 54.7 | 33.9 | 47.7 | 54.9 | 30.4 | 55.7 | 49.7 | 54.7 | 50.7 | 71.7 | 72 | 98.9 | |
| 16. | SEQID 168 | 57.2 | 34.8 | 54.8 | 34 | 47.9 | 55.1 | 30.2 | 55.6 | 51 | 54.8 | 50.8 | 71.9 | 72.2 | 98.7 | 99.7 |
| 17. | SEQID 170 | 41.2 | 47.2 | 38.9 | 46.4 | 41.3 | 38.4 | 46.8 | 38.6 | 36.9 | 37.5 | 38.3 | 40.8 | 40.8 | 39.6 | 40 |
| 18. | SEQID 172 | 51.9 | 34.8 | 52.1 | 38.9 | 52 | 54.4 | 32.2 | 50.4 | 43.9 | 49.9 | 52.9 | 46.6 | 46.6 | 44.3 | 43.7 |
| 19. | SEQID 174 | 46.4 | 34.9 | 43.4 | 40.8 | 51.2 | 46.7 | 31.5 | 48.2 | 40.9 | 42.8 | 44 | 43.3 | 43.6 | 42.2 | 42.7 |
| 20. | SEQID 176 | 50 | 38.6 | 49.6 | 40.1 | 52.7 | 49 | 32.8 | 48.2 | 44.7 | 47.6 | 45.5 | 48 | 48 | 44.6 | 45.1 |
| 21. | SEQID 178 | 41.7 | 46.9 | 44.5 | 47.7 | 47 | 45 | 44.3 | 42.5 | 39.6 | 42.5 | 44.6 | 45 | 45.3 | 39.6 | 40.3 |
| 22. | SEQID 180 | 47.5 | 36.5 | 40.8 | 35.9 | 44.9 | 42.1 | 32.8 | 44.1 | 38.1 | 39.4 | 42.8 | 46.6 | 46.6 | 42.7 | 42.4 |
| 23. | SEQID 182 | 32.3 | 50 | 32.7 | 50.7 | 36.8 | 33.2 | 47 | 32.1 | 31.1 | 31.5 | 32.2 | 31.8 | 31.8 | 32.5 | 32.8 |
| 24. | SEQID 184 | 39 | 55.1 | 37.2 | 53.5 | 43.1 | 39.8 | 44.9 | 37.3 | 36.9 | 36.6 | 39.8 | 36.3 | 36.3 | 34.3 | 34.7 |
| 25. | SEQID 186 | 40.6 | 35.7 | 42 | 41.3 | 50.6 | 42.1 | 32 | 44.4 | 42.7 | 42.5 | 40.7 | 39.7 | 39.7 | 40.1 | 39.5 |
| 26. | SEQID 188 | 31.8 | 37.9 | 34.1 | 41.4 | 39.2 | 33.8 | 38.4 | 32.1 | 33.1 | 33 | 35.8 | 34.1 | 34.1 | 33 | 33.6 |
| 27. | SEQID 190 | 60.2 | 34.1 | 59.1 | 36.8 | 48.1 | 59.4 | 33.6 | 58.9 | 54.3 | 57.5 | 54.6 | 59.7 | 59.7 | 60.7 | 61.3 |
| 28. | SEQID 192 | 39.5 | 41.2 | 38 | 42 | 38.6 | 39.8 | 38 | 38.9 | 37.4 | 36.9 | 39.5 | 39.4 | 39.4 | 38 | 39.5 |
| 29. | SEQID 194 | 53.9 | 37 | 54.6 | 38.2 | 47.9 | 55.3 | 34.9 | 50.1 | 46.7 | 53.2 | 53.6 | 57.5 | 57.5 | 58.3 | 57.6 |
| 30. | SEQID 196 | 44.2 | 48.8 | 44.5 | 51.2 | 41.3 | 43.8 | 49.2 | 40.5 | 37.6 | 41.4 | 41.9 | 39.1 | 39.1 | 41.2 | 40 |
| 31. | SEQID 198 | 29.6 | 35.5 | 33.5 | 34 | 33.2 | 32.7 | 32 | 32.3 | 29.5 | 34.1 | 35.2 | 33.8 | 33.8 | 31.1 | 31.5 |
| 32. | SEQID 200 | 56.6 | 32.8 | 55.6 | 37 | 49.9 | 55.3 | 33.3 | 56.1 | 52.3 | 54 | 51.4 | 61.5 | 61.5 | 62.8 | 62.8 |
| 33. | SEQID 202 | 56.6 | 34.9 | 57.4 | 37.6 | 49.7 | 57.9 | 31 | 60.8 | 56.8 | 55.3 | 53.4 | 63.8 | 64 | 64.1 | 64.3 |
| 34. | SEQID 204 | 56.5 | 34.1 | 57.6 | 37.6 | 49.3 | 57.6 | 30.7 | 60.5 | 55.1 | 56.3 | 53.3 | 62.7 | 62.9 | 62.8 | 63.5 |
| 35. | SEQID 206 | 60 | 34.1 | 57.9 | 36.5 | 50.4 | 58.9 | 32.5 | 57.6 | 52.5 | 56.5 | 53.3 | 59.5 | 59.5 | 59.6 | 60.3 |
| 36. | SEQID 208 | 59.1 | 32.8 | 56.6 | 35.2 | 49.3 | 57.7 | 32 | 56.6 | 51.3 | 55.3 | 52 | 58.5 | 58.5 | 58.6 | 59.2 |
| 37. | SEQID 210 | 44.2 | 48.5 | 43.4 | 52.3 | 42.8 | 43.3 | 45.8 | 42.7 | 39.6 | 40.8 | 42.5 | 43.6 | 43.6 | 41.7 | 41.3 |
| 38. | SEQID 212 | 55.4 | 34.9 | 51.8 | 34.6 | 50 | 53.3 | 33.1 | 57.4 | 57.6 | 50 | 47.7 | 64.6 | 64.9 | 65.6 | 65.4 |
| 39. | SEQID 214 | 50.3 | 37 | 49.6 | 38.6 | 46.1 | 51.3 | 37 | 48.8 | 44.9 | 47.3 | 52.1 | 54.2 | 54.5 | 53.6 | 54.1 |
| 40. | SEQID 216 | 53.5 | 30.8 | 50.8 | 34.1 | 49 | 51.8 | 33.1 | 52 8 | 55.6 | 49.2 | 48.2 | 59.8 | 60.1 | 59.8 | 59.6 |
| 41. | SEQID 218 | 39 | 43.3 | 40 | 45.6 | 41.6 | 37.2 | 42.1 | 40 | 37.9 | 37.7 | 41 | 39.9 | 39.7 | 39.3 | 38.4 |
| 42. | SEQID 220 | 38.7 | 45.3 | 42 | 47.3 | 42.5 | 40.4 | 46.5 | 37.5 | 40.7 | 37.2 | 40.4 | 40.5 | 40.5 | 40.4 | 40 |
| 43. | SEQID 222 | 32.6 | 47.7 | 32.7 | 49.3 | 35.3 | 33 | 51.5 | 32.9 | 31.3 | 30.4 | 31.3 | 33.2 | 33.2 | 33.5 | 34.7 |
| 44. | SEQID 224 | 39.2 | 45.9 | 40.8 | 45.1 | 40.4 | 39.5 | 45.9 | 38.9 | 39.4 | 39.4 | 37.7 | 41.1 | 41.1 | 38.8 | 38.9 |
| 45. | SEQID 228 | 40.6 | 43.5 | 39.4 | 45.4 | 41.9 | 40.1 | 42.4 | 40.8 | 38.9 | 37.5 | 39.8 | 39.1 | 39.1 | 39.8 | 40.5 |
| 46. | SEQID 230 | 58 | 34.1 | 53.5 | 38.1 | 48 | 57 | 32 | 59.3 | 59.3 | 51.7 | 53.5 | 64.8 | 64.8 | 64.6 | 64.6 |
| 47. | SEQID 232 | 56.6 | 33.9 | 51.3 | 36.1 | 47.4 | 51.6 | 31.8 | 56.1 | 53.8 | 52.1 | 49.2 | 60.3 | 60 | 61.3 | 61.1 |
| 48. | SEQID 234 | 56.9 | 35.1 | 52.7 | 35.3 | 48.1 | 53.2 | 31.7 | 53.8 | 54.5 | 50.9 | 50.6 | 57.4 | 57.4 | 61 | 61.3 |
| 49. | SEQID 236 | 55.7 | 35.2 | 52.9 | 35.4 | 45.8 | 53.1 | 33.3 | 57 | 56.8 | 49.7 | 50.8 | 55.7 | 56 | 59.4 | 59.1 |
| 50. | SEQID 240 | 40.6 | 46.3 | 42.5 | 47.2 | 40.4 | 41.3 | 46.3 | 39.5 | 36.9 | 39.4 | 41.3 | 39.7 | 39.7 | 39.8 | 40.3 |
| 51. | SEQID 242 | 42.5 | 40.7 | 44.8 | 43.9 | 40.7 | 44.7 | 43 | 42.2 | 37.6 | 43.1 | 43.4 | 44.1 | 44.1 | 43 | 43.7 |
| 52. | SEQID 244 | 33.4 | 40.8 | 29.6 | 38.3 | 32.9 | 32.1 | 41.7 | 32.9 | 27.8 | 28.7 | 31.9 | 35.2 | 35.5 | 32.5 | 32.8 |
| 53. | SEQID 246 | 46.1 | 42.6 | 41.4 | 47.3 | 42.8 | 41.3 | 41.6 | 41.6 | 39.4 | 41.7 | 40.4 | 41.6 | 41.1 | 40.6 | 40.8 |
| 54. | SEQID 248 | 42.3 | 46.6 | 40.6 | 45.1 | 42.2 | 40.7 | 43.6 | 37.8 | 37.9 | 39.2 | 39.8 | 42.2 | 42.2 | 41.2 | 41.1 |
| 55. | SEQID 250 | 43.4 | 40.1 | 44.8 | 43.2 | 48.5 | 45.6 | 37.7 | 45.5 | 42.7 | 43.7 | 44.3 | 43 | 43 | 42.5 | 42.9 |
| 56. | SEQID 252 | 46.7 | 38.4 | 47.3 | 39.6 | 60.5 | 47.9 | 34.1 | 47.9 | 42.4 | 45.9 | 48.5 | 44.7 | 45.8 | 42.5 | 42.9 |
| 57. | SEQID 254 | 43.4 | 49.5 | 44.2 | 51.6 | 44.9 | 43.8 | 46.2 | 40.5 | 40.2 | 42.5 | 44 | 43 | 43 | 40.1 | 40 |
| 58. | SEQID 256 | 36.2 | 44.8 | 38.3 | 45.6 | 40.4 | 39.5 | 45.2 | 36.4 | 36.6 | 36.3 | 38.9 | 38.3 | 38.3 | 38 | 38.7 |
| | | 16 | 17 | 18 | 19 | 20 | 21 | 22 | 23 | 24 | 25 | 26 | 27 | 28 | 29 | 30 |
| 1. | SEQID 132 | 37.6 | 29.6 | 38.3 | 30.1 | 34 | 26.5 | 29.6 | 26 | 27.2 | 27.9 | 23.7 | 41.4 | 27.7 | 38.4 | 32.2 |
| 2. | SEQID 140 | 27.8 | 34.1 | 28.7 | 27.7 | 27.6 | 34.1 | 26.1 | 40.3 | 45.8 | 28.5 | 26.6 | 26.9 | 32.5 | 28.7 | 36.7 |
| 3. | SEQID 142 | 37.4 | 28.5 | 35.2 | 30.7 | 32.4 | 29.1 | 26.6 | 25.8 | 28.2 | 27.8 | 23.5 | 40.1 | 25.3 | 36.7 | 32.8 |
| 4. | SEQID 144 | 27.5 | 35.9 | 30.6 | 32 | 31.3 | 33 | 29.8 | 41.6 | 39.3 | 31.4 | 28.8 | 29.4 | 30.2 | 31.2 | 43.5 |
| 5. | SEQID 146 | 32.5 | 29.3 | 36.3 | 35.7 | 37.5 | 29.5 | 30.2 | 27.5 | 30.3 | 35.9 | 27.6 | 35.4 | 29.7 | 35.4 | 30.3 |
| 6. | SEQID 148 | 38 | 28.4 | 36.9 | 31.6 | 32 | 30.3 | 26.9 | 26.9 | 28.7 | 29.3 | 23.5 | 40.9 | 26.5 | 38 | 33.3 |
| 7. | SEQID 150 | 25.7 | 33.3 | 25.1 | 24 | 24.6 | 31.7 | 21.8 | 36.7 | 31 | 24.2 | 26.9 | 28 | 28 | 25.4 | 38.1 |
| 8. | SEQID 152 | 36.4 | 28.3 | 37.2 | 32 | 31.6 | 30.1 | 27.9 | 25.8 | 28.6 | 32.2 | 23.4 | 42 | 28.7 | 37.5 | 31.1 |
| 9. | SEQID 154 | 35.7 | 25.3 | 31.7 | 30.1 | 31 | 26.2 | 23.8 | 24.7 | 28.8 | 28.1 | 23.6 | 37.8 | 26.2 | 33.6 | 27.5 |
| 10. | SEQID 156 | 36.4 | 27.3 | 33.2 | 30.2 | 31.4 | 27.7 | 24.6 | 24.7 | 24.5 | 26.2 | 22.4 | 39 | 24.2 | 35.3 | 30.5 |
| 11. | SEQID 158 | 34.5 | 28 | 33.7 | 29.3 | 28.3 | 29.1 | 25.3 | 25.8 | 27.8 | 26.6 | 23.6 | 37.1 | 25.9 | 34.9 | 30.1 |
| 12. | SEQID 160 | 63 | 28.5 | 32.4 | 28 | 30.2 | 31.3 | 27.3 | 24.9 | 25.7 | 27.2 | 24.9 | 43.4 | 29.1 | 42.9 | 28.9 |
| 13. | SEQID 162 | 63 | 28.5 | 32.4 | 28.2 | 30.2 | 31 | 27.3 | 24.9 | 25.7 | 27.2 | 24.7 | 43.7 | 29.1 | 42.9 | 28.9 |
| 14. | SEQID 164 | 98.7 | 29.3 | 32.6 | 27.9 | 29.9 | 27.7 | 26.1 | 24.5 | 26.3 | 27.1 | 24.1 | 42.5 | 27.9 | 44 | 29.1 |
| 15. | SEQID 166 | 99.7 | 29.6 | 31.9 | 28.2 | 30.2 | 28.3 | 26.4 | 24.8 | 26.6 | 27.9 | 24.9 | 42.9 | 29.6 | 43.9 | 28.3 |
| 16. | SEQID 168 | | 29.7 | 31.9 | 28.3 | 30.3 | 28.1 | 26.7 | 24.9 | 26.7 | 28.5 | 23.9 | 43 | 29.4 | 44.8 | 29.5 |
| 17. | SEQID 170 | 40.1 | | 30.7 | 29.6 | 29.3 | 31.6 | 24.8 | 36.1 | 35.6 | 28.5 | 26.5 | 28.9 | 30.4 | 31.6 | 34.9 |
| 18. | SEQID 172 | 43.6 | 40.6 | | 31.4 | 32.6 | 30.1 | 28.3 | 28.8 | 28.5 | 32.5 | 24.4 | 33.8 | 26.4 | 32.5 | 28.1 |
| 19. | SEQID 174 | 42.8 | 42.7 | 46.2 | | 47.1 | 30.5 | 30.9 | 30.5 | 28.9 | 35.3 | 24.5 | 29.3 | 28.8 | 27.4 | 28.7 |
| 20. | SEQID 176 | 45.2 | 41.3 | 50.3 | 59.3 | | 28.9 | 30.3 | 27.9 | 29.2 | 34.3 | 24.6 | 29.8 | 28.5 | 29.9 | 29.7 |
| 21. | SEQID 178 | 40.1 | 45 | 45.6 | 44.5 | 45.9 | | 27 | 30.2 | 28.6 | 26.1 | 27.9 | 31.4 | 28.4 | 31.2 | 33.7 |
| 22. | SEQID 180 | 42.8 | 38 | 42.1 | 47.2 | 47.1 | 41.4 | | 25.8 | 25 | 26.5 | 26.1 | 28 | 27.5 | 30 | 24.7 |
| 23. | SEQID 182 | 32.9 | 46.8 | 39.2 | 38 | 37.7 | 42.7 | 35 | | 37.4 | 27.6 | 28.6 | 23.9 | 33.5 | 26.6 | 31.3 |
| 24. | SEQID 184 | 34.5 | 48.8 | 38.6 | 40.5 | 38.6 | 43.1 | 37.1 | 44.4 | | 30.6 | 27.2 | 24.6 | 30.6 | 28.3 | 35.4 |
| 25. | SEQID 186 | 39.6 | 40.7 | 45 | 51.2 | 50.2 | 39.8 | 38 | 33.9 | 37.9 | | 26.9 | 29.2 | 28.5 | 27.3 | 26.6 |
| 26. | SEQID 188 | 33.7 | 44 | 33 | 37.4 | 36.8 | 41.6 | 35.3 | 37.9 | 39.5 | 39.8 | | 23.3 | 41.3 | 25.7 | 25.2 |
| 27. | SEQID 190 | 61.5 | 39.2 | 49.7 | 44.9 | 45.2 | 46.2 | 45.4 | 32.5 | 34.7 | 43 | 33.9 | | 27.1 | 52.8 | 32.8 |
| 28. | SEQID 192 | 39.6 | 43.2 | 37.1 | 39.9 | 39.8 | 47.7 | 41.4 | 40.8 | 43.6 | 37.6 | 56.4 | 37.4 | | 29.5 | 26.5 |
| 29. | SEQID 194 | 59.1 | 42.2 | 45.3 | 47.1 | 46.5 | 45.3 | 45 | 36.4 | 38.8 | 43.1 | 37 | 66.9 | 41.9 | | 31 |
| 30. | SEQID 196 | 41.4 | 51.2 | 39.8 | 41.4 | 42.2 | 53.1 | 39.6 | 41.5 | 50.8 | 38.2 | 42.7 | 45.4 | 44.6 | 43.4 | |
| 31. | SEQID 198 | 31.6 | 43.8 | 33.9 | 35.2 | 30.4 | 42 | 34 | 36.3 | 40.6 | 33.5 | 39.5 | 34.4 | 41 | 32.1 | 41.5 |
| 32. | SEQID 200 | 62 | 40.8 | 47.8 | 41.3 | 44.4 | 44.2 | 45 | 32.6 | 32.6 | 40.3 | 33.9 | 80.4 | 37.5 | 73.9 | 42.4 |
| 33. | SEQID 202 | 64.3 | 38.4 | 47.9 | 45.8 | 47.9 | 44.7 | 46.6 | 32.5 | 36.8 | 41.3 | 35.2 | 71.7 | 35.2 | 62.2 | 44.7 |
| 34. | SEQID 204 | 63.5 | 38.1 | 48.3 | 45.6 | 47.5 | 43.5 | 45.9 | 31.7 | 36 | 41.1 | 33.6 | 71.7 | 35.7 | 61.3 | 45.1 |
| 35. | SEQID 206 | 60.3 | 41.9 | 50.7 | 44 | 46.4 | 45.1 | 44.8 | 33.9 | 36 | 39.7 | 36.3 | 90.7 | 38.4 | 66.7 | 46.1 |
| 36. | SEQID 208 | 59.4 | 41.7 | 49.6 | 43.9 | 47.4 | 45.5 | 46.1 | 34.4 | 36.3 | 40.1 | 36.6 | 89.2 | 38.8 | 67.8 | 46.1 |
| 37. | SEQID 210 | 42.2 | 55.7 | 43 | 43.6 | 43.2 | 50.8 | 40.5 | 41.3 | 51.1 | 37.9 | 40.5 | 45.7 | 46.2 | 43.4 | 81.4 |
| 38. | SEQID 212 | 65.9 | 40.3 | 47.4 | 43.1 | 45.9 | 43.6 | 43.1 | 33.3 | 33.8 | 41.3 | 33.3 | 67.2 | 35.4 | 57.4 | 44.4 |
| 39. | SEQID 214 | 54.3 | 40.8 | 44.7 | 43.9 | 44.1 | 47.6 | 41.1 | 34.5 | 37 | 41.9 | 32.9 | 58.9 | 36.7 | 53.5 | 48.6 |
| 40. | SEQID 216 | 59.8 | 35.6 | 49.2 | 43.4 | 45.7 | 42.7 | 42.2 | 30.3 | 32.6 | 39.9 | 33.1 | 63.9 | 35.9 | 55.1 | 41.4 |
| 41. | SEQID 218 | 38.2 | 62.5 | 39.2 | 39.9 | 41.6 | 50.8 | 44.5 | 44.4 | 40.6 | 37.9 | 41.8 | 40.1 | 42.5 | 44.3 | 50.6 |
| 42. | SEQID 220 | 40.6 | 59.4 | 38 | 38.6 | 40.1 | 48.1 | 40.5 | 47.3 | 45.3 | 38.5 | 44.1 | 38.4 | 43 | 43.4 | 50.8 |
| 43. | SEQID 222 | 34.5 | 50.8 | 32.5 | 30.8 | 35 | 42.7 | 35.9 | 51 | 40.7 | 34.5 | 44.4 | 32.5 | 41.2 | 38.5 | 41.9 |
| 44. | SEQID 224 | 38.8 | 60.4 | 38 | 39.6 | 40.4 | 47.3 | 41.4 | 45.9 | 45.9 | 38.2 | 42 | 38.2 | 43.9 | 42.5 | 50 |
| 45. | SEQID 228 | 40.4 | 62.6 | 40.1 | 39.6 | 41.3 | 50.4 | 42.3 | 44.3 | 42 | 39.8 | 43.5 | 41.1 | 42.7 | 45 | 51.1 |
| 46. | SEQID 230 | 64.3 | 39.9 | 50.4 | 45.1 | 46.5 | 45.9 | 44.4 | 33.3 | 34.9 | 42 | 36 | 69.3 | 36.2 | 61.2 | 43.6 |
| 47. | SEQID 232 | 61.8 | 39.2 | 46.3 | 40.8 | 43.4 | 43.7 | 43.4 | 31.3 | 35.8 | 38.4 | 32.6 | 67.4 | 36.8 | 56.8 | 42.1 |
| 48. | SEQID 234 | 61.3 | 39 | 43.9 | 42.3 | 47 | 44.2 | 47.3 | 32.5 | 36.1 | 39 | 33 | 63.4 | 37.7 | 54.3 | 42.3 |
| 49. | SEQID 236 | 59.4 | 39.6 | 45.6 | 44.5 | 46.9 | 43.2 | 41.9 | 33.3 | 37 | 39.1 | 33.6 | 63.5 | 35.4 | 53.1 | 41.9 |
| 50. | SEQID 240 | 40.4 | 94.4 | 39.8 | 40.2 | 42.9 | 44.7 | 40.5 | 45.9 | 50 | 37.6 | 43.1 | 41.1 | 42 | 43.4 | 51.9 |
| 51. | SEQID 242 | 43.6 | 47.2 | 43.6 | 42.1 | 41.3 | 44.9 | 43.3 | 37.4 | 42.3 | 37.9 | 34.1 | 48.4 | 39 | 48.3 | 58 |
| 52. | SEQID 244 | 31.8 | 47.6 | 29.5 | 33.3 | 33.1 | 45 | 33.1 | 35.4 | 39.1 | 32.3 | 42.9 | 33.6 | 39.6 | 36.4 | 41.2 |
| 53. | SEQID 246 | 41.2 | 46 | 41.8 | 41.7 | 41.9 | 47.7 | 44.5 | 35.2 | 43.6 | 37.6 | 37.6 | 48.4 | 41.6 | 45 | 81.2 |
| 54. | SEQID 248 | 40.4 | 62.1 | 43 | 39.6 | 42.2 | 51.1 | 43.6 | 46.2 | 47 | 41.3 | 42.4 | 41.9 | 47 | 47.1 | 53.8 |
| 55. | SEQID 250 | 43 | 45.9 | 47.4 | 45.3 | 45 | 42.6 | 44.4 | 37.7 | 43.8 | 44.7 | 35.6 | 50.5 | 40.4 | 48 | 55.3 |
| 56. | SEQID 252 | 43.9 | 40.9 | 47.7 | 45.4 | 51.4 | 46 | 43.9 | 36.6 | 40.2 | 45.7 | 35.4 | 46.8 | 39.9 | 46.6 | 41.8 |
| 57. | SEQID 154 | 40.4 | 55.7 | 41.8 | 44.2 | 43.2 | 52 | 41.1 | 41 | 46.9 | 38.2 | 38.5 | 45.2 | 42.9 | 42.8 | 63.7 |
| 58. | SEQID 156 | 39.3 | 58.3 | 37.4 | 38.6 | 41.3 | 46.2 | 40.8 | 44 | 46 | 40.4 | 40.9 | 36.8 | 42.5 | 42.8 | 50 |
| | | 31 | 32 | 33 | 34 | 35 | 36 | 37 | 38 | 39 | 40 | 41 | 42 | 43 | 44 | 45 |
| 1. | SEQID 132 | 20.3 | 41.3 | 41.2 | 41 | 40.3 | 39.4 | 31.4 | 42 | 34.5 | 38.4 | 26.8 | 29.5 | 24.2 | 28.2 | 27 |
| 2. | SEQID 140 | 24 | 25.8 | 27 | 26.4 | 25.1 | 26.3 | 36.6 | 26.9 | 26.8 | 25.5 | 33.1 | 33.3 | 35.4 | 33.1 | 33.7 |
| 3. | SEQID 142 | 21.5 | 40.3 | 41.2 | 40.5 | 38.7 | 37 | 29.9 | 38.4 | 34.3 | 35.7 | 26.3 | 28 | 24 | 27.9 | 26.2 |
| 4. | SEQID 144 | 24.1 | 30.1 | 30.4 | 30.2 | 28.9 | 28.5 | 42.8 | 27.8 | 29.4 | 27.3 | 34.2 | 37.5 | 33.9 | 37 | 34.8 |
| 5. | SEQID 146 | 21.5 | 36.8 | 34.5 | 33.8 | 36.9 | 36.2 | 29.7 | 35 | 29.4 | 34.9 | 29.3 | 29.1 | 25.1 | 28.3 | 27.8 |
| 6. | SEQID 148 | 21.2 | 41.3 | 43.1 | 42 | 40.6 | 38.9 | 31.2 | 39.9 | 35.6 | 37.2 | 27.4 | 27.8 | 24.4 | 27.7 | 27.2 |
| 7. | SEQID 150 | 23 | 26.9 | 26.1 | 25.8 | 26.7 | 26.3 | 39.4 | 25.6 | 26.3 | 25.3 | 33.7 | 34.8 | 35.8 | 34.8 | 32.8 |
| 8. | SEQID 152 | 21 | 41.1 | 43 | 43 | 41.1 | 40.1 | 29.8 | 43.2 | 35.9 | 39.7 | 27 | 29.1 | 25.2 | 28 | 27.2 |
| 9. | SEQID 154 | 20.8 | 37.1 | 40 | 38.7 | 35.9 | 35.5 | 28.3 | 38.8 | 30.8 | 38.1 | 27.2 | 29.4 | 23.5 | 27.9 | 26.7 |
| 10. | SEQID 156 | 21.3 | 39.5 | 39.9 | 39.5 | 38.5 | 36.8 | 28 | 36.9 | 32.5 | 34.6 | 25.8 | 26.2 | 22.6 | 27.6 | 25.1 |
| 11. | SEQID 158 | 23.7 | 38.1 | 40 | 38.8 | 36.8 | 35.1 | 29.6 | 35.4 | 35.7 | 34.2 | 25.7 | 26.8 | 22.7 | 26.5 | 26.5 |
| 12. | SEQID 160 | 23.2 | 47.2 | 48.8 | 47.9 | 44.3 | 43.6 | 32.2 | 52.1 | 39.1 | 46.6 | 28.4 | 30.7 | 25.4 | 30.1 | 28.4 |
| 13. | SEQID 162 | 23.2 | 46.9 | 48.8 | 47.6 | 44.1 | 43.3 | 32.2 | 52.1 | 39.1 | 46.6 | 28.4 | 30.9 | 25.4 | 30.4 | 28.4 |
| 14. | SEQID 164 | 21.8 | 45.8 | 47 | 45.3 | 43.9 | 43.9 | 29.8 | 50.9 | 38.2 | 45.8 | 28.5 | 30.3 | 24.8 | 30 | 29 |
| 15. | SEQID 166 | 21.5 | 46 | 47.3 | 45.5 | 43.8 | 43.8 | 30.1 | 51.1 | 38.8 | 46 | 28 | 30.1 | 24.3 | 29.8 | 28.3 |
| 16. | SEQID 168 | 21.3 | 45.9 | 47.6 | 45.9 | 44.4 | 44.4 | 30.2 | 52.5 | 39.2 | 46.6 | 28.3 | 31 | 24.3 | 29.9 | 28.3 |
| 17. | SEQID 170 | 24.5 | 30.8 | 28.1 | 28.3 | 30.8 | 30.2 | 36.9 | 28.5 | 27.3 | 26.1 | 45 | 44.1 | 39.2 | 44.3 | 45.2 |
| 18. | SEQID 172 | 21.6 | 34.3 | 32.2 | 32.2 | 34 | 33.2 | 28.9 | 34.3 | 28.8 | 36.2 | 27 | 28.4 | 25.6 | 25.4 | 27.5 |
| 19. | SEQID 174 | 23.5 | 26.9 | 30.2 | 29.9 | 28.3 | 27.8 | 28.4 | 28 | 24.9 | 27.8 | 25 | 25.9 | 22 | 27.9 | 24.9 |
| 20. | SEQID 176 | 19 | 30.4 | 31.2 | 31.3 | 29.7 | 29.8 | 28.5 | 30.1 | 25.9 | 30.3 | 25.2 | 26.7 | 23.1 | 28 | 25.8 |
| 21. | SEQID 178 | 23.9 | 32.5 | 33.2 | 33.2 | 31.4 | 31.9 | 32.7 | 32 | 31.5 | 31.5 | 33.9 | 31 | 28.6 | 31.4 | 33.1 |
| 22. | SEQID 180 | 20.5 | 27.9 | 29.8 | 29.2 | 27.6 | 28.3 | 24.8 | 27.3 | 22.1 | 25.2 | 27.2 | 26.4 | 23 | 26.1 | 25.8 |
| 23. | SEQID 182 | 24.2 | 24 | 24.3 | 24.5 | 24.8 | 25.1 | 32 | 24.4 | 24.5 | 23.7 | 35.2 | 37.5 | 33.2 | 37.4 | 35.5 |
| 24. | SEQID 184 | 24.9 | 24.5 | 27.1 | 26.5 | 24.9 | 25.6 | 36 | 25.4 | 25.4 | 24.4 | 29.6 | 30.5 | 26.8 | 31 | 29.4 |
| 25. | SEQID 186 | 23.1 | 27.6 | 28 | 27.7 | 27.3 | 26.2 | 25.2 | 28.5 | 24.2 | 27.9 | 25.8 | 24.8 | 23.1 | 25.5 | 25.5 |
| 26. | SEQID 188 | 23.8 | 24.7 | 23.3 | 22 | 24.9 | 25.3 | 25.1 | 24.3 | 20.9 | 23.6 | 28.2 | 27.8 | 26.9 | 25.6 | 27 |
| 27. | SEQID 190 | 21 | 69.5 | 54.9 | 54.5 | 84.3 | 82.1 | 33.6 | 51.3 | 44.6 | 49.5 | 28.7 | 27.6 | 23.9 | 28.2 | 29 |
| 28. | SEQID 192 | 26.7 | 26.7 | 25.2 | 25.2 | 26.5 | 27.2 | 27.1 | 26.1 | 23.5 | 27 | 27.4 | 29.1 | 27.9 | 28.4 | 27 |
| 29. | SEQID 194 | 21.3 | 68.5 | 48.7 | 48.3 | 54.5 | 55.4 | 31.7 | 44.9 | 35.9 | 44.2 | 30.4 | 31.6 | 28.4 | 31.3 | 30.7 |
| 30. | SEQID 196 | 21.9 | 32.3 | 32.5 | 32.7 | 35.2 | 34 | 72.7 | 32.3 | 34.2 | 28.9 | 32.3 | 33.9 | 28.7 | 33 | 32.9 |
| 31. | SEQID 198 | | 20.9 | 18.9 | 18.8 | 20.2 | 20.5 | 23 | 22.1 | 21 | 20.9 | 24.3 | 24.4 | 26.5 | 24.9 | 25.3 |
| 32. | SEQID 200 | 31.5 | | 59.9 | 59.5 | 69.4 | 67.4 | 32.3 | 55.1 | 45.4 | 52.9 | 28.1 | 28.8 | 23.5 | 28.5 | 27.3 |
| 33. | SEQID 202 | 30.7 | 72.9 | | 96.3 | 56.1 | 55.1 | 33.2 | 66.1 | 54 | 58.6 | 30.5 | 28.5 | 24.1 | 28.9 | 30.7 |
| 34. | SEQID 204 | 30.7 | 71.8 | 97.1 | | 55.8 | 54.7 | 33.5 | 65.1 | 53 | 57.1 | 29.7 | 28.9 | 24.3 | 27.7 | 30.4 |
| 35. | SEQID 206 | 32 | 81.1 | 72.2 | 72 | | 97.3 | 34.4 | 53.5 | 45.8 | 49.5 | 28.8 | 28.9 | 25.3 | 27.2 | 28.8 |
| 36. | SEQID 208 | 32.8 | 79.1 | 70.6 | 70.4 | 97.9 | | 33.5 | 52.5 | 44.7 | 48 | 28.4 | 28.1 | 24.7 | 27.1 | 28.4 |
| 37. | SEQID 210 | 43.6 | 43.2 | 43.7 | 43.2 | 45.9 | 46.6 | | 32.1 | 32 | 30.6 | 35.8 | 36.5 | 32.5 | 32.4 | 36 |
| 38. | SEQID 212 | 31.8 | 69 | 79.7 | 78.7 | 68.7 | 67.4 | 42.8 | | 75.1 | 69.5 | 27.9 | 29 | 25.4 | 29.2 | 29.5 |
| 39. | SEQID 214 | 33.5 | 56.1 | 66.9 | 66.1 | 58.7 | 58.3 | 46.4 | 77.7 | | 52.9 | 25.4 | 26.9 | 23.4 | 25.9 | 26.7 |
| 40. | SEQID 216 | 32.3 | 64.9 | 72.5 | 70.7 | 63.9 | 61.6 | 40.9 | 83.1 | 64.4 | | 27 | 29.5 | 24 | 29.2 | 27.5 |
| 41. | SEQID 218 | 43.7 | 42.6 | 41.3 | 40 | 40.5 | 39.3 | 51.1 | 40.3 | 40.1 | 37.6 | | 62.8 | 65.8 | 61.9 | 95.8 |
| 42. | SEQID 220 | 41.8 | 40.6 | 37.8 | 38.1 | 41.1 | 39 | 54.5 | 36.9 | 39.2 | 36.4 | 74.3 | | 47.3 | 94.1 | 64.2 |
| 43. | SEQID 222 | 38.7 | 35.1 | 33.1 | 33.3 | 33.1 | 33.3 | 44.3 | 34.1 | 33.2 | 32.8 | 69 | 54.3 | | 46.5 | 68.8 |
| 44. | SEQID 224 | 40.6 | 40.3 | 39.4 | 38.1 | 36.8 | 37.4 | 49.2 | 37.9 | 38.9 | 36.9 | 72.8 | 96.9 | 54.1 | | 62.4 |
| 45. | SEQID 228 | 42.4 | 43.2 | 42.9 | 41.9 | 40.3 | 38.8 | 52.3 | 41.8 | 41.1 | 38.6 | 97.3 | 74.8 | 70.2 | 73.3 | |
| 46. | SEQID 230 | 31.5 | 72.1 | 79.5 | 76.9 | 70.1 | 68 | 44.4 | 77.7 | 65.1 | 70.5 | 39.6 | 39.1 | 34.4 | 36.5 | 40.9 |
| 47. | SEQID 232 | 30.5 | 68.2 | 74.5 | 72.9 | 68.2 | 66.3 | 42.4 | 74.1 | 62.6 | 65.4 | 38.4 | 40.5 | 31.6 | 38.9 | 40.5 |
| 48. | SEQID 234 | 28.8 | 65.6 | 68.3 | 66.5 | 63.4 | 61.3 | 43.9 | 65.9 | 54.5 | 64.4 | 39.7 | 41.6 | 33 | 38.7 | 39.7 |
| 49. | SEQID 236 | 28.6 | 63.8 | 69.8 | 67.2 | 65.4 | 63.5 | 44.5 | 70.3 | 57.8 | 64.6 | 40.4 | 40.1 | 34.4 | 39.6 | 41.9 |
| 50. | SEQID 240 | 42.6 | 39.5 | 38.4 | 38.7 | 42.4 | 42.3 | 54.5 | 37.2 | 40.4 | 36.4 | 61.3 | 58.2 | 52 | 60.8 | 63.4 |
| 51. | SEQID 242 | 36.1 | 47.3 | 45.5 | 45.3 | 46.7 | 46.6 | 59 | 46.9 | 46.7 | 43.4 | 45.6 | 45.9 | 37 | 45.2 | 45.6 |
| 52. | SEQID 244 | 40.2 | 32.6 | 31 | 31.2 | 33.9 | 33.9 | 42.8 | 31.5 | 35.1 | 31.1 | 42.9 | 43.8 | 40.8 | 42.4 | 40.8 |
| 53. | SEQID 246 | 39.3 | 42.9 | 44.4 | 44.5 | 47.5 | 47.4 | 75.5 | 46.7 | 51.4 | 42.9 | 43.3 | 47 | 37.9 | 46.3 | 45 |
| 54. | SEQID 248 | 40.2 | 42.9 | 40.2 | 39.5 | 41.3 | 40.7 | 55.3 | 40.3 | 44.2 | 37.4 | 56.1 | 62.1 | 49.2 | 61.4 | 58 |
| 55. | SEQID 250 | 34.3 | 46.8 | 45.2 | 46.9 | 46.9 | 46.3 | 55.6 | 46.7 | 47.4 | 43.4 | 43.2 | 43.8 | 34 | 41.9 | 42.6 |
| 56. | SEQID 252 | 36 | 43.7 | 45.8 | 43.2 | 45.1 | 45 | 40.9 | 41.5 | 43 | 43.4 | 38.7 | 39 | 29.9 | 39.9 | 36.6 |
| 57. | SEQID 254 | 39.9 | 42.9 | 45.5 | 45.1 | 46.1 | 45.5 | 64.1 | 44.4 | 47 | 43.2 | 48 | 49.1 | 38.5 | 48 | 48.7 |
| 58. | SEQID 256 | 41.8 | 39.3 | 41.3 | 41.3 | 37.3 | 37.4 | 51.9 | 40 | 39.8 | 38.1 | 62.5 | 66 | 50.4 | 67.5 | 61.1 |

| | | 46 | 47 | 48 | 49 | 50 | 51 | 52 | 53 | 54 | 55 | 56 | 57 | 58 |
|---|---|---|---|---|---|---|---|---|---|---|---|---|---|---|
| 1. | SEQID 132 | 41 | 41.4 | 40.8 | 41.6 | 28.6 | 29.8 | 21.5 | 33.4 | 27.9 | 32.1 | 33.3 | 31.5 | 26.6 |
| 2. | SEQID 140 | 28.1 | 27.1 | 26.8 | 27 | 36.4 | 31.3 | 26.8 | 31.8 | 36.4 | 31.1 | 28.4 | 37.8 | 33.2 |
| 3. | SEQID 142 | 38.8 | 38.3 | 38.6 | 36.2 | 30.3 | 30.5 | 20.2 | 27.2 | 29 | 30.2 | 31.3 | 32.4 | 26.8 |
| 4. | SEQID 144 | 31.3 | 28.4 | 29.2 | 31.1 | 35.1 | 33.7 | 23.5 | 37.8 | 34.6 | 35 | 30.5 | 43.8 | 36.2 |
| 5. | SEQID 146 | 34.8 | 34.4 | 35.8 | 34.5 | 27.8 | 30.4 | 21.5 | 28.1 | 28.1 | 31.9 | 49.7 | 31.5 | 29.6 |
| 6. | SEQID 148 | 43.4 | 39.7 | 39.8 | 37.9 | 29.5 | 32.2 | 21.5 | 29.4 | 28.9 | 30.3 | 31 | 34.5 | 26.7 |
| 7. | SEQID 150 | 26.2 | 25 | 24.9 | 25.3 | 33.2 | 33.7 | 29.1 | 34.1 | 35.1 | 31 | 25.5 | 37.7 | 35.7 |
| 8. | SEQID 152 | 45.3 | 42.4 | 40.8 | 39.5 | 28.3 | 30.4 | 22.1 | 28.6 | 28.1 | 30.7 | 35.7 | 31 | 25.4 |
| 9. | SEQID 154 | 43.1 | 37.4 | 36.8 | 39.7 | 26 | 27.8 | 19.8 | 27.1 | 27.3 | 30.6 | 30.2 | 28.7 | 27.8 |
| 10. | SEQID 156 | 39.2 | 37.9 | 37.5 | 34.5 | 28.7 | 27.7 | 19.9 | 27.4 | 27 | 29.1 | 28.8 | 30.8 | 26.8 |
| 11. | SEQID 158 | 40.9 | 37 | 38.6 | 37 | 29.5 | 29.8 | 21.1 | 27 | 27.4 | 29.2 | 31 | 32.9 | 26.5 |
| 12. | SEQID 160 | 49.3 | 46.3 | 44.8 | 44.4 | 28.3 | 30.8 | 24.1 | 29.9 | 29.7 | 29.3 | 31.1 | 32.2 | 27 |
| 13. | SEQID 162 | 49.3 | 46 | 45.1 | 44.4 | 28.3 | 30.8 | 24.4 | 29.1 | 30 | 29.3 | 31.6 | 32.5 | 27 |
| 14. | SEQID 164 | 46.6 | 42.3 | 43.2 | 42.6 | 29.7 | 31 | 22.6 | 28.3 | 29.2 | 29.9 | 30.6 | 29.8 | 28.8 |
| 15. | SEQID 166 | 47.1 | 42.7 | 43.6 | 42.8 | 30.1 | 31.8 | 21.8 | 28.8 | 29.3 | 30.2 | 30.8 | 29.6 | 28.8 |
| 16. | SEQID 168 | 46.7 | 42.7 | 44 | 43.1 | 30.1 | 31.4 | 21.7 | 28.6 | 28.8 | 30.3 | 31.9 | 30.2 | 28.6 |
| 17. | SEQID 170 | 30.2 | 29.1 | 29.1 | 28.4 | 90.8 | 32.5 | 29 | 34.3 | 41.2 | 31.4 | 27.5 | 35.4 | 42.8 |
| 18. | SEQID 172 | 34.9 | 31.7 | 31.5 | 33.5 | 29.8 | 28.7 | 19.5 | 26.9 | 29.7 | 31.5 | 36 | 30.1 | 27.9 |
| 19. | SEQID 174 | 31.1 | 28 | 28 | 30.8 | 29 | 28.3 | 21.1 | 26.9 | 29.4 | 28.8 | 32.4 | 25.5 | 28.6 |
| 20. | SEQID 176 | 31.6 | 29.9 | 32.2 | 33 | 28.7 | 26.5 | 22.5 | 28 | 25.5 | 27.1 | 35.9 | 30.8 | 27.7 |
| 21. | SEQID 178 | 35.5 | 33.3 | 31.4 | 32.2 | 31.2 | 28.9 | 26.6 | 30.1 | 31.4 | 29.3 | 30 | 35 | 30.6 |
| 22. | SEQID 180 | 28.2 | 26.4 | 29.6 | 29 | 26.7 | 27.1 | 19.6 | 24.1 | 25.7 | 24.1 | 27.4 | 26.8 | 27.5 |
| 23. | SEQID 182 | 24.1 | 24.5 | 25.7 | 26.8 | 36.2 | 28.9 | 23.6 | 27 | 33 | 28 | 27.7 | 33.3 | 35.2 |
| 24. | SEQID 184 | 27.4 | 25.7 | 25.6 | 26.2 | 34.9 | 31.8 | 23.7 | 32.8 | 33.6 | 30.6 | 29.8 | 34.2 | 33 |
| 25. | SEQID 186 | 30 | 28.3 | 27 | 29 | 28.2 | 25.4 | 22.6 | 25.6 | 25.8 | 28 | 32.4 | 26.9 | 28.3 |
| 26. | SEQID 188 | 23.6 | 21.3 | 22.1 | 24.8 | 27.5 | 23.1 | 27.9 | 23 | 29.1 | 23.2 | 25.8 | 23.4 | 28.3 |
| 27. | SEQID 190 | 52 | 49.7 | 46.2 | 45.1 | 28.6 | 37.1 | 22.2 | 32.2 | 28.9 | 36.1 | 32.2 | 35.1 | 27.2 |
| 28. | SEQID 192 | 27.5 | 26.1 | 27.8 | 26.9 | 29.1 | 26.4 | 24.7 | 24.6 | 30.4 | 24.9 | 28.7 | 27.4 | 29.2 |
| 29. | SEQID 194 | 46.2 | 45.3 | 41.8 | 40.1 | 31.5 | 32.8 | 26.6 | 31.3 | 33.2 | 30.9 | 32.6 | 31.4 | 30.7 |
| 30. | SEQID 196 | 34.1 | 31.4 | 31 | 30.9 | 37.3 | 44.6 | 26.2 | 77.7 | 33.2 | 46.2 | 28.8 | 51.1 | 34.9 |
| 31. | SEQID 198 | 18.9 | 19.2 | 19.9 | 19.9 | 23.7 | 20 | 24.9 | 21.4 | 22.5 | 23.7 | 22.8 | 22.1 | 25.1 |
| 32. | SEQID 200 | 57.4 | 55.4 | 48.4 | 46.9 | 30.1 | 35.5 | 22.1 | 32.4 | 31.5 | 34.5 | 32 | 34.5 | 29.7 |
| 33. | SEQID 202 | 67.1 | 59.2 | 53.5 | 54.4 | 28.6 | 33.8 | 20.4 | 31.5 | 30.3 | 33.2 | 31.1 | 35 | 31 |
| 34. | SEQID 204 | 66 | 58 | 51.1 | 52.5 | 28.8 | 31.1 | 20.3 | 31.2 | 28.6 | 34 | 30.5 | 34.6 | 31 |
| 35. | SEQID 206 | 51.8 | 49 | 45.6 | 45.3 | 30.2 | 34.7 | 22.3 | 33.5 | 29.5 | 34.5 | 30.7 | 36.8 | 28.3 |
| 36. | SEQID 208 | 50.3 | 47.8 | 44.2 | 44.2 | 29.6 | 34.8 | 22.4 | 32.7 | 28.9 | 33.4 | 30.6 | 36 | 28.7 |
| 37. | SEQID 210 | 33.6 | 31.4 | 33.9 | 34 | 38.2 | 44.3 | 26.8 | 67.9 | 37.6 | 46.3 | 29.5 | 48.6 | 33.9 |
| 38. | SEQID 212 | 63.7 | 57 | 49.9 | 52.2 | 28 | 34 | 22.3 | 31.6 | 28.4 | 33.8 | 31.2 | 34.3 | 29.6 |
| 39. | SEQID 214 | 50.8 | 48.2 | 40.3 | 43.5 | 26.1 | 32.4 | 20.4 | 30.6 | 28.4 | 31 | 25.6 | 34 | 25.4 |
| 40. | SEQID 216 | 57.9 | 53.2 | 49.5 | 48.5 | 27.3 | 32.3 | 19.9 | 29.9 | 27.7 | 31.8 | 31.6 | 31 | 27.4 |
| 41. | SEQID 218 | 28.6 | 26.6 | 28.8 | 30.1 | 44.4 | 31.5 | 26.3 | 29.1 | 44.6 | 29.4 | 25.3 | 32.2 | 50 |
| 42. | SEQID 220 | 30.4 | 30.2 | 30.1 | 31.2 | 45.8 | 32.3 | 25 | 31.5 | 48 | 30.6 | 27.9 | 32.4 | 52.1 |
| 43. | SEQID 222 | 26.5 | 24.5 | 24.9 | 25.6 | 37.7 | 26.9 | 28.8 | 25.3 | 37.5 | 25.6 | 21 | 27.1 | 41.8 |
| 44. | SEQID 224 | 28.1 | 29.1 | 29.3 | 29.6 | 44.5 | 31.1 | 24.7 | 29.9 | 46.9 | 29.5 | 27.5 | 30.5 | 51.3 |
| 45. | SEQID 228 | 29.4 | 27.6 | 29.3 | 30.7 | 45.3 | 31 | 24.8 | 29.7 | 45 | 30.3 | 24.9 | 31.8 | 49.4 |
| 46. | SEQID 230 | | 68.5 | 52.9 | 51.9 | 30.8 | 33.3 | 22.1 | 31.9 | 31.4 | 35.1 | 32.9 | 36 | 29.1 |
| 47. | SEQID 232 | 80.1 | | 46 | 47.6 | 27.8 | 29.5 | 20.5 | 30 | 29.7 | 35.6 | 32.9 | 32.8 | 27.4 |
| 48. | SEQID 234 | 68.3 | 62.6 | | 69.4 | 27.5 | 34 | 20.3 | 31.7 | 28.4 | 32.5 | 29.7 | 32.9 | 26 |
| 49. | SEQID 236 | 69.8 | 66.4 | 80.5 | | 28.6 | 31.9 | 21.2 | 30.8 | 29.4 | 33.9 | 30.8 | 33.1 | 27.8 |
| 50. | SEQID 240 | 39.9 | 37.9 | 37.4 | 41.1 | | 31.3 | 28.6 | 33.9 | 42 | 32.3 | 28.4 | 36.6 | 40.4 |
| 51. | SEQID 242 | 45.4 | 42.4 | 46 | 43.8 | 45.9 | | 22.3 | 41.3 | 33.2 | 41.7 | 25.4 | 52.8 | 31.3 |
| 52. | SEQID 244 | 31.8 | 31.1 | 31.9 | 31.3 | 47.2 | 38 | | 23.3 | 27 | 23.3 | 22.2 | 23.2 | 28 |
| 53. | SEQID 246 | 45.9 | 41.1 | 44.2 | 43 | 49.3 | 60.7 | 38.9 | | 32.8 | 43.1 | 26 | 47.5 | 31.1 |
| 54. | SEQID 248 | 44.6 | 40 | 39.5 | 40.9 | 60.2 | 47.2 | 39.4 | 48.7 | | 30.5 | 29.3 | 33.2 | 39.9 |
| 55. | SEQID 250 | 44.9 | 48.7 | 44.9 | 47.1 | 44.4 | 53.8 | 33.7 | 56.5 | 42.9 | | 29.2 | 46.7 | 30.2 |
| 56. | SEQID 252 | 47.2 | 45.8 | 39.5 | 43.5 | 40.2 | 40.9 | 32.6 | 42.4 | 41.8 | 44.4 | | 26.5 | 26.9 |
| 57. | SEQID 254 | 45.4 | 41.6 | 43.1 | 44.5 | 57.9 | 63.6 | 39.6 | 61.1 | 49.8 | 56.5 | 38.4 | | 31.9 |
| 58. | SEQID 256 | 39.6 | 36.6 | 37.1 | 35.9 | 58.3 | 46.9 | 42.1 | 45 | 54.2 | 41.9 | 39.9 | 46.5 | |

### Example 12

### Identification of Domains Comprised in Polypeptide Sequences Useful in Performing the Methods of the Invention

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, ProDom and Pfam, Smart and TIGRFAMs. Interpro is hosted at the European Bioinformatics Institute in the United Kingdom.

The results of the InterPro scan of the polypeptide sequence as represented by SEQ ID NO: 132 resented in Table 16.

TABLE 16
InterPro scan results of the polypeptide sequence as represented
by SEQ ID NO: 132

| Database | Accession number | Accession name |
|---|---|---|
| PRODOM | PD001423 | Q9SP16_ORYSA_Q9SP16; |
| PRINTS | PR00367 | ETHRSPELEMNT |
| GENE3D | G3DSA:3.30.730.10 | no description |
| PFAM | PF00847 | AP2 |
| SMART | SM00380 | AP2 |
| PROFILE | PS51032 | AP2_ERF |
| SUPERFAMILY | SSF54171 | DNA binding domain |

### Example 13

### Topology Prediction of the Polypeptide Sequences Useful in Performing the Methods of the Invention (Subcellular Localization, Transmembrane . . . )

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

A number of parameters were selected, such as organism group (non-plant or plant), cutoff sets (none, predefined set of cutoffs, or user-specified set of cutoffs), and the calculation of prediction of cleavage sites (yes or no).

The results of TargetP 1.1 analysis of the polypeptide sequence as represented by SEQ ID NO: 132 are presented Table 17. The “plant” organism group has been selected, no cutoffs defined, and the predicted length of the transit peptide requested. The subcellular localization of the polypeptide sequence as represented by SEQ ID NO: 132 may be the cytoplasm or nucleus, no transit peptide is predicted.

TABLE 17
TargetP 1.1 analysis of the polypeptide sequence as represented
by SEQ ID NO: 132

| | |
|---|---|
| Length (AA) | 130 |
| Chloroplastic transit peptide | 0.098 |
| Mitochondrial transit peptide | 0.339 |
| Secretory pathway signal peptide | 0.035 |
| Other subcellular targeting | 0.797 |
| Predicted Location | / |
| Reliability class | 3 |
| Predicted transit peptide length | / |

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark

### Example 14

### Expression Vector Construction Using the Nucleic Acid Sequence as Represented by SEQ ID NO: 131

Unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

The Oryza sativa AP2-2 gene was amplified by PCR from a rice cDNA library. The PCR fragment of the expected length was purified and subsequently cloned in a Gateway® vector using standard technology. The entry clone comprising SEQ ID NO: 131 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 39) for root specific expression was located upstream of this Gateway cassette.

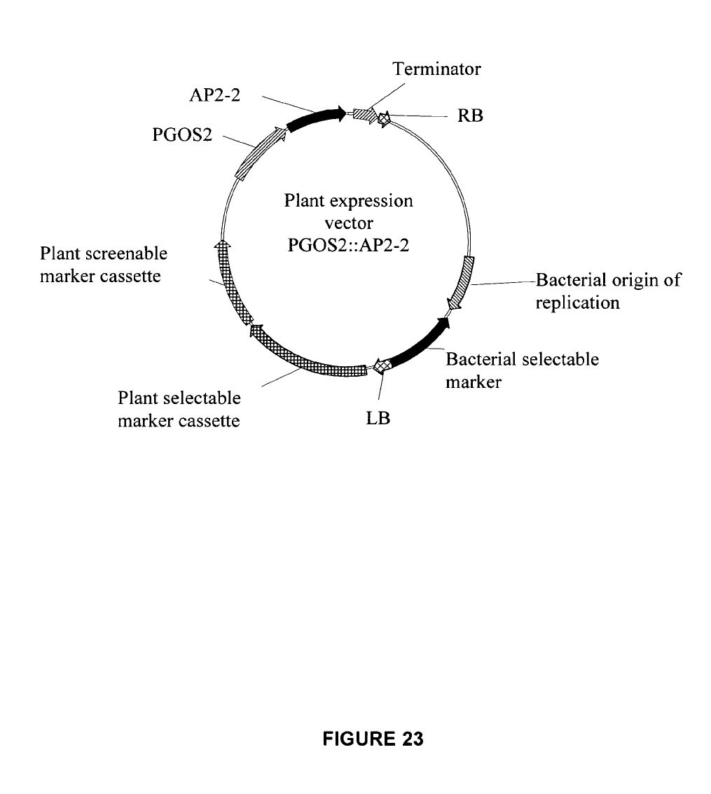

After the LR recombination step, the resulting expression vector pGOS2::AP2-2 (FIG. 23) was transformed into Agrobacterium strain LBA4044 according to methods well known in the art.

### Example 15

### Plant Transformation

### Rice Transformation

The Agrobacterium containing the expression vector was used to transform Oryza sativa plants. Mature dry seeds of the rice japonica cultivar Nipponbare were dehusked. Sterilization was carried out by incubating for one minute in 70% ethanol, followed by 30 minutes in 0.2% HgCl₂, followed by a 6 times 15 minutes wash with sterile distilled water. The sterile seeds were then germinated on a medium containing 2,4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were sub-cultured on fresh medium 3 days before co-cultivation (to boost cell division activity).

Agrobacterium strain LBA4404 containing the expression vector was used for co-cultivation. Agrobacterium was inoculated on AB medium with the appropriate antibiotics and cultured for 3 days at 28° C. The bacteria were then collected and suspended in liquid co-cultivation medium to a density (OD₆₀₀) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent T0 rice transformants were generated for one construct. The primary transformants were transferred from a tissue culture chamber to a greenhouse. After a quantitative PCR analysis to verify copy number of the T-DNA insert, only single copy transgenic plants that exhibit tolerance to the selection agent were kept for harvest of T1 seed. Seeds were then harvested three to five months after transplanting. The method yielded single locus transformants at a rate of over 50% (Aldemita and Hodges 1996, Chan et al. 1993, Hiei et al. 1994).

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radicle and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7% Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MS0) for root induction. The rooted shoots are transplanted to soil in the greenhouse. T1 seeds are produced from plants that exhibit tolerance to the selection agent and that contain a single copy of the T-DNA insert.

### Alfalfa Transformation

A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown D C W and A Atanassov (1985. Plant Cell Tissue Organ Culture 4: 111-112). Alternatively, the RA3 variety (University of Wisconsin) has been selected for use in tissue culture (Walker et al., 1978 μm J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K2SO4, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings were transplanted into pots and grown in a greenhouse. T1 seeds are produced from plants that exhibit tolerance to the selection agent and that contain a single copy of the T-DNA insert.

### Example 16

### Phenotypic Evaluation Procedure

### 16.1 Evaluation Setup

Approximately 35 independent T0 rice transformants were generated. The primary transformants were transferred from a tissue culture chamber to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%.

Four T1 events were further evaluated in the T2 generation following the same evaluation procedure as for the T1 generation but with more individuals per event. From the stage of sowing until the stage of maturity the plants were passed several times through a digital imaging cabinet. At each time point digital images (2048×1536 pixels, 16 million colours) were taken of each plant from at least 6 different angles.

### 16.2 Statistical Analysis: F-Test

A two factor ANOVA (analysis of variants) was used as a statistical model for the overall evaluation of plant phenotypic characteristics. An F-test was carried out on all the parameters measured of all the plants of all the events transformed with the gene of the present invention. The F-test was carried out to check for an effect of the gene over all the transformation events and to verify for an overall effect of the gene, also known as a global gene effect. The threshold for significance for a true global gene effect was set at a 5% probability level for the F-test. A significant F-test value points to a gene effect, meaning that it is not only the mere presence or position of the gene that is causing the differences in phenotype.

### 16.3 Parameters Measured

### Biomass-Related Parameter Measurement

From the stage of sowing until the stage of maturity the plants were passed several times through a digital imaging cabinet. At each time point digital images (2048×1536 pixels, 16 million colours) were taken of each plant from at least 6 different angles.

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass. The early vigour is the plant (seedling) aboveground area three weeks post-germination. Increase in root biomass is expressed as an increase in total root biomass (measured as maximum biomass of roots observed during the lifespan of a plant); or as an increase in the root/shoot index (measured as the ratio between root mass and shoot mass in the period of active growth of root and shoot).

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

### Example 17

### Results of the Phenotypic Evaluation of the Transgenic Plants

The results of the evaluation of transgenic rice plants expressing the AP2-2 nucleic acid are as follows.

There was an increase in yield compared to corresponding nullizygotes (controls), in particular seed yield, expressed as Thousand Kernel Weight and Harvest Index, was statistically significant increased compared to the control plants. For the TKW, an overall increase of 2% in T1 and of 3.4% in T2 was observed compared to control plants. In the case of harvest index, the overall increase compared to control plants was respectively 9.2% (T1) and 36.2% (T2).

### D) APETELA2-70-Like (AP2-70-Like) Polypeptide

### Example 18

### Identification of AP2-70-Like Sequences

Sequences (full length cDNA, ESTs or genomic) related to SEQ ID NO: 257 and/or protein sequences related to SEQ ID NO: 258 were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program was used to find regions of local similarity between sequences by comparing nucleic acid or polypeptide sequences to sequence databases and by calculating the statistical significance of matches. The polypeptide encoded by SEQ ID NO: 257 was used for the TBLASTN algorithm, with default settings and the filter to ignore low complexity sequences set off. The output of the analysis was viewed by pairwise comparison, and ranked according to the probability score (E-value), where the score reflects the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search.

Table 18 provides a list of nucleic acid and protein sequences related to the nucleic acid sequence as represented by SEQ ID NO: 257 and the protein sequence represented by SEQ ID NO: 258.

TABLE 18
AP2-70-encoding nucleic acid sequences and AP2-70 polypeptides useful
in the methods of the present invention.

| | | Nucleic acid | Poly-peptide | |
|---|---|---|---|---|
| Name | Source organism | SEQ ID NO: | SEQ ID NO: | Status |
| | Oryza sativa | 257 | 258 | Full length |
| | Oryza sativa | 259 | 260 | Full length |
| NP_001049235.1 | Oryza sativa | 261 | 262 | full length |
| ABB90886.1 | Triticum aestivum | 263 | 264 | Full length |
| AAZ08560.1\| | Triticum aestivum | 265 | 266 | Full length |
| AAX13274.1 | Triticum aestivum | 267 | 268 | Full length |
| NM_001054677.1 | Oryza sativa | 269 | 270 | Full length |
| ABK28776.1 | Arabidopsis thaliana | 271 | 272 | Full length |
| BAD37688.1 | Oryza sativa | 273 | 274 | Full length |
| ABB89754.1 | Asparagus officinalis | 275 | 276 | Full length |
| ABJ09421.1 | Aloe vera | 277 | 278 | Full length |
| AAT39542.1 | Gossypium hirsutum | 279 | 280 | Full length |
| AAF17691.1 | Arabidopsis thaliana | 281 | 282 | Full length |
| NP_177931.1 | Arabidopsis thaliana | 283 | 284 | Full length |
| NP_195688.1 | Arabidopsis thaliana | 285 | 286 | Full length |
| AAM08622.1 | Oryza sativa | 287 | 288 | Full length |
| ABB89755.1 | Broussonetia papyrifera | 289 | 290 | Full length |
| AAZ14831.1 | Jatropha curcas | 291 | 292 | Full length |
| AAC49770.1 | Arabidopsis thaliana | 293 | 294 | Full length |
| NP_001063013.1 | Oryza sativa | 295 | 296 | Full length |
| NP_001061779.1 | Oryza sativa | 297 | 298 | Full length |
| AAO13360.1 | Lycopersicon esculentum | 299 | 300 | Full length |
| AAM80486.1 | Zea mays | 301 | 302 | Full length |
| AAP56252.1 | Oryza sativa | 303 | 304 | Full length |
| AAF76898.1 | Atriplex hortensis | 305 | 306 | Full length |
| NP_173638.1 | Arabidopsis thaliana | 307 | 308 | Full length |
| NP_179810.1 | Arabidopsis thaliana | 309 | 310 | Full length |
| AAZ03388.1 | Glycine max | 311 | 312 | Full length |
| NP_564468.1 | Arabidopsis thaliana | 313 | 314 | Full length |
| NP_179685.1 | Arabidopsis thaliana | 315 | 316 | Full length |
| NP_193098.1 | Arabidopsis thaliana | 317 | 318 | Full length |
| BAD25342.1 | Oryza sativa | 319 | 320 | Full length |
| NP_001053379.1 | Oryza sativa | 321 | 322 | Full length |
| CAD41199.2 | Oryza sativa | 323 | 324 | Full length |
| NP_176620.1 | Arabidopsis thaliana | 325 | 326 | Full length |
| 11978.m07152 | Oryza sativa | 327 | 328 | Full length |
| 11975.m09040 | Oryza sativa | 329 | 330 | Full length |

### Example 19

### Alignment of AP2-70-Like Polypeptide Sequences

Alignment of polypeptide sequences was performed using the AlignX programme from the Vector NTI (Invitrogen) which is based on the popular Clustal algorithm of progressive alignment (Thompson et al. (1997) Nucleic Acids Res 25:4876-4882; Chenna et al. (2003). Nucleic Acids Res 31:3497-3500). Default values are for the gap open penalty of 10, for the gap extension penalty of 0,1 and the selected weight matrix is Blosum 62 (if polypeptides are aligned).

A phylogenetic tree of AP2-70-like polypeptides was constructed using a neighbour-joining clustering algorithm as provided in the AlignX programme from the Vector NTI (Invitrogen).

### Example 20

### Cloning and Vector Construction

Unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

The Oryza sativa AP2-70 gene was amplified by PCR from a rice cDNA library. The PCR fragment of the expected length was purified and subsequently cloned in a Gateways vector using standard technology. The entry clone comprising SEQ ID NO: 257 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 39) for constitutive expression was located upstream of this Gateway cassette.

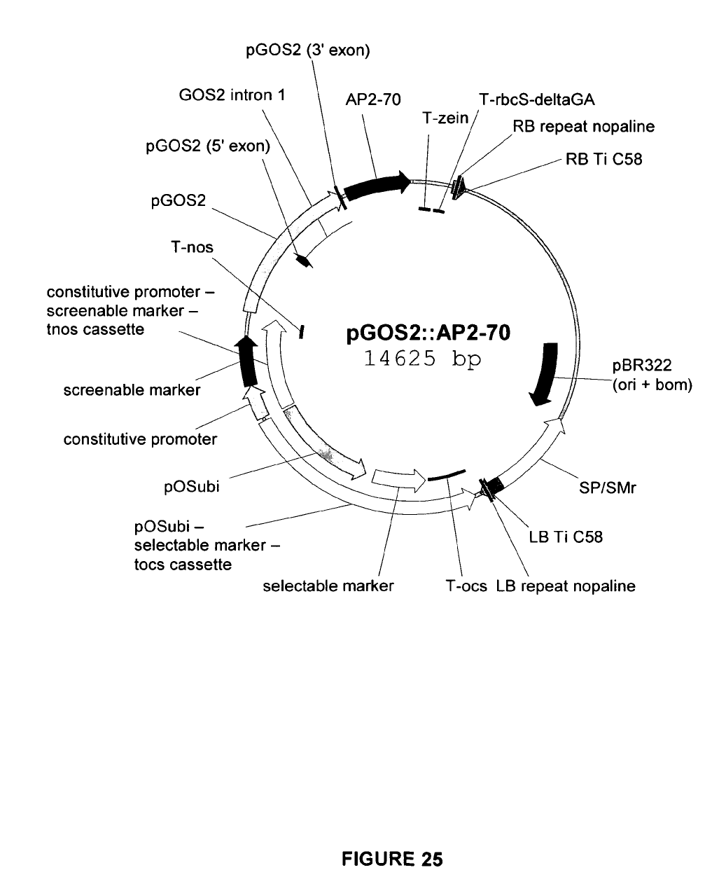

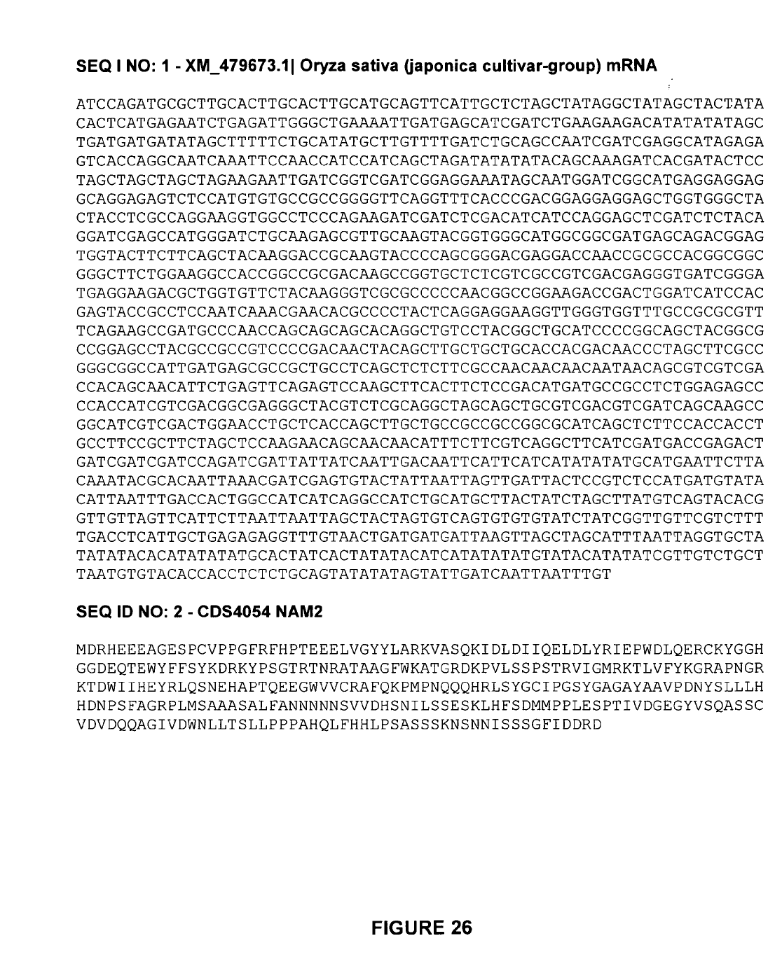

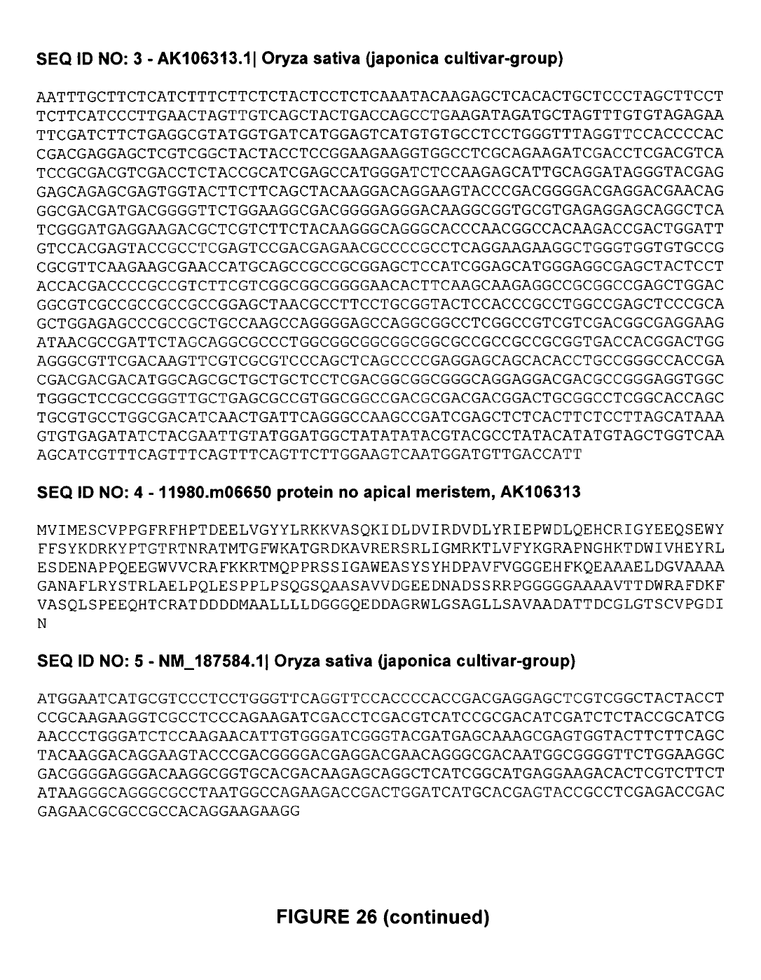

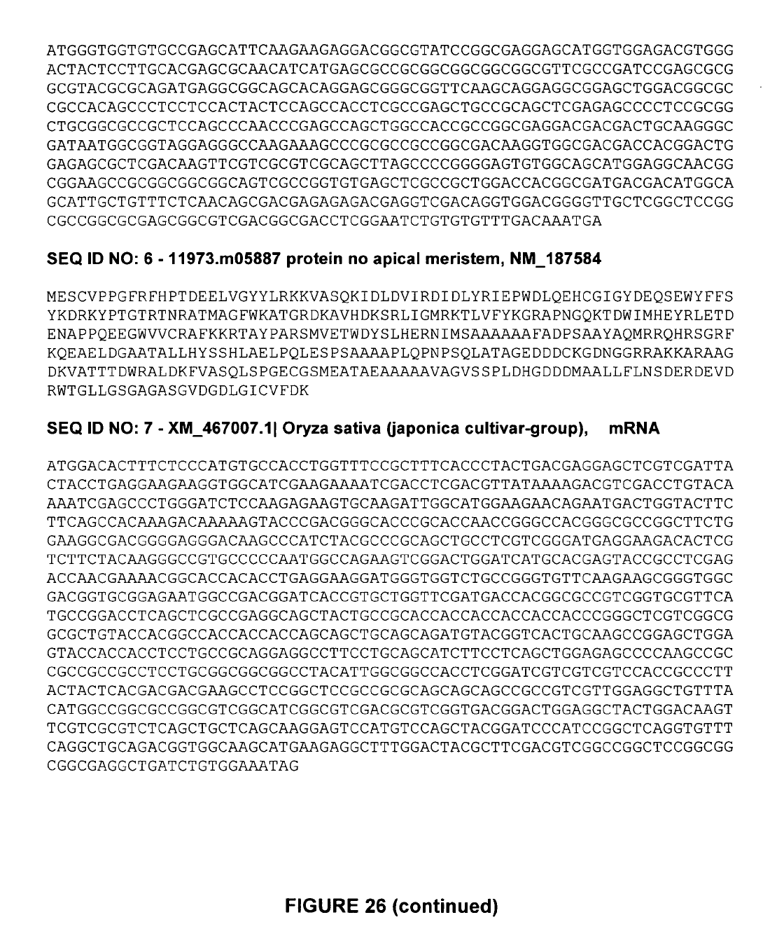

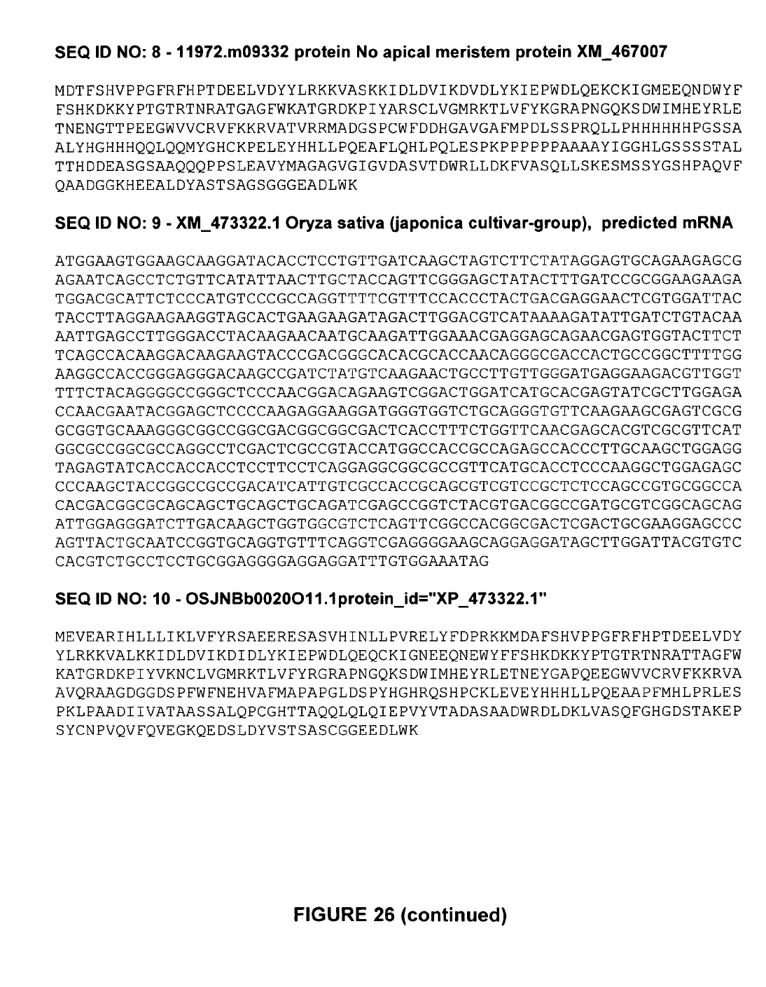

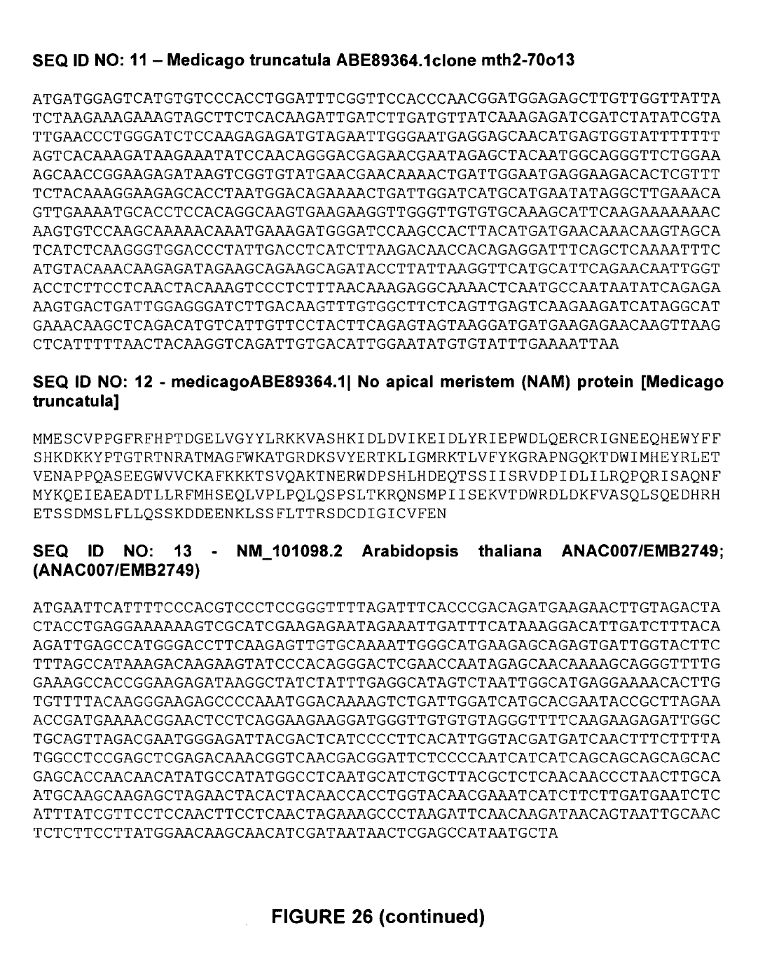

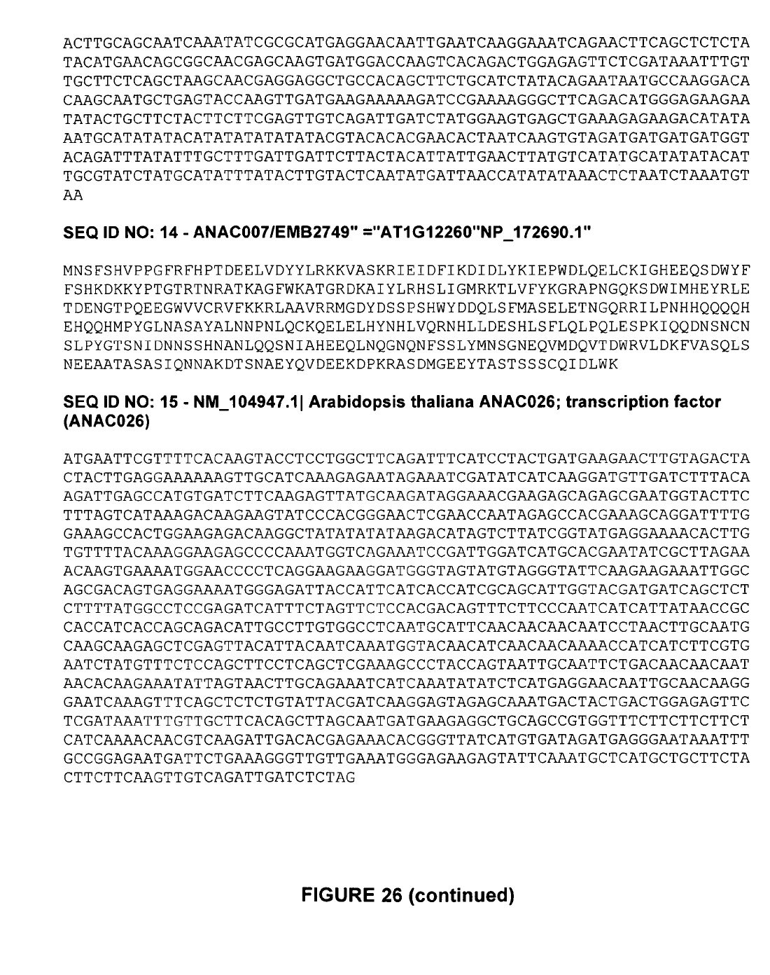

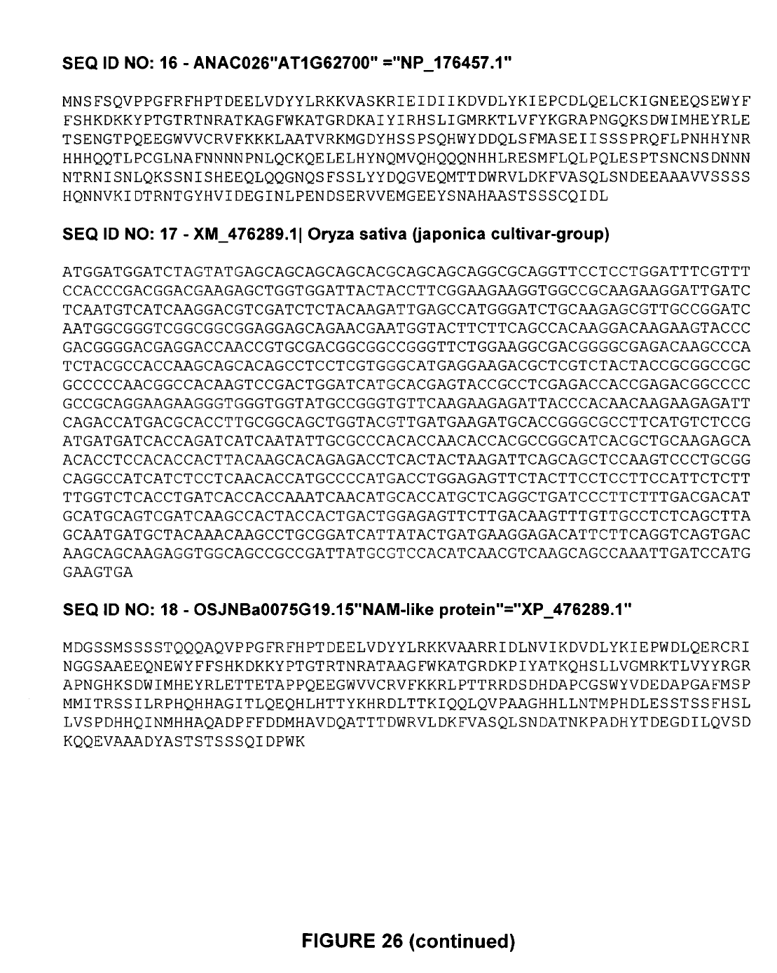

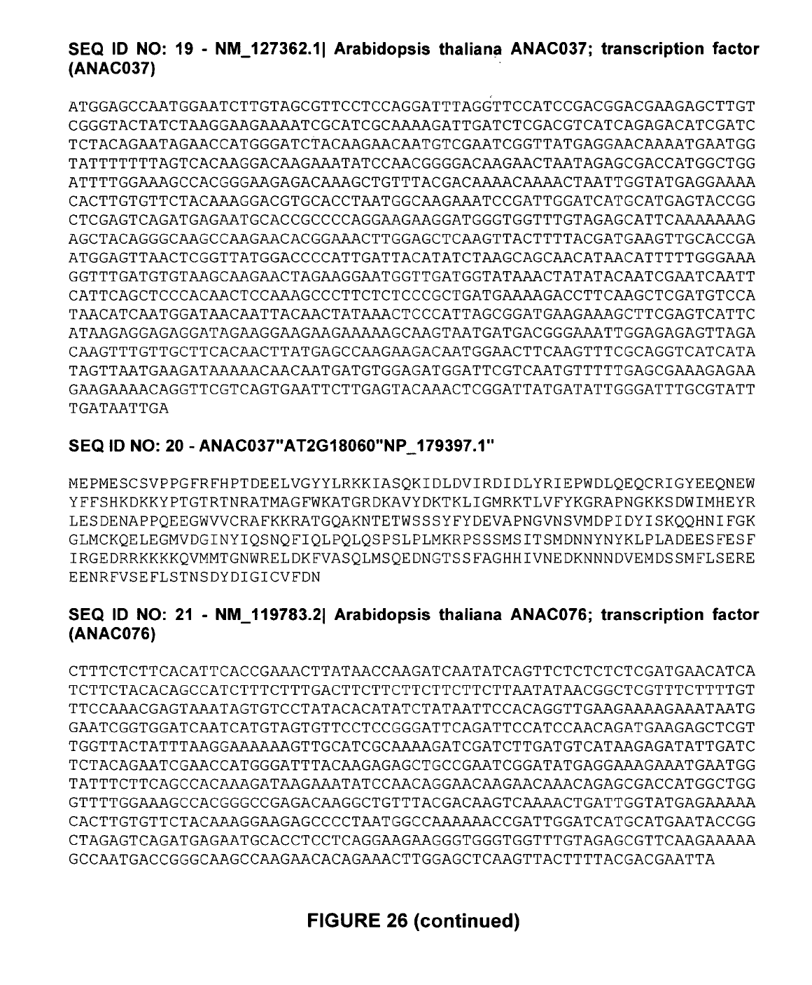

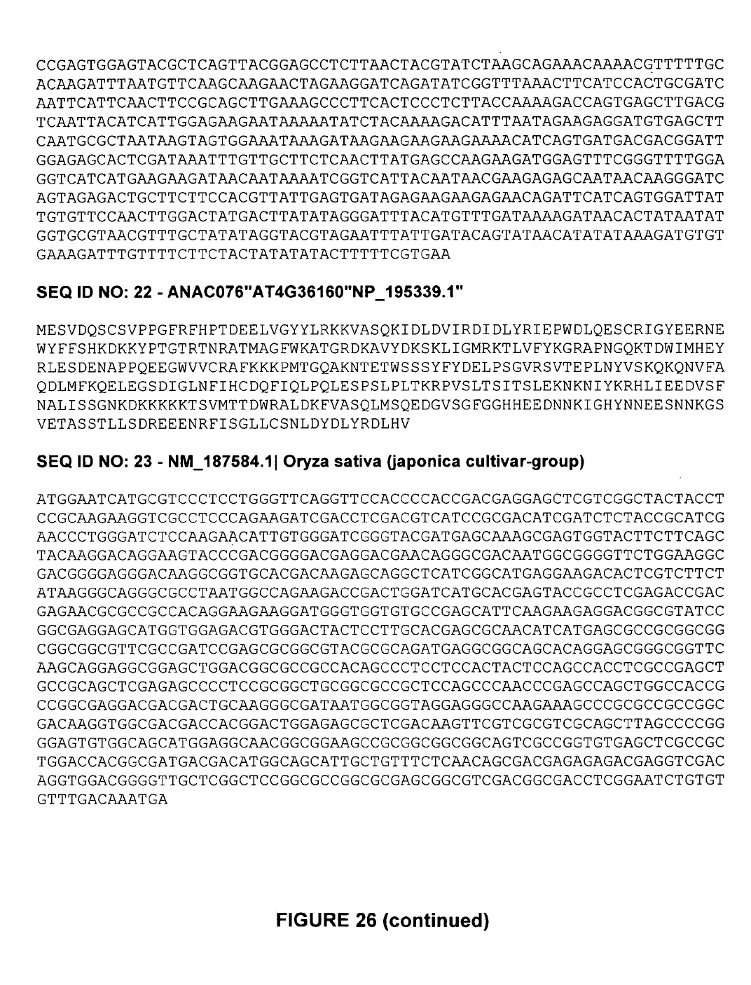

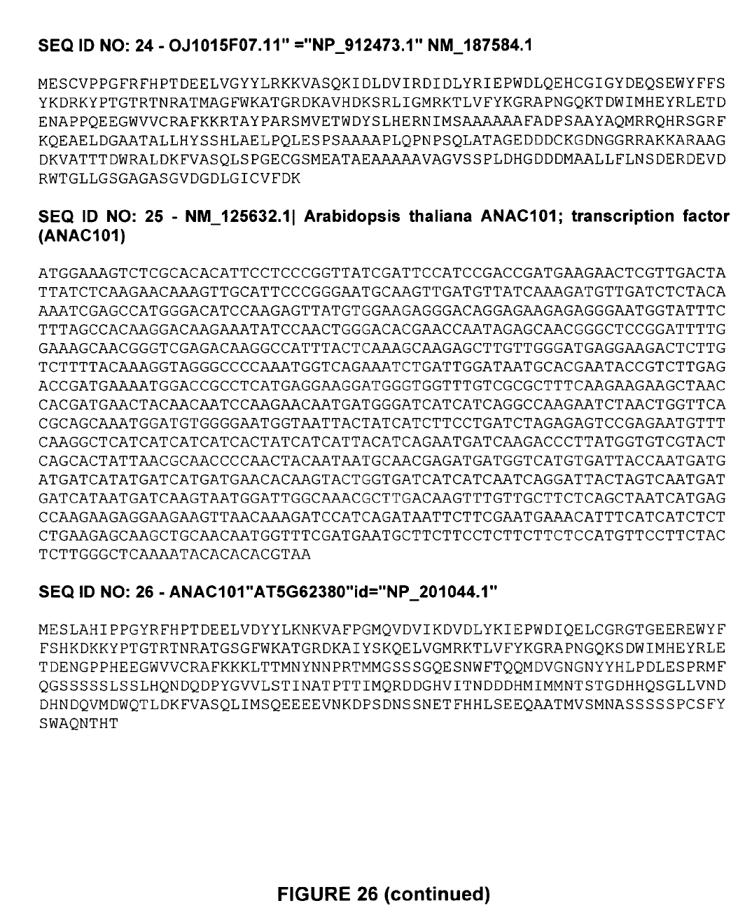

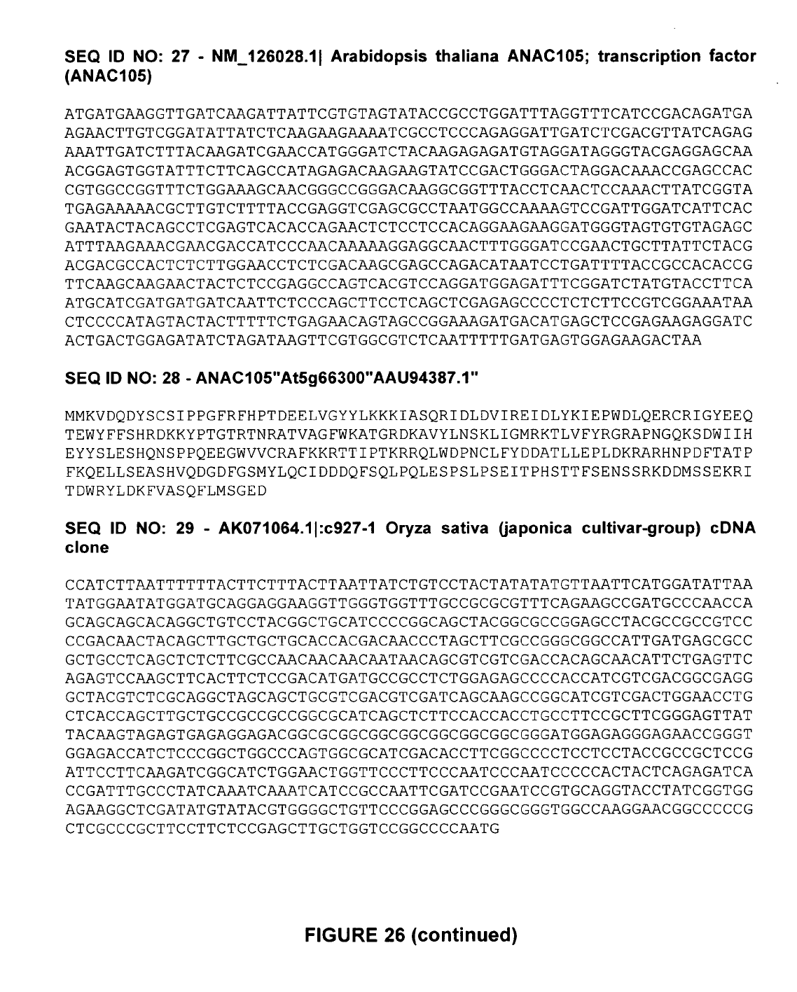

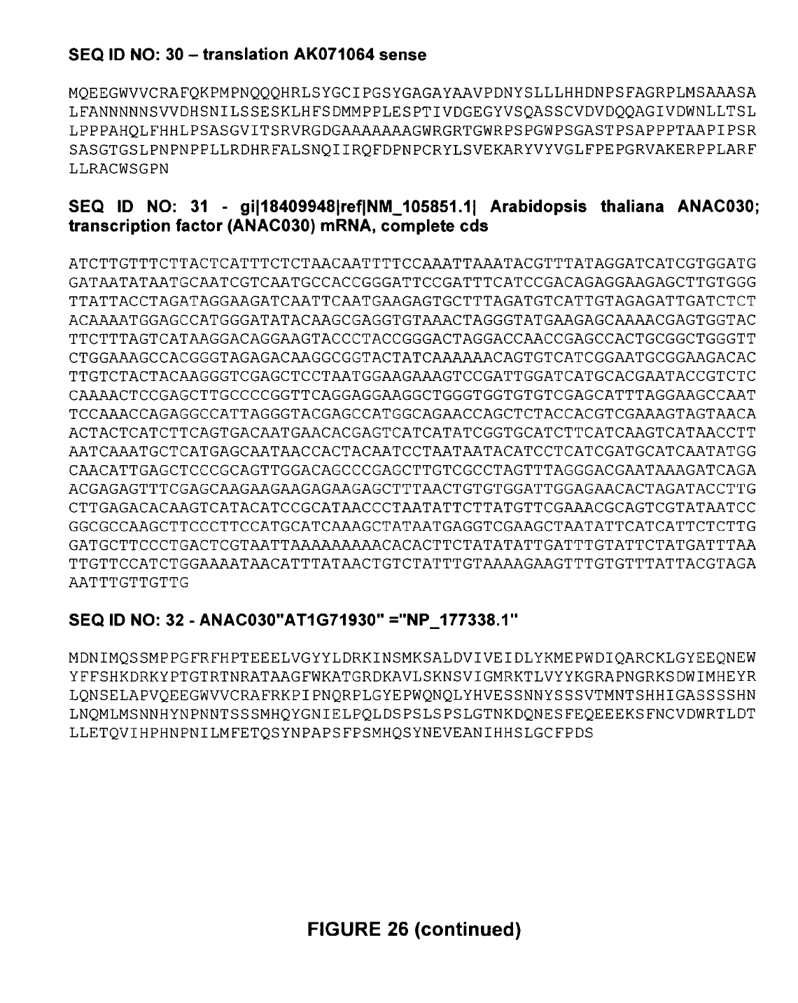

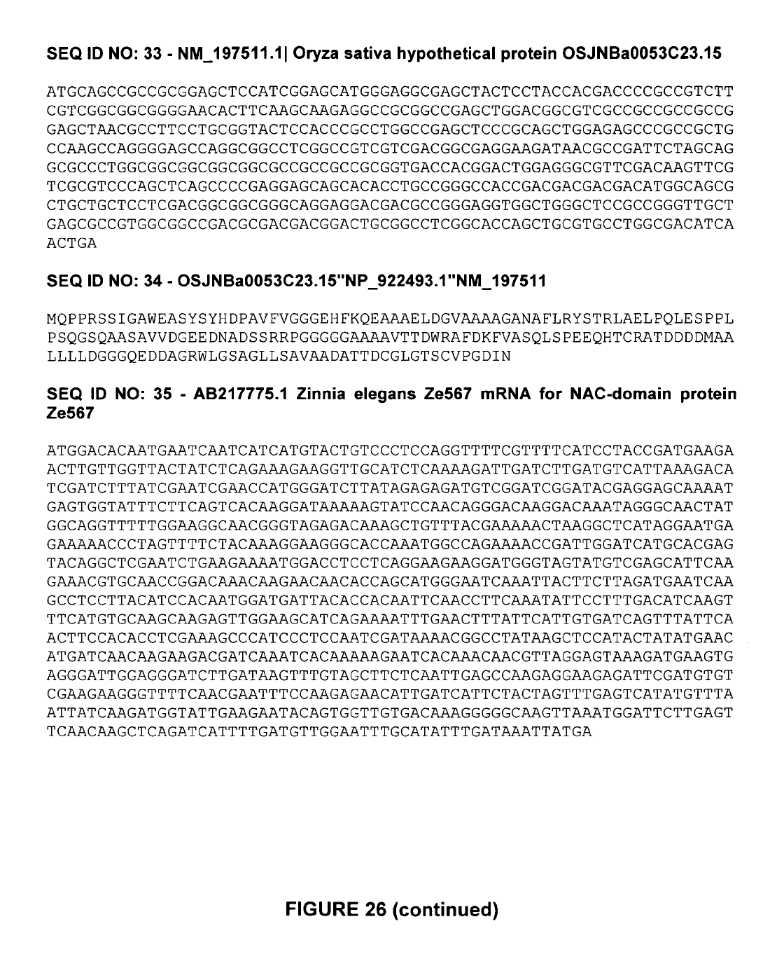

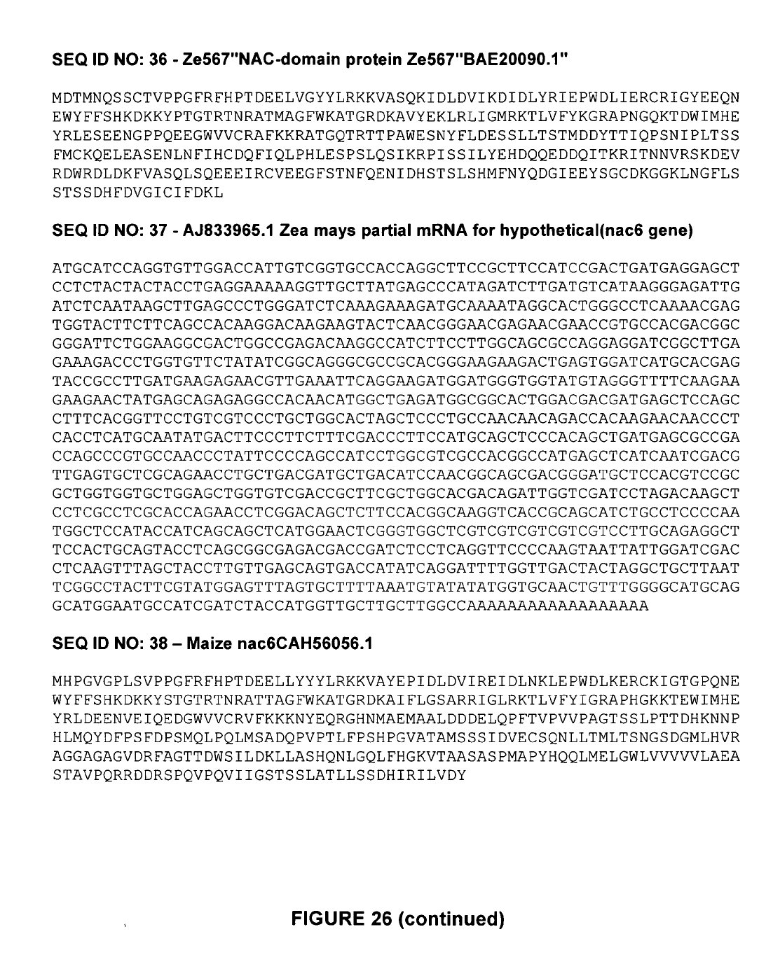

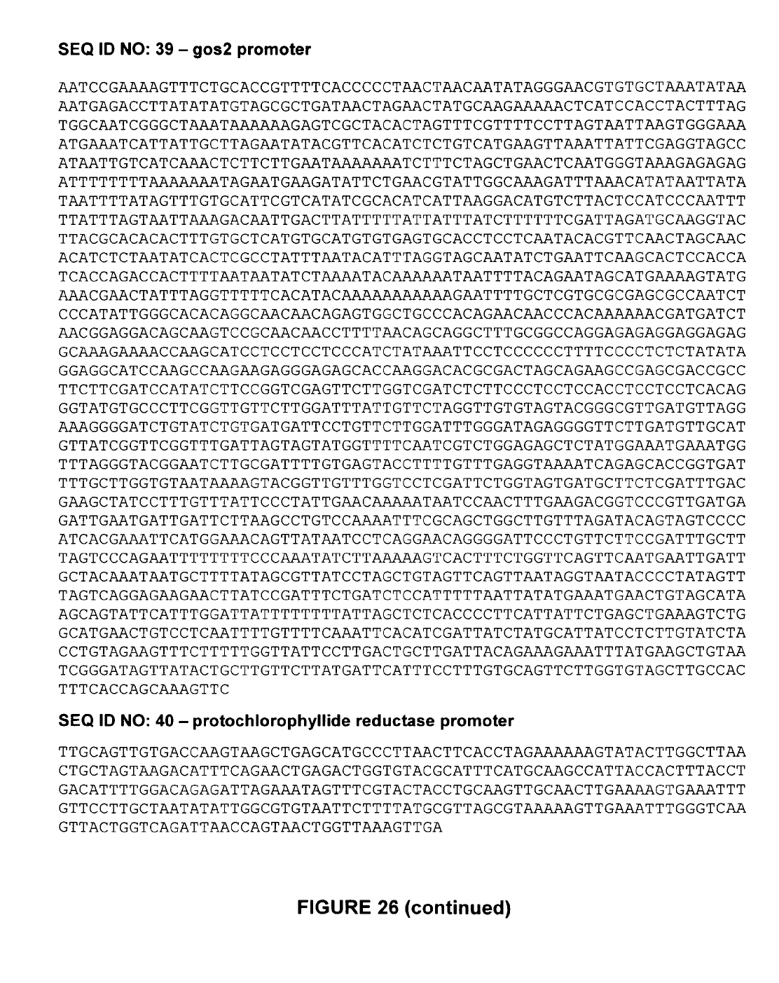

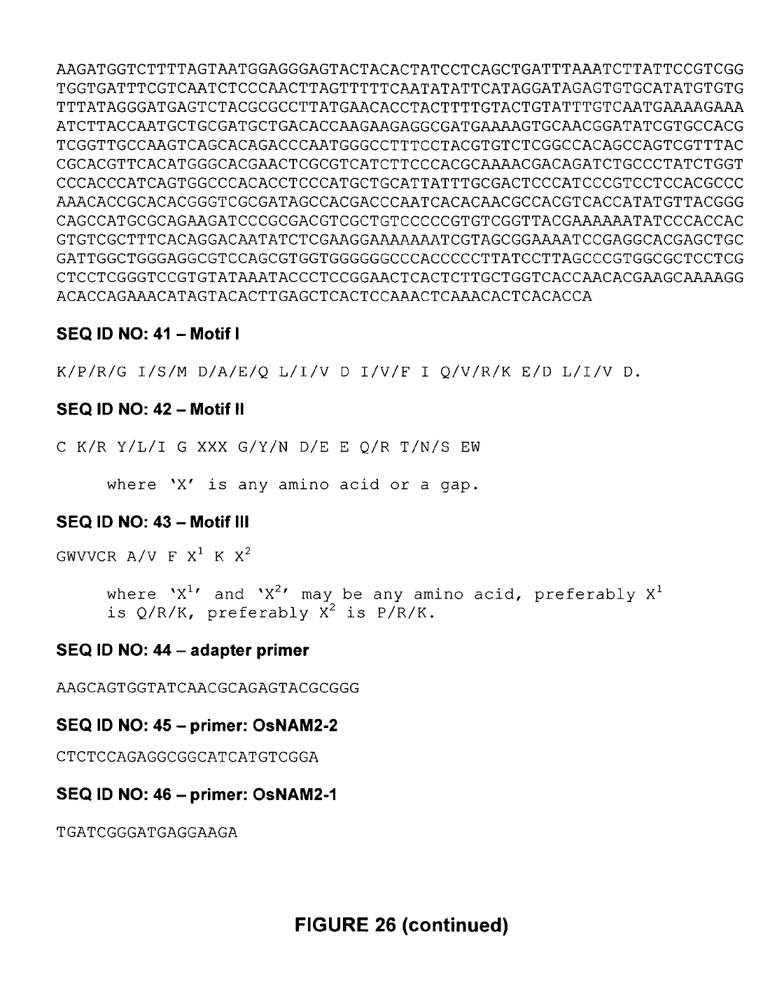

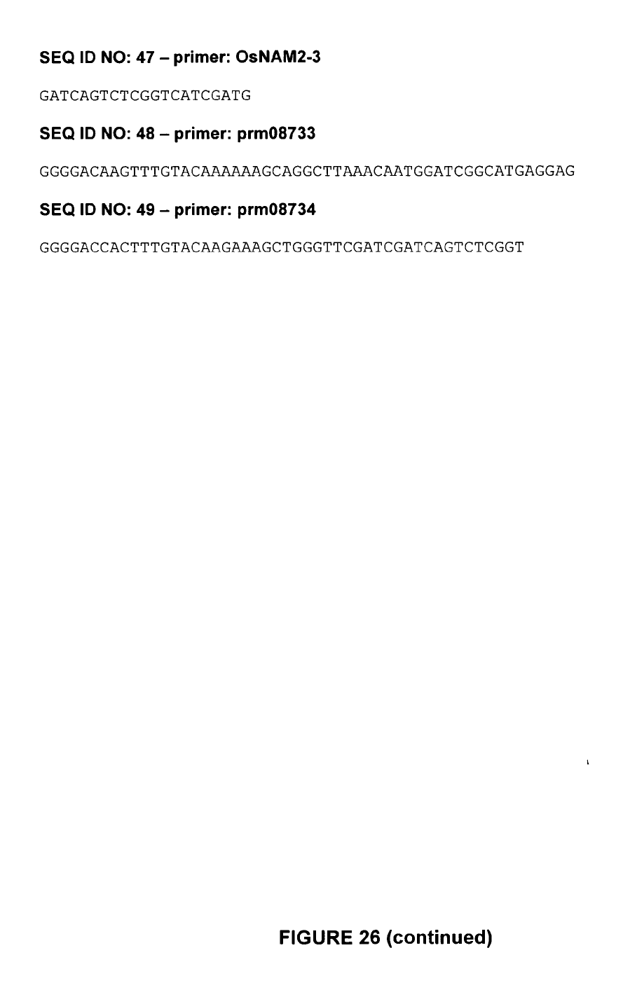

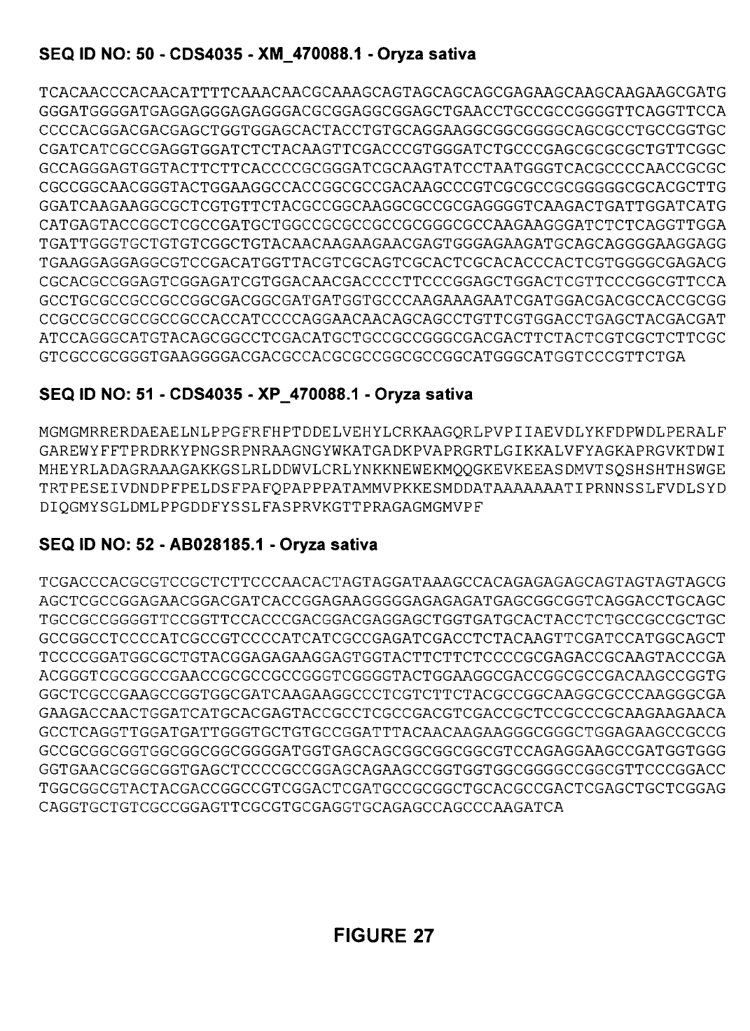

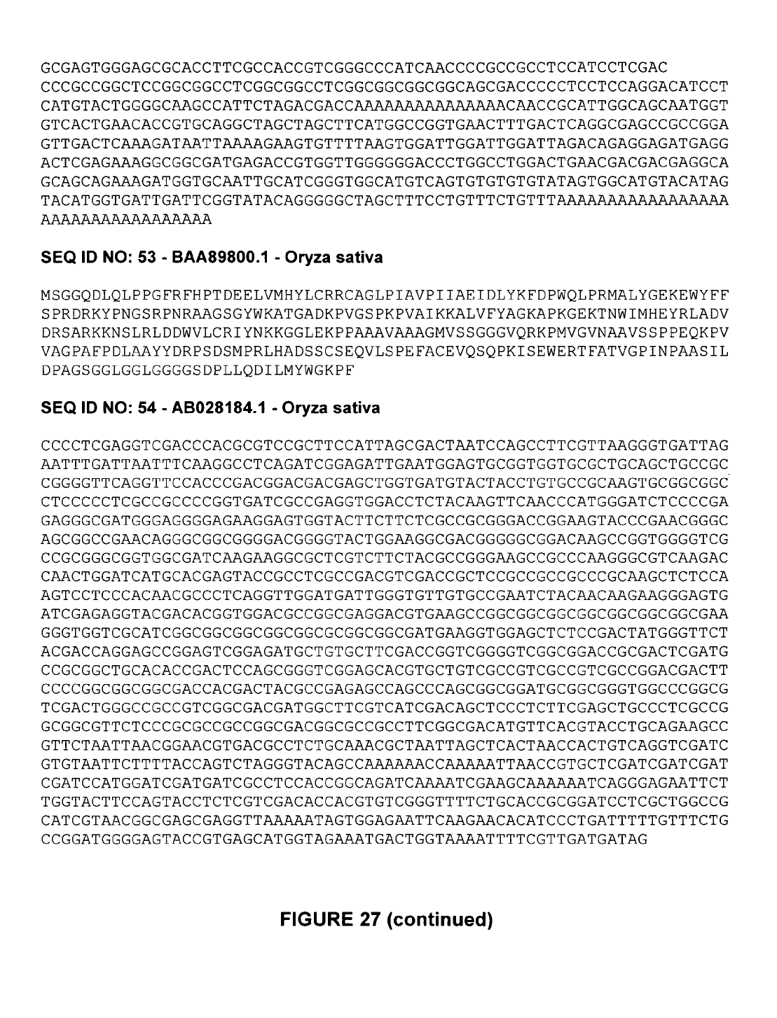

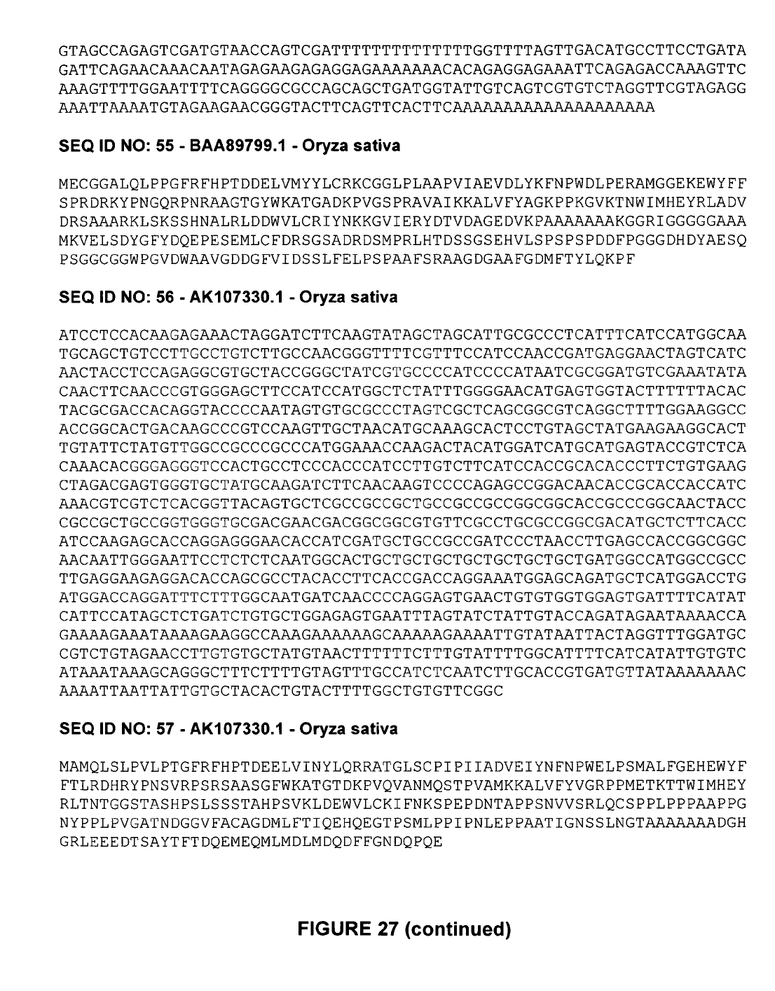

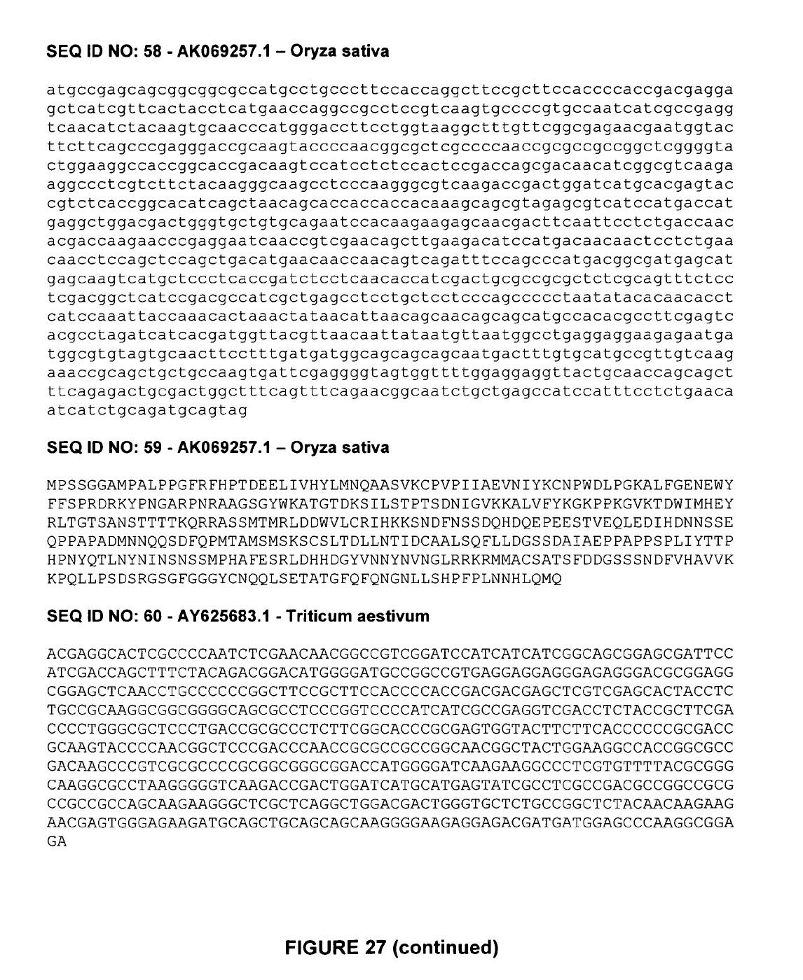

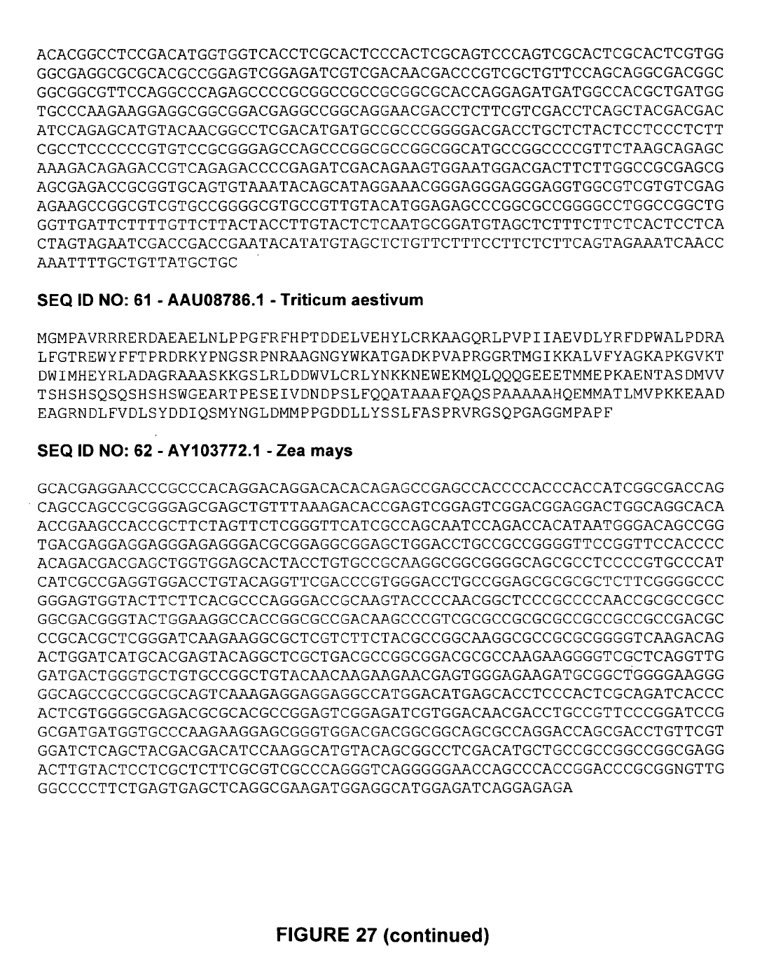

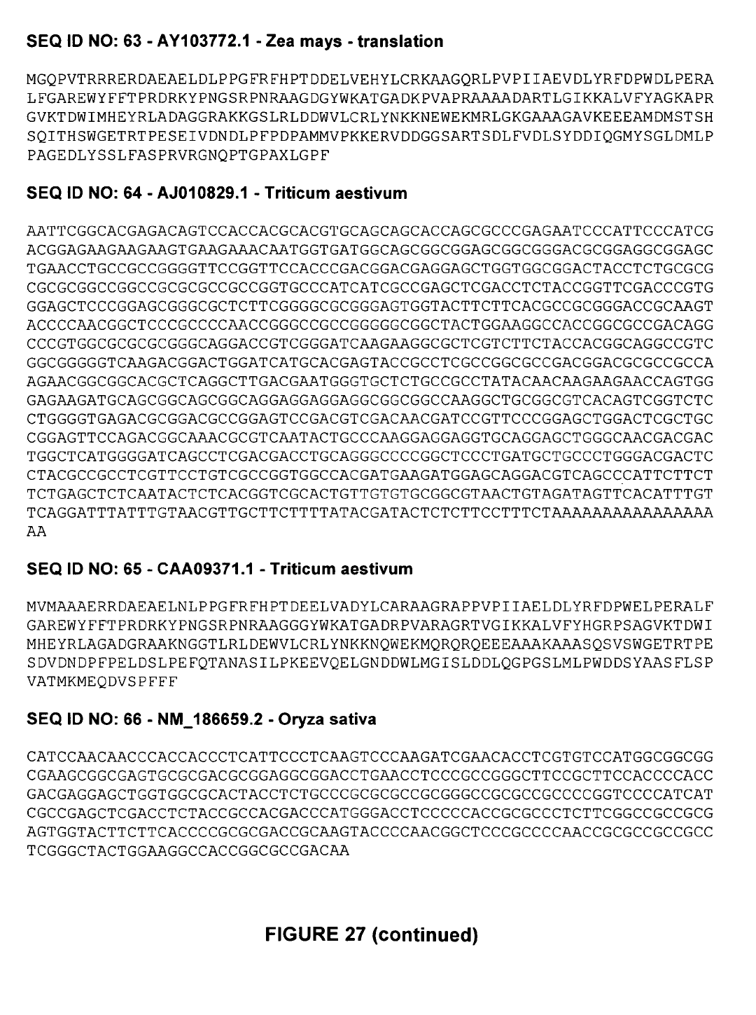

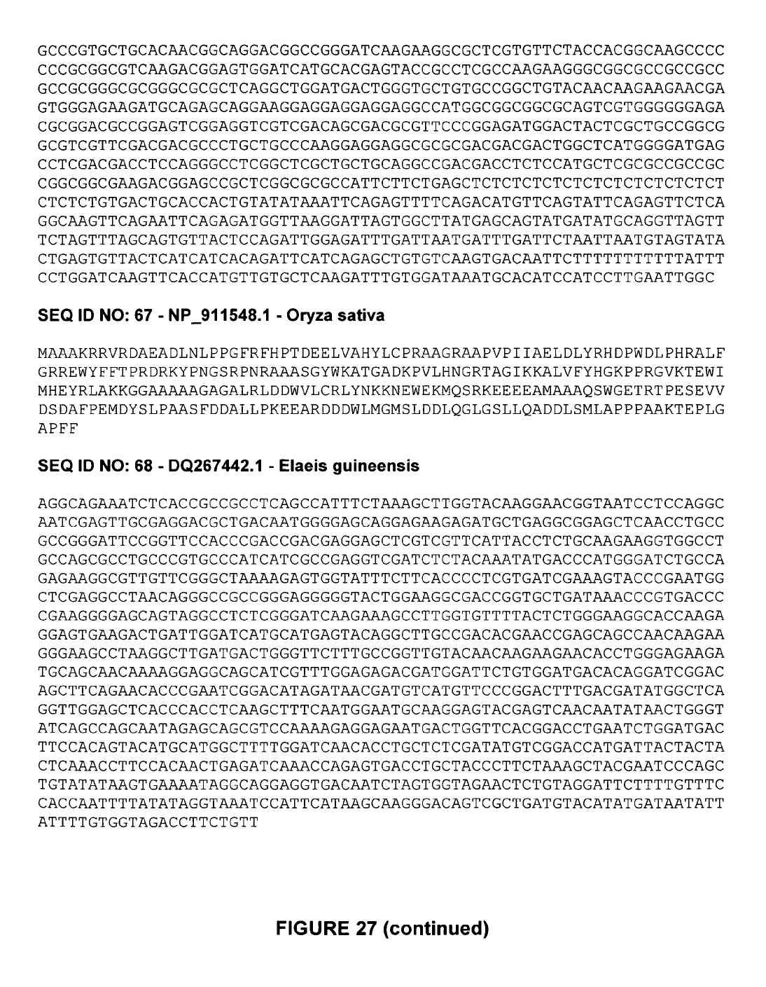

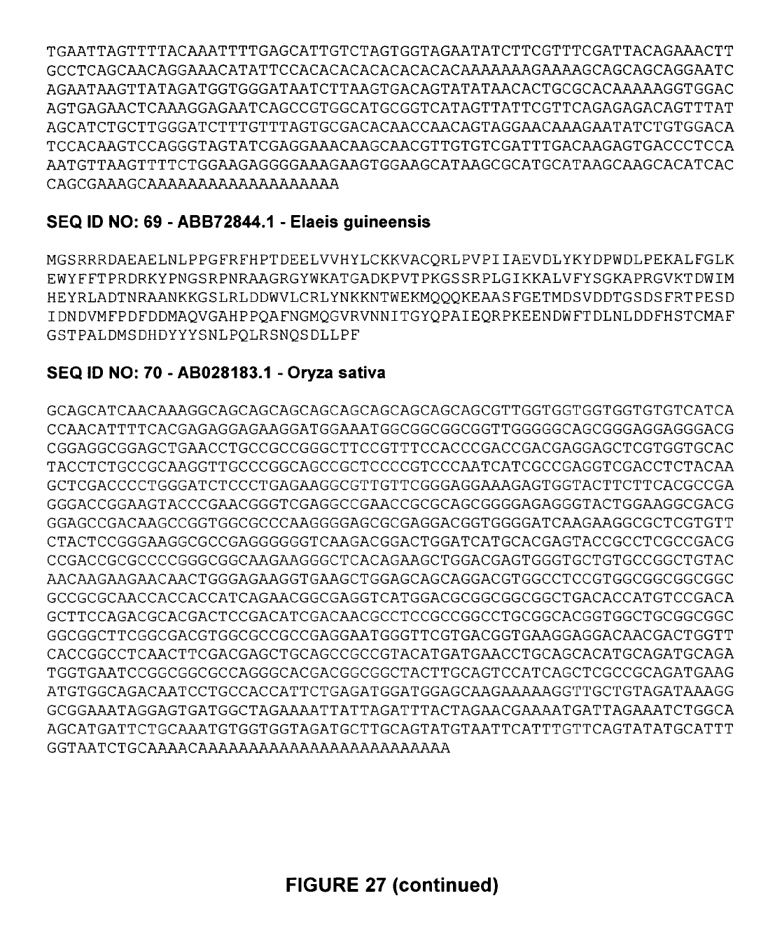

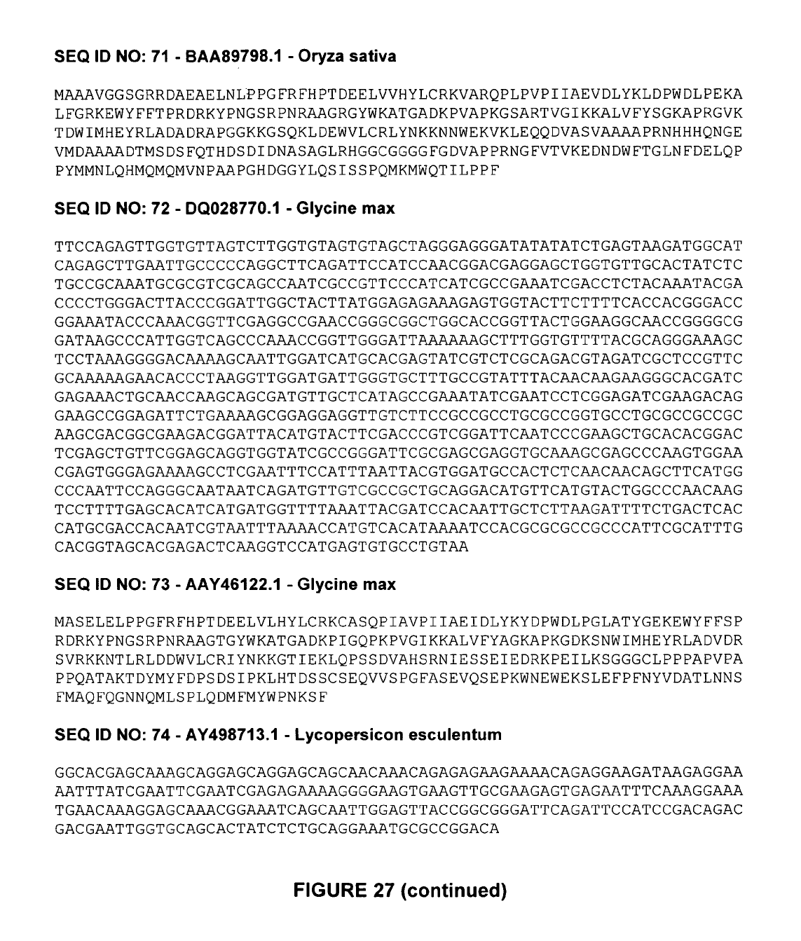

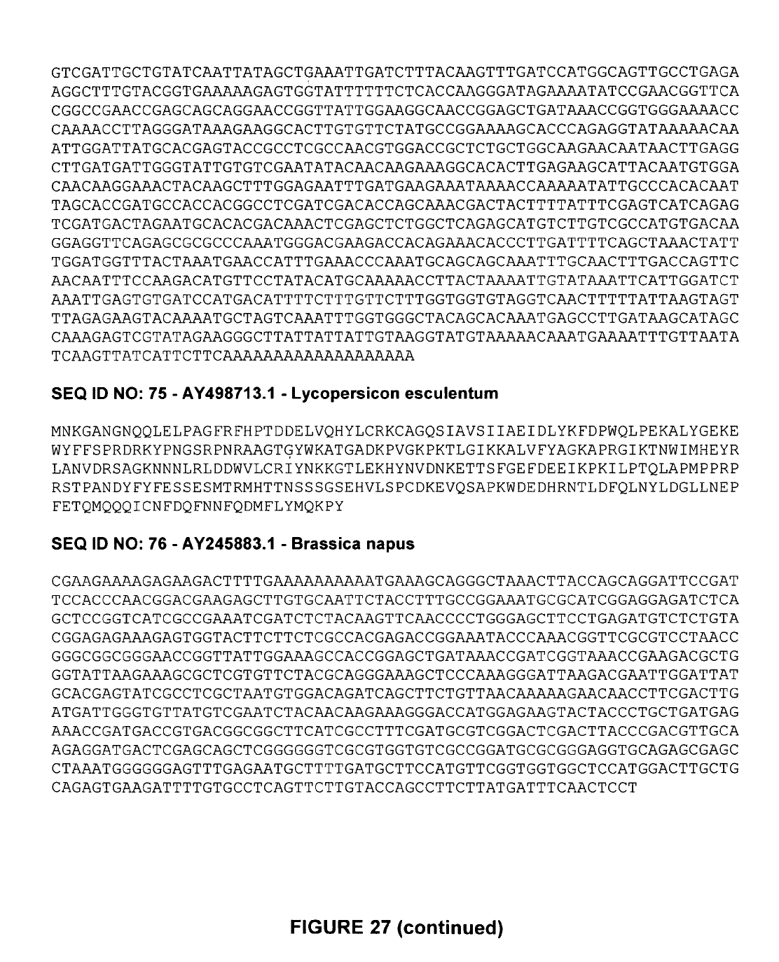

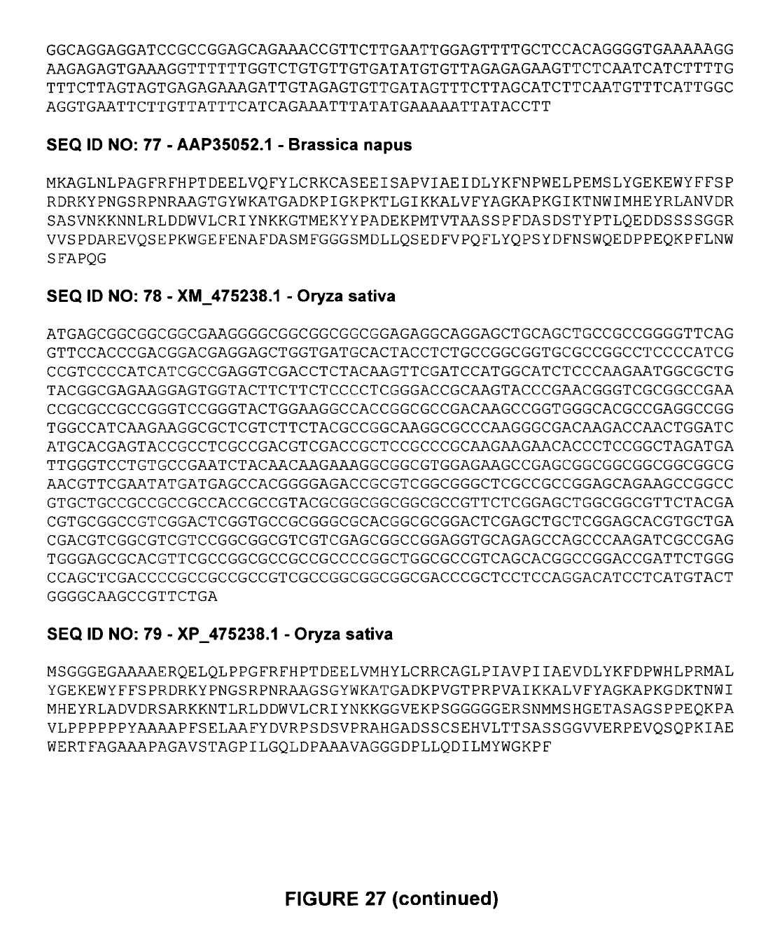

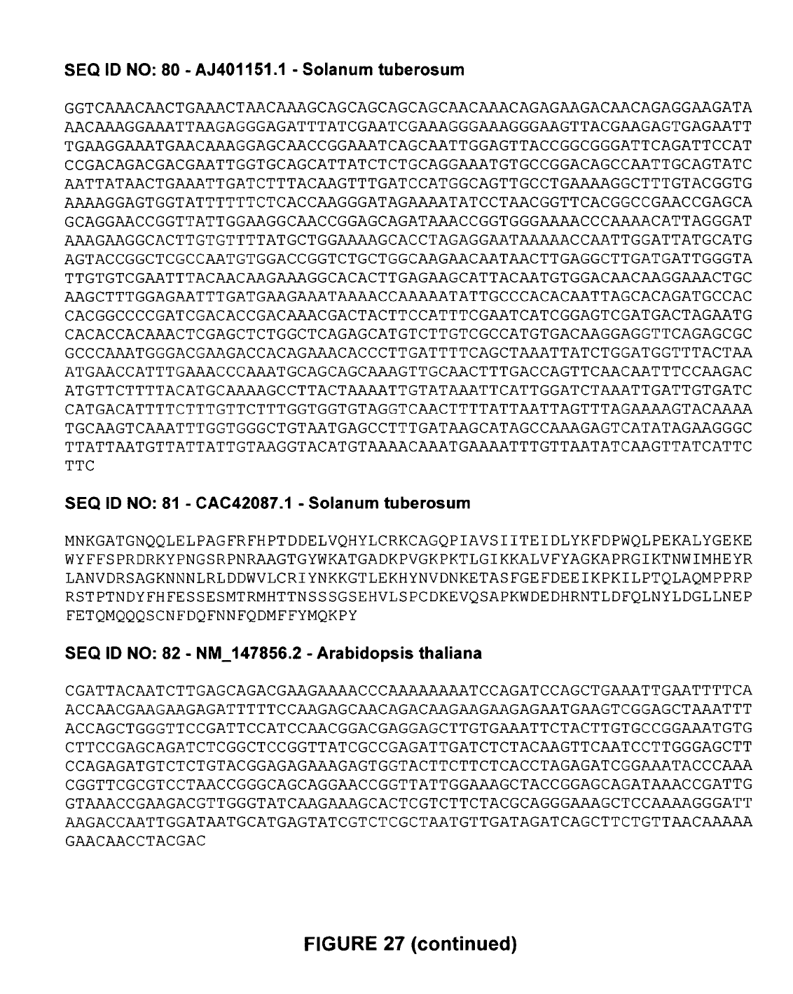

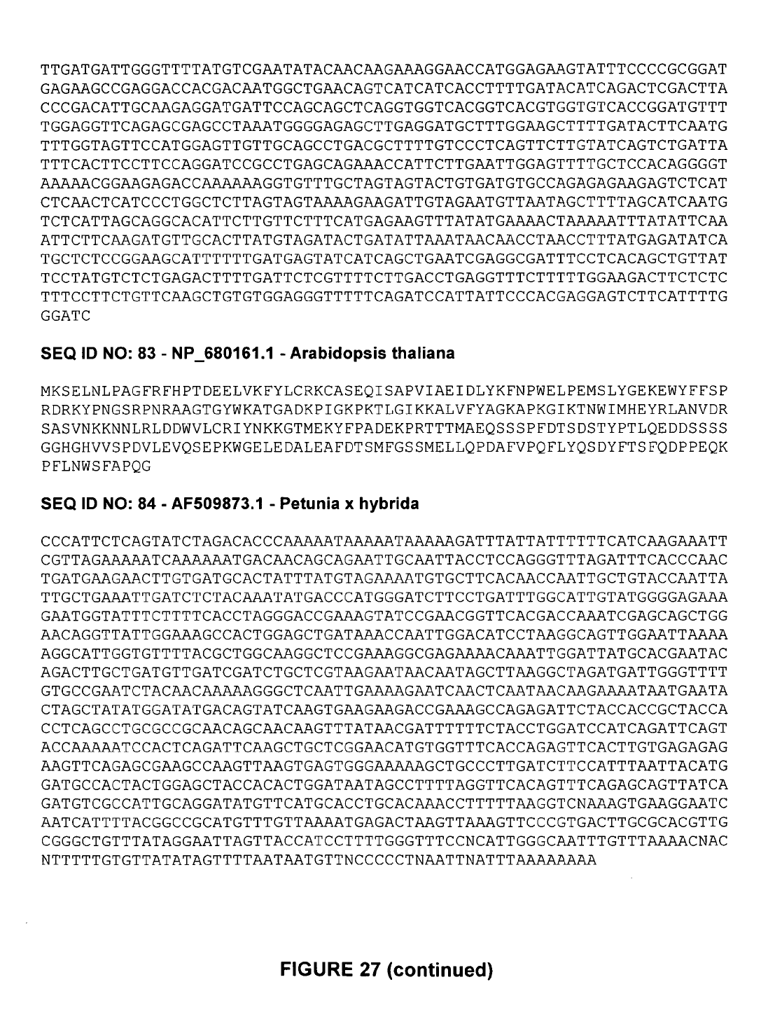

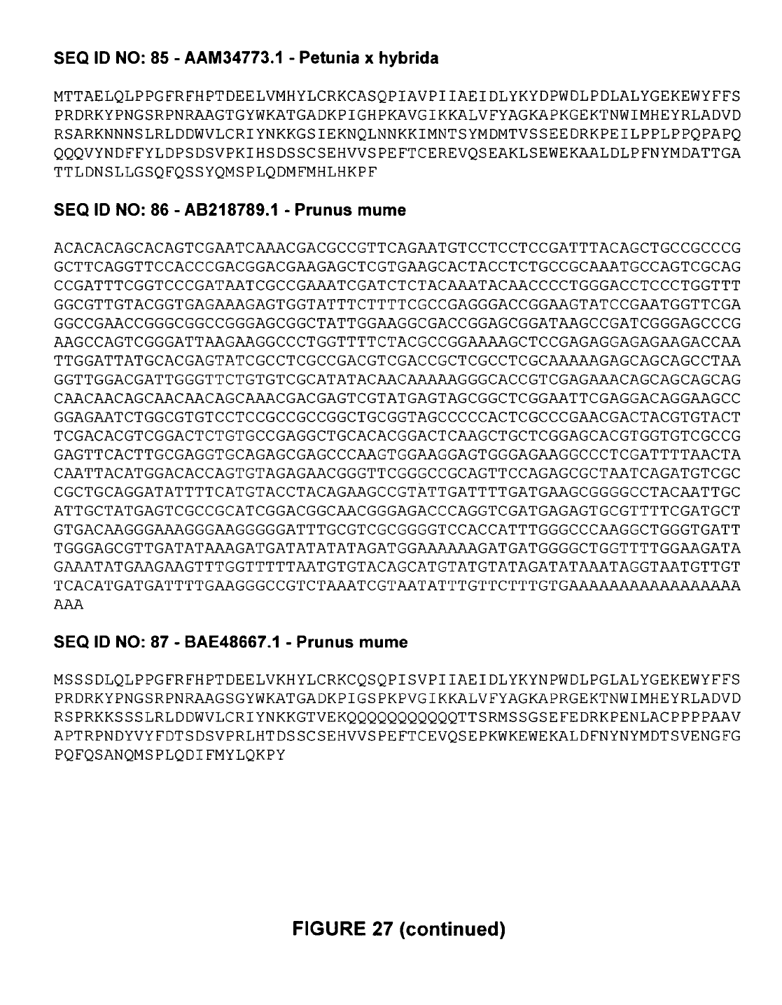

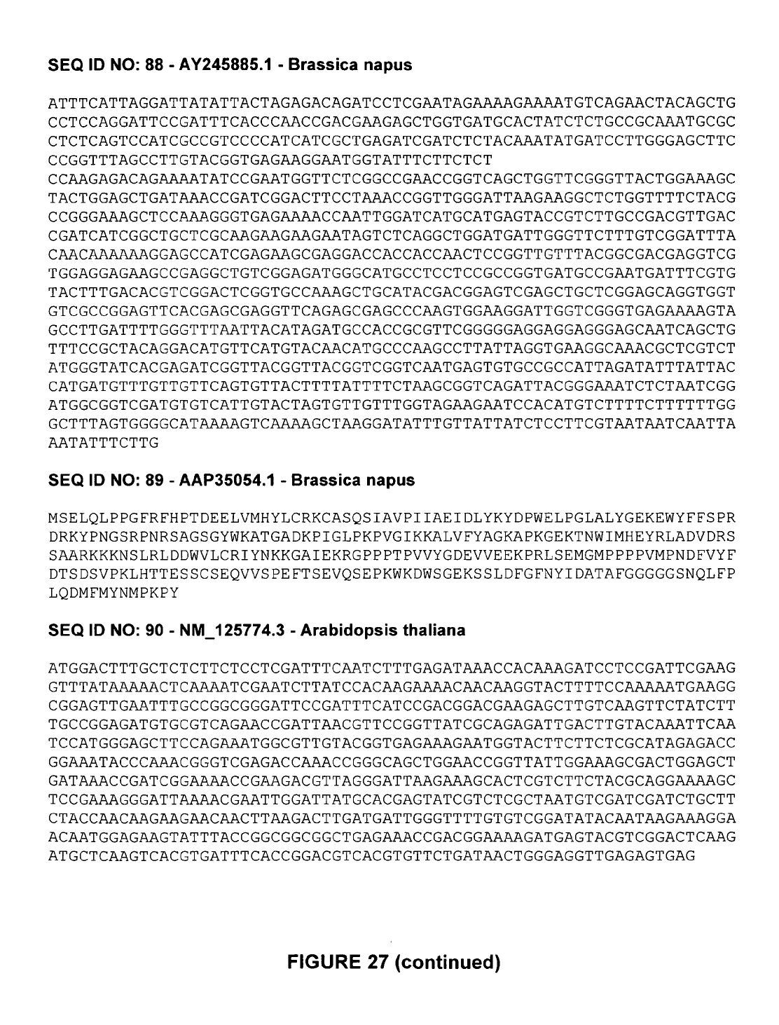

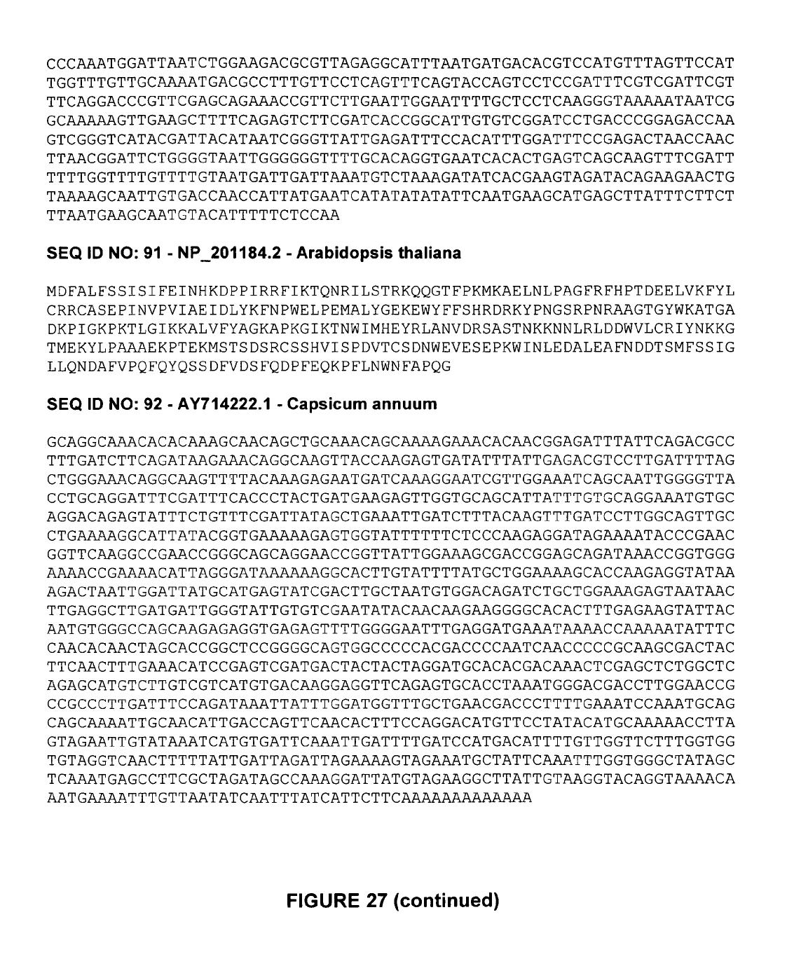

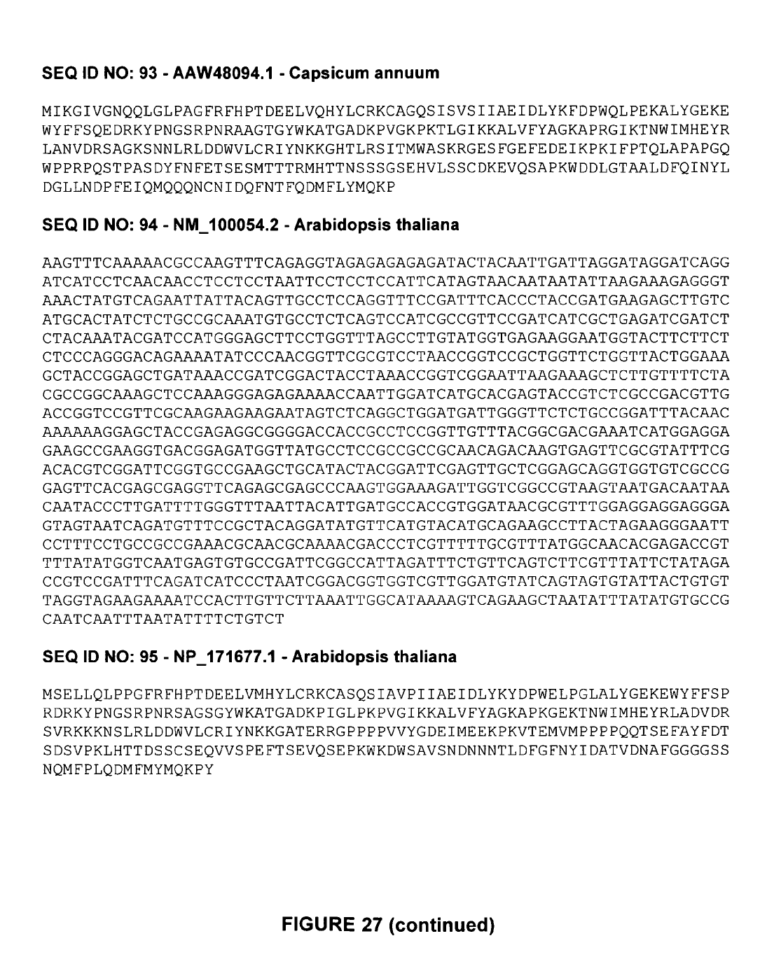

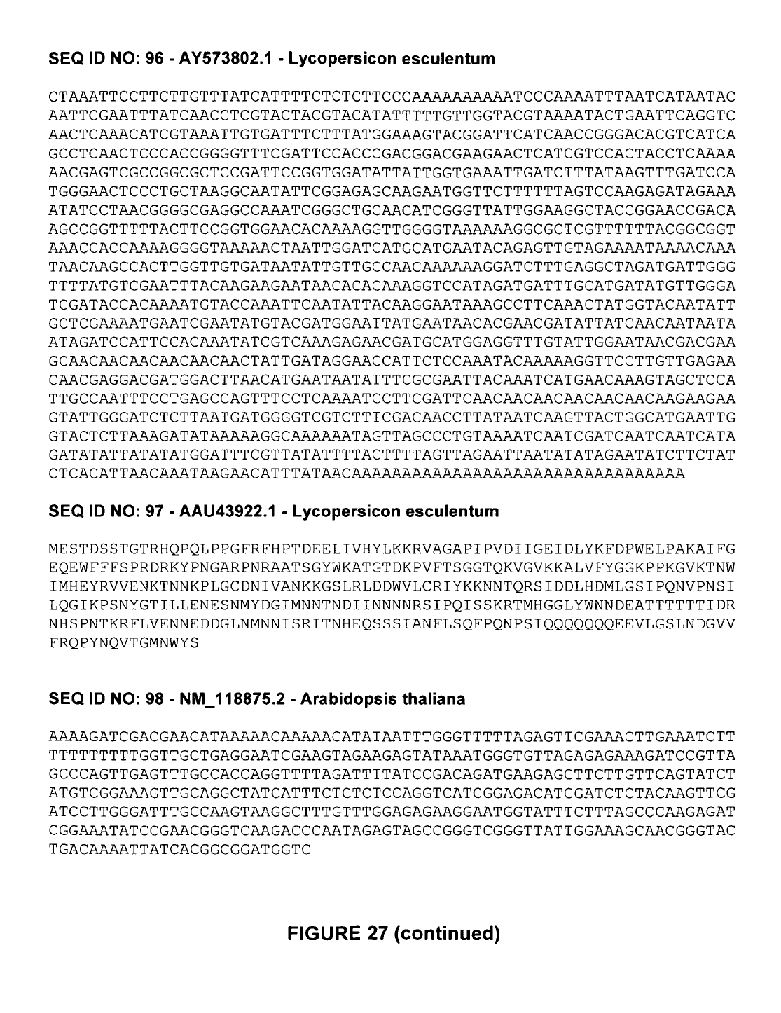

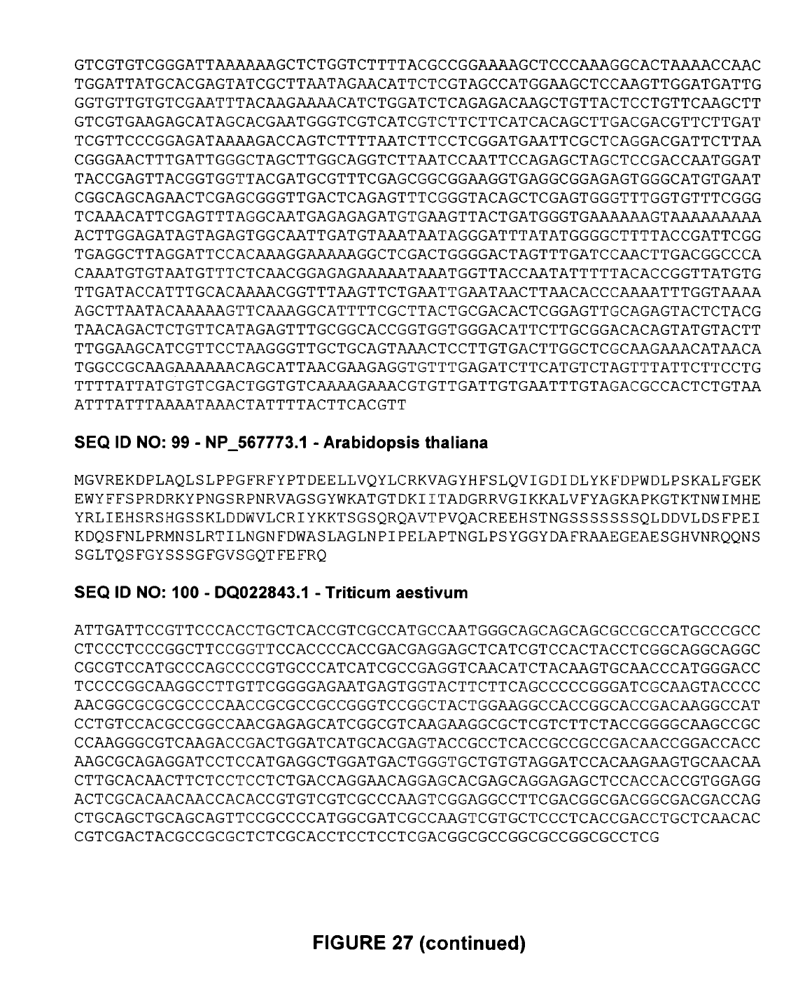

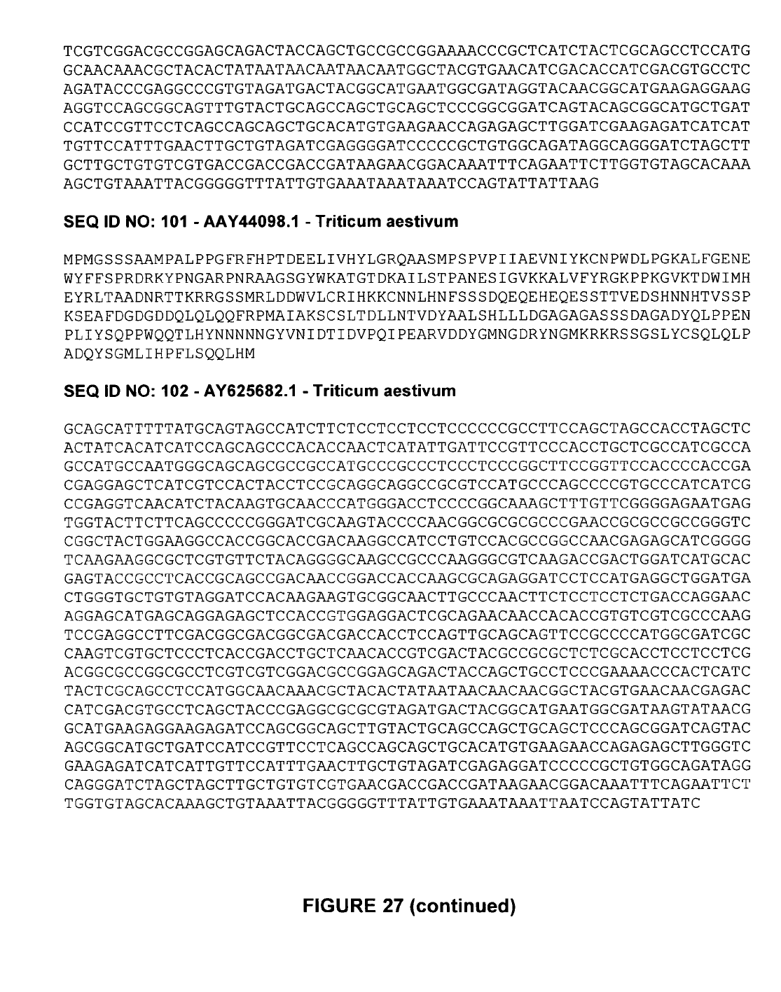

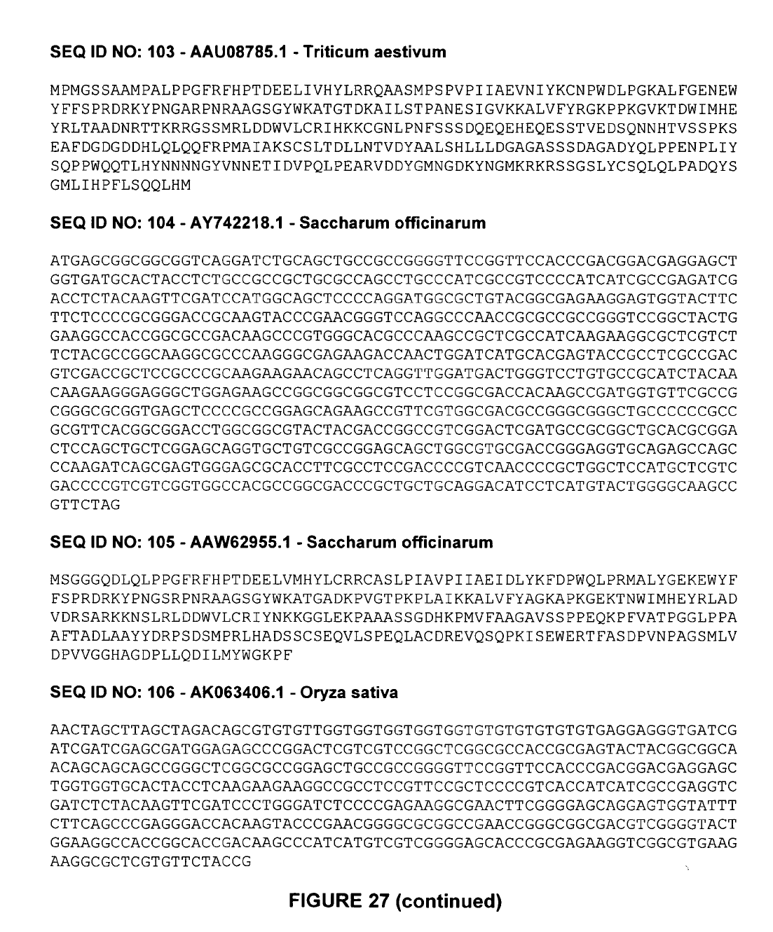

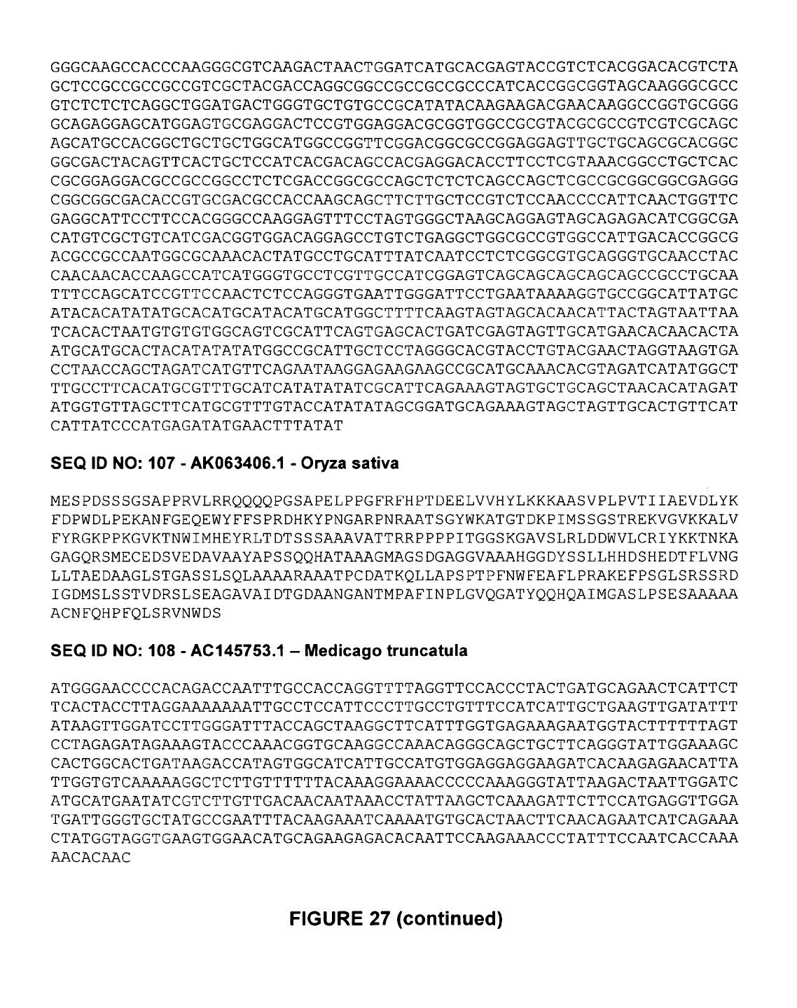

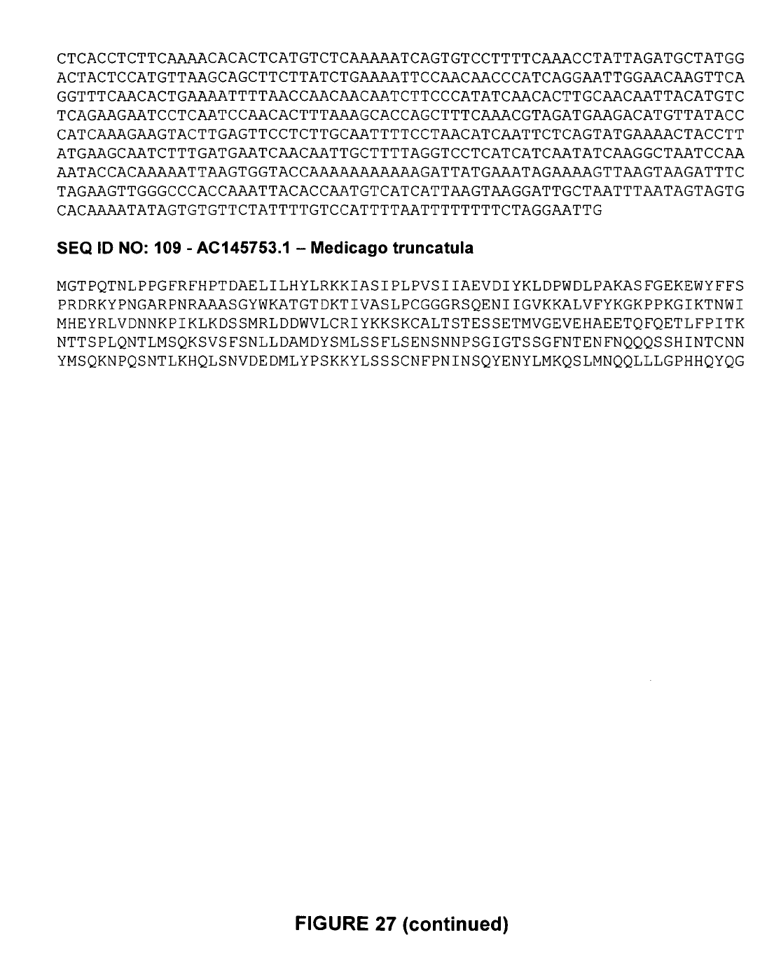

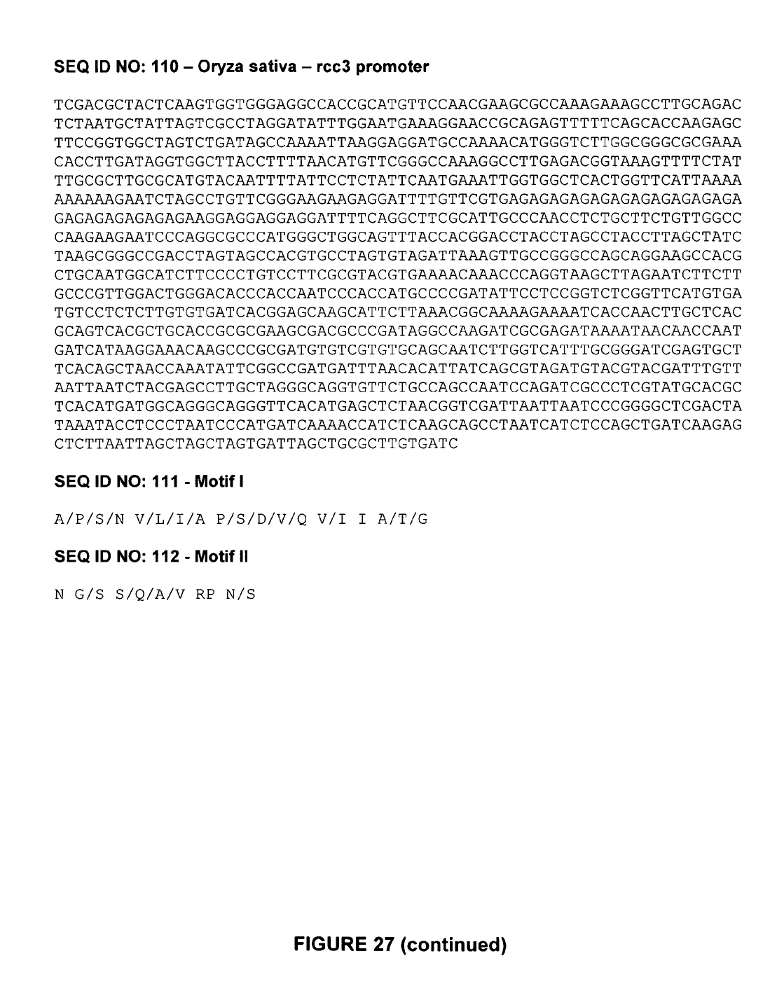

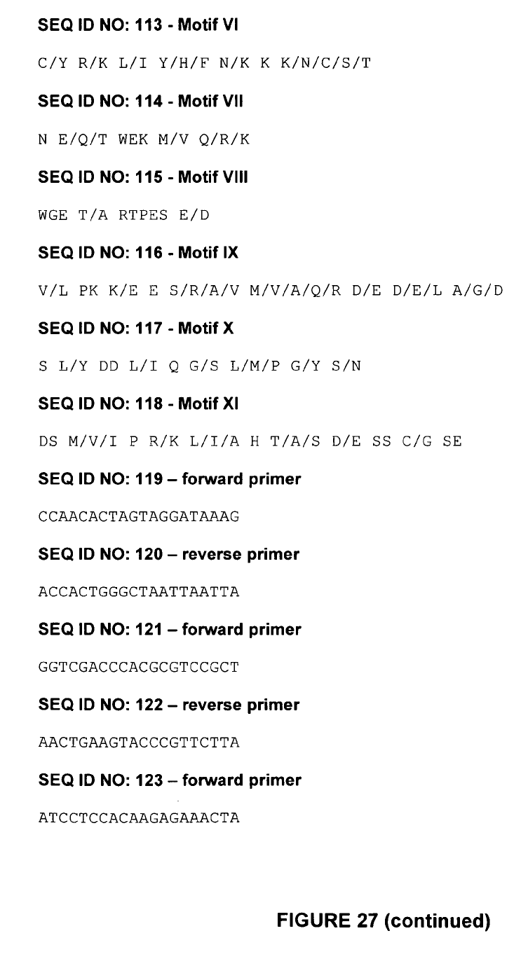

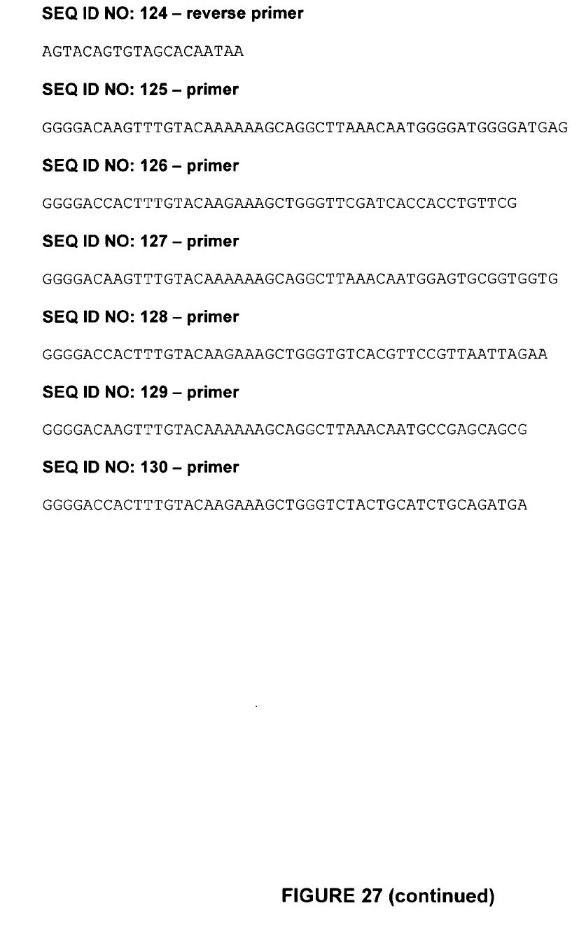

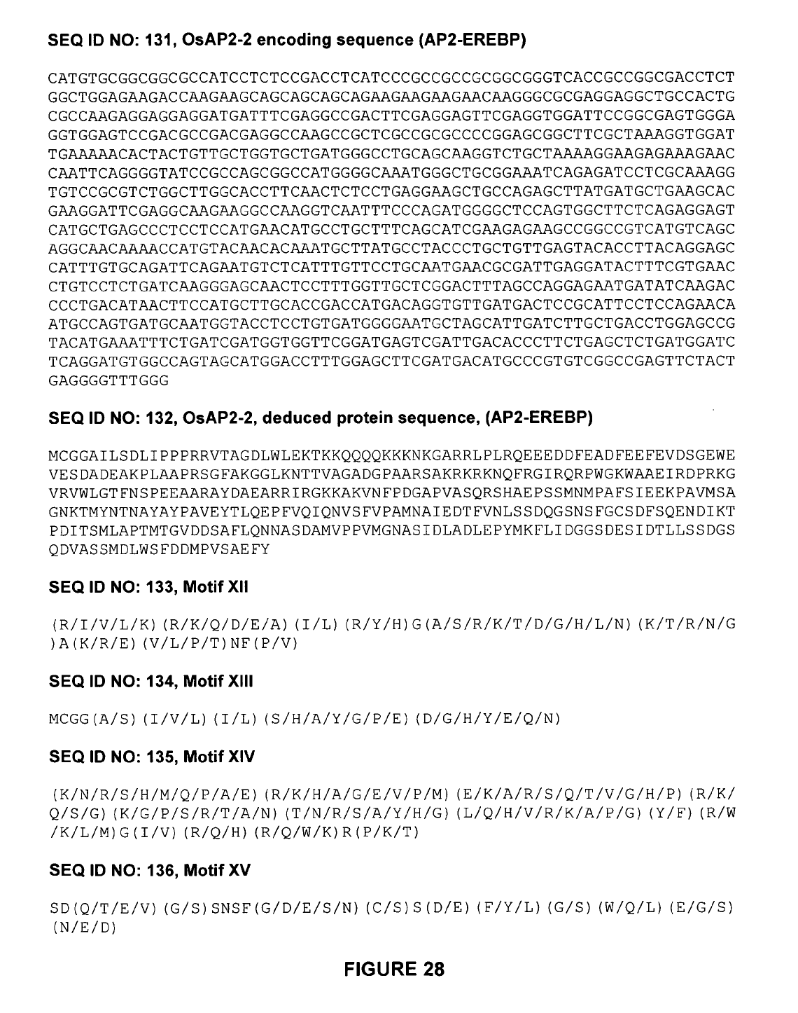

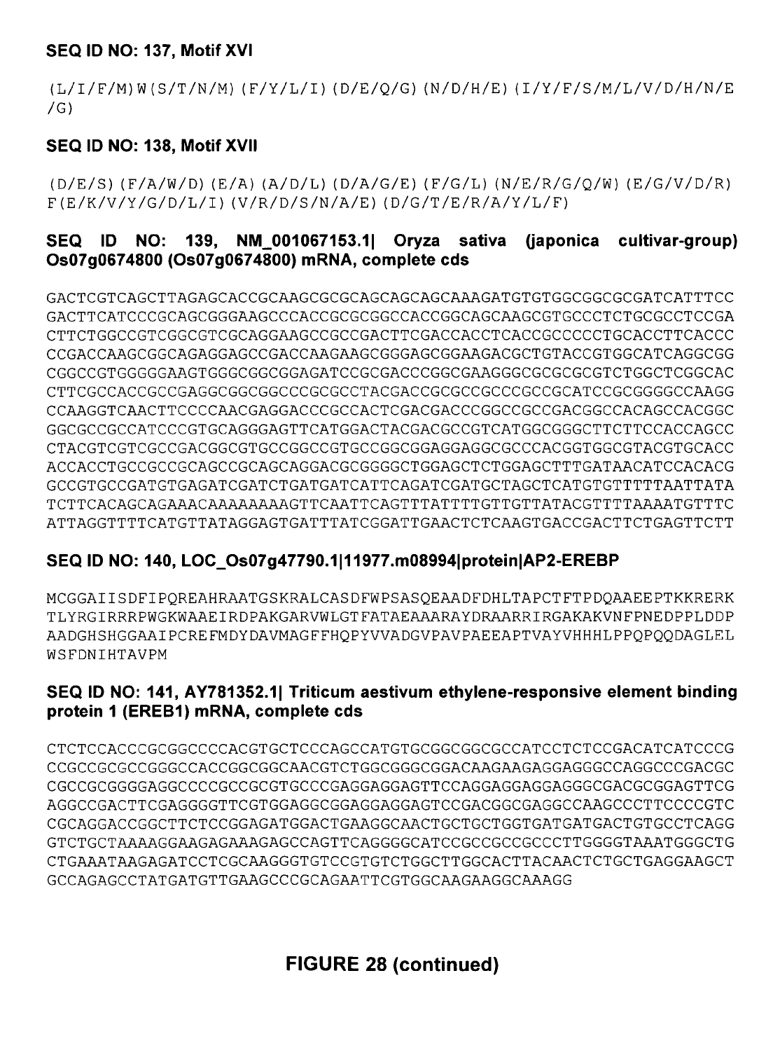

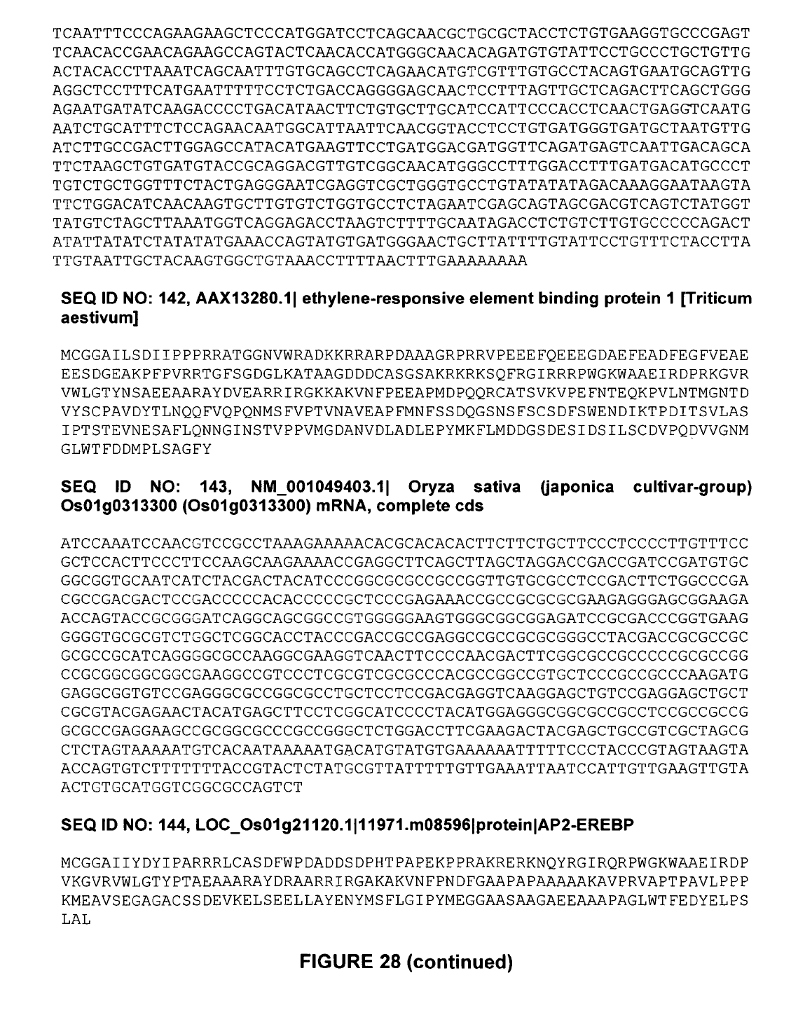

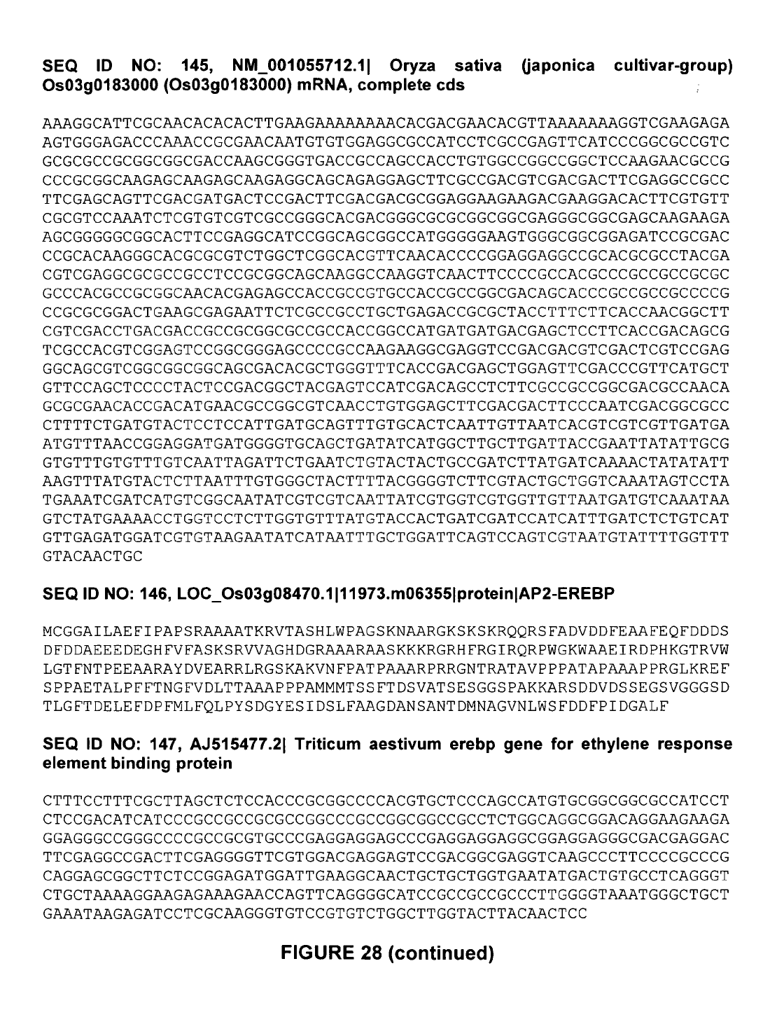

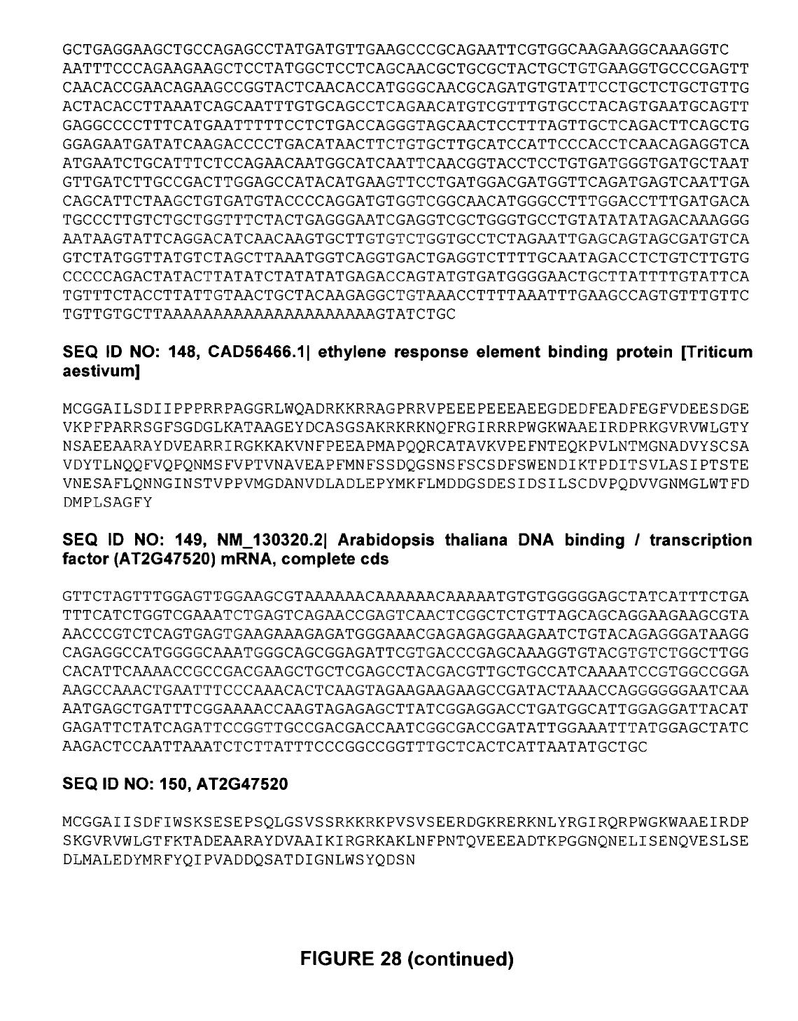

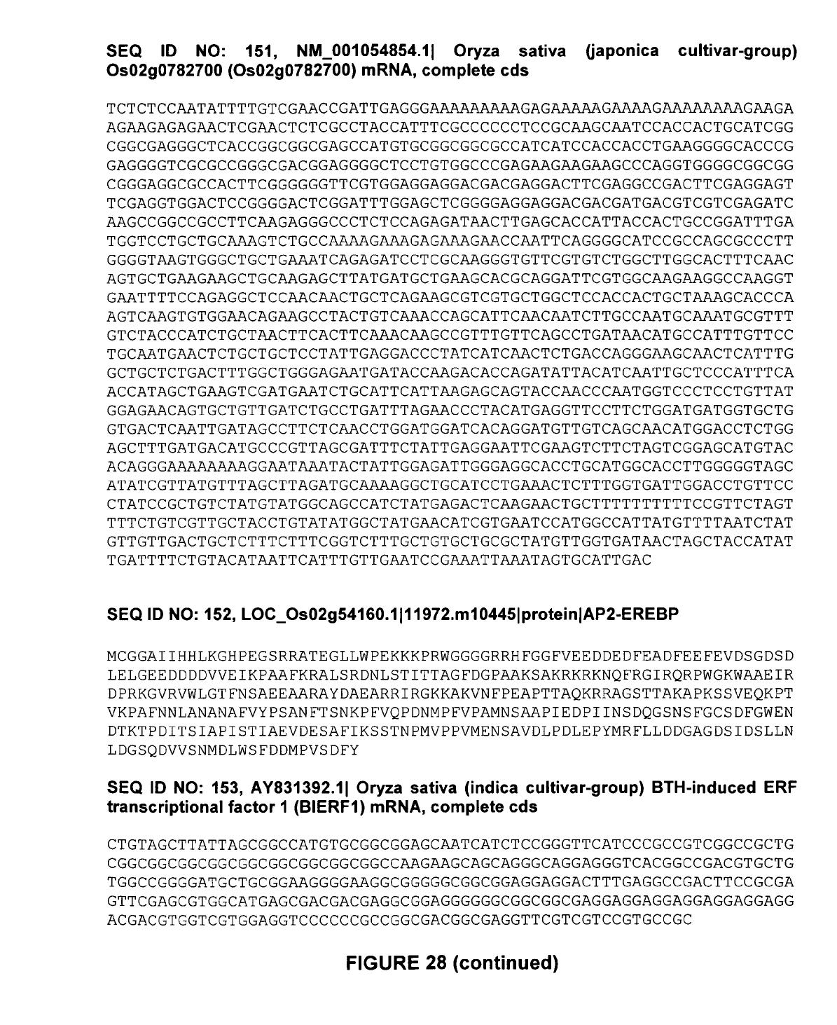

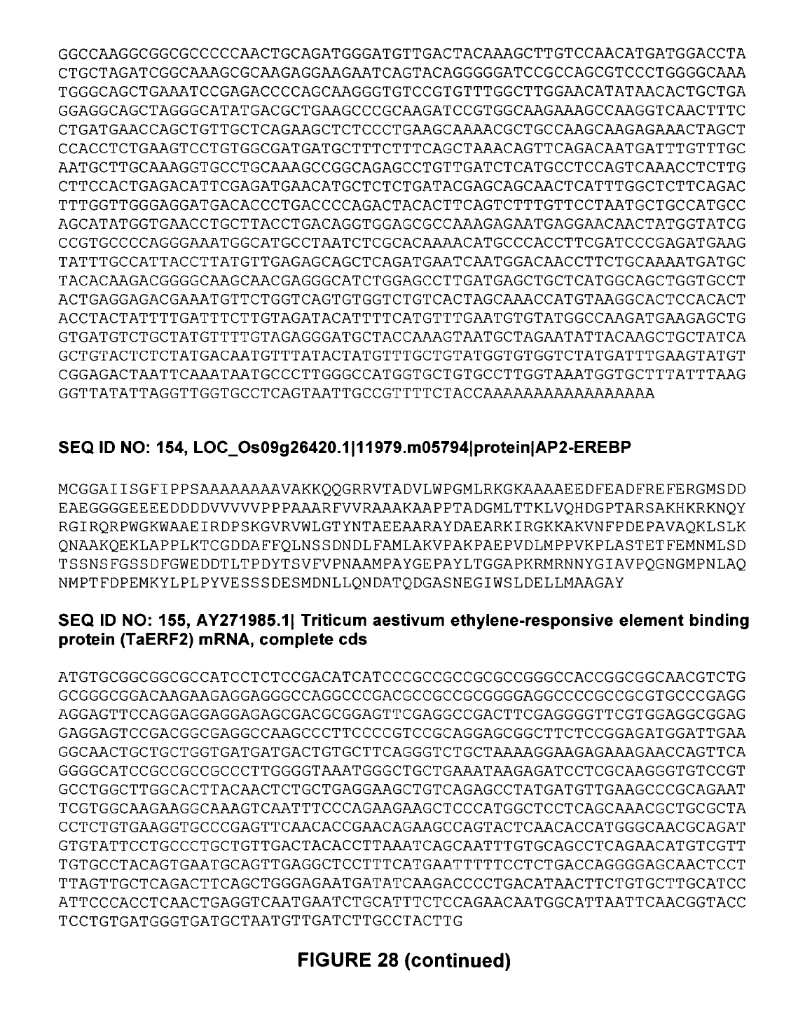

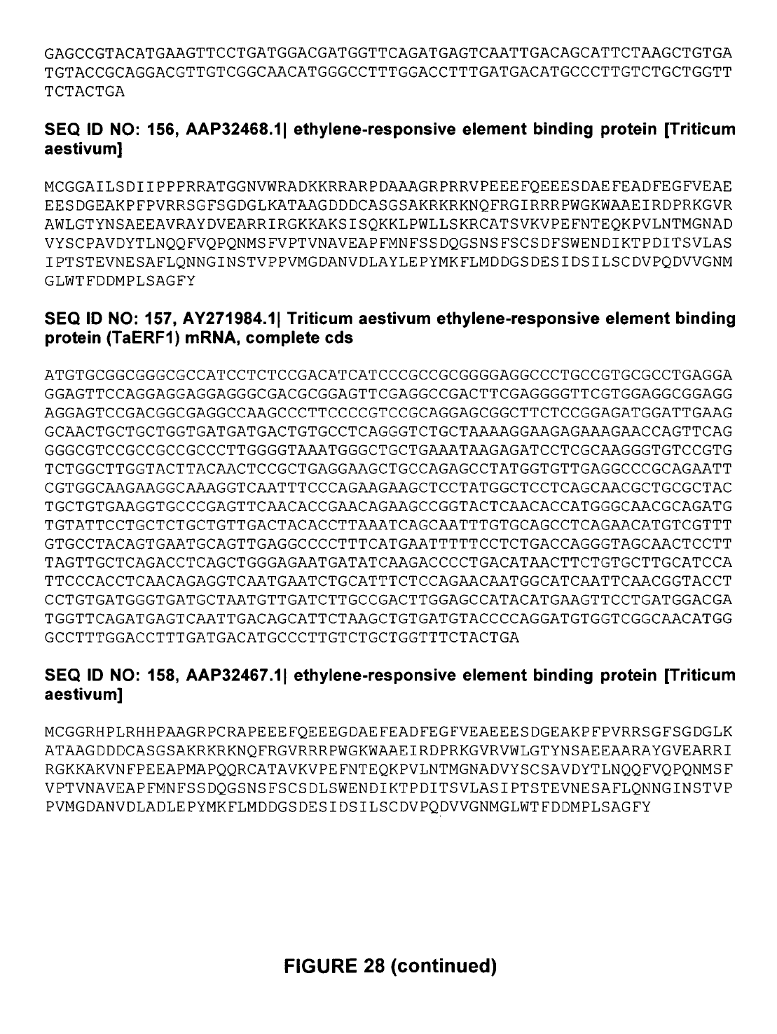

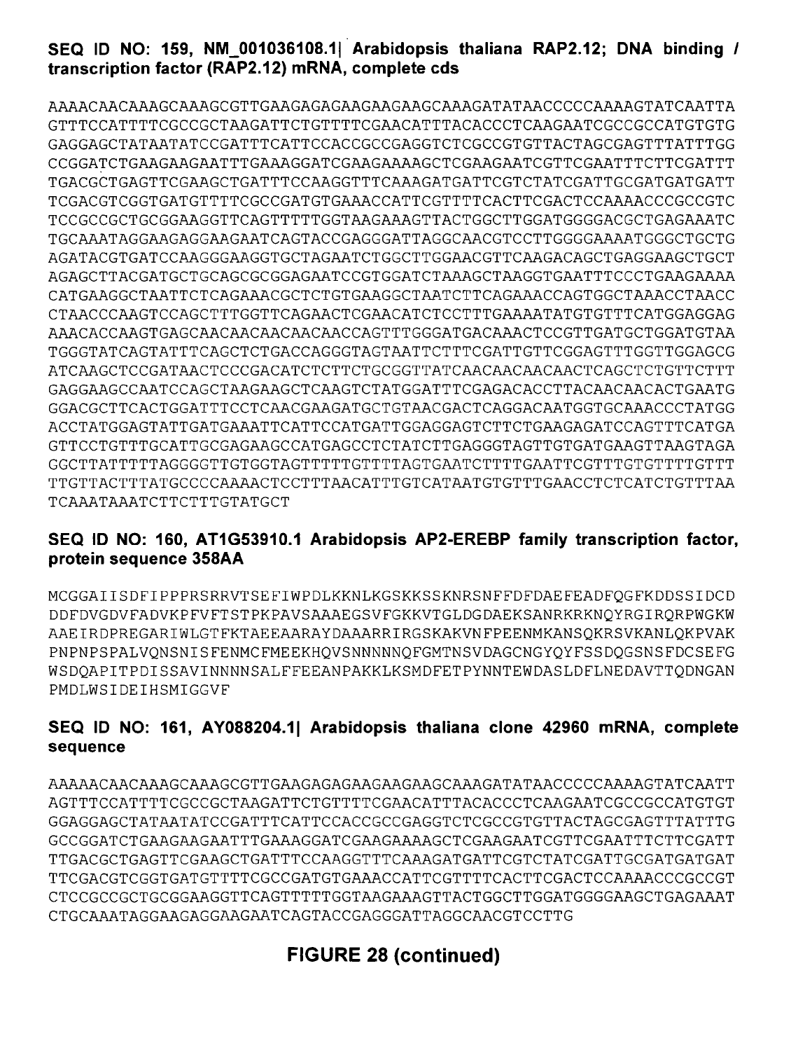

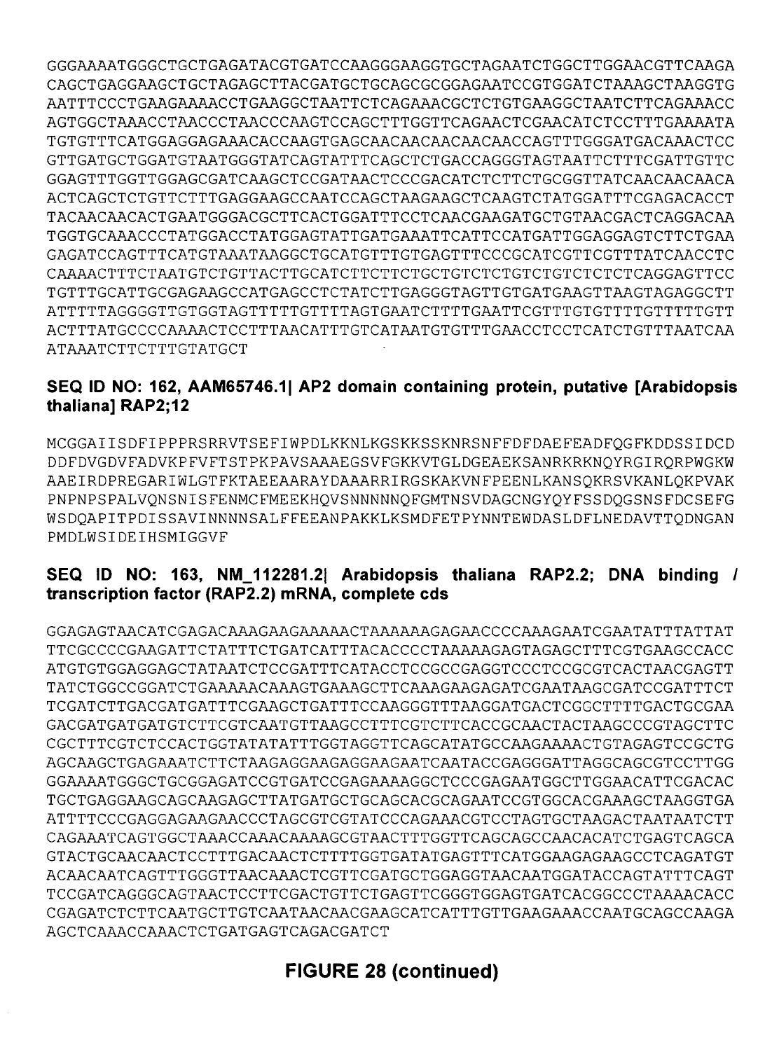

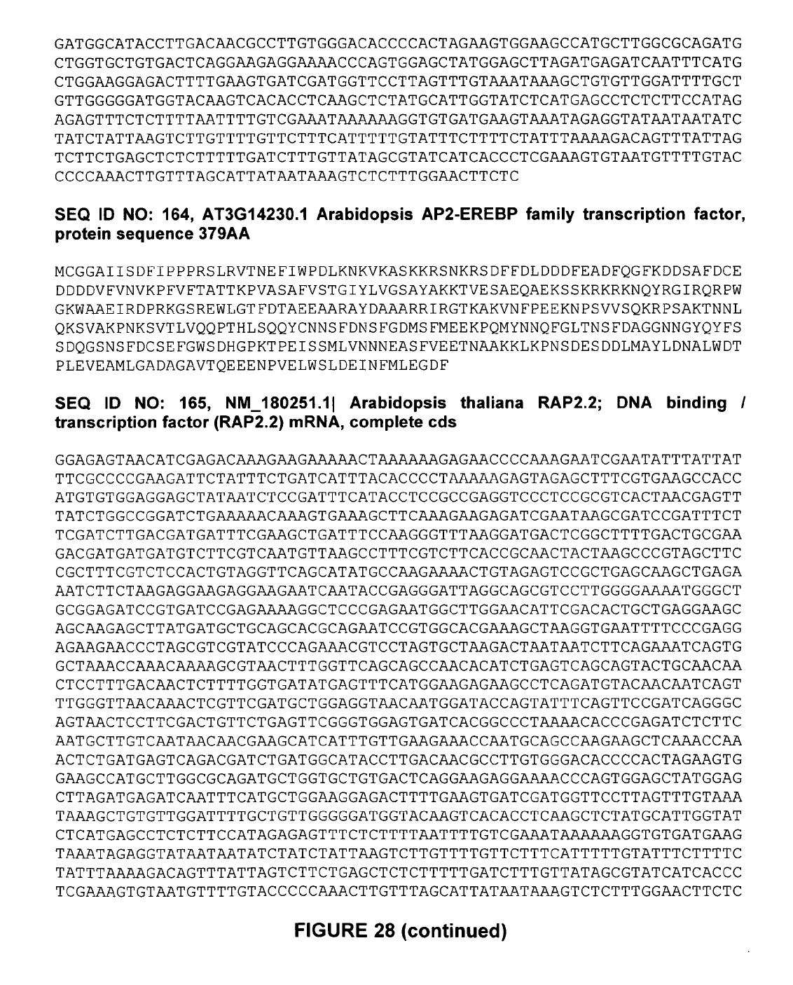

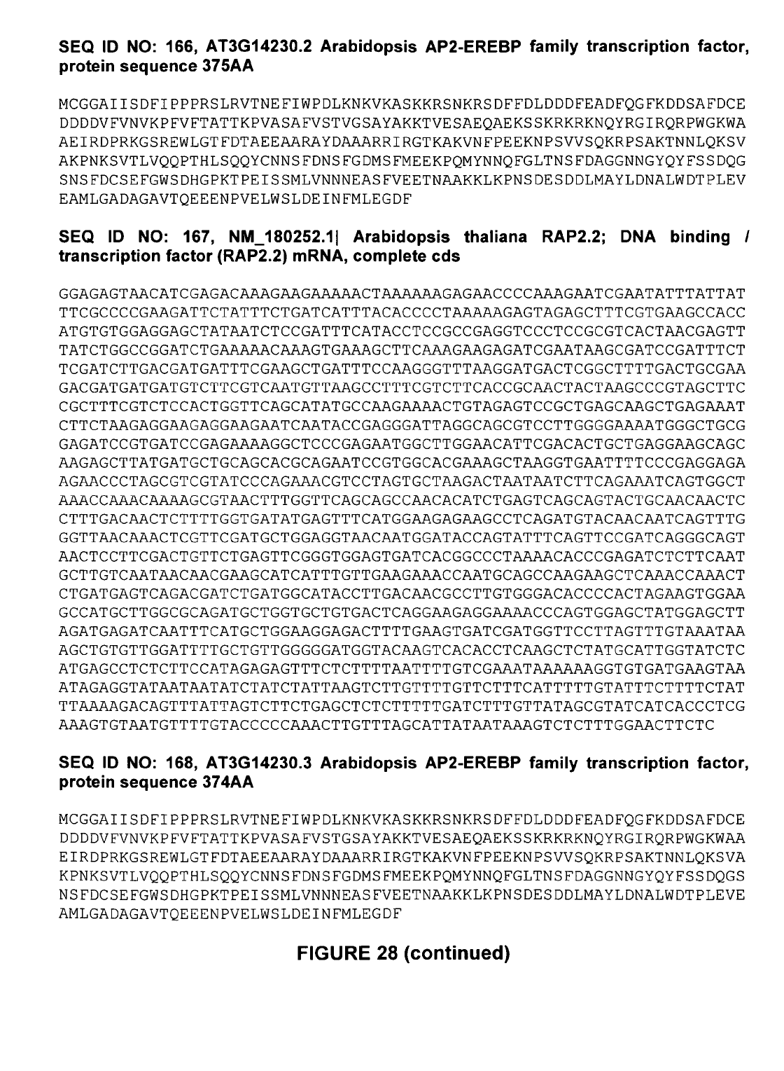

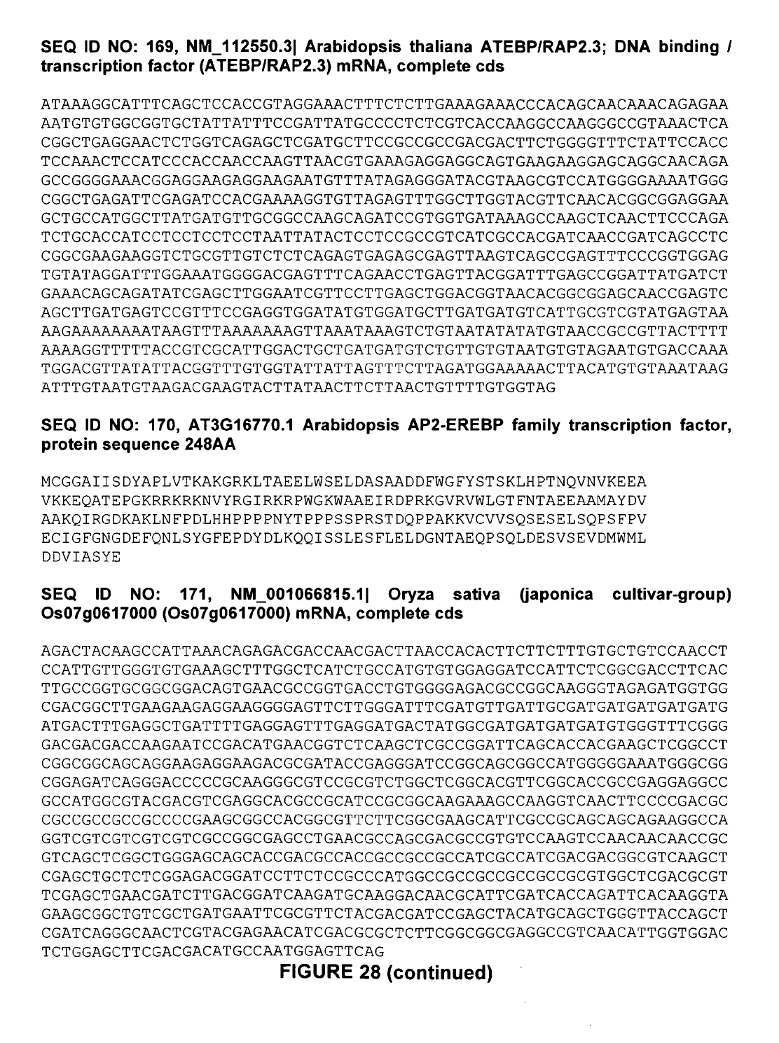

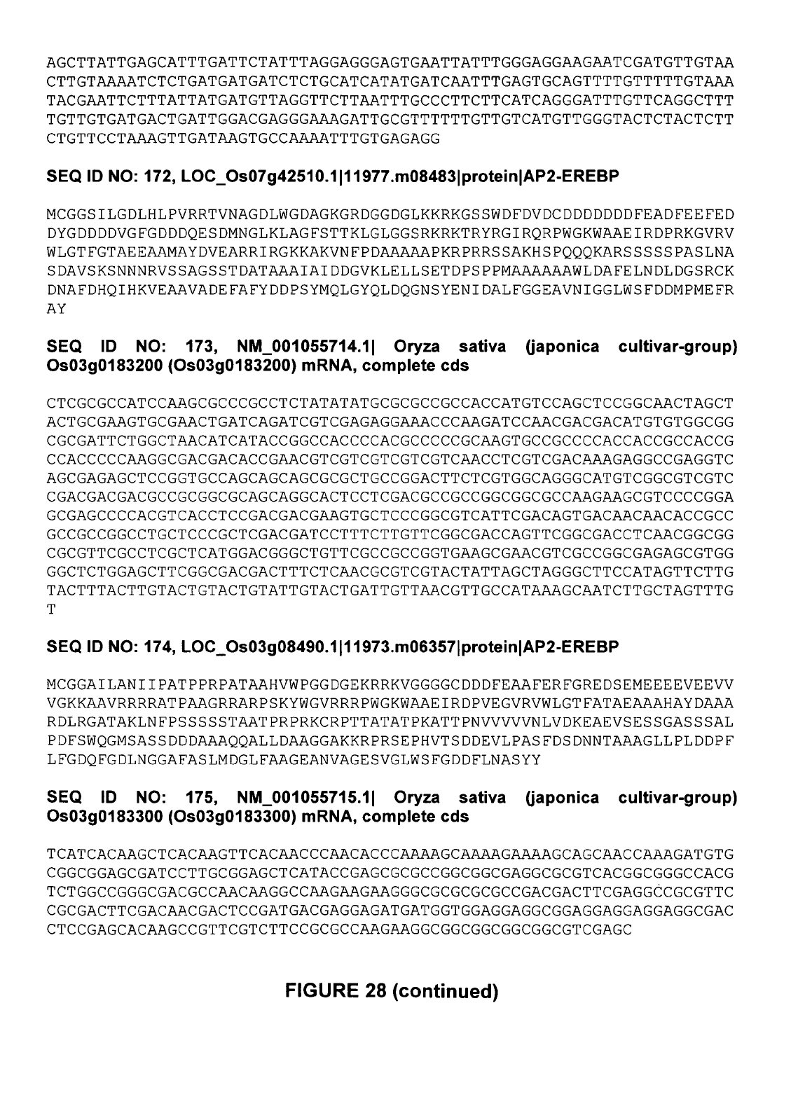

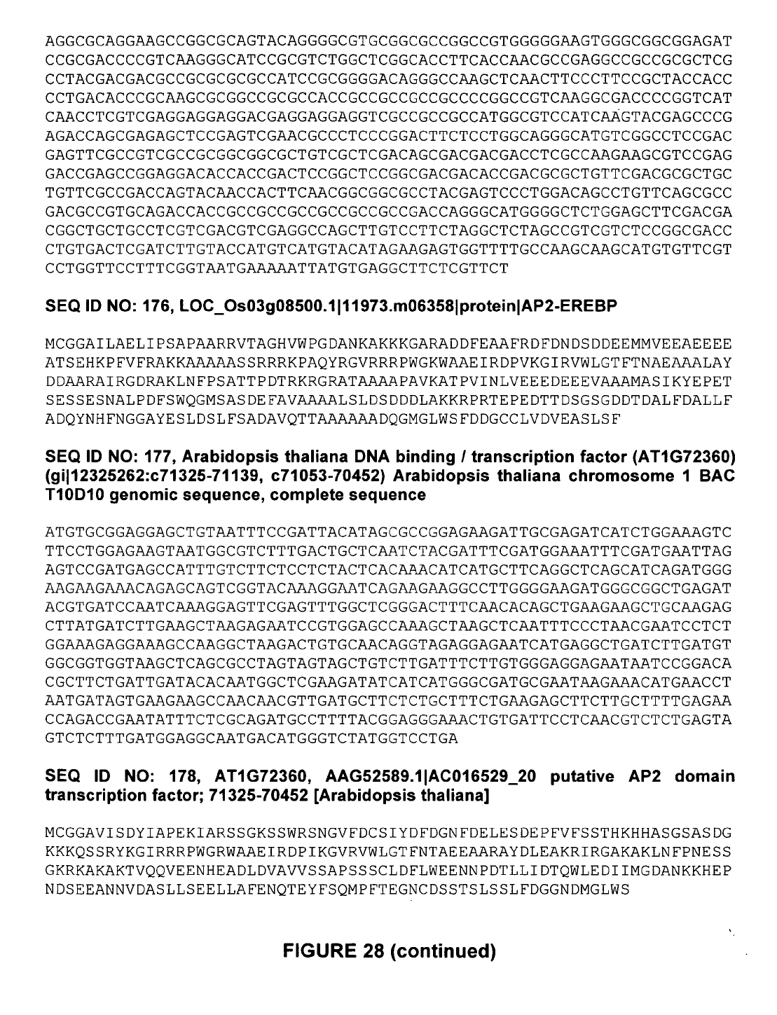

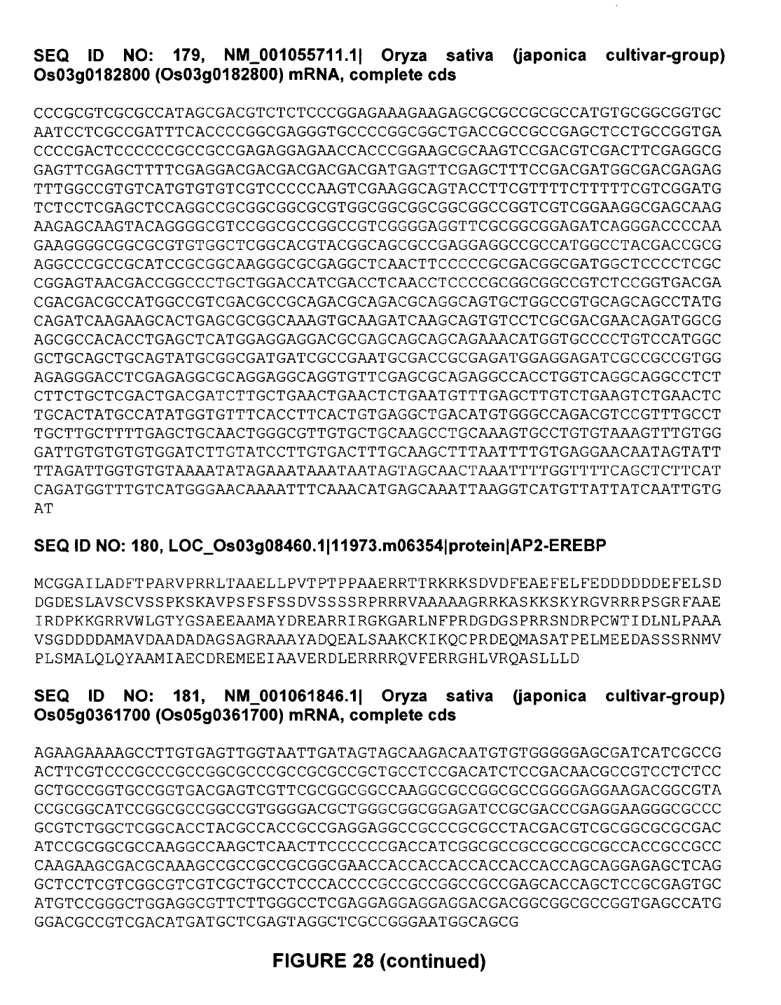

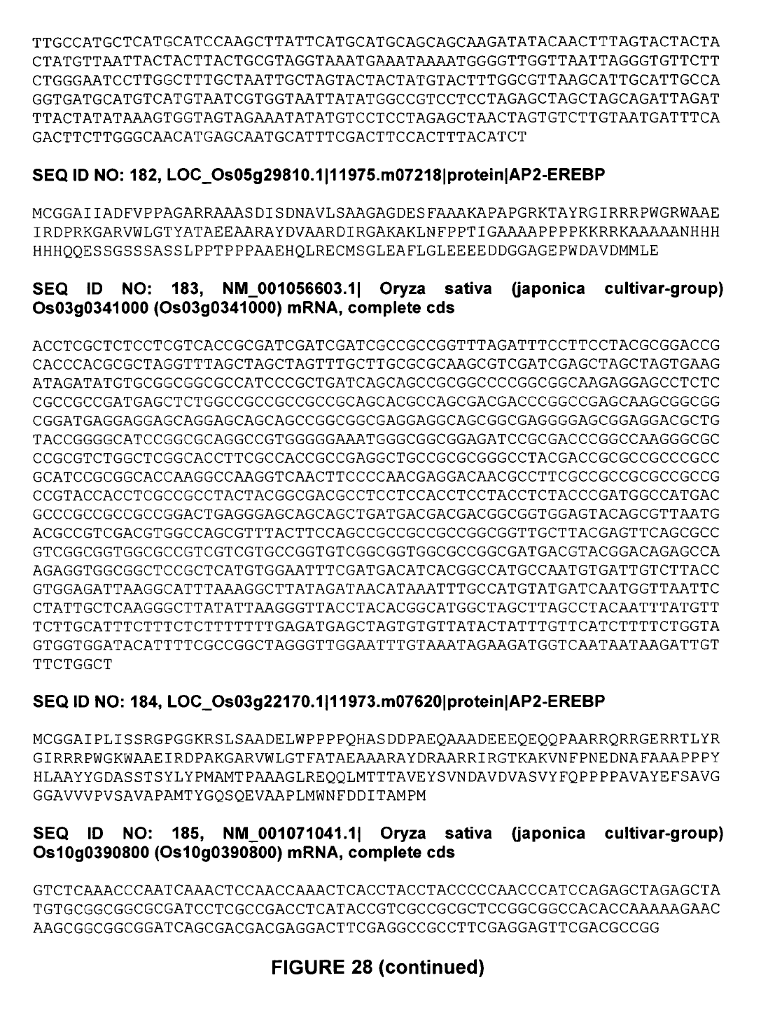

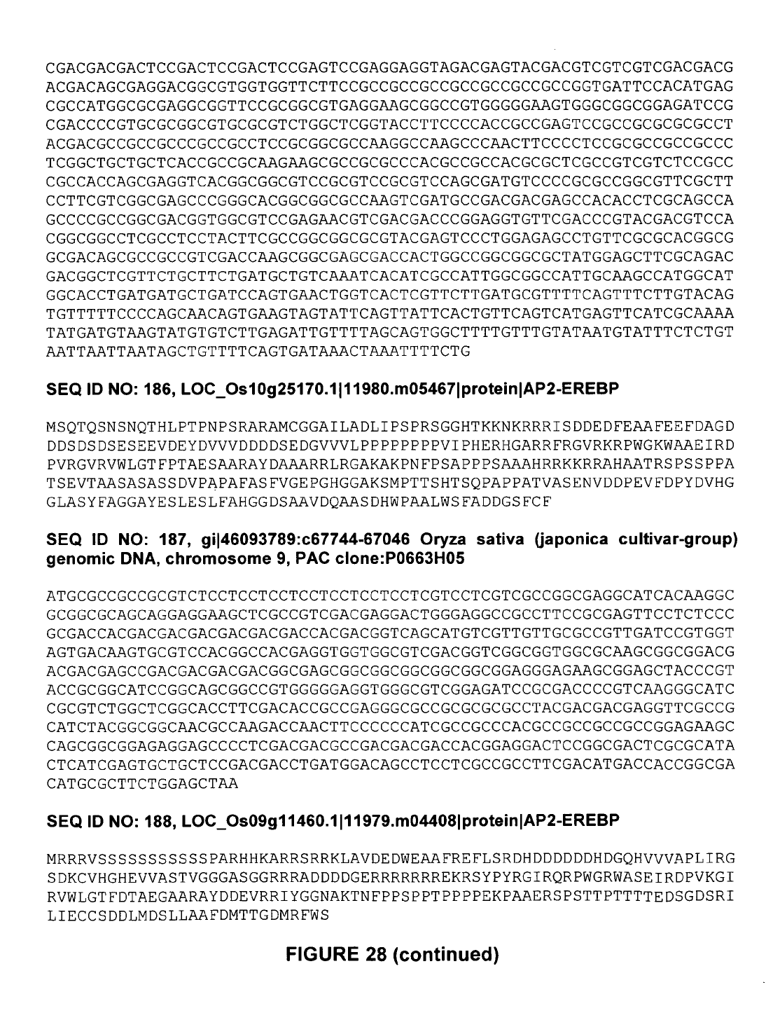

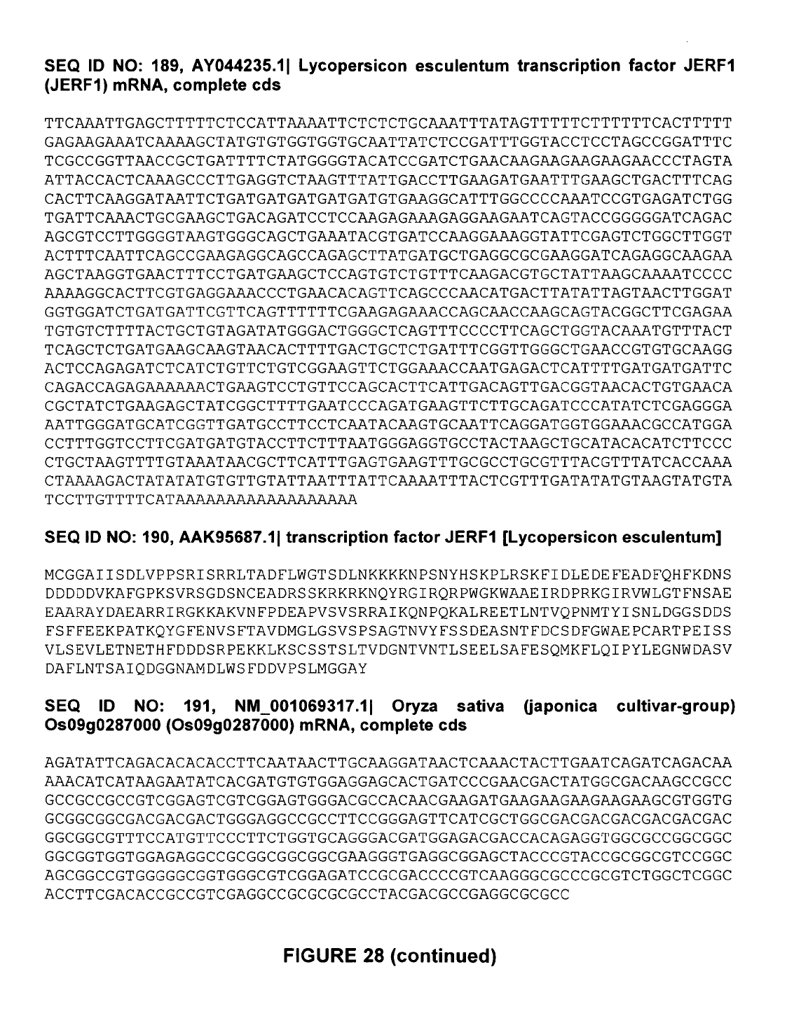

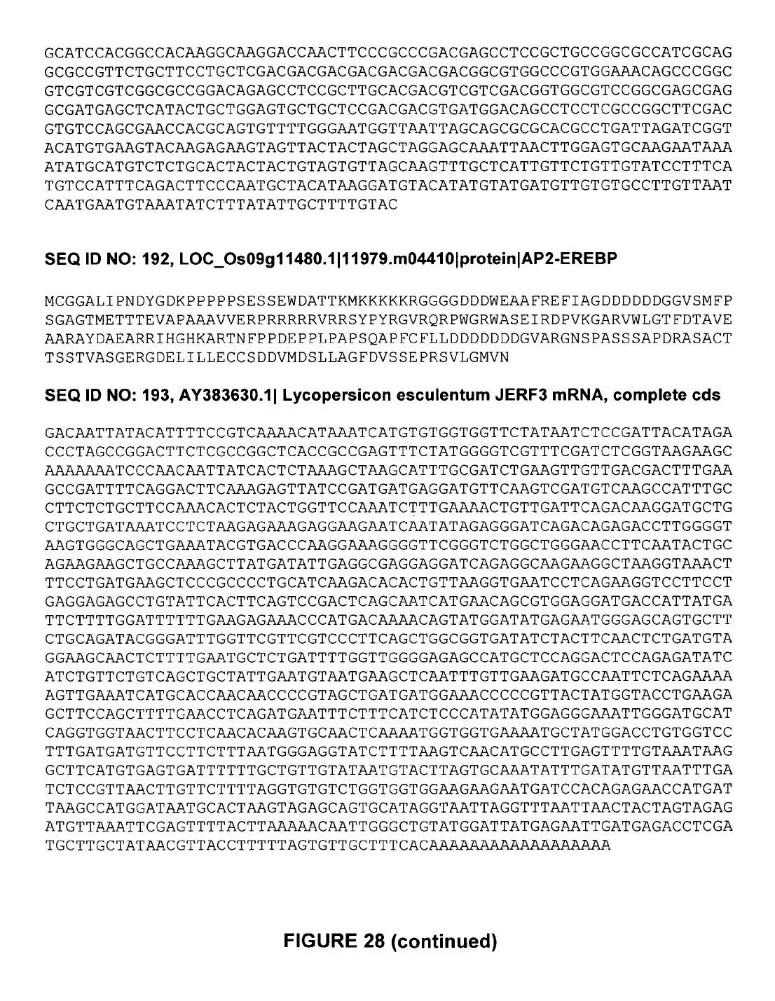

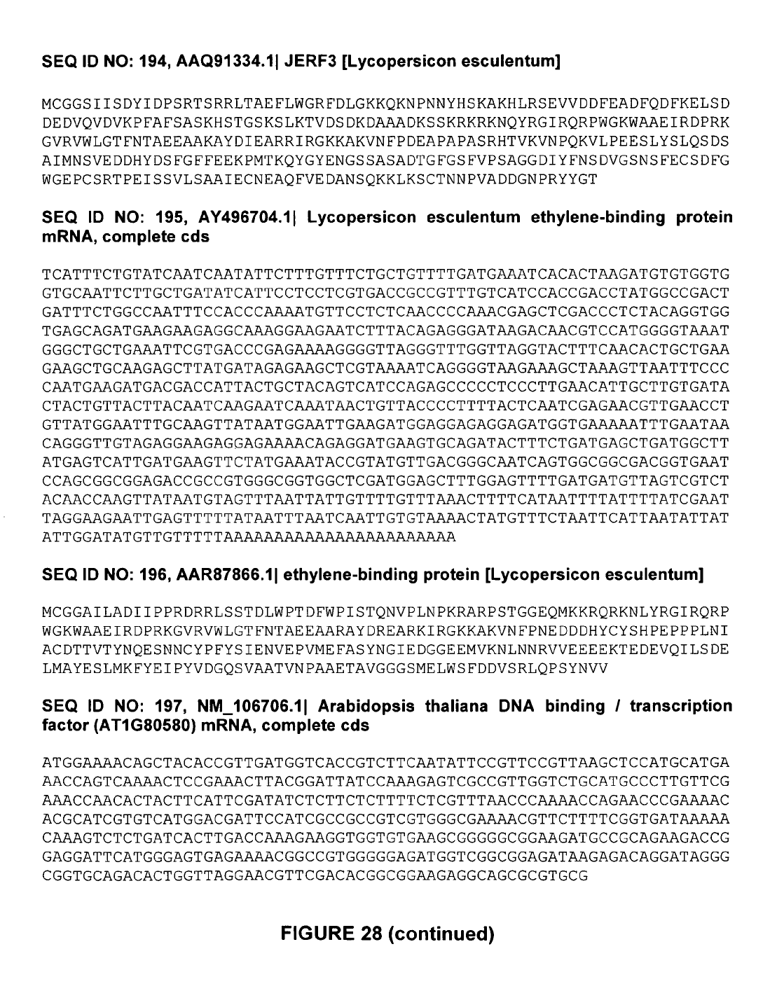

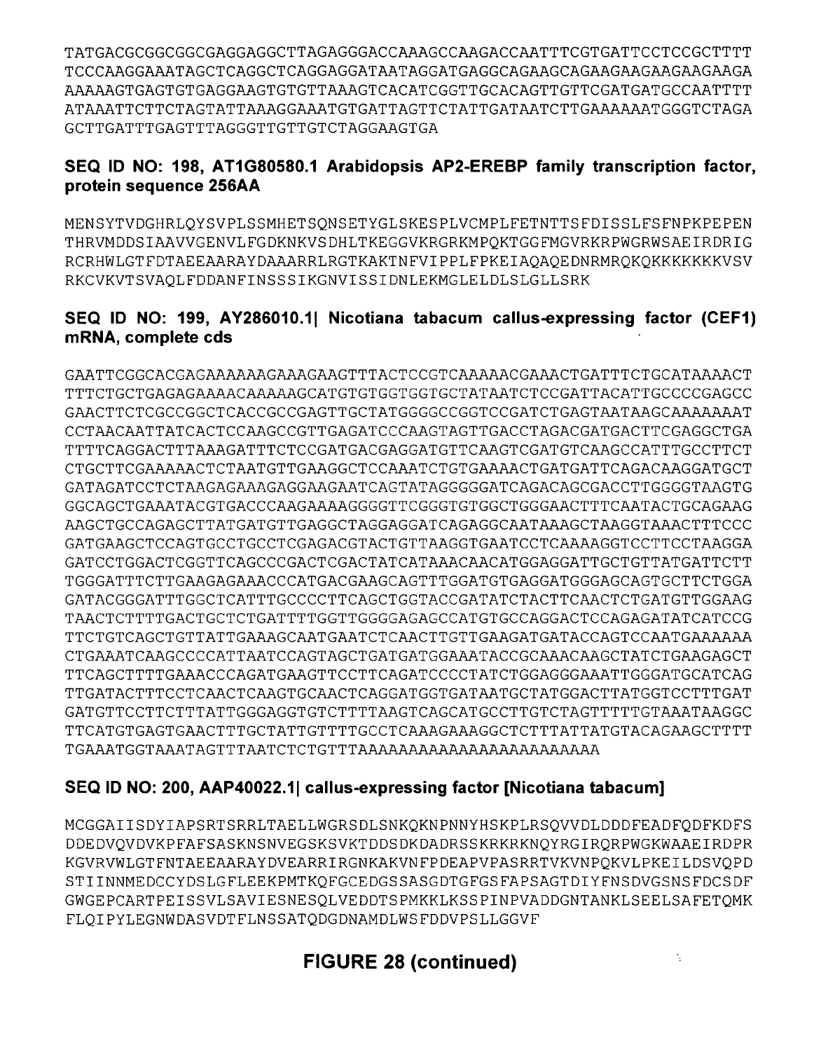

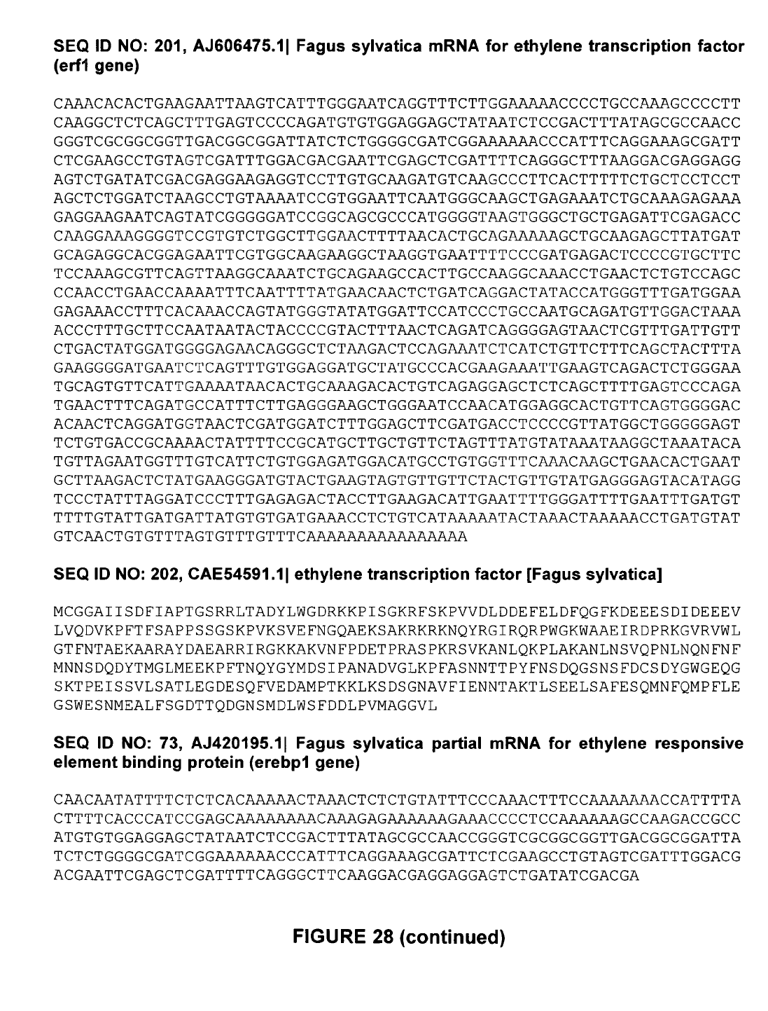

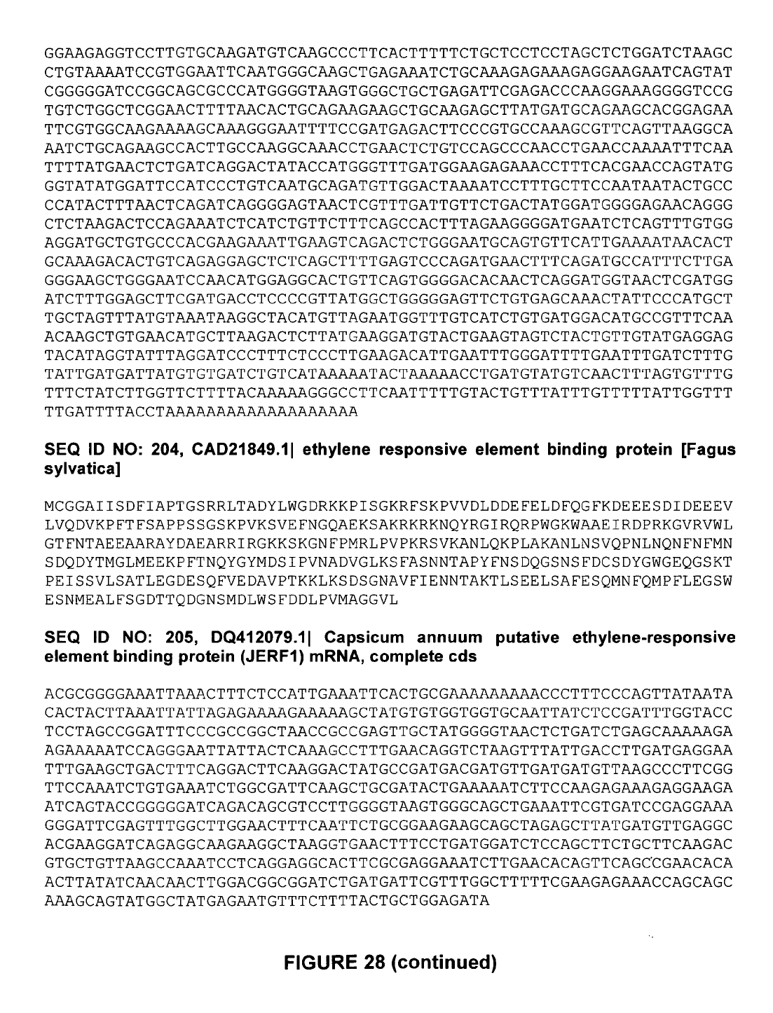

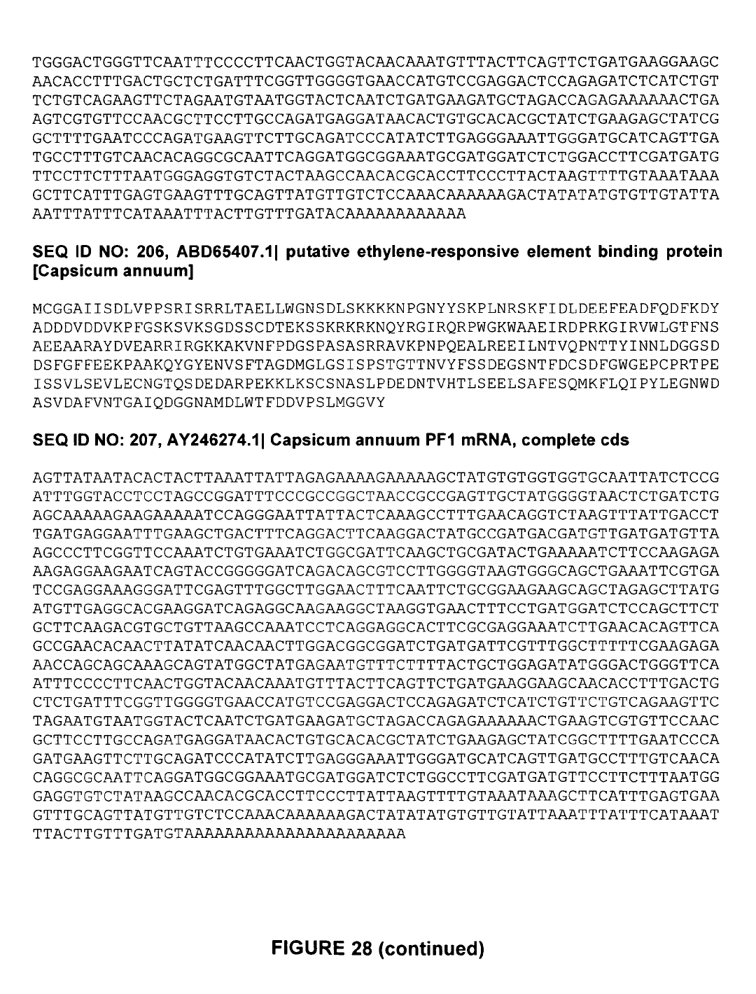

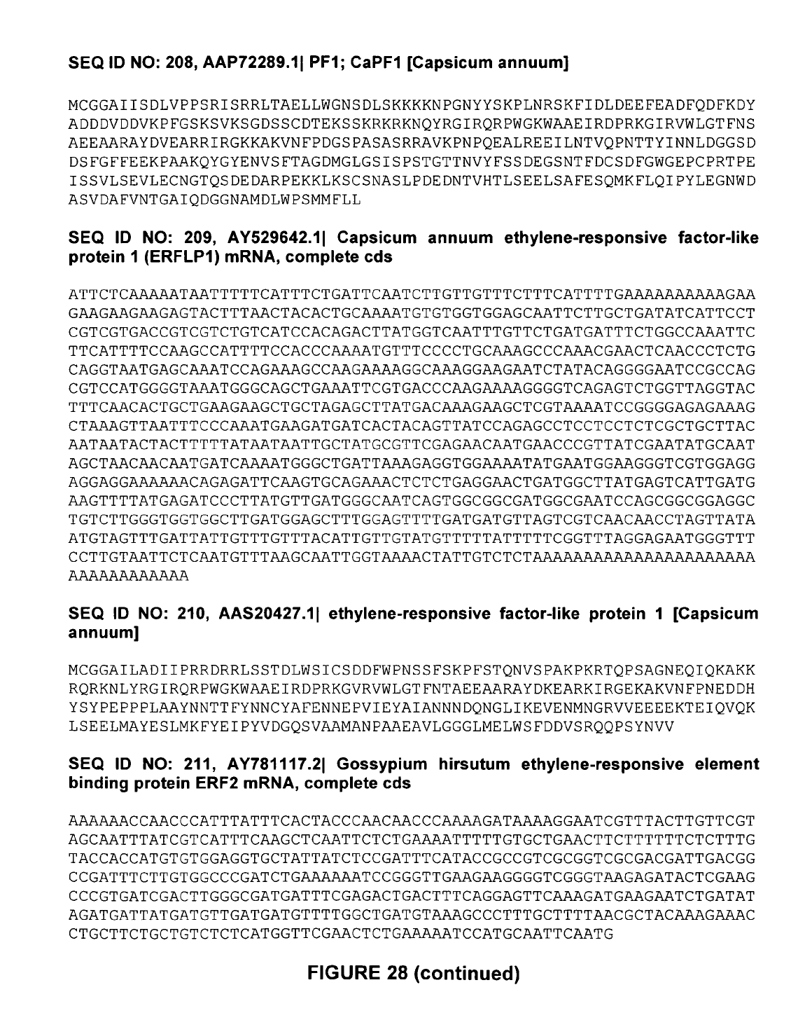

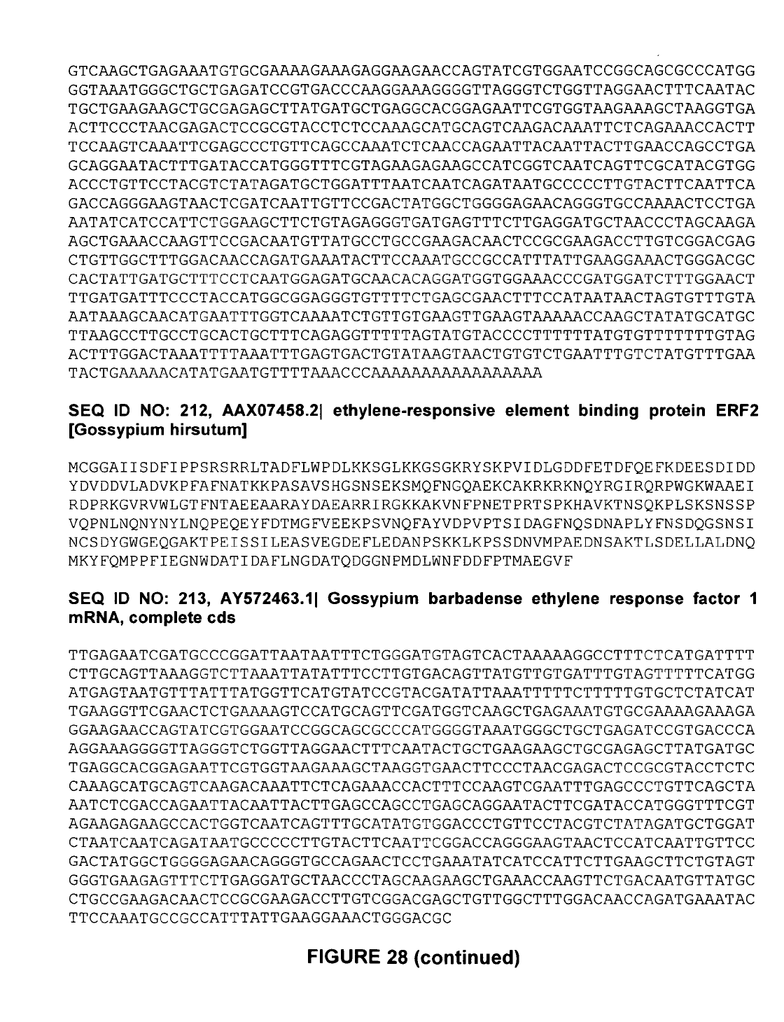

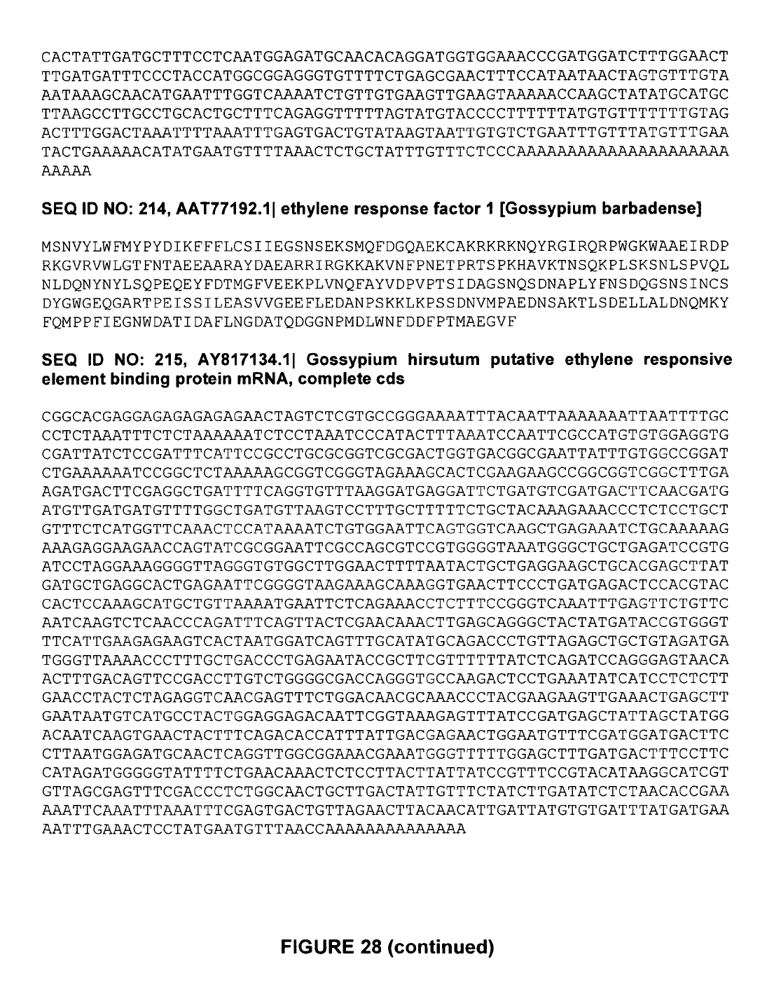

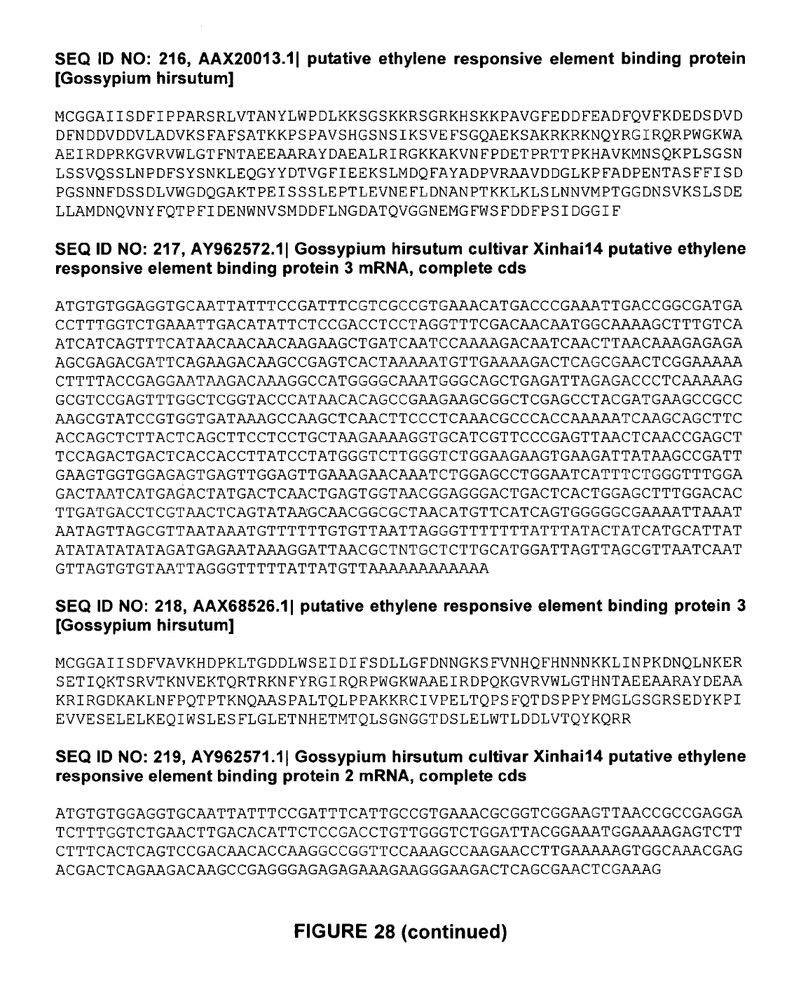

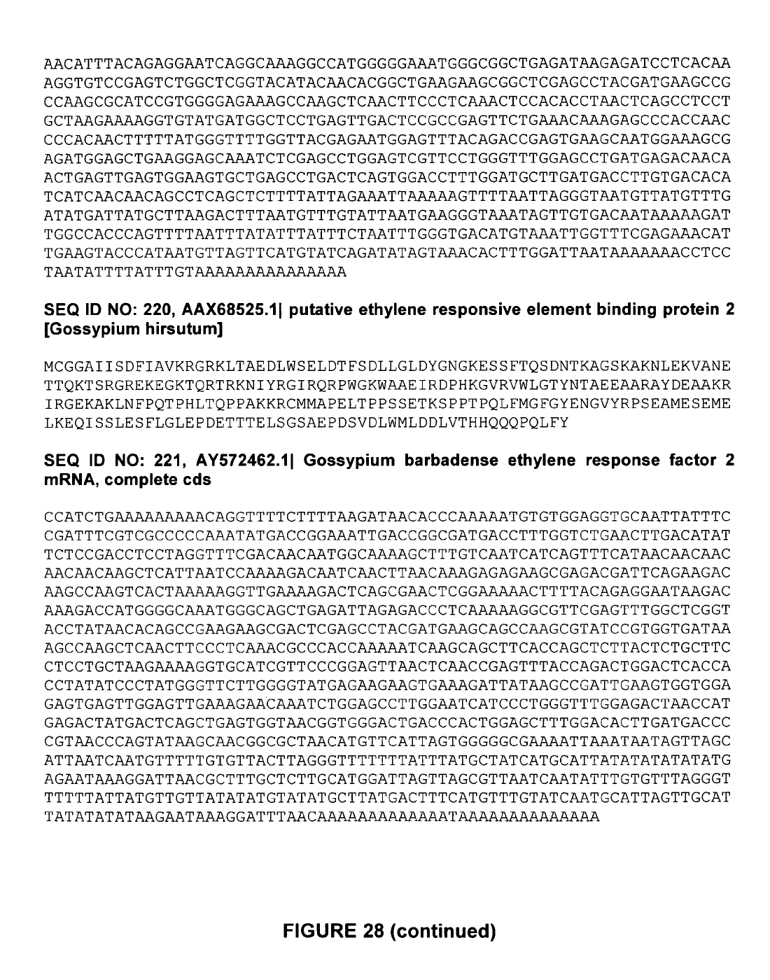

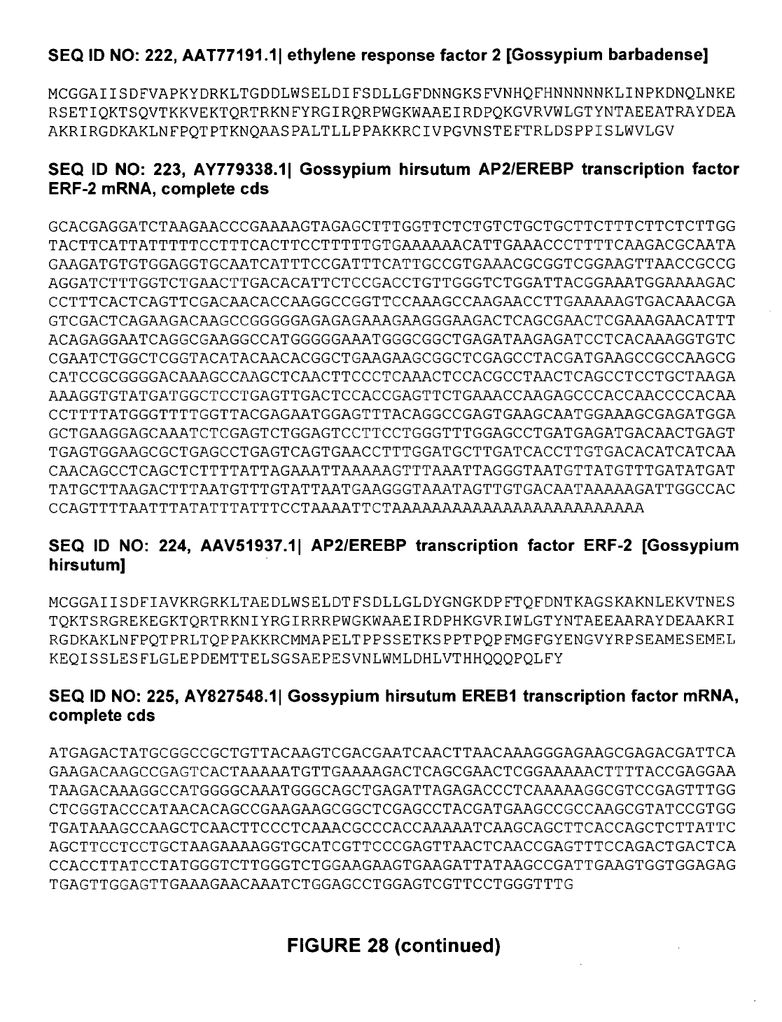

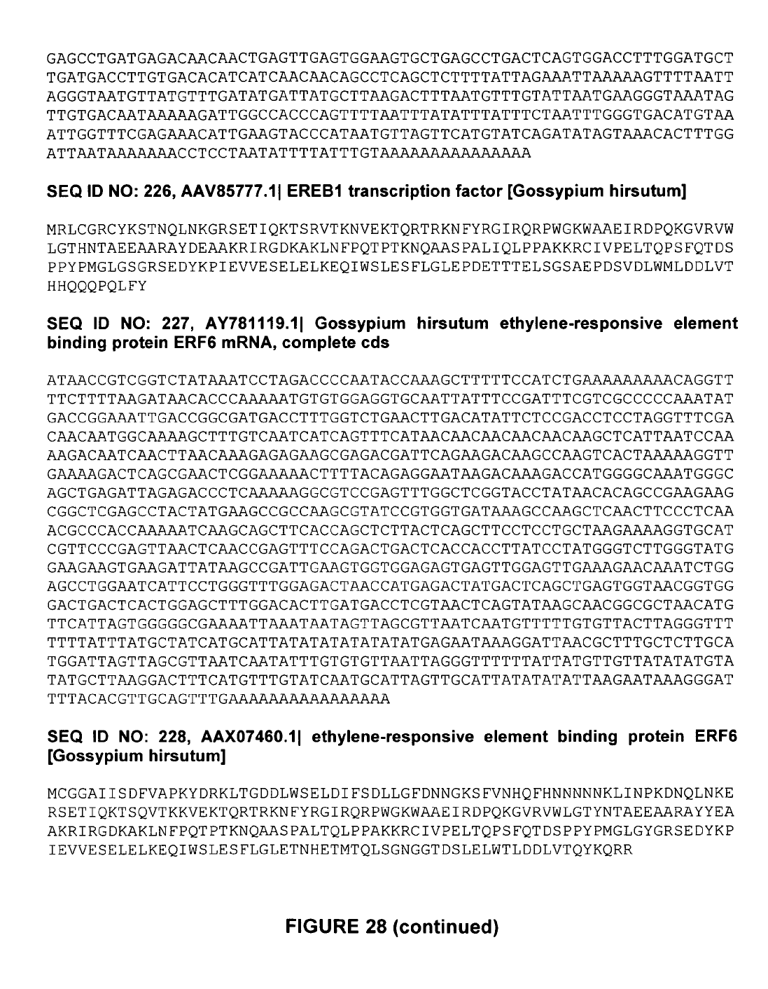

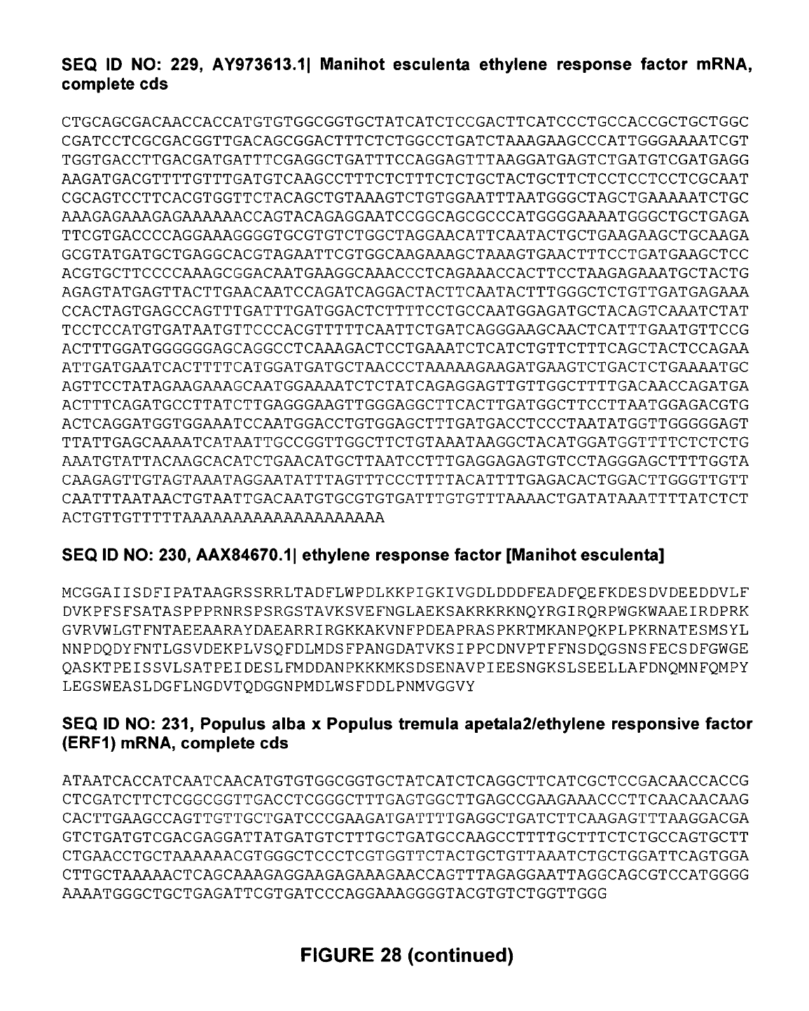

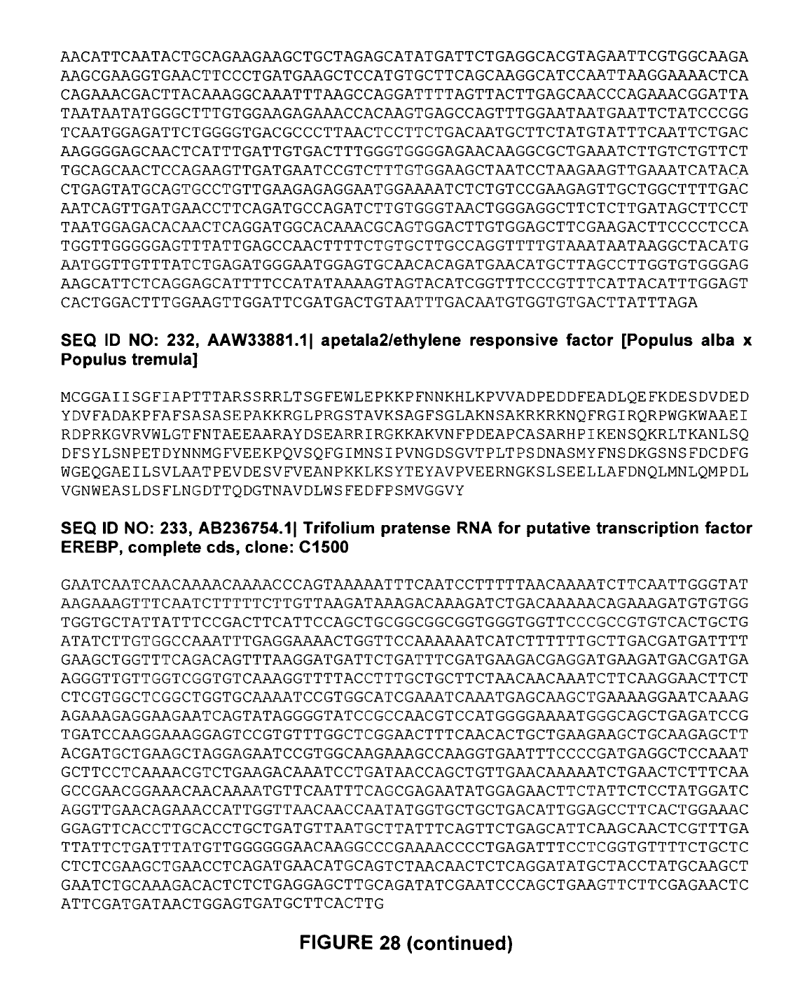

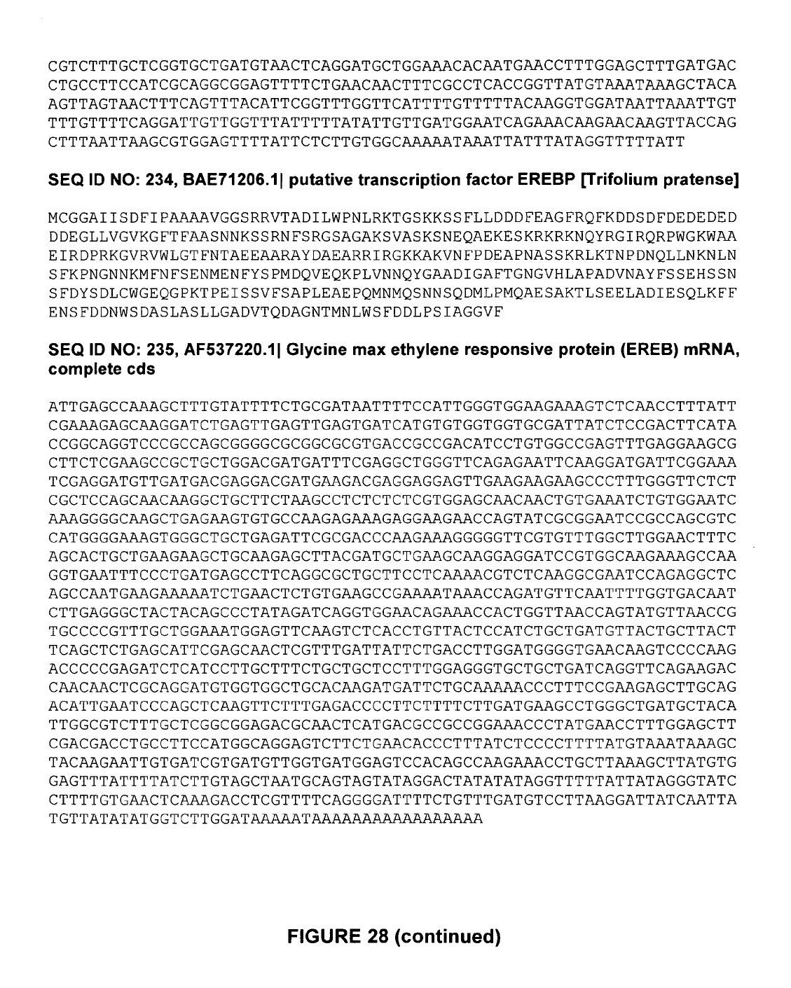

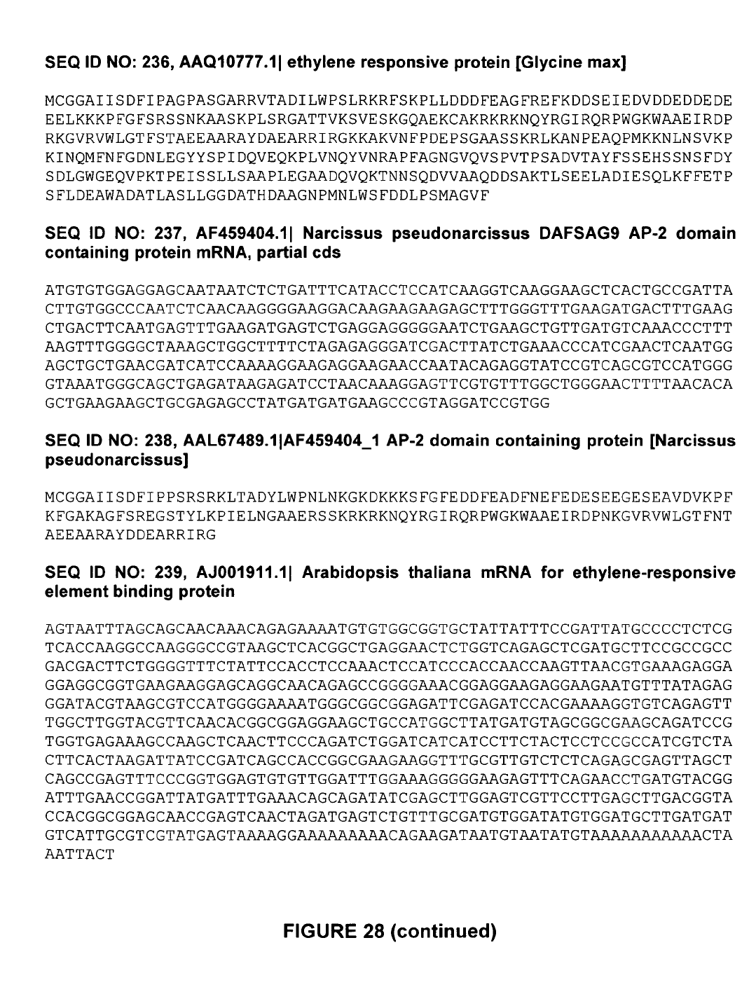

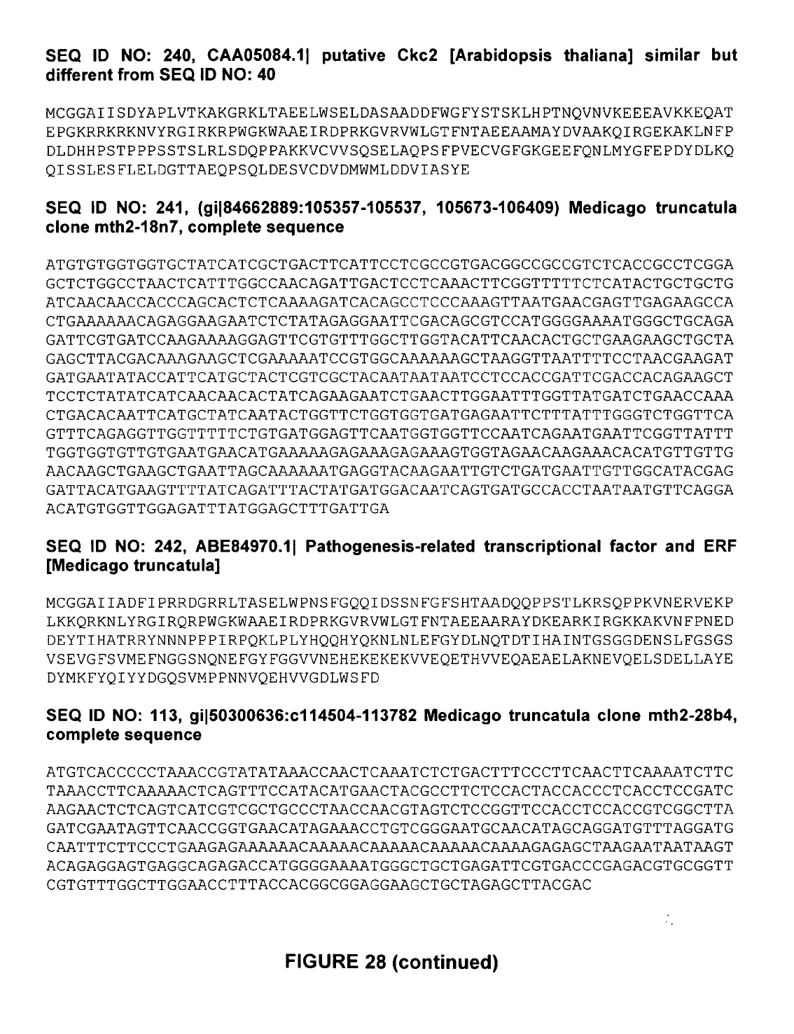

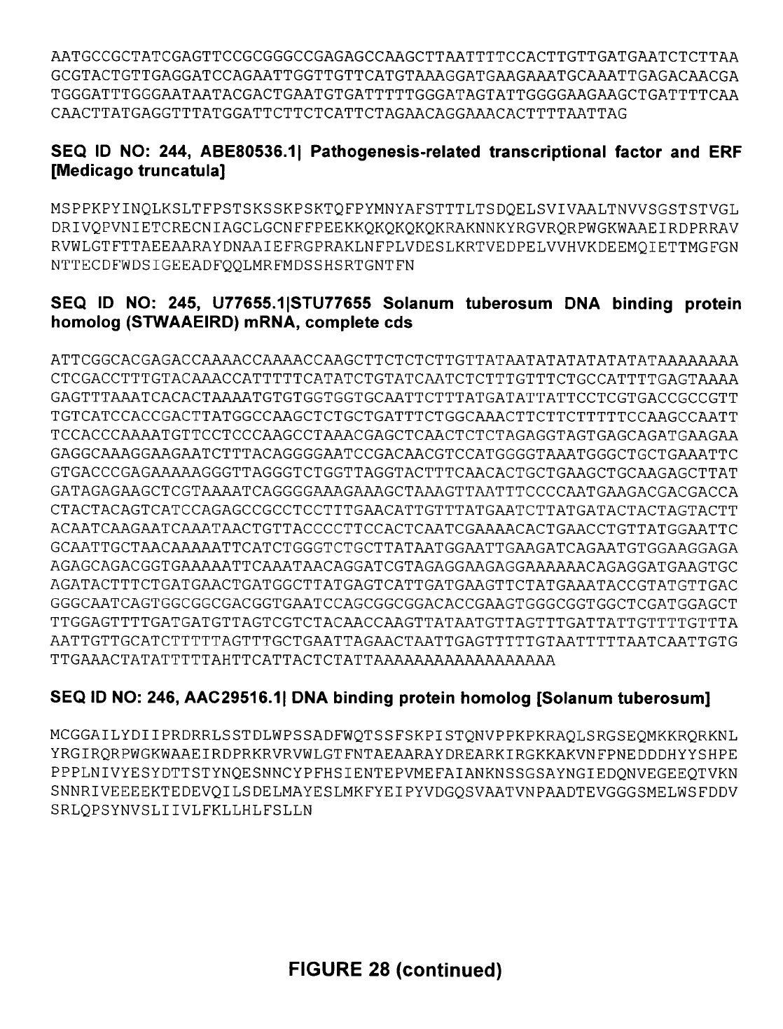

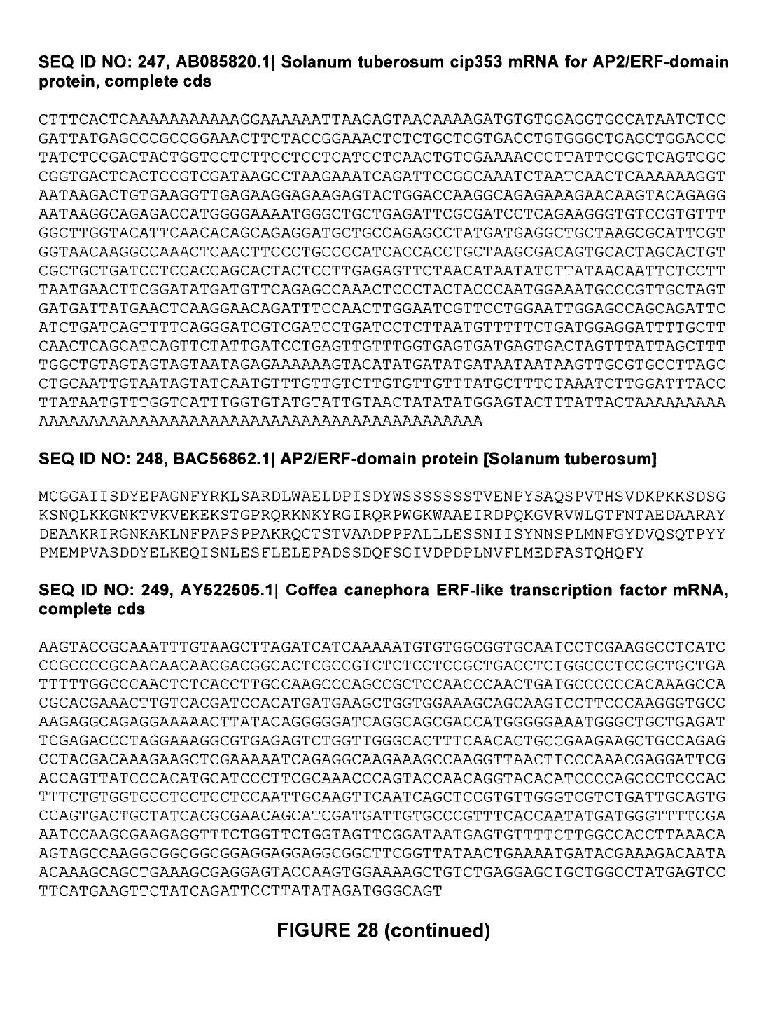

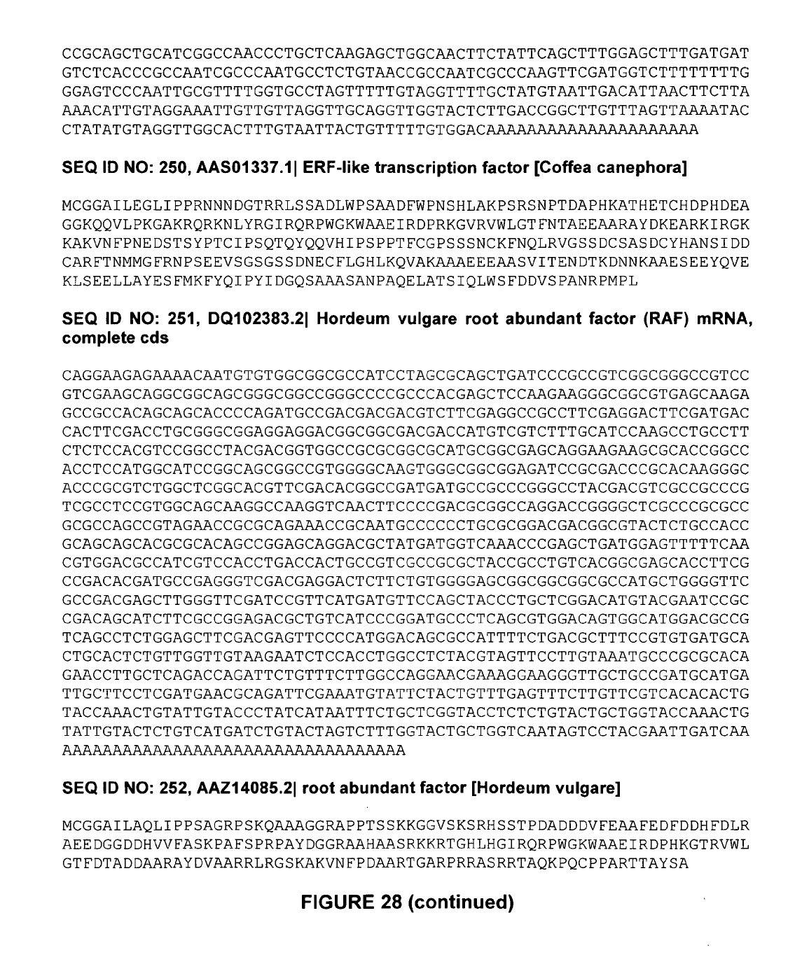

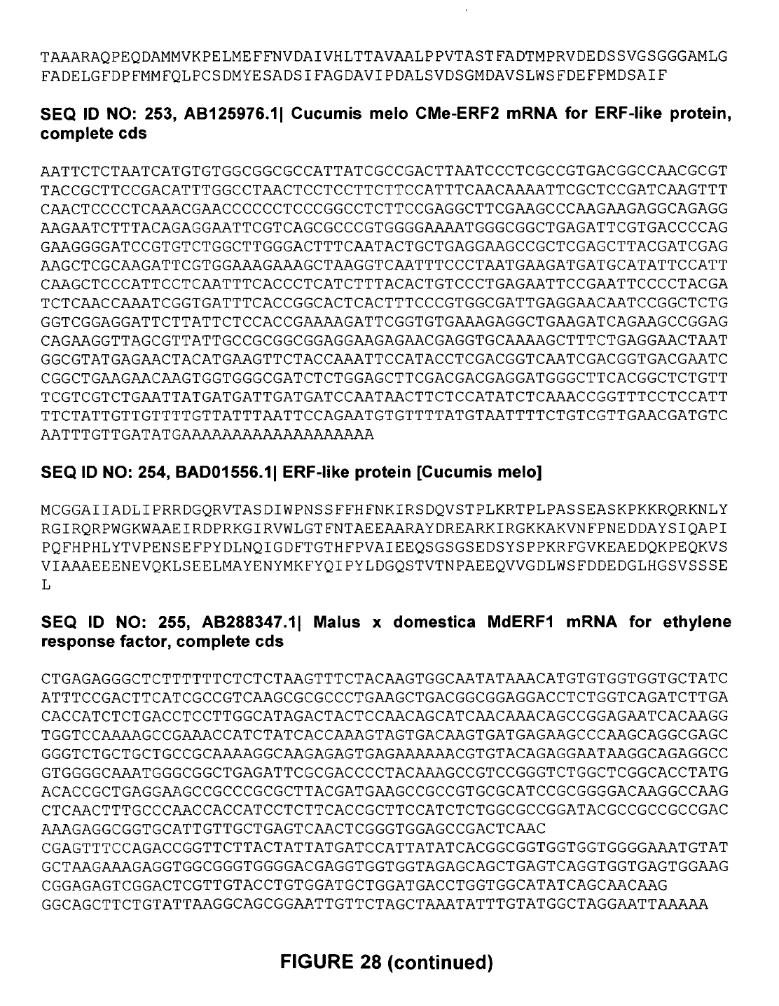

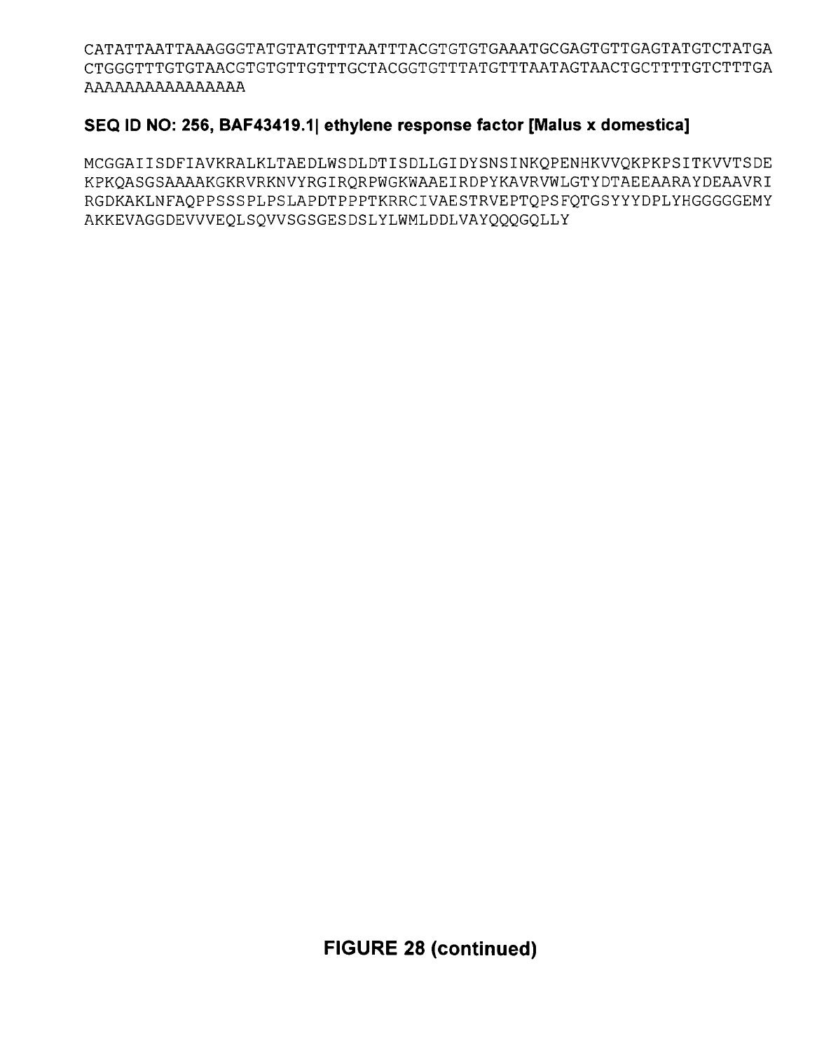

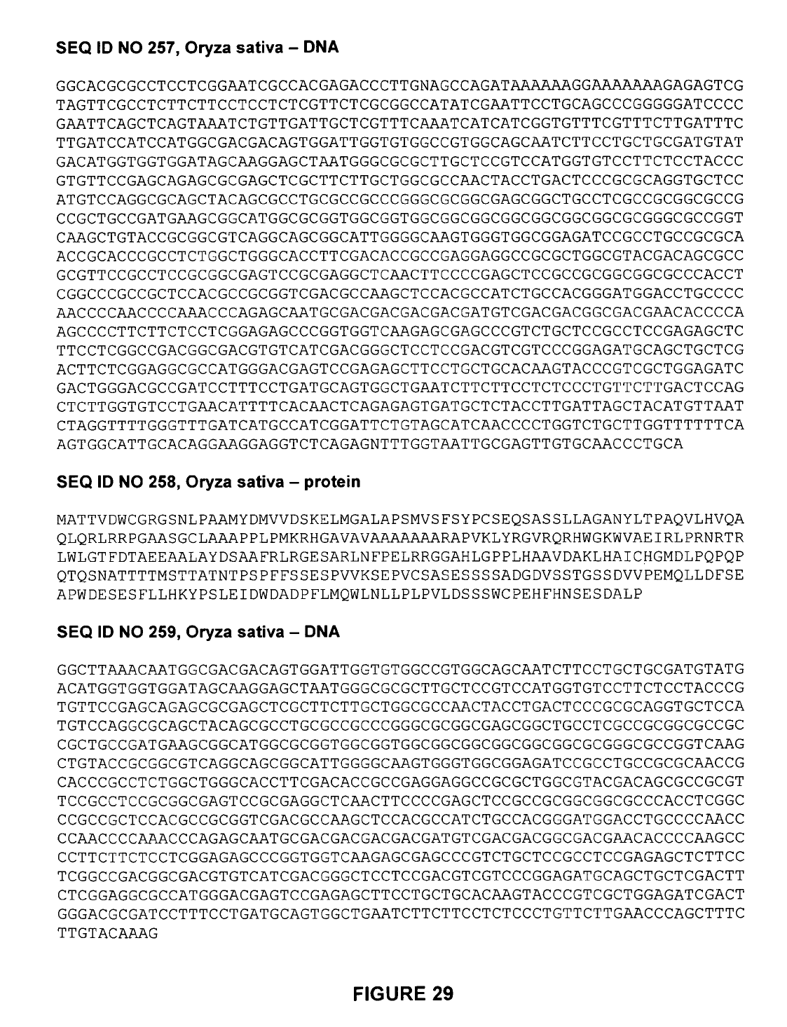

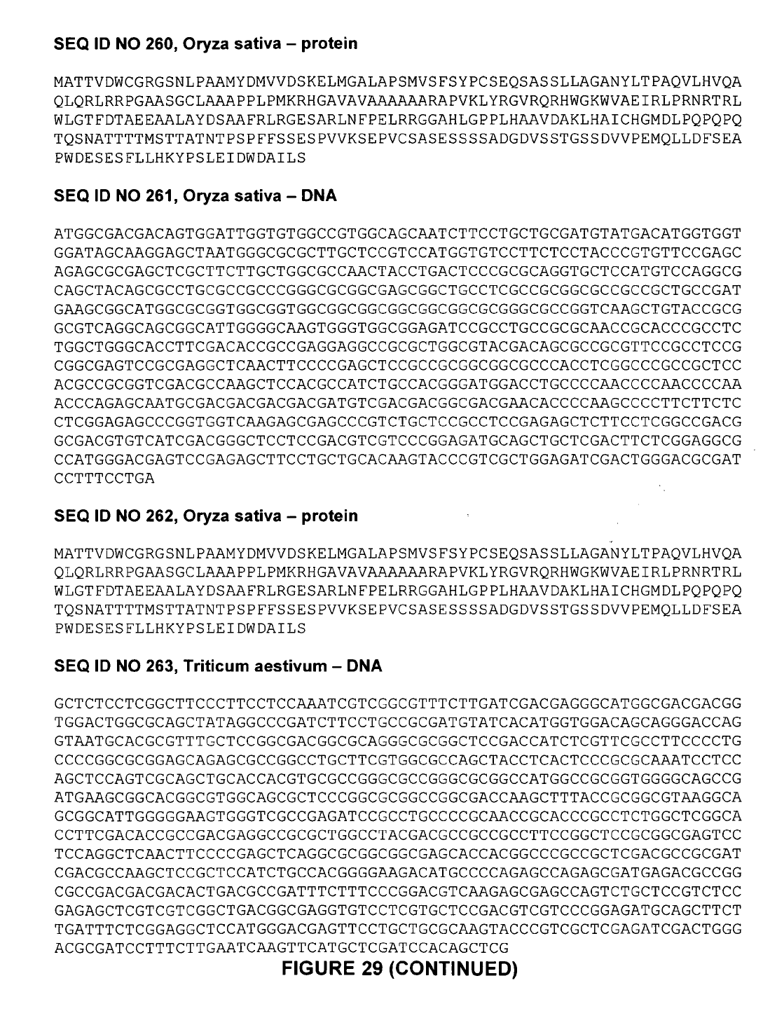

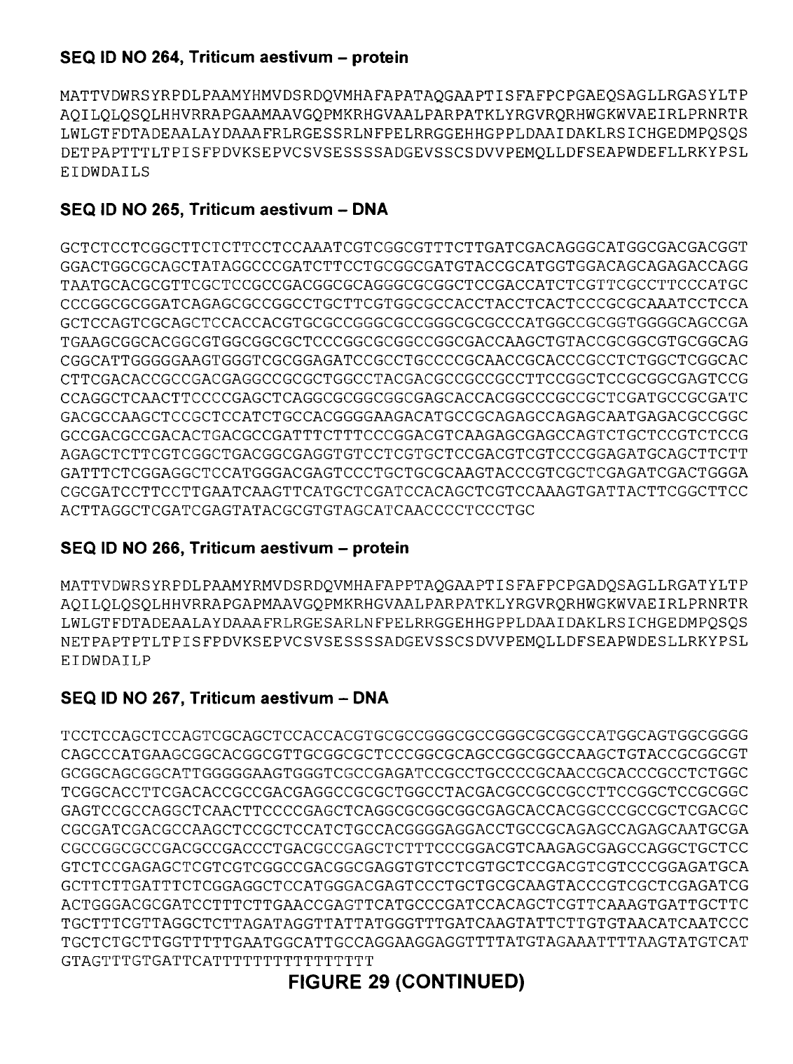

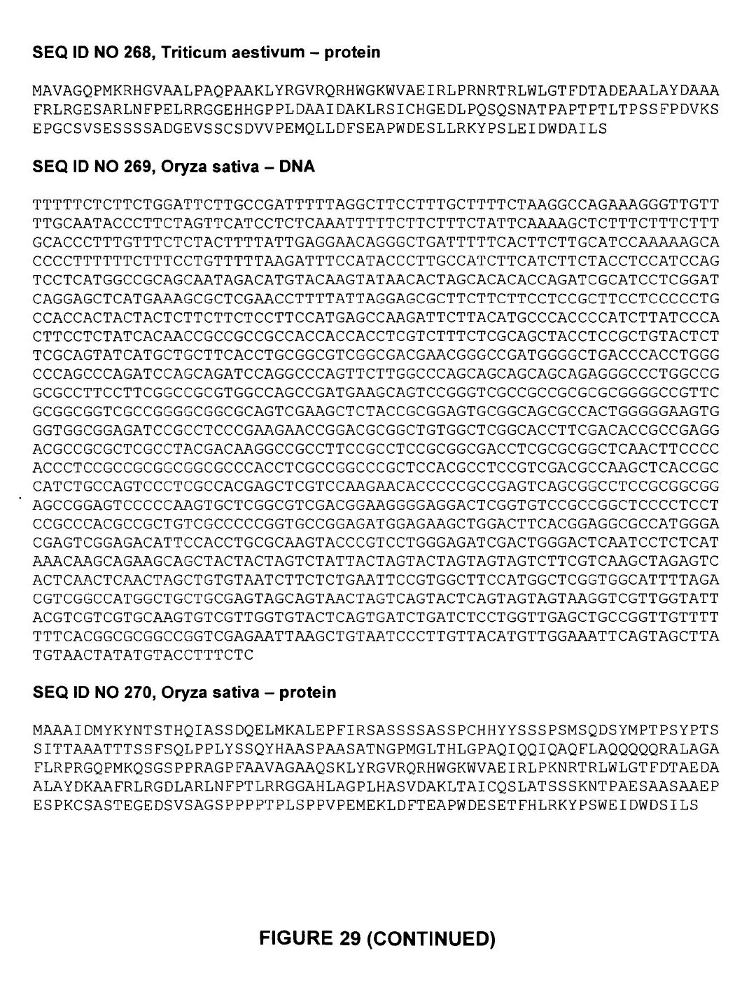

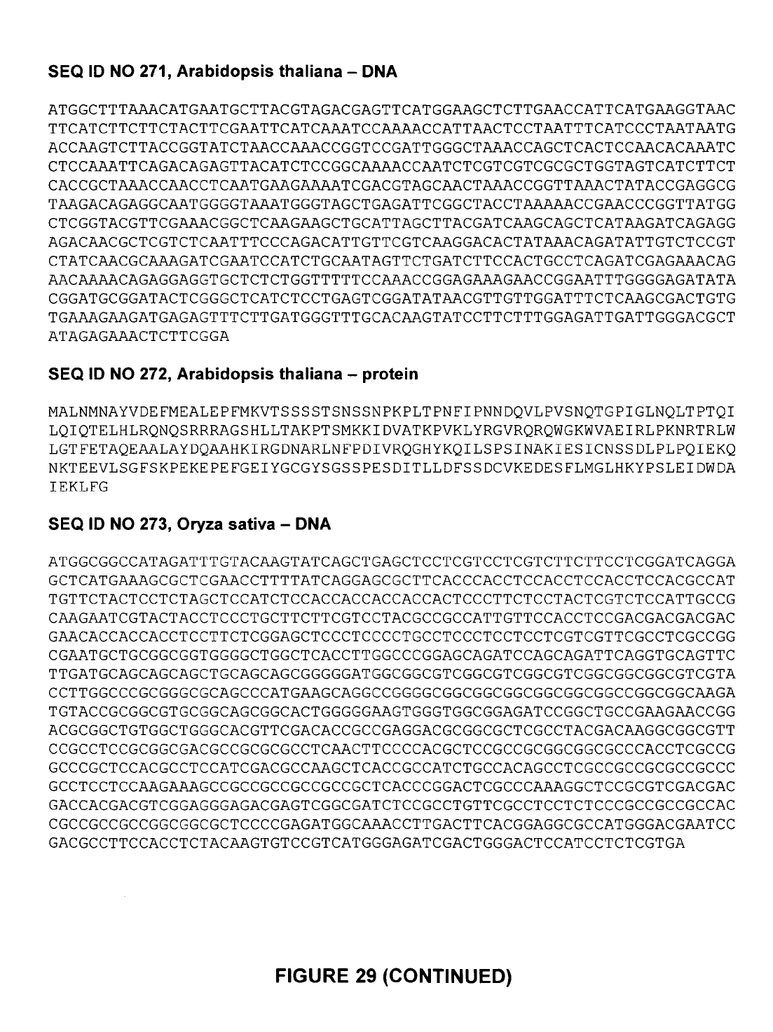

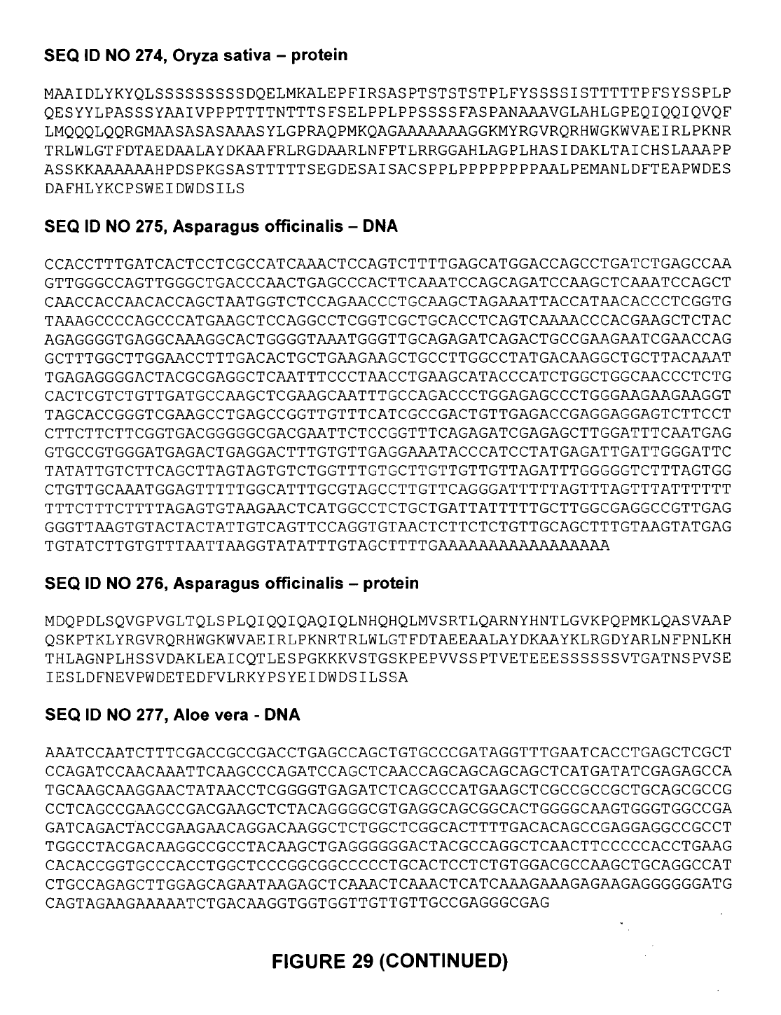

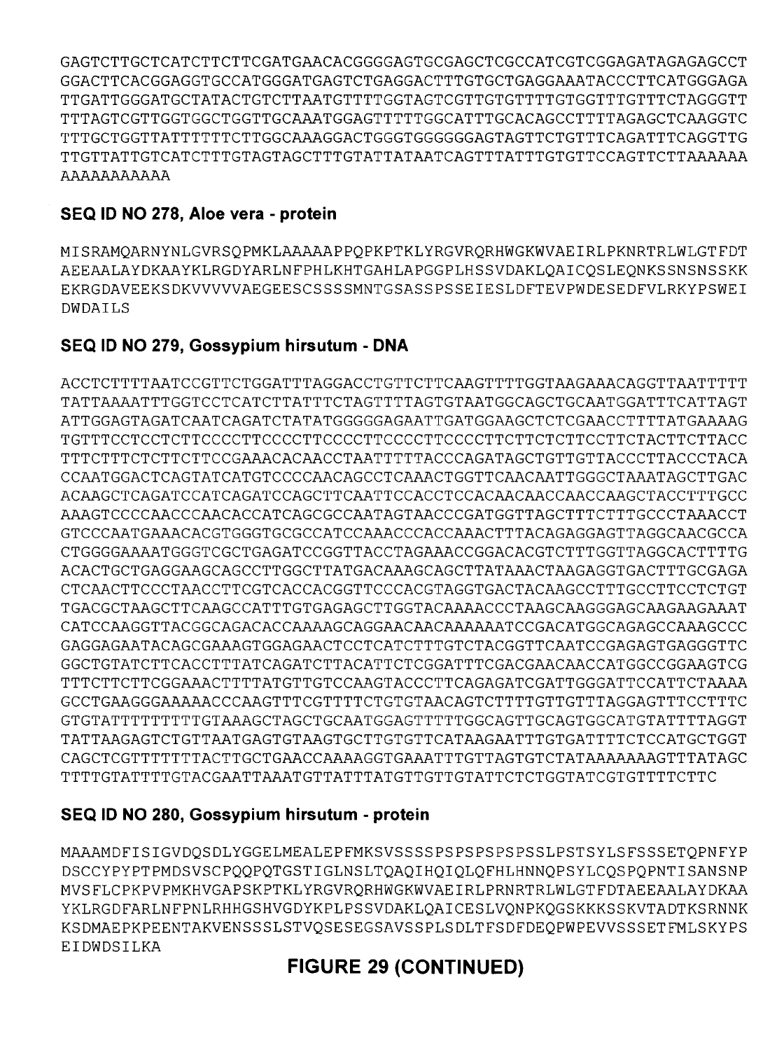

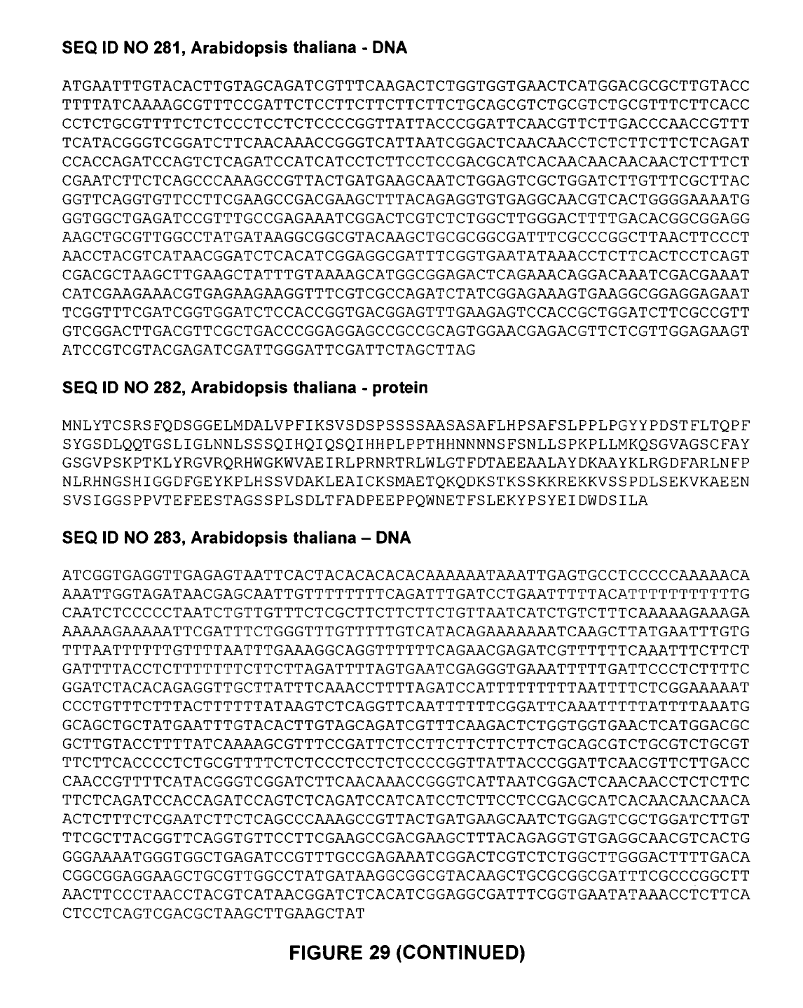

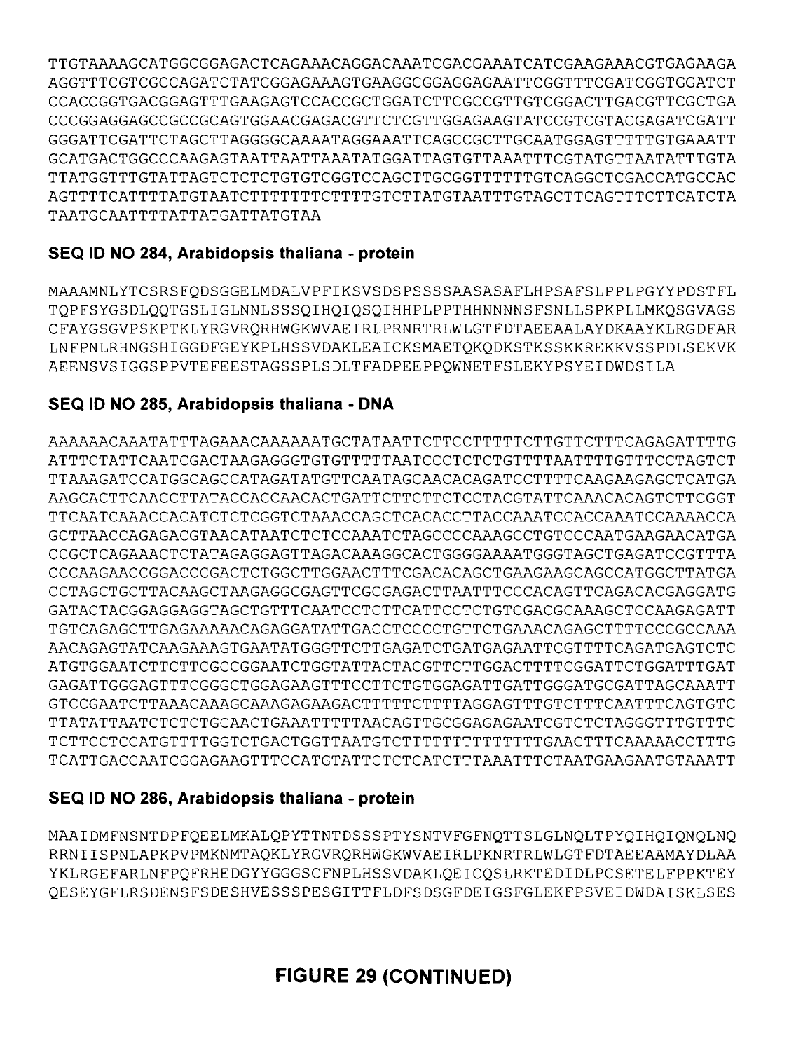

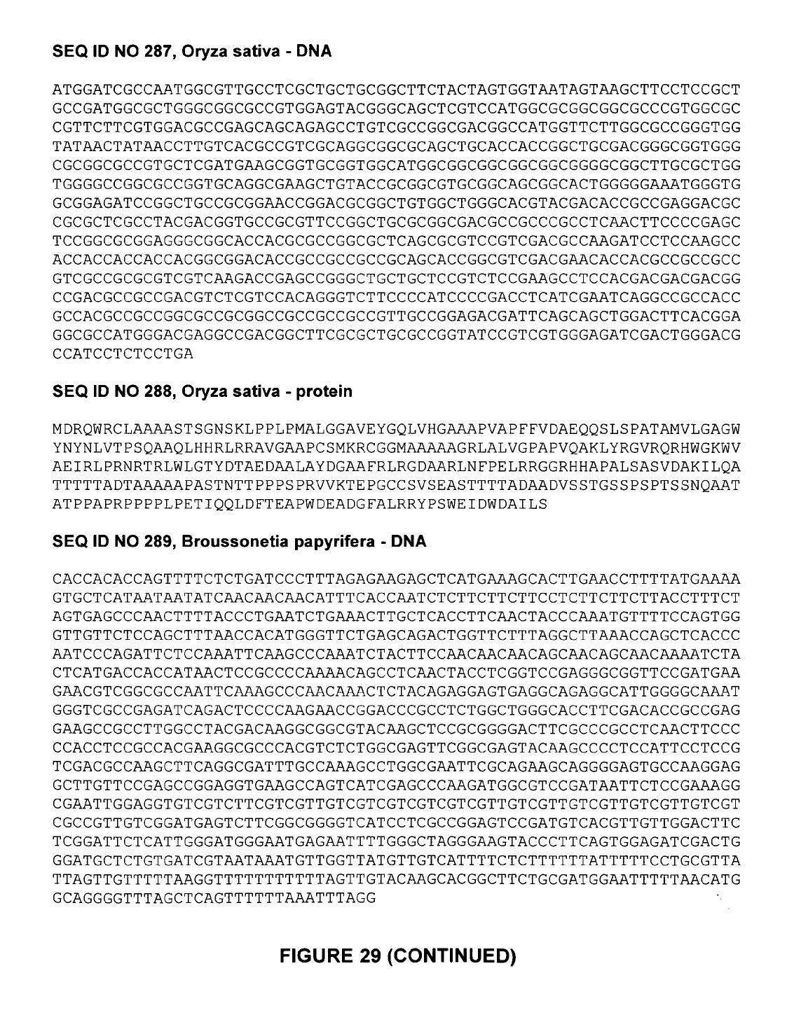

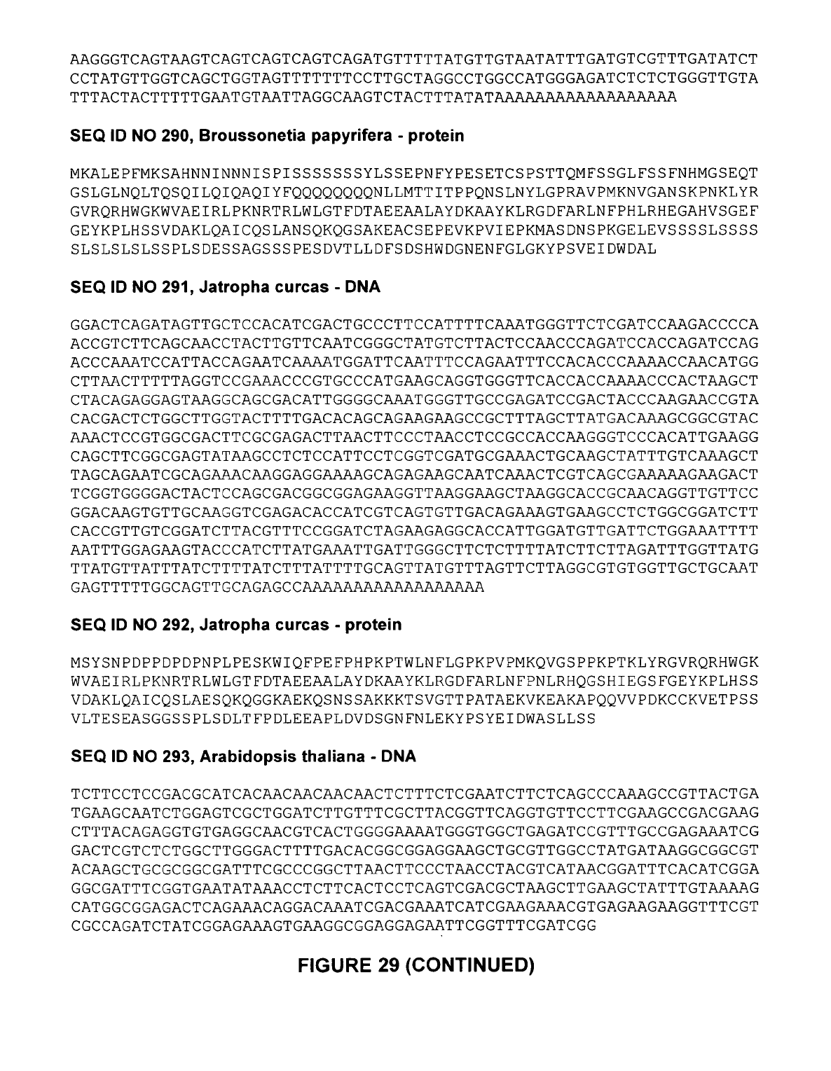

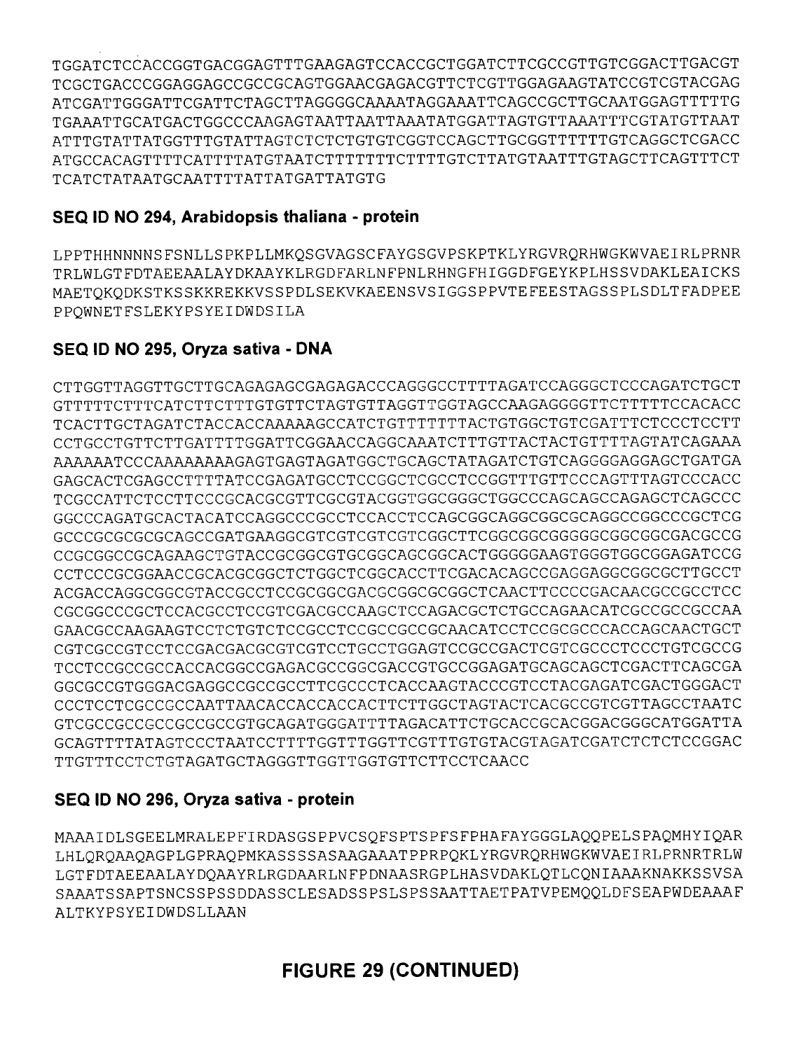

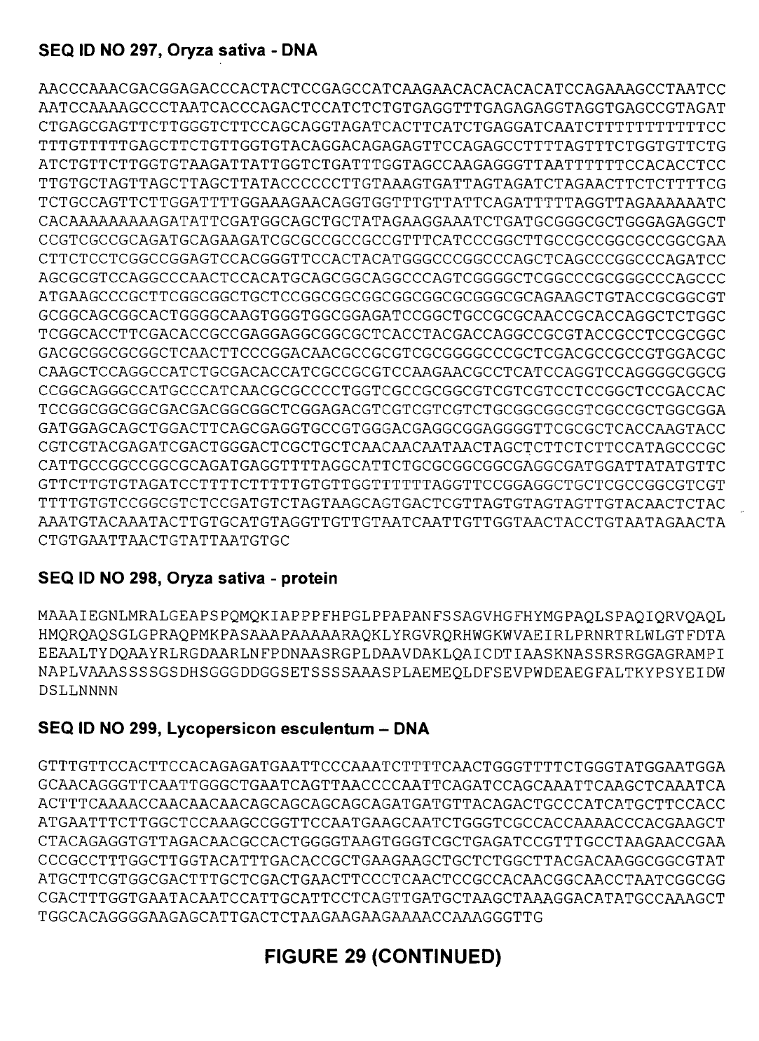

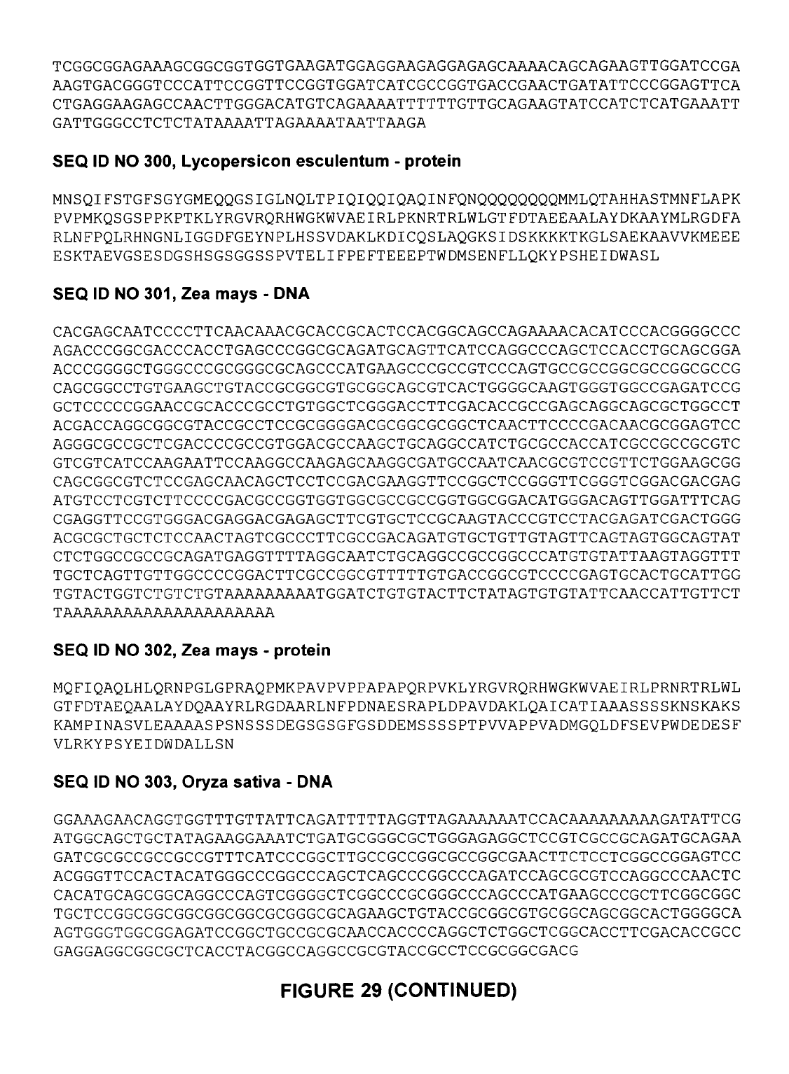

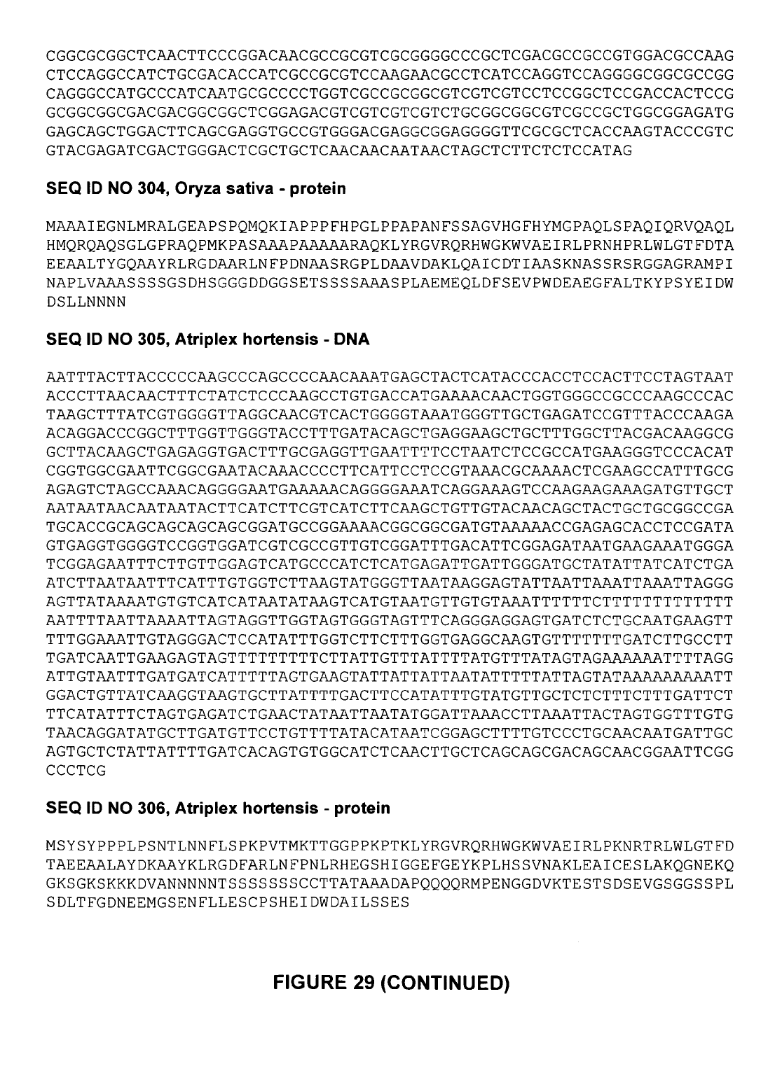

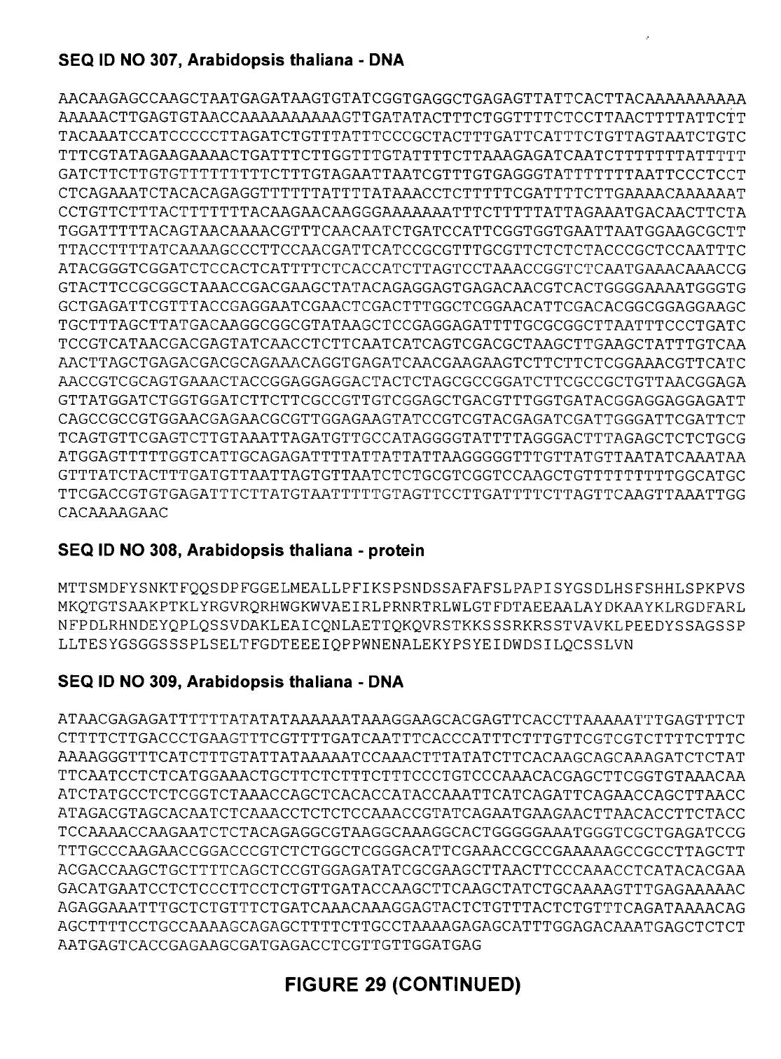

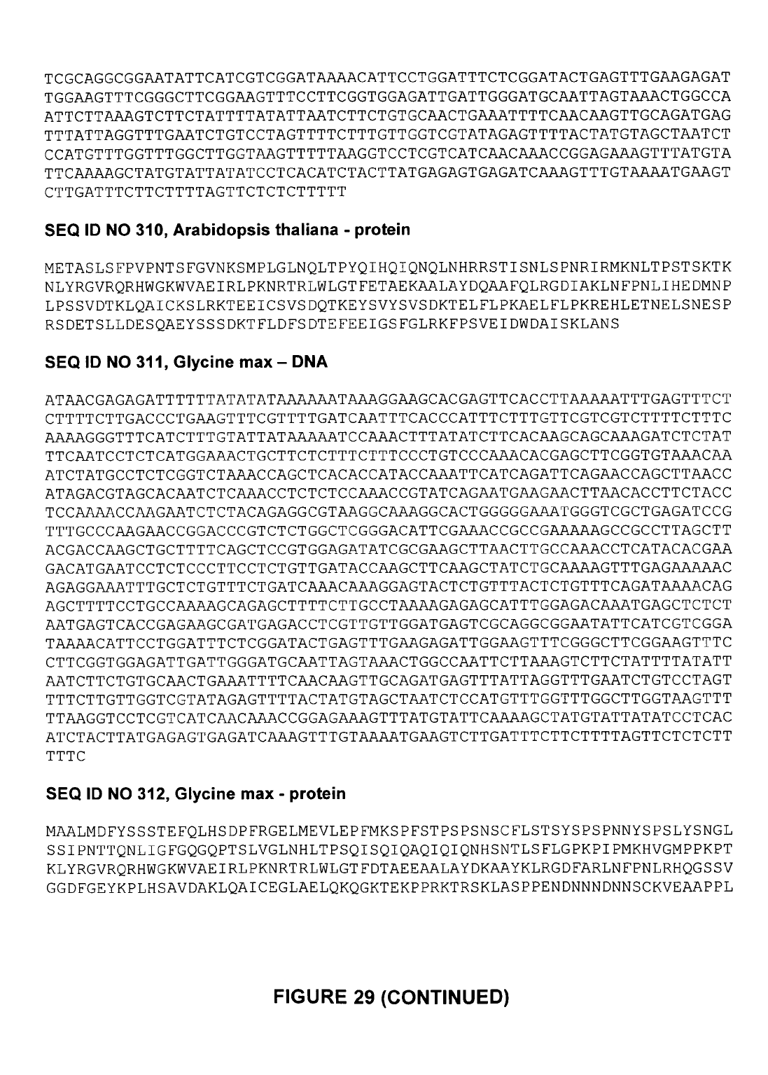

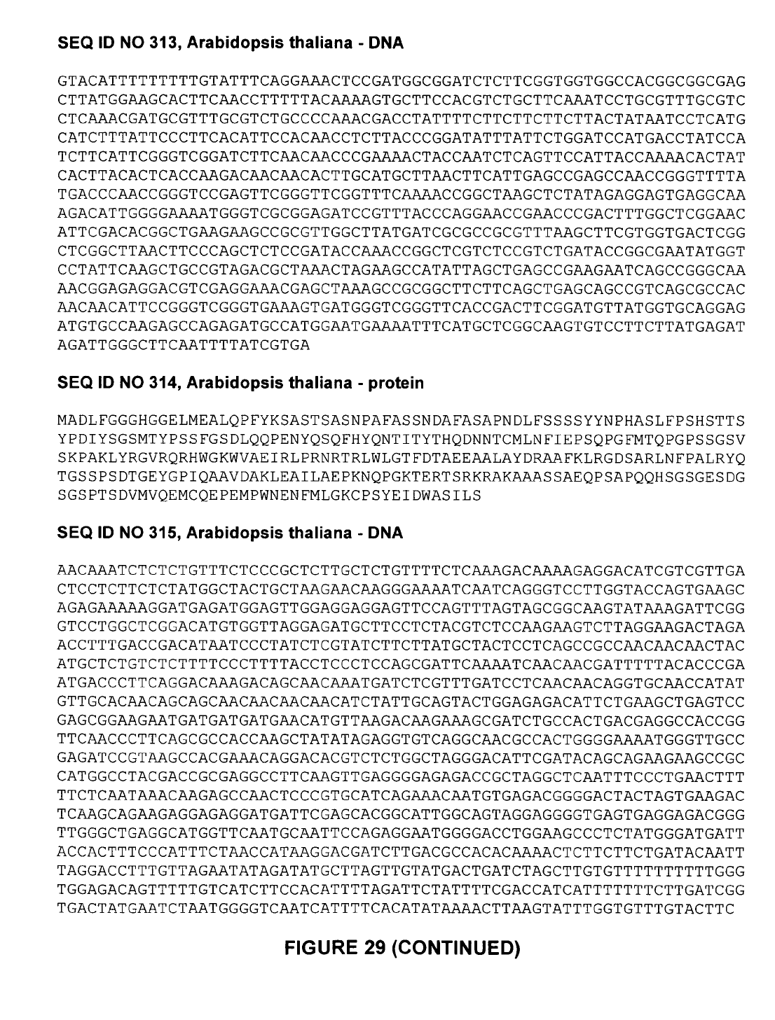

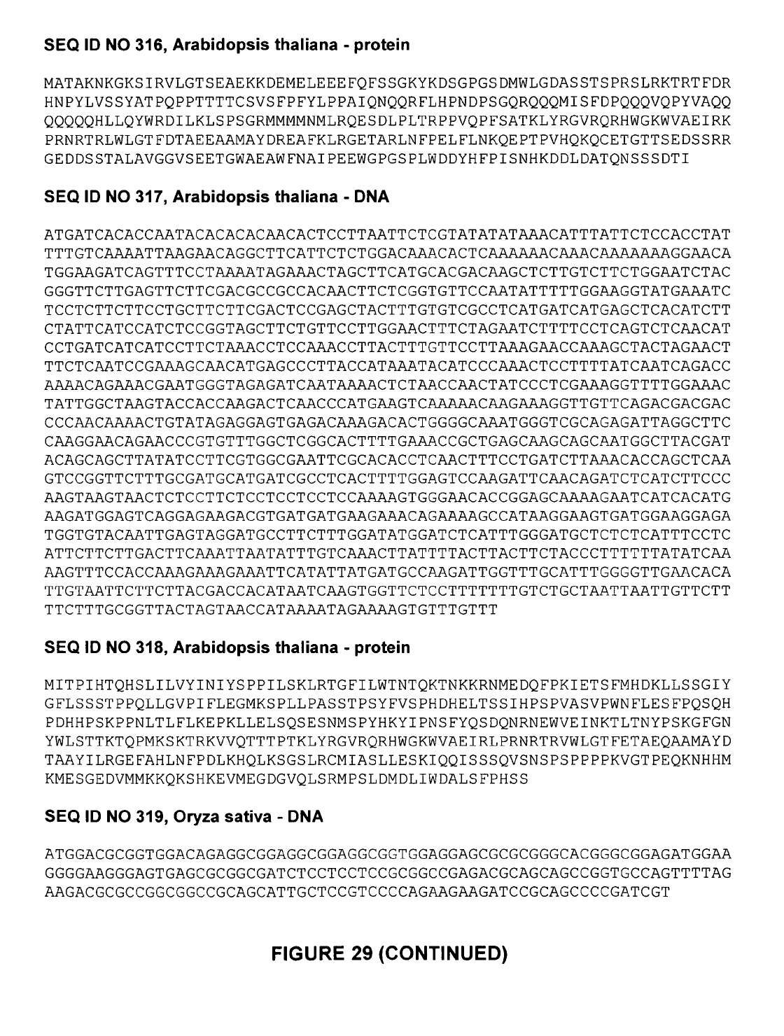

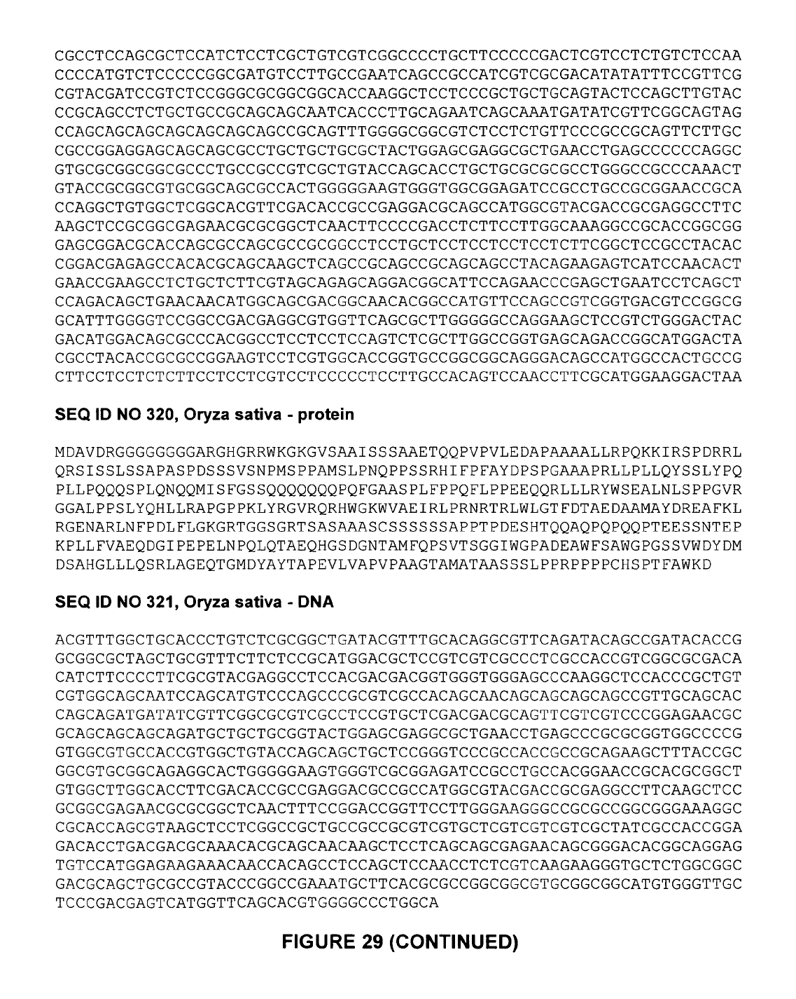

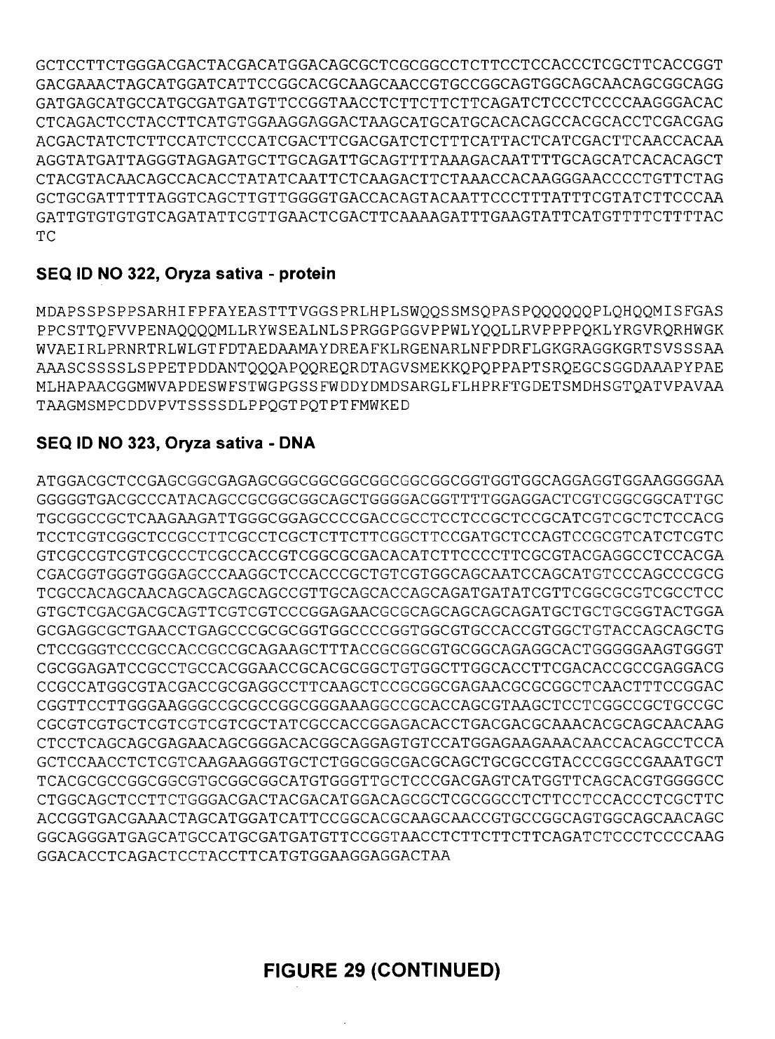

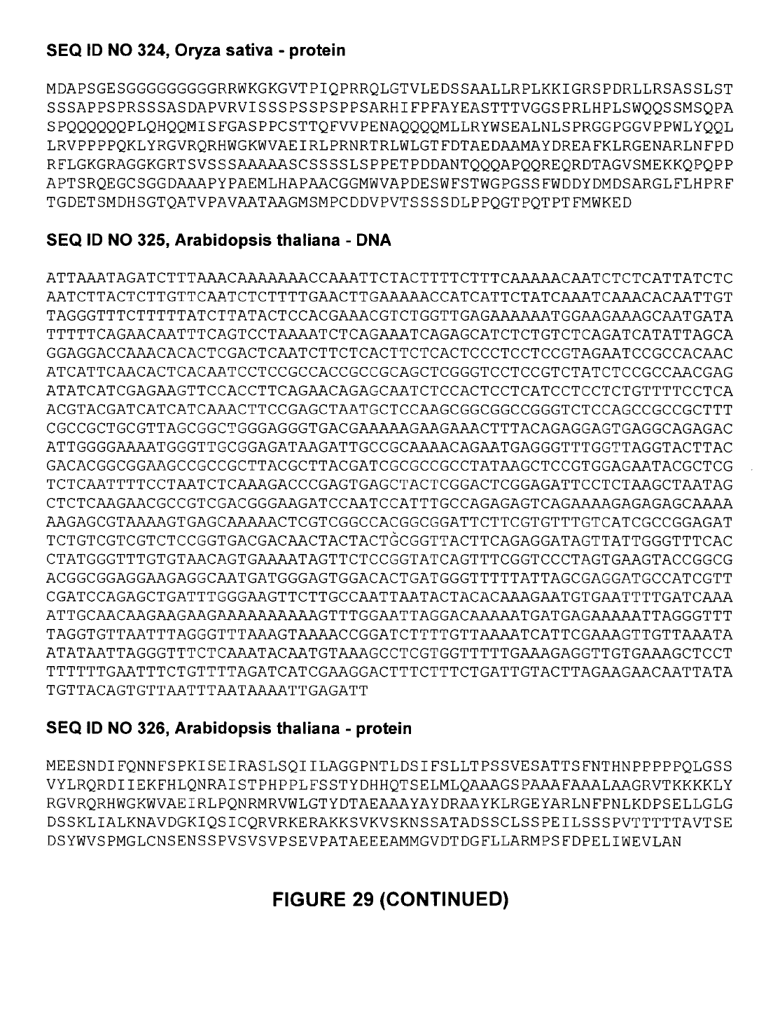

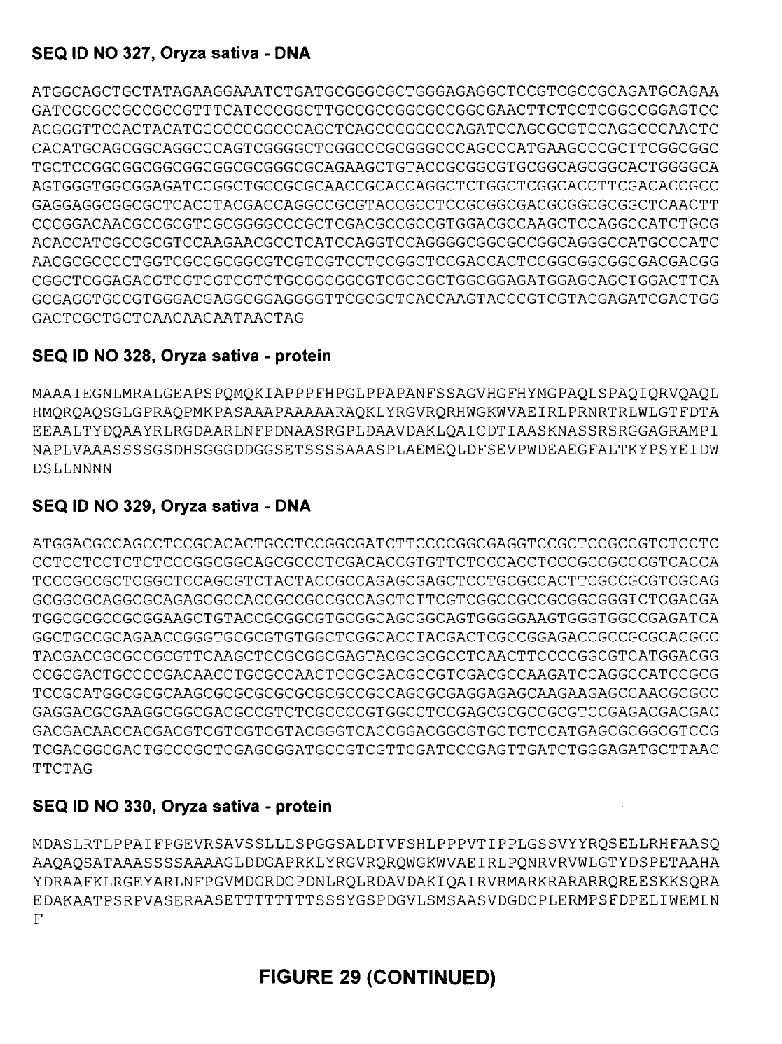

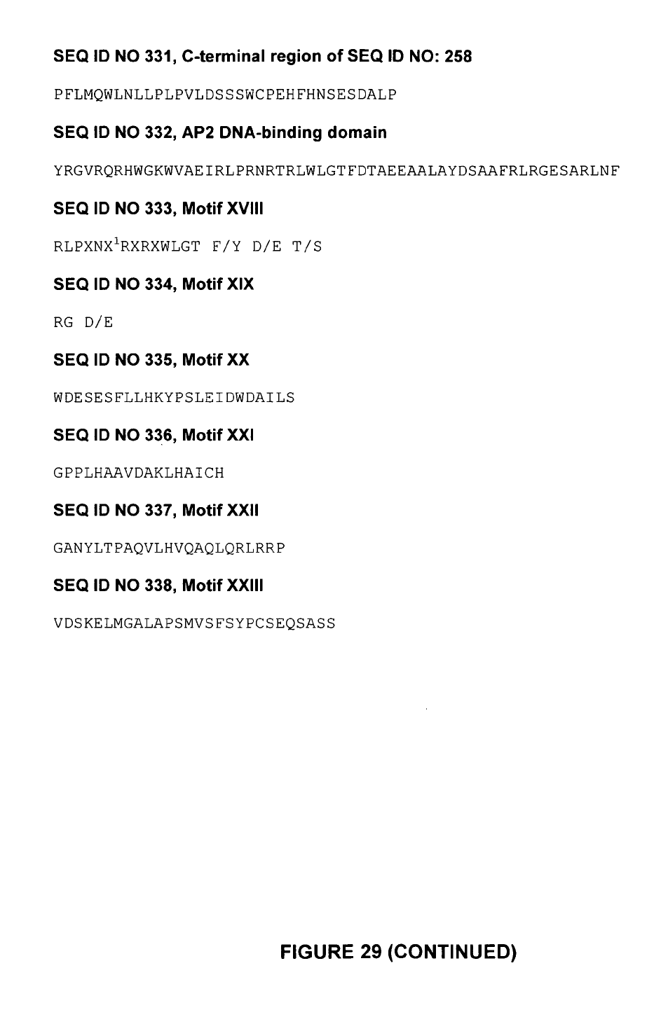

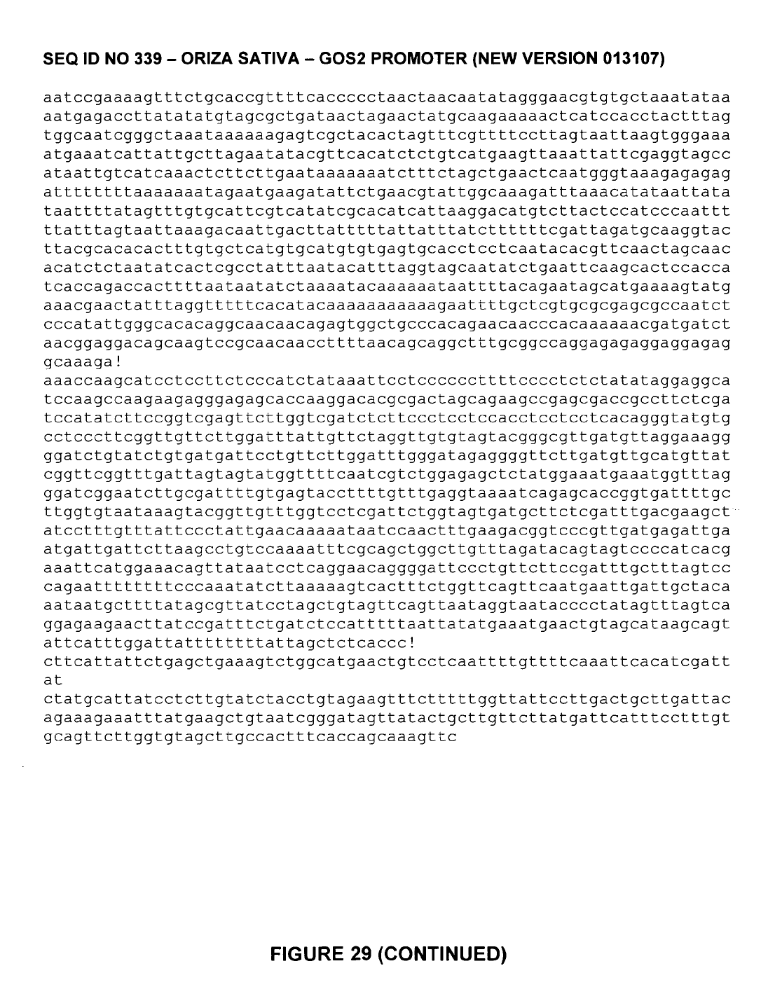

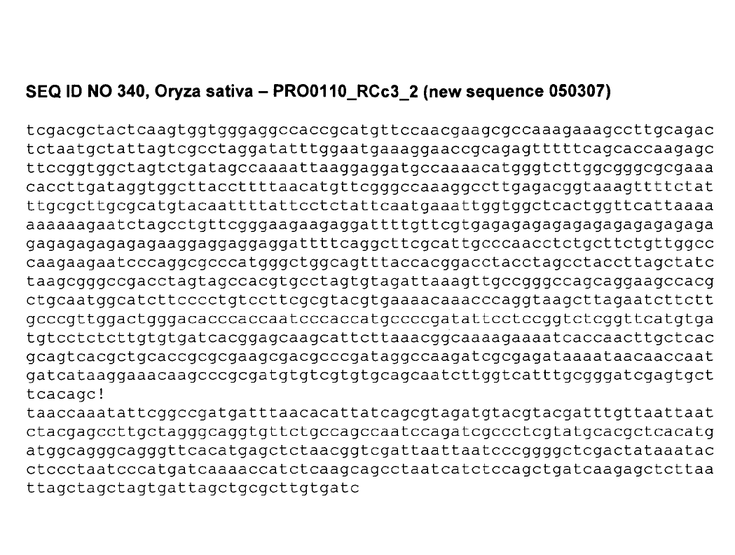

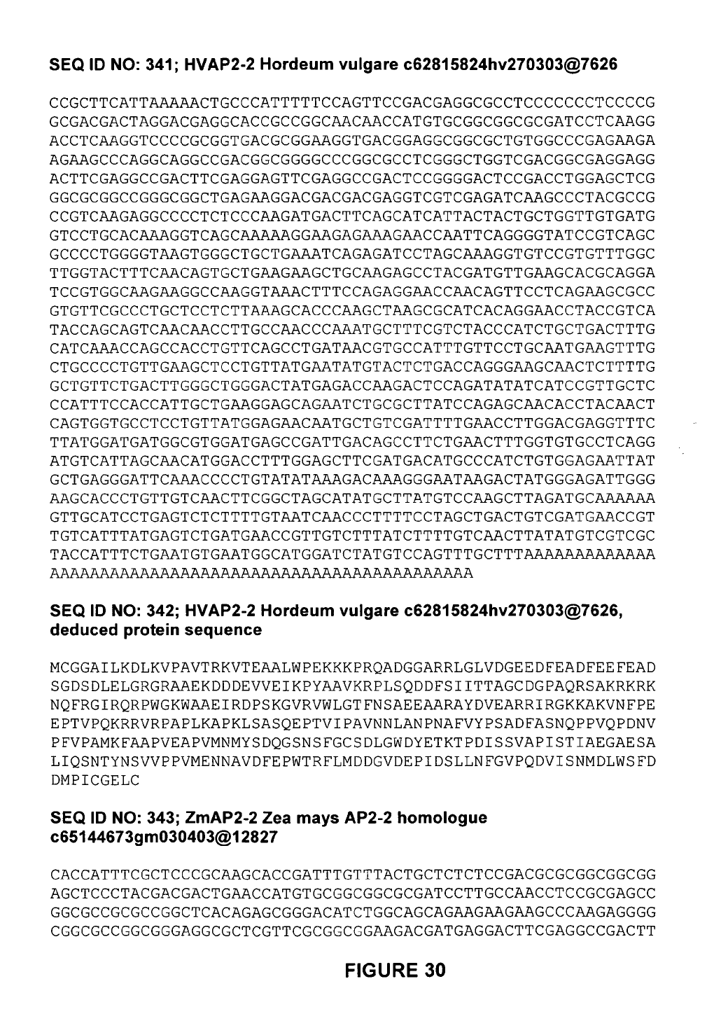

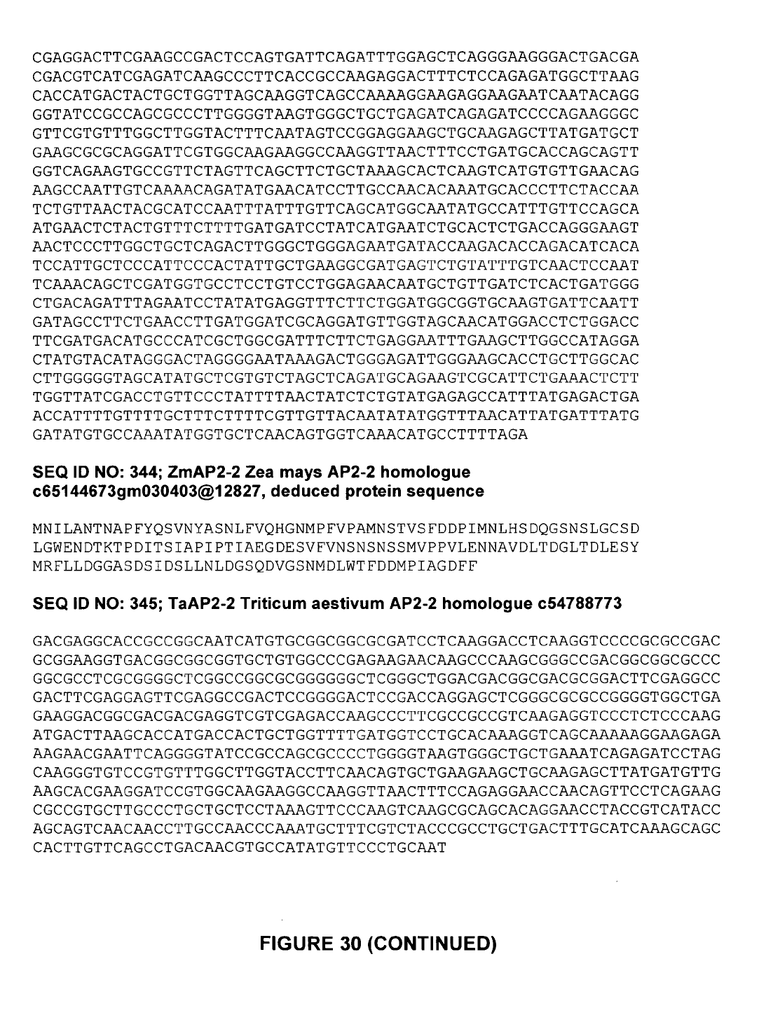

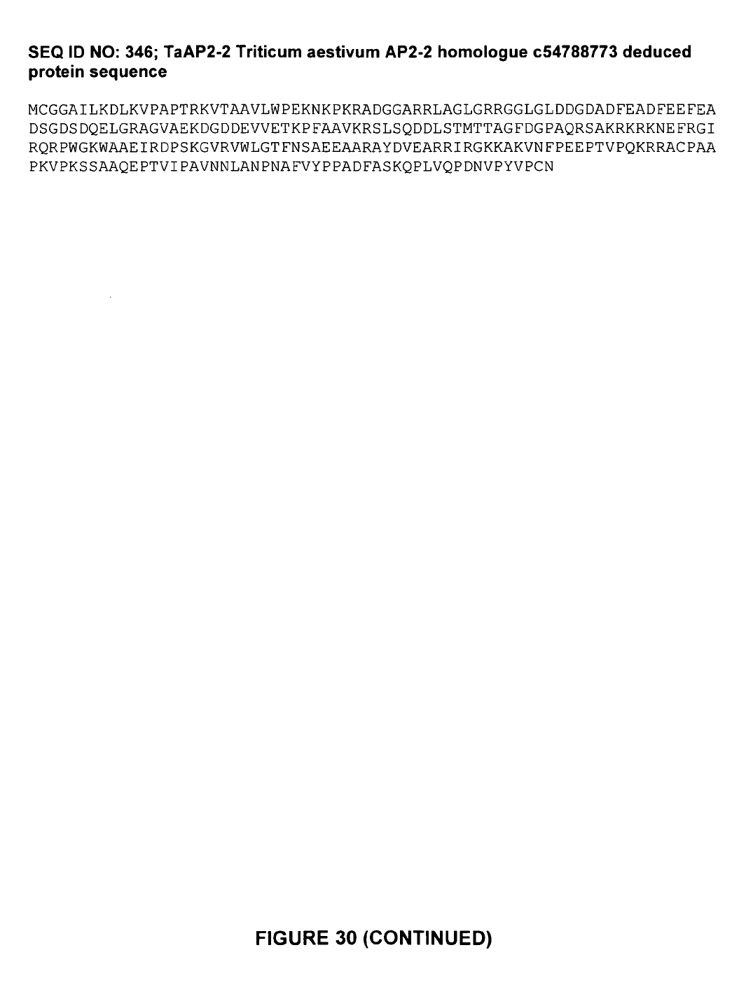

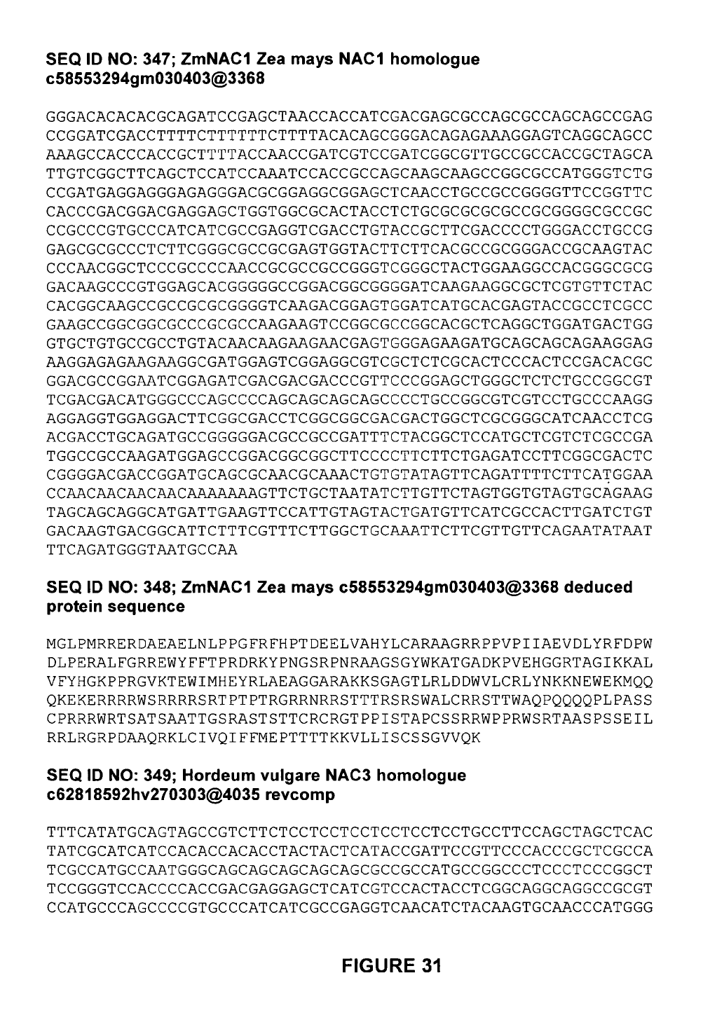

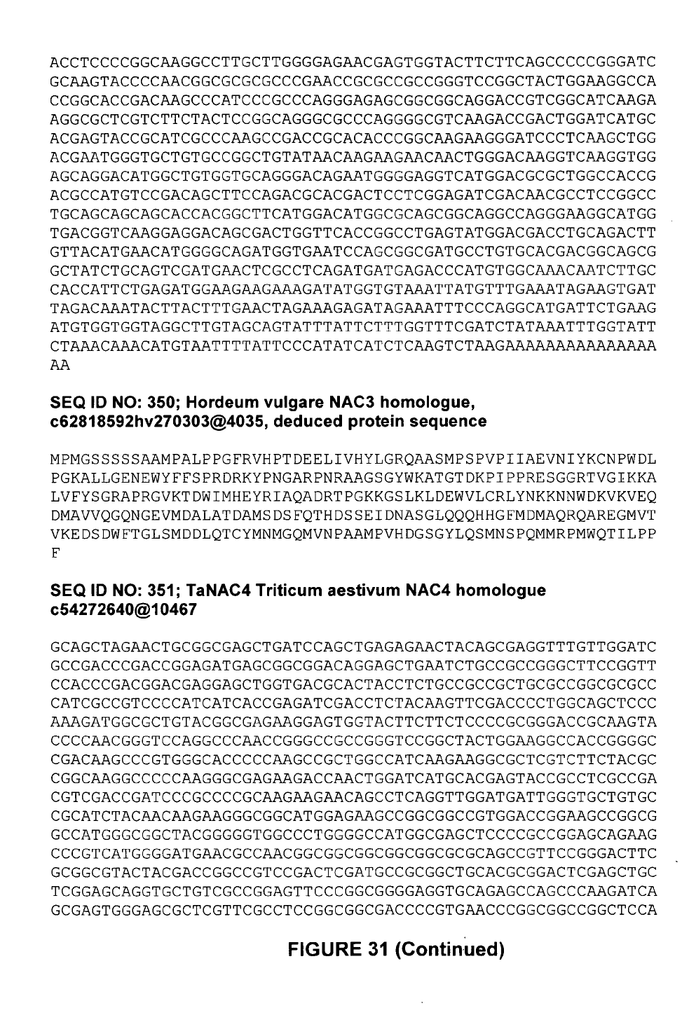

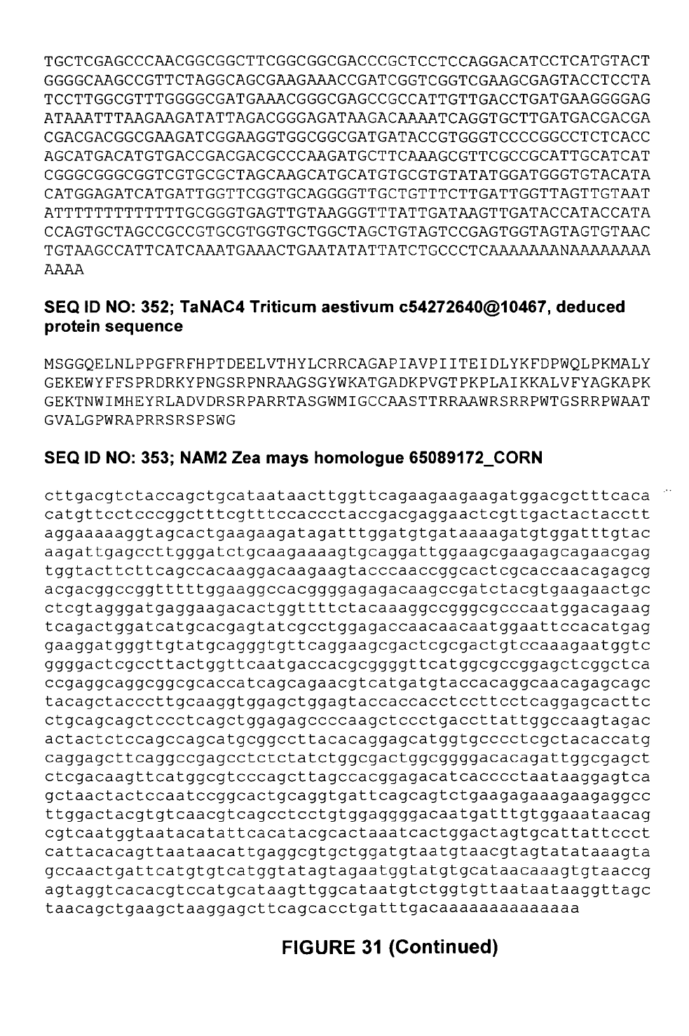

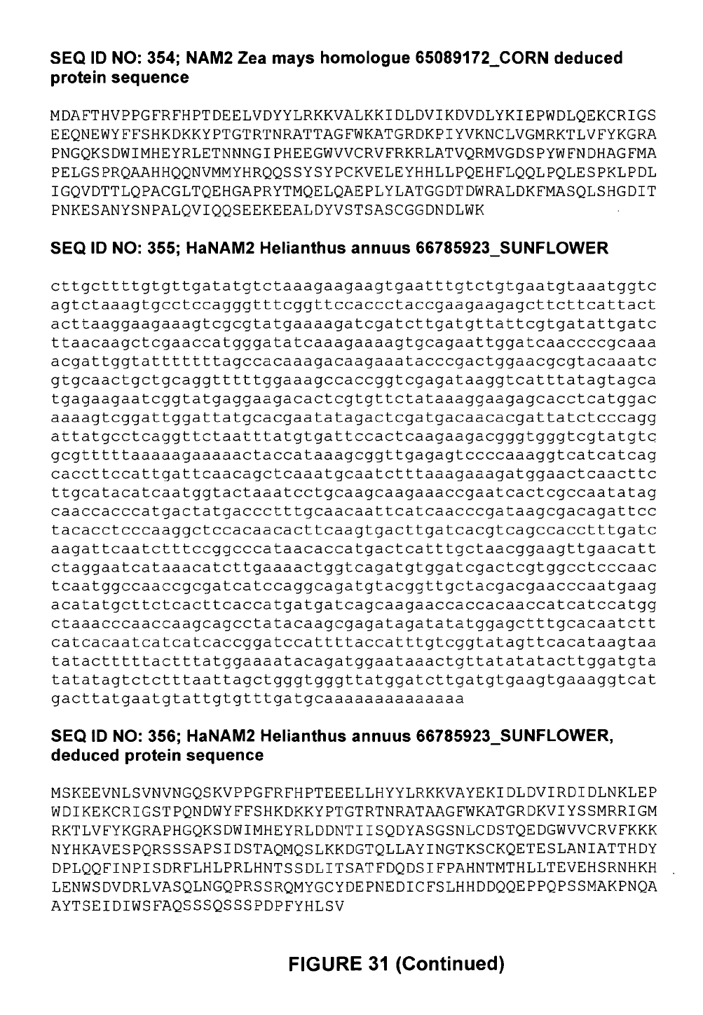

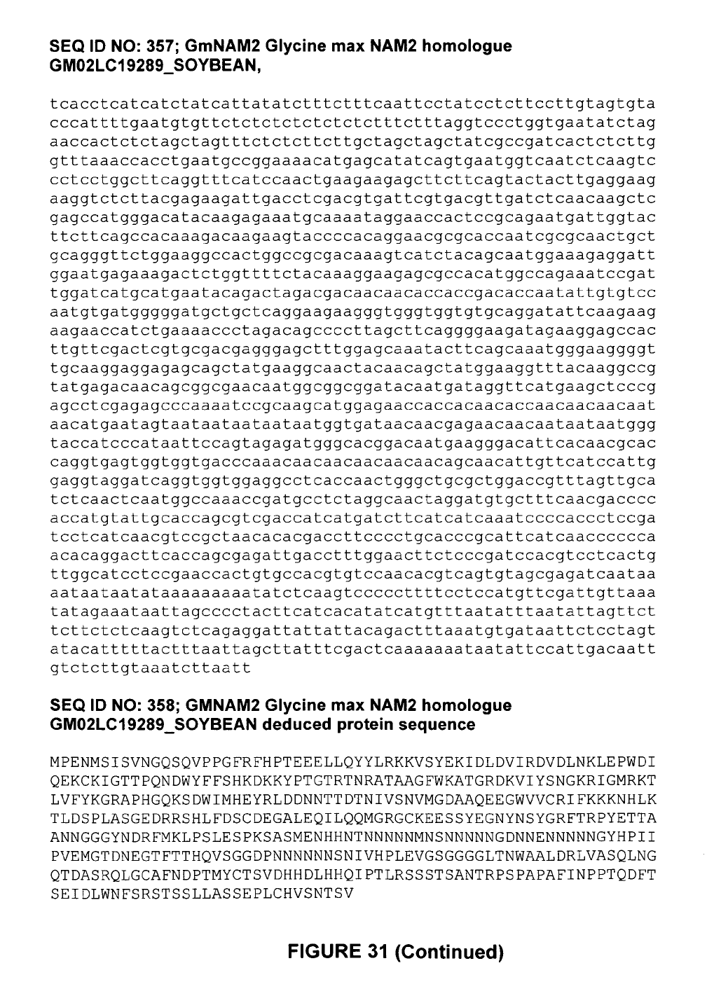

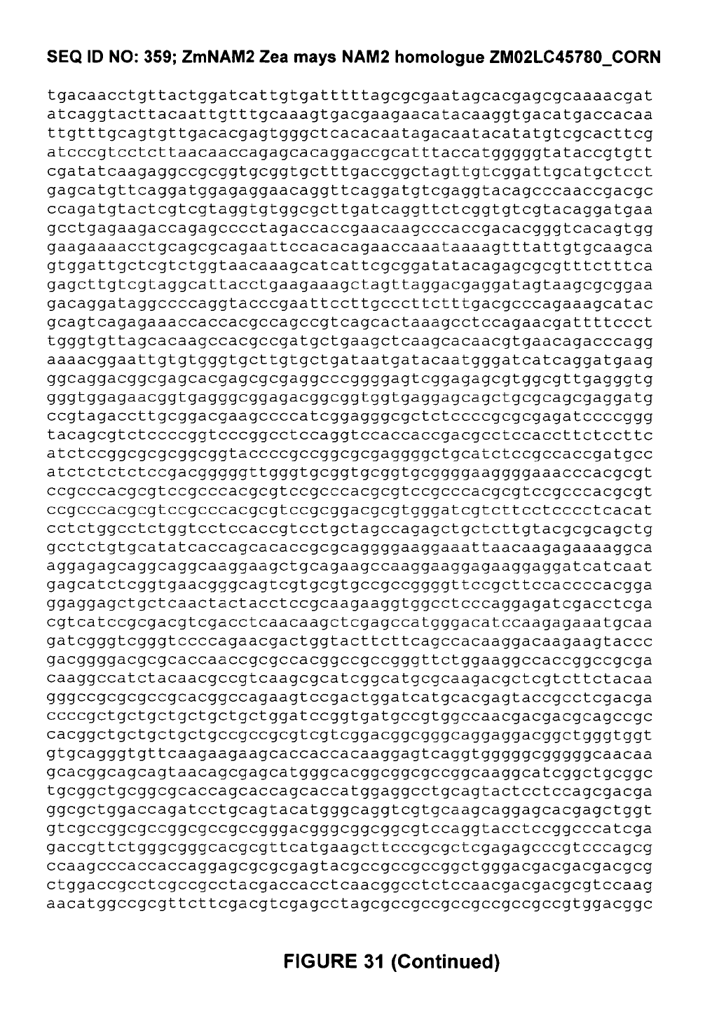

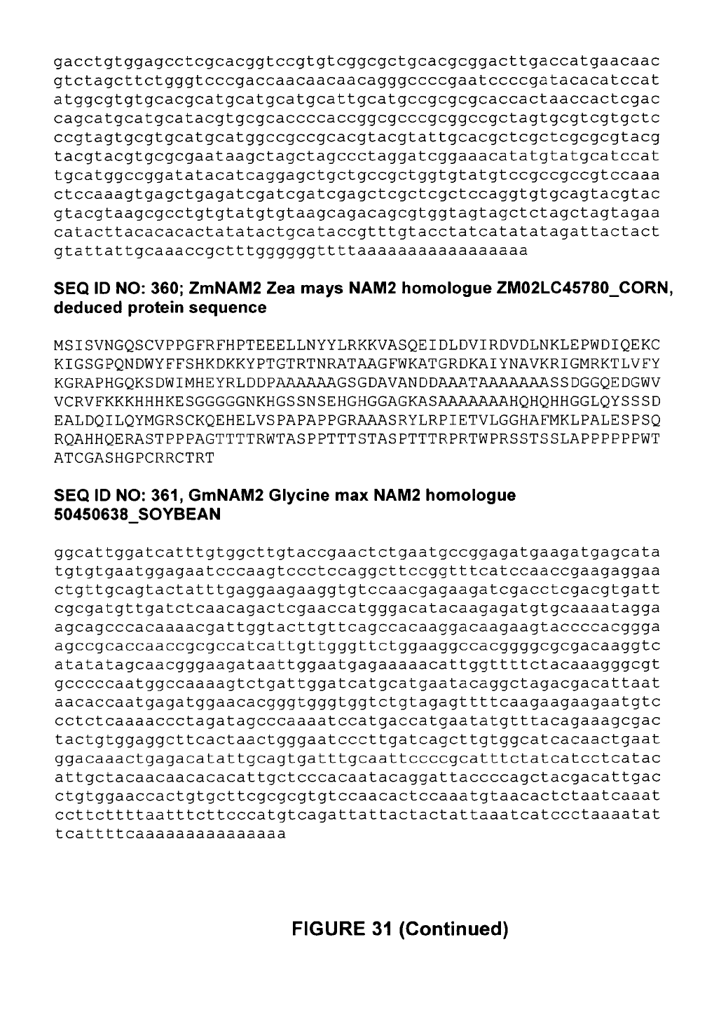

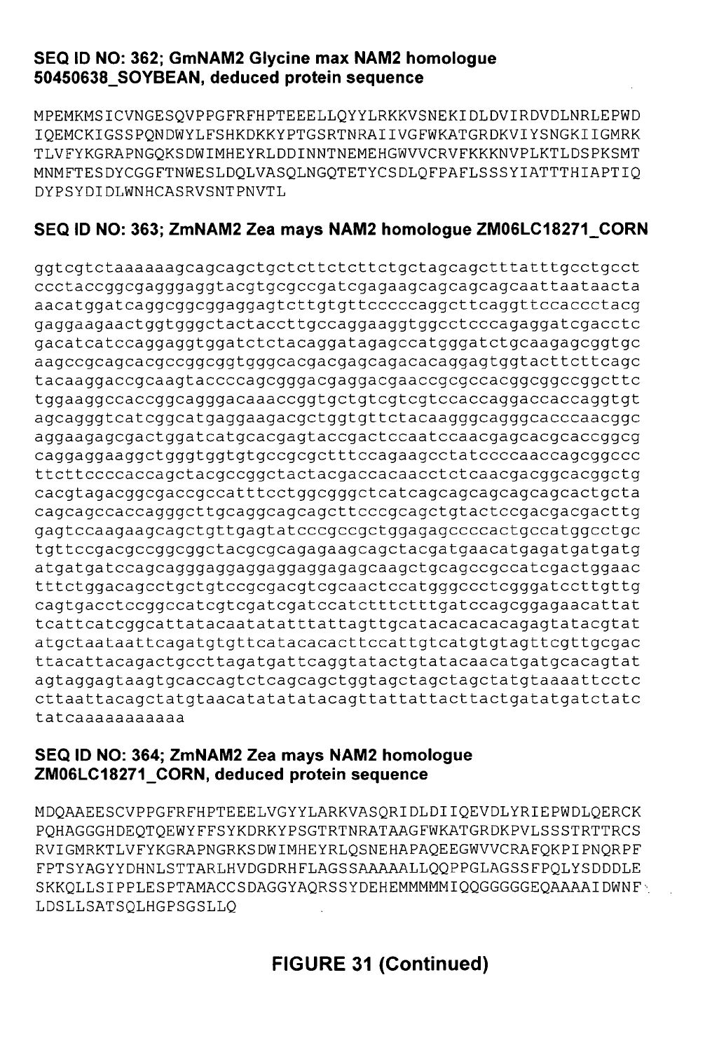

After the LR recombination step, the resulting expression vector pGOS2::AP2-70 (FIG. 25) was transformed into Agrobacterium strain LBA4044 according to methods well known in the art.

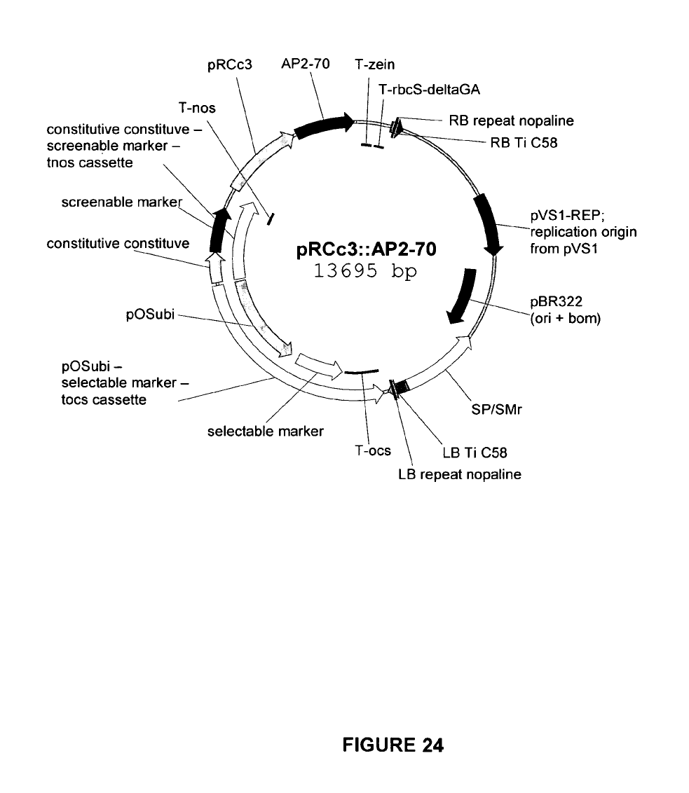

A second vector identical to above except comprising an RCc3 promoter (SEQ ID NO: 110) for root-specific expression was also made. Again, after the LR recombination step, the resulting expression vector pRCc3::AP2-70 (FIG. 24) was transformed into Agrobacterium strain LBA4044 according to methods well known in the art.

### Example 21

### Plant Transformation

### Rice Transformation

The Agrobacterium containing the expression vector was used to transform Oryza sativa plants. Mature dry seeds of the rice japonica cultivar Nipponbare were dehusked. Sterilization was carried out by incubating for one minute in 70% ethanol, followed by 30 minutes in 0.2% HgCl₂, followed by a 6 times 15 minutes wash with sterile distilled water. The sterile seeds were then germinated on a medium containing 2,4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were sub-cultured on fresh medium 3 days before co-cultivation (to boost cell division activity).

Agrobacterium strain LBA4404 containing the expression vector was used for co-cultivation. Agrobacterium was inoculated on AB medium with the appropriate antibiotics and cultured for 3 days at 28° C. The bacteria were then collected and suspended in liquid co-cultivation medium to a density (OD₆₀₀) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent T0 rice transformants were generated for one construct. The primary transformants were transferred from a tissue culture chamber to a greenhouse. After a quantitative PCR analysis to verify copy number of the T-DNA insert, only single copy transgenic plants that exhibit tolerance to the selection agent were kept for harvest of T1 seed. Seeds were then harvested three to five months after transplanting. The method yielded single locus transformants at a rate of over 50% (Aldemita and Hodges 1996, Chan et al. 1993, Hiei et al. 1994).

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radicle and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7% Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MS0) for root induction. The rooted shoots are transplanted to soil in the greenhouse. T1 seeds are produced from plants that exhibit tolerance to the selection agent and that contain a single copy of the T-DNA insert.

### Alfalfa Transformation

A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown D C W and A Atanassov (1985. Plant Cell Tissue Organ Culture 4: 111-112). Alternatively, the RA3 variety (University of Wisconsin) has been selected for use in tissue culture (Walker et al., 1978 μm J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K2S04, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings were transplanted into pots and grown in a greenhouse. T1 seeds are produced from plants that exhibit tolerance to the selection agent and that contain a single copy of the T-DNA insert.

### Example 22

### Phenotypic Evaluation Procedure

### 22.1 Evaluation Setup

Approximately 35 independent T0 rice transformants were generated. The primary transformants were transferred from a tissue culture chamber to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%.

Four T1 events were further evaluated in the T2 generation following the same evaluation procedure as for the T1 generation but with more individuals per event. From the stage of sowing until the stage of maturity the plants were passed several times through a digital imaging cabinet. At each time point digital images (2048×1536 pixels, 16 million colours) were taken of each plant from at least 6 different angles.

### 22.2 Statistical Analysis: F-Test

A two factor ANOVA (analysis of variants) was used as a statistical model for the overall evaluation of plant phenotypic characteristics. An F-test was carried out on all the parameters measured of all the plants of all the events transformed with the gene of the present invention. The F-test was carried out to check for an effect of the gene over all the transformation events and to verify for an overall effect of the gene, also known as a global gene effect. The threshold for significance for a true global gene effect was set at a 5% probability level for the F-test. A significant F-test value points to a gene effect, meaning that it is not only the mere presence or position of the gene that is causing the differences in phenotype.

### 22.3 Parameters Measured

### Biomass-Related Parameter Measurement

From the stage of sowing until the stage of maturity the plants were passed several times through a digital imaging cabinet. At each time point digital images (2048×1536 pixels, 16 million colours) were taken of each plant from at least 6 different angles.

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass. The early vigour is the plant (seedling) aboveground area three weeks post-germination. Increase in root biomass is expressed as an increase in total root biomass (measured as maximum biomass of roots observed during the lifespan of a plant); or as an increase in the root/shoot index (measured as the ratio between root mass and shoot mass in the period of active growth of root and shoot).

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

### Example 23

### Results of the Phenotypic Evaluation of the Transgenic Plants

The results of the evaluation of transgenic rice plants expressing an AP2-70 nucleic acid are presented below. The percentage difference between the transgenics and the corresponding nullizygotes is also shown.

TABLE 19

| | | |
|---|---|---|
| Trait | Positive events | Average difference |

results of expression of pGOS2::AP2-70

| | | |
|---|---|---|
| Root Shoot Index | 3 events | 21.3% |
| | | overall +14% |
| Total Seed Yield | 2 events | 27% |
| Fill rate | 3 events | 27% |
| TKW | 4 events | 8.75% |
| | | overall +6% |
| Harvest Index | 4 events | 30.5% |
| | | overall +16% |
| Root thick | 2 events | 10.5% |
| Height | 2 events | 8% |
| | | overall +4% |

results of expression of pRCc3::AP2-70

| | | |
|---|---|---|
| Area max | 2 events | 16.5% |
| | | Overall +7% |
| Total seed yield | 2 events | 233% |
| | | Overall +89% |
| Number filled seed | 2 events | 184.5% |
| | | Overall +68% |
| Fill rate | 2 events | 206% |
| | | Overall +87% |
| Harvest Index | 2 events | 238.5% |
| | | Overall +86% |
| TKW | 3 events | 11.3% |
| | | Overall +7% |
| Height | 2 events | 12.5% |
| | | Overall +7% |

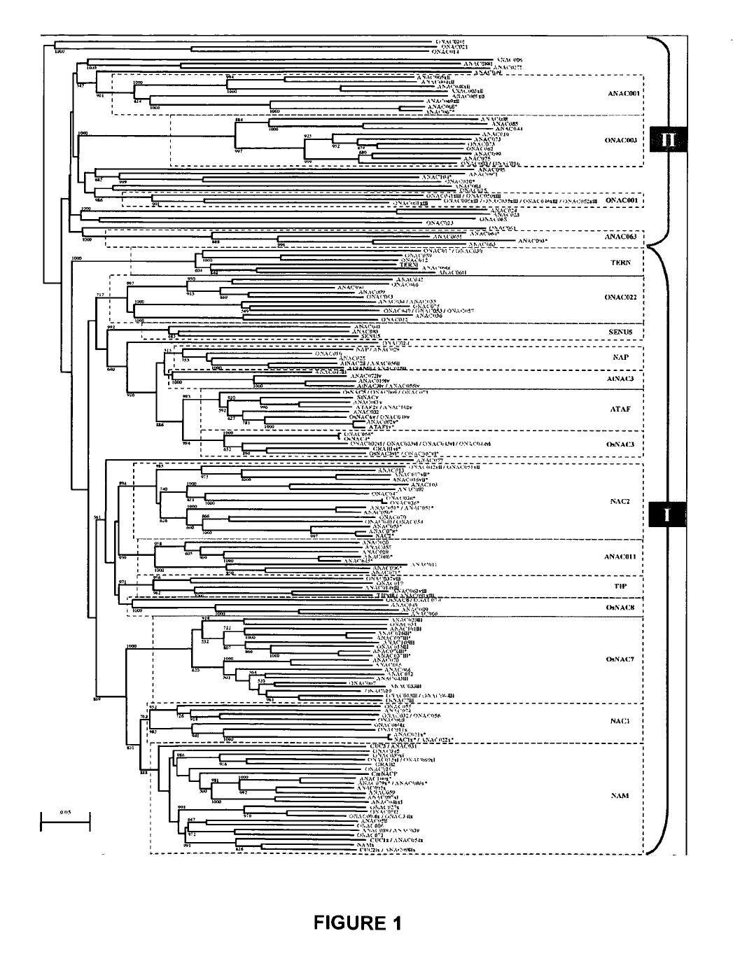

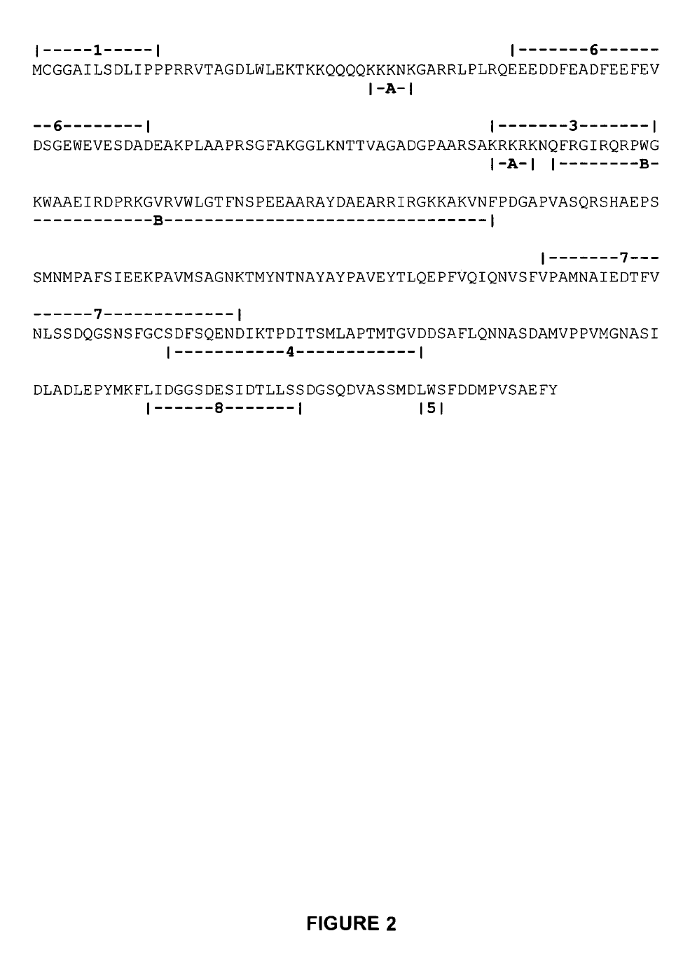

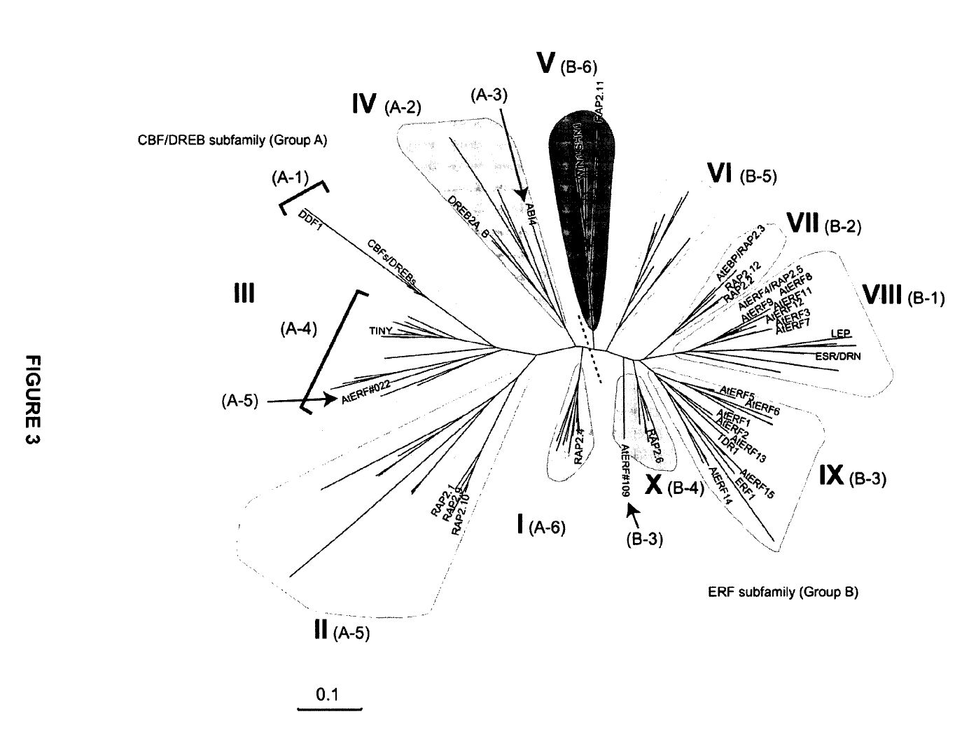

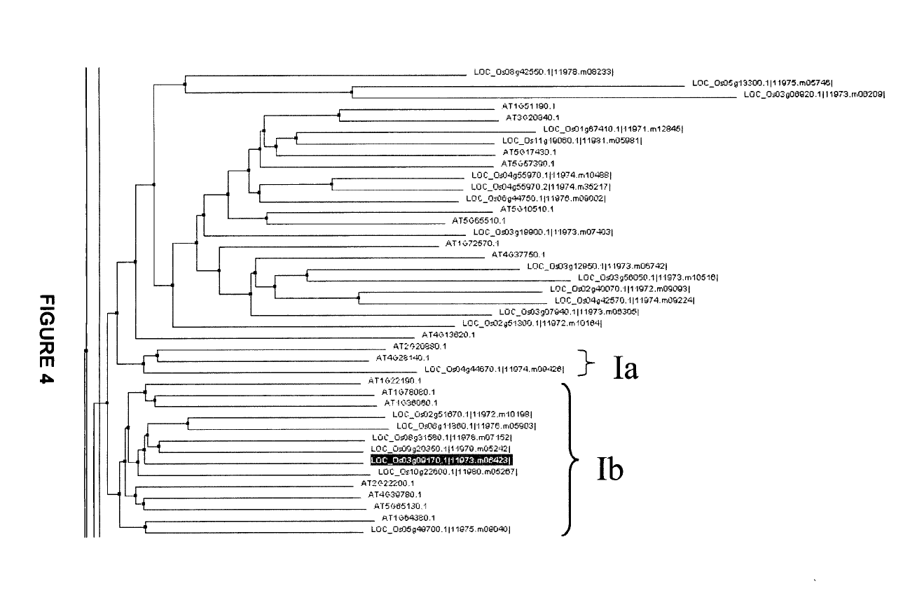

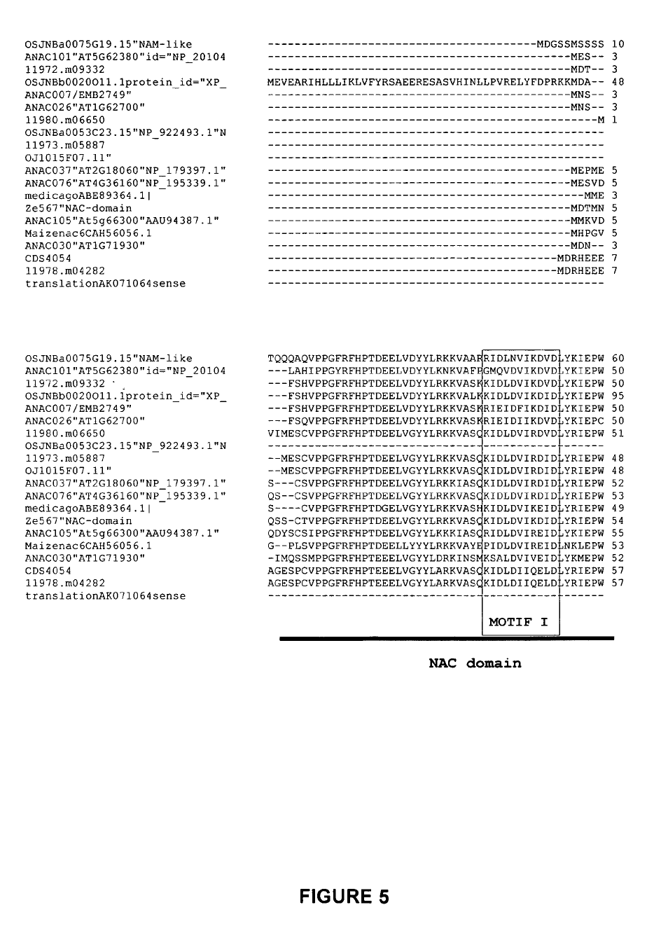

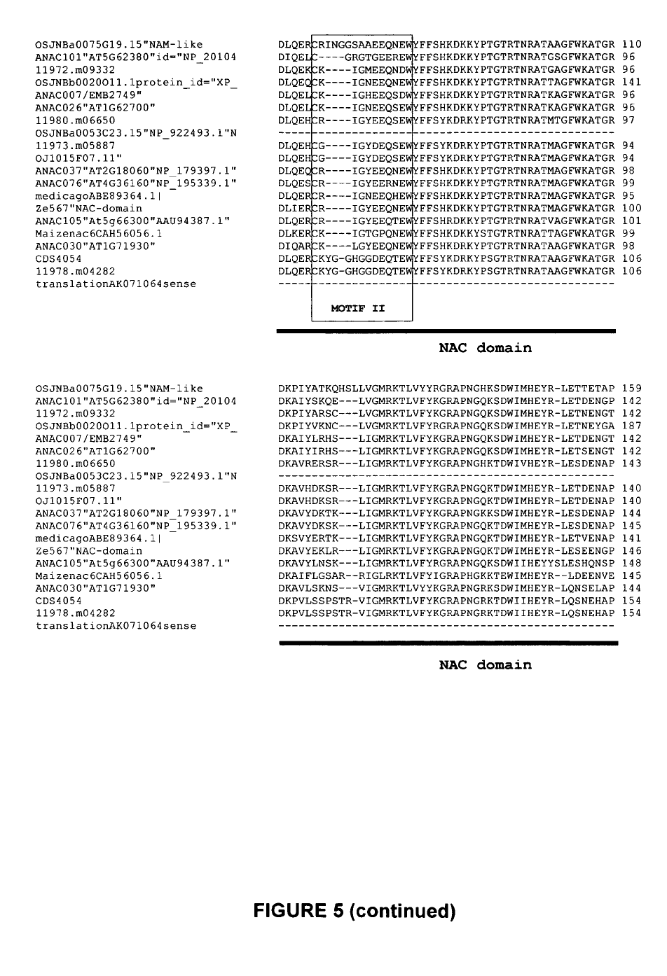

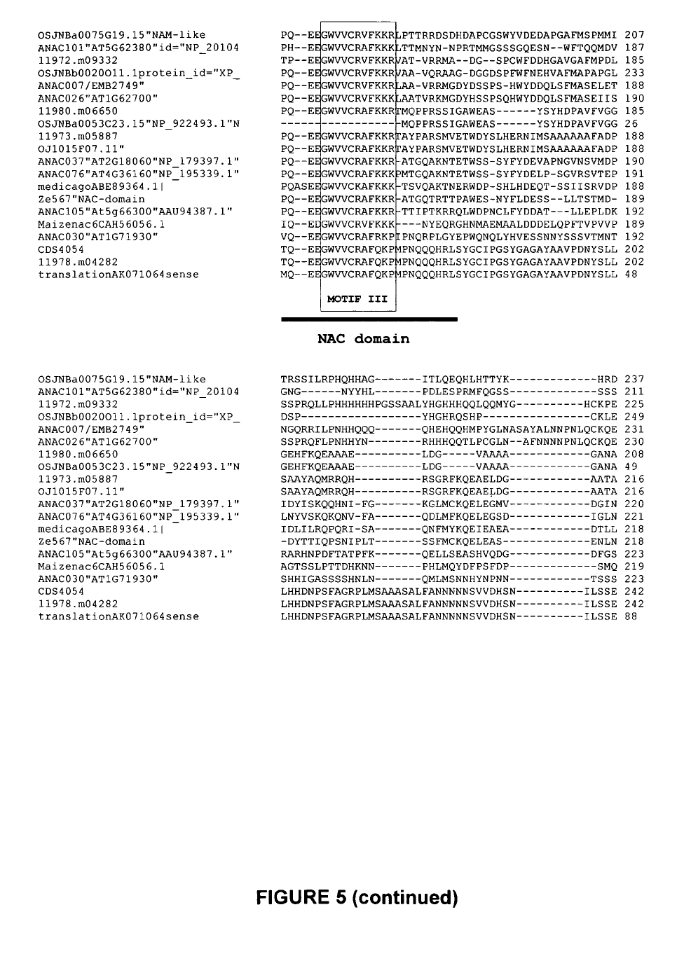

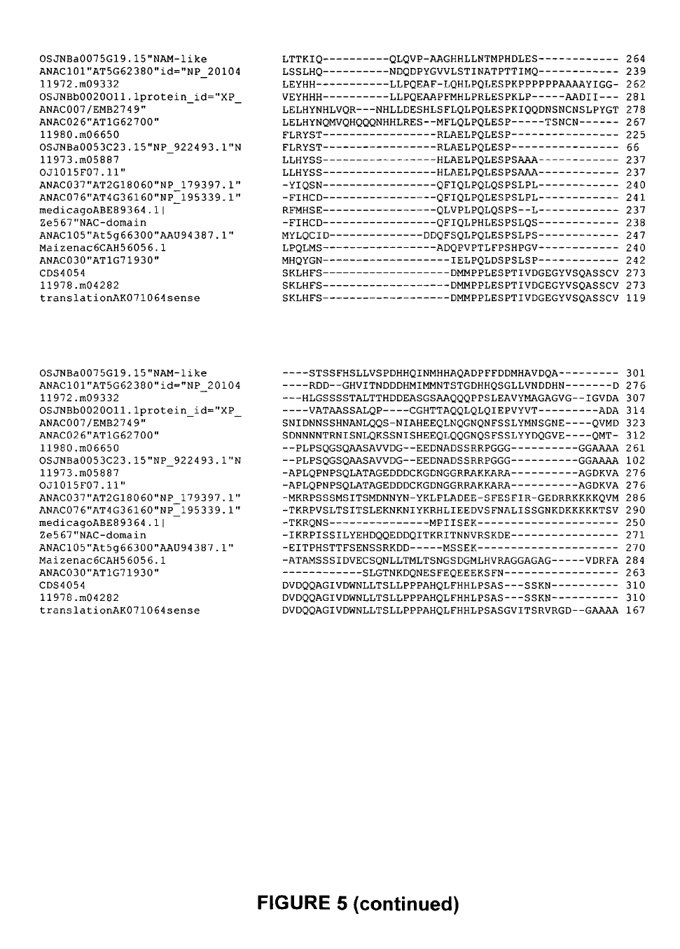

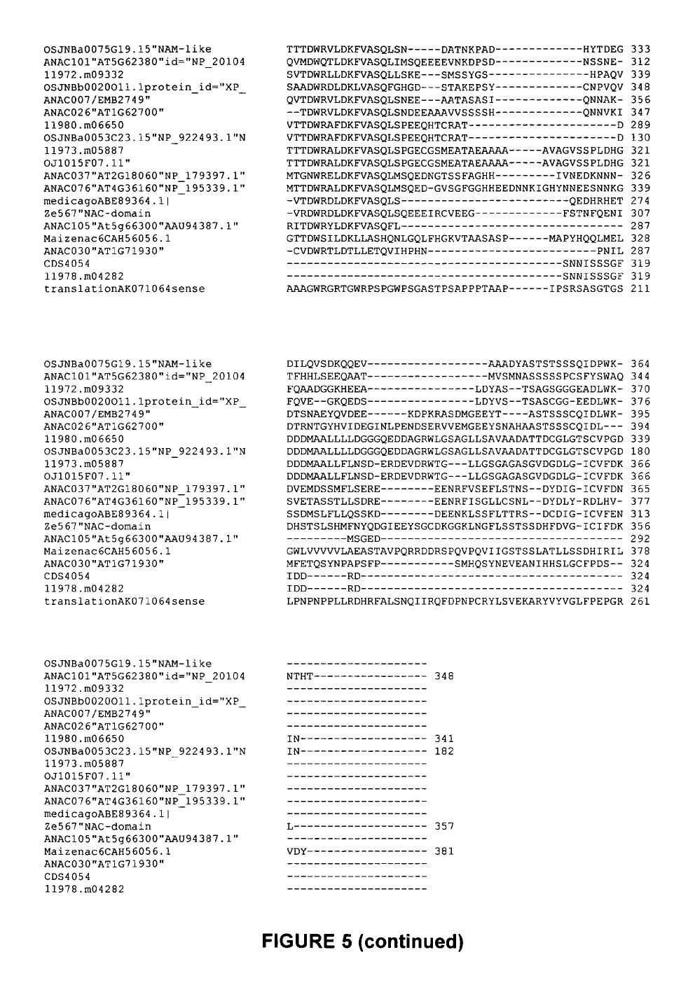

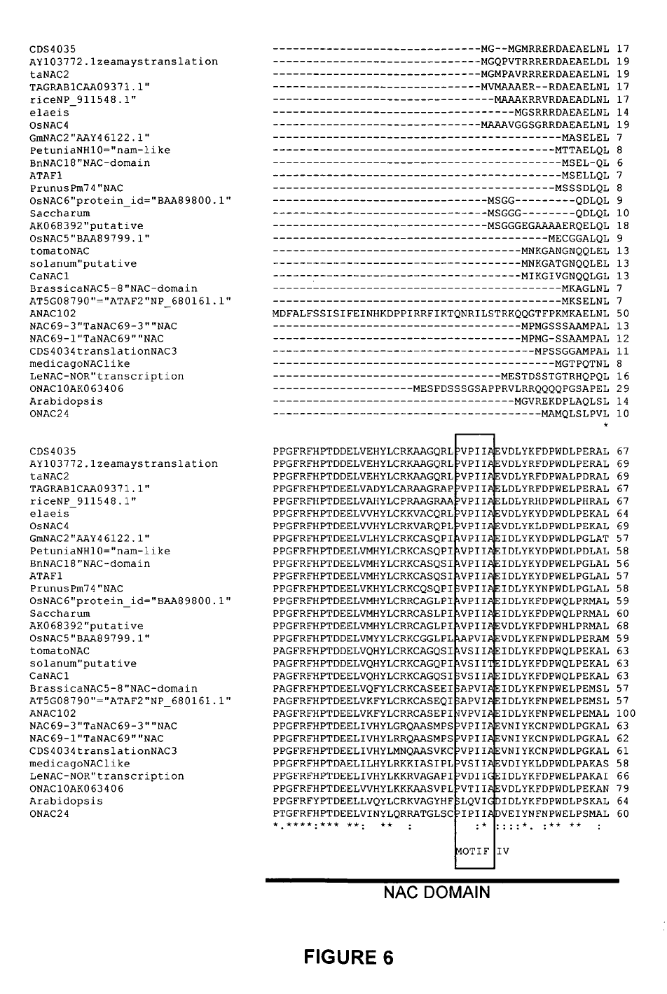

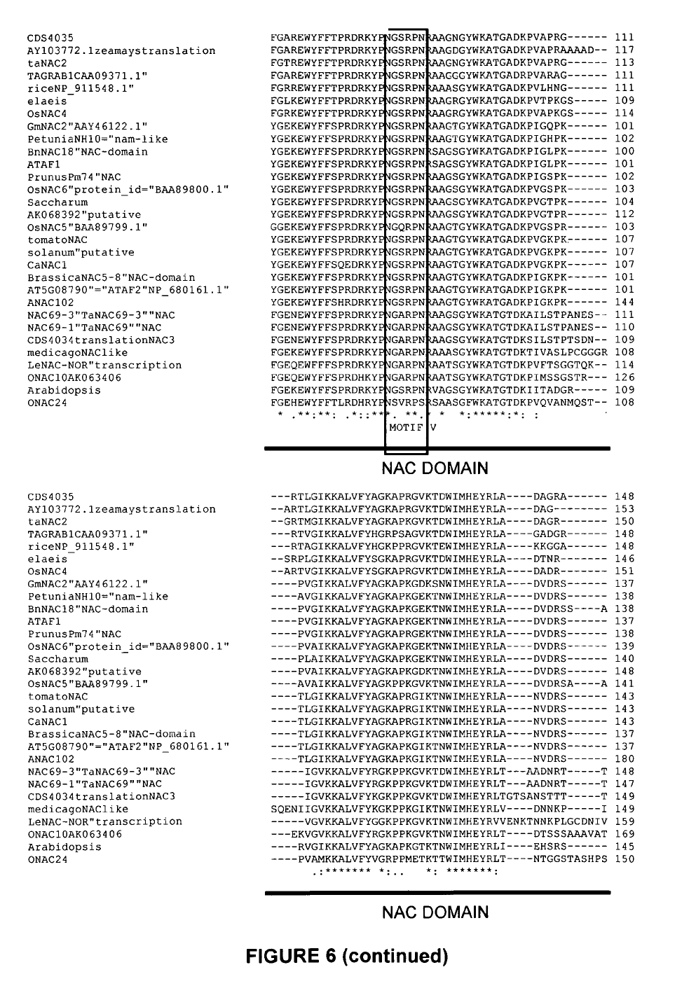

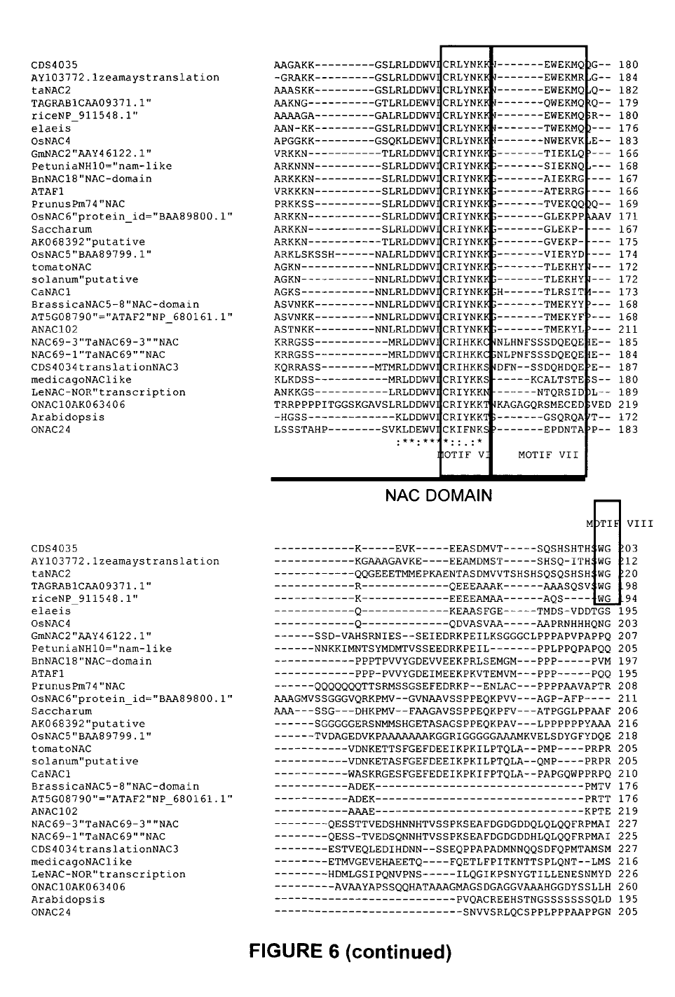

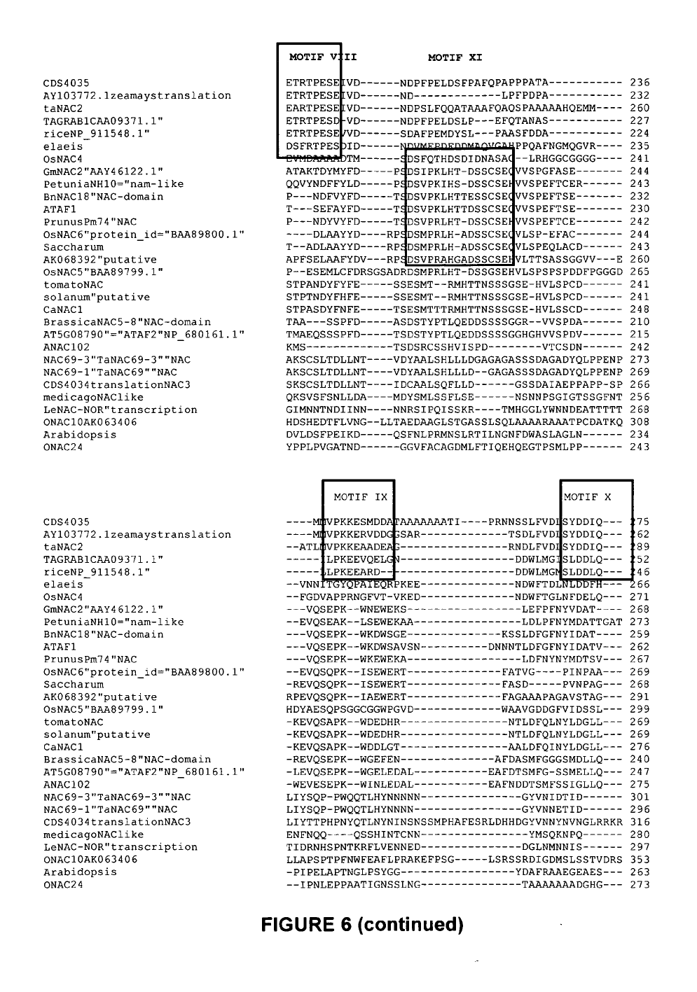

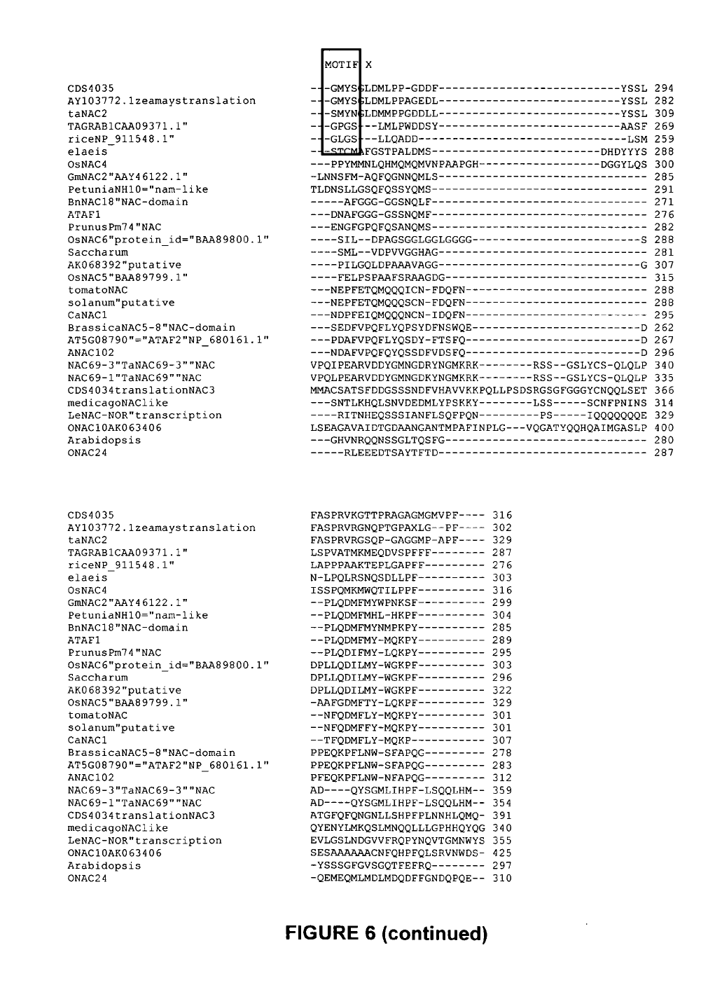

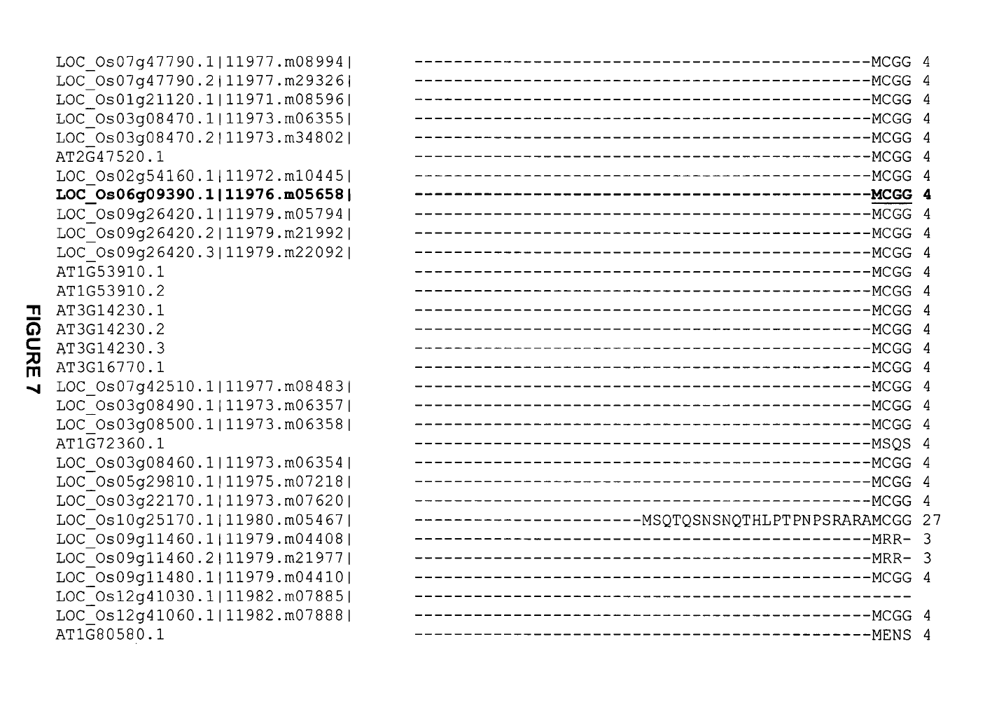

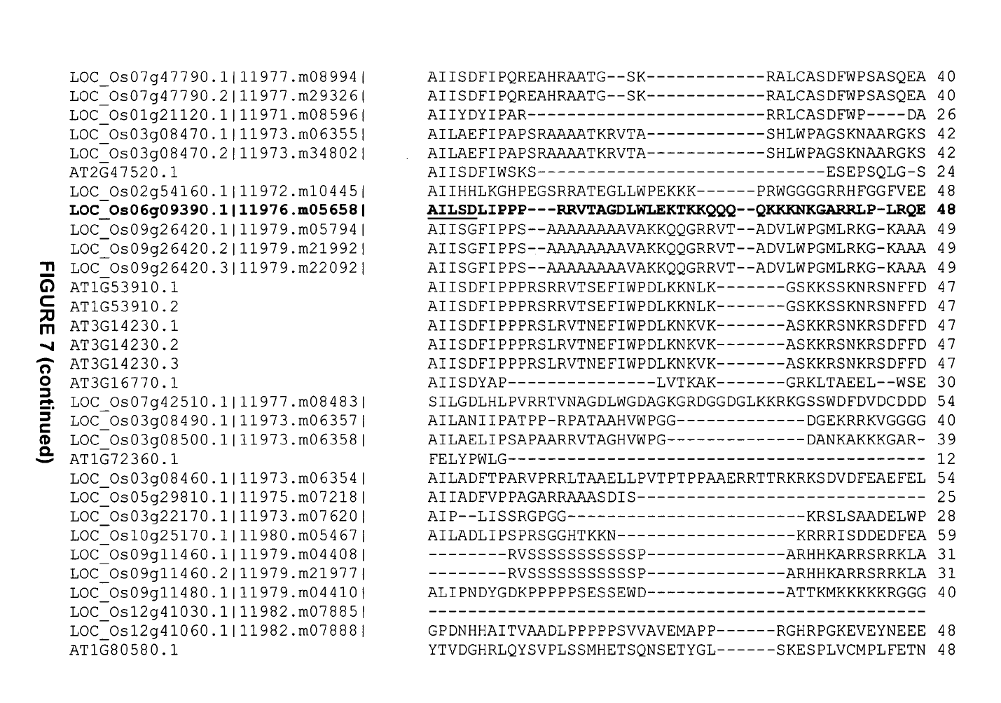

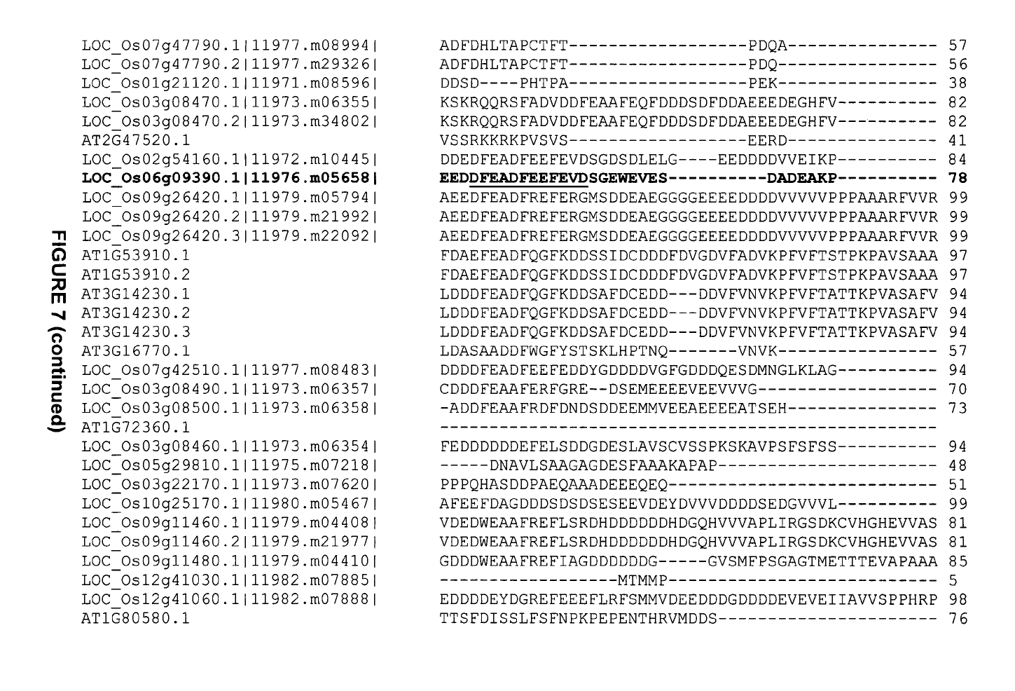

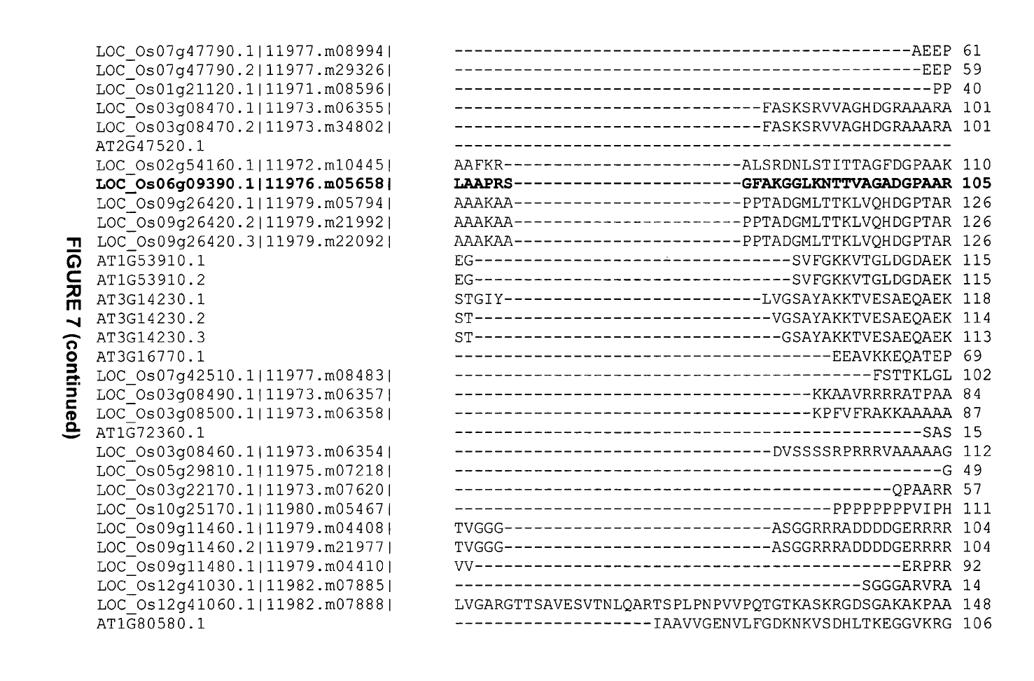

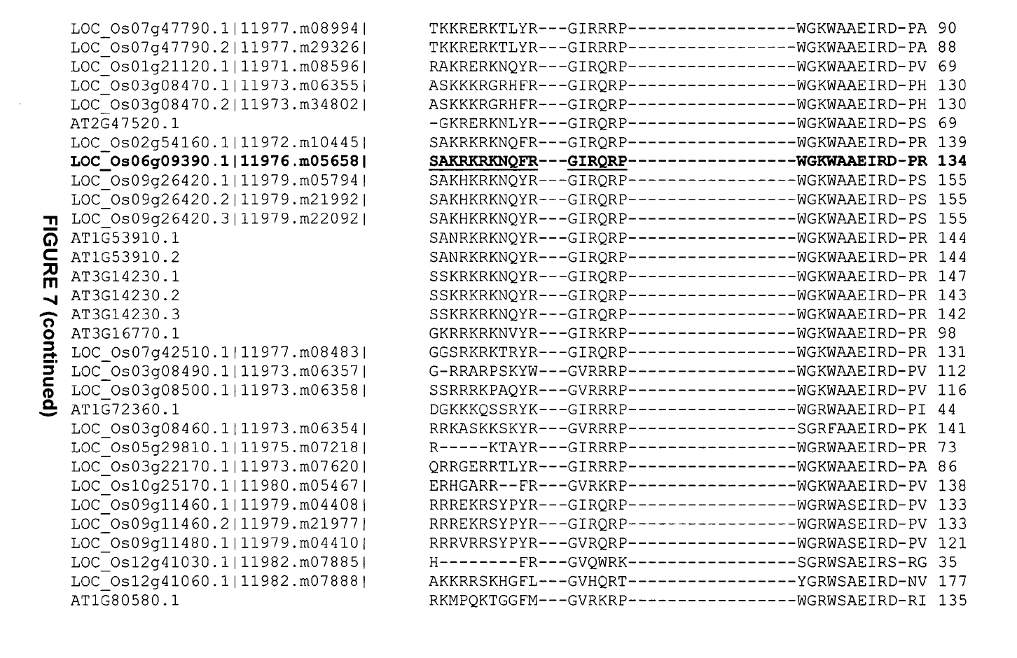

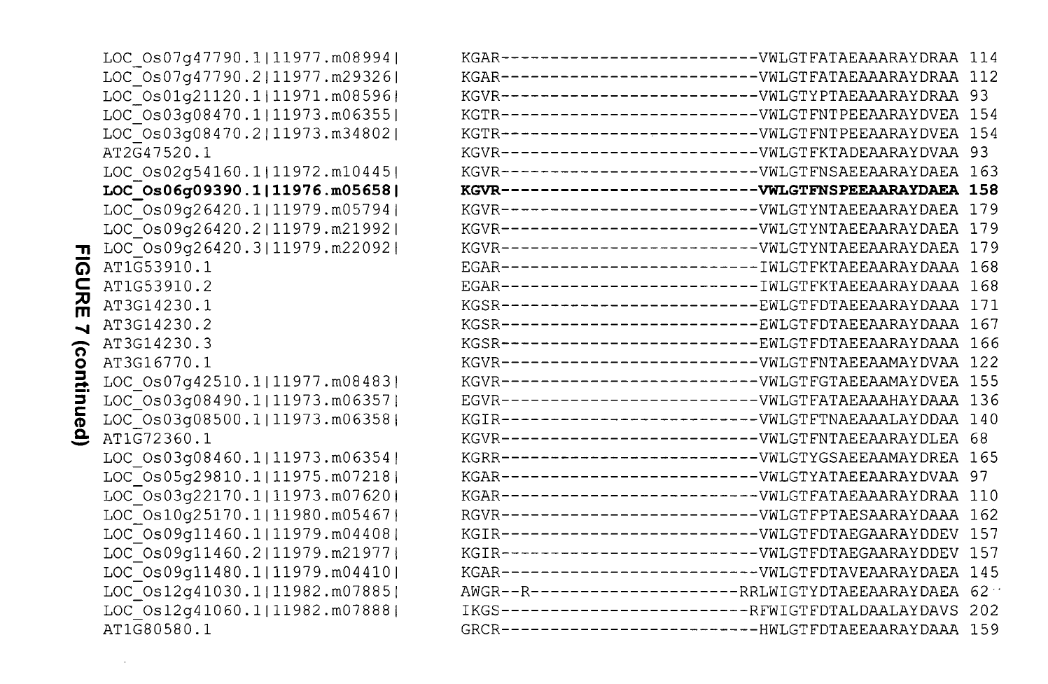

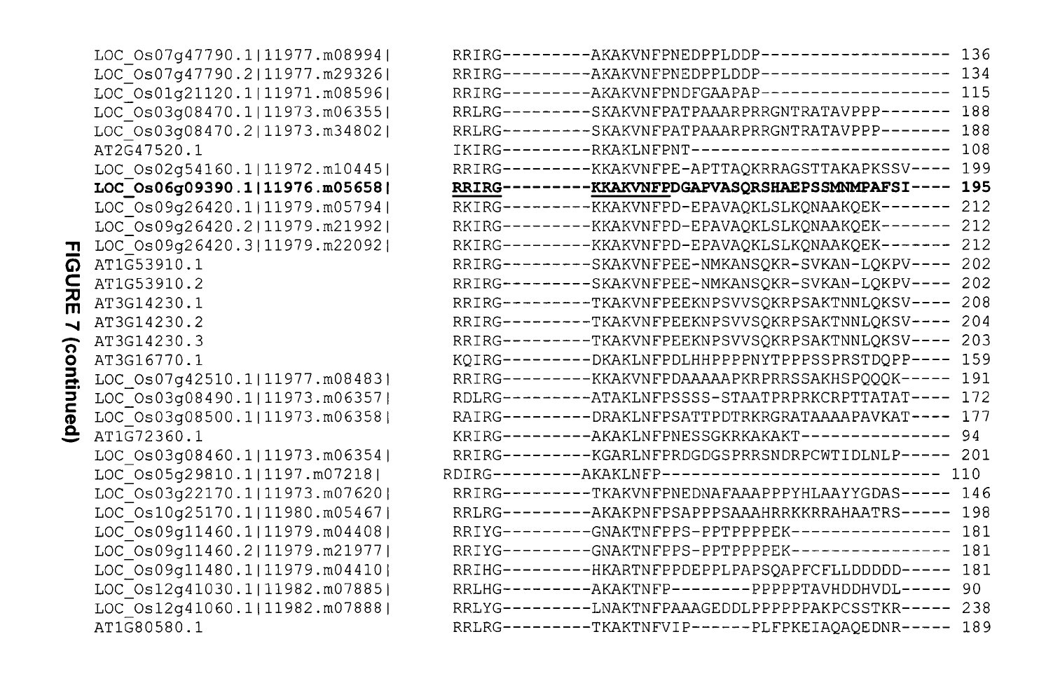

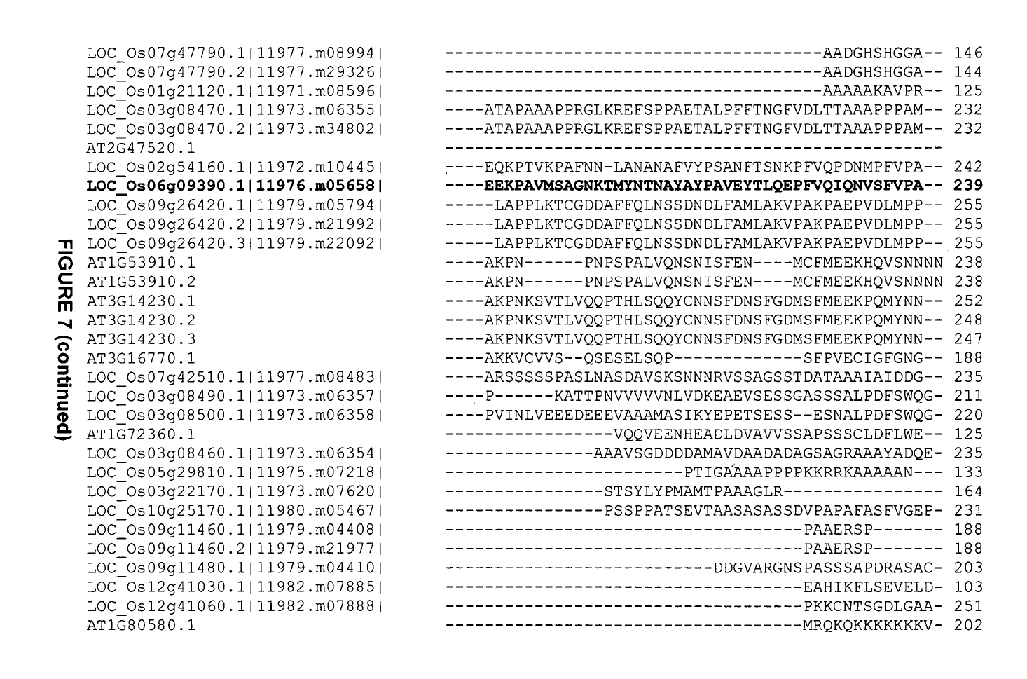

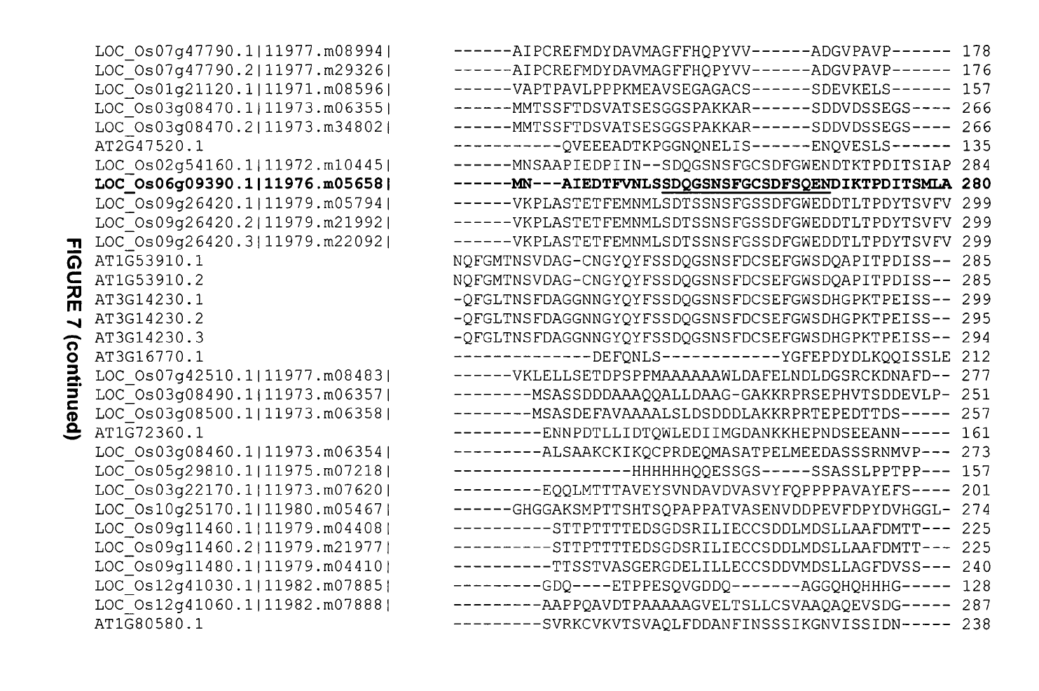

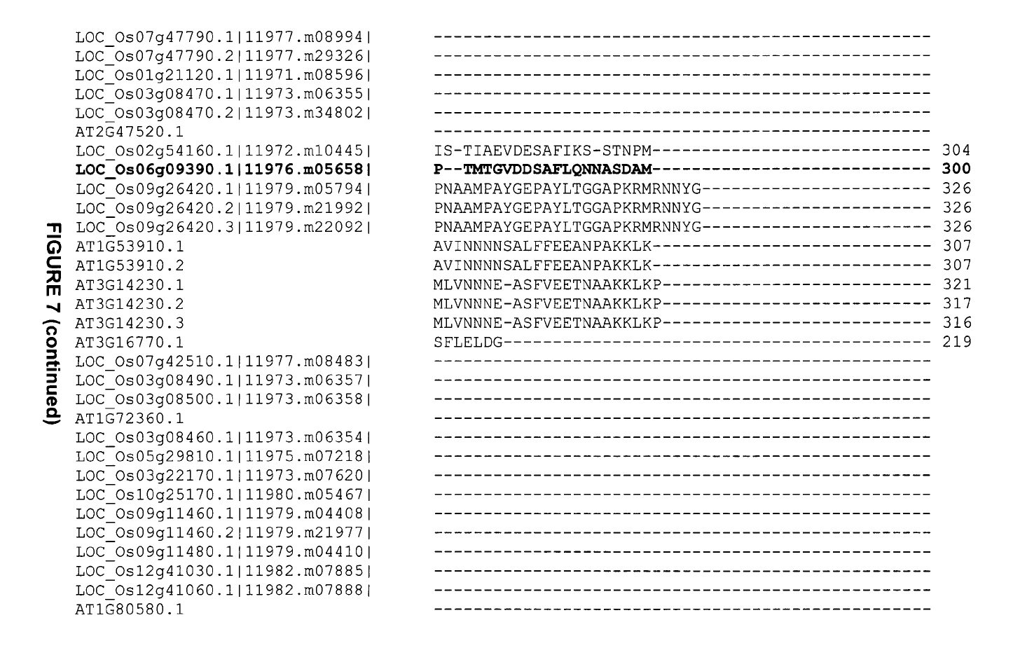

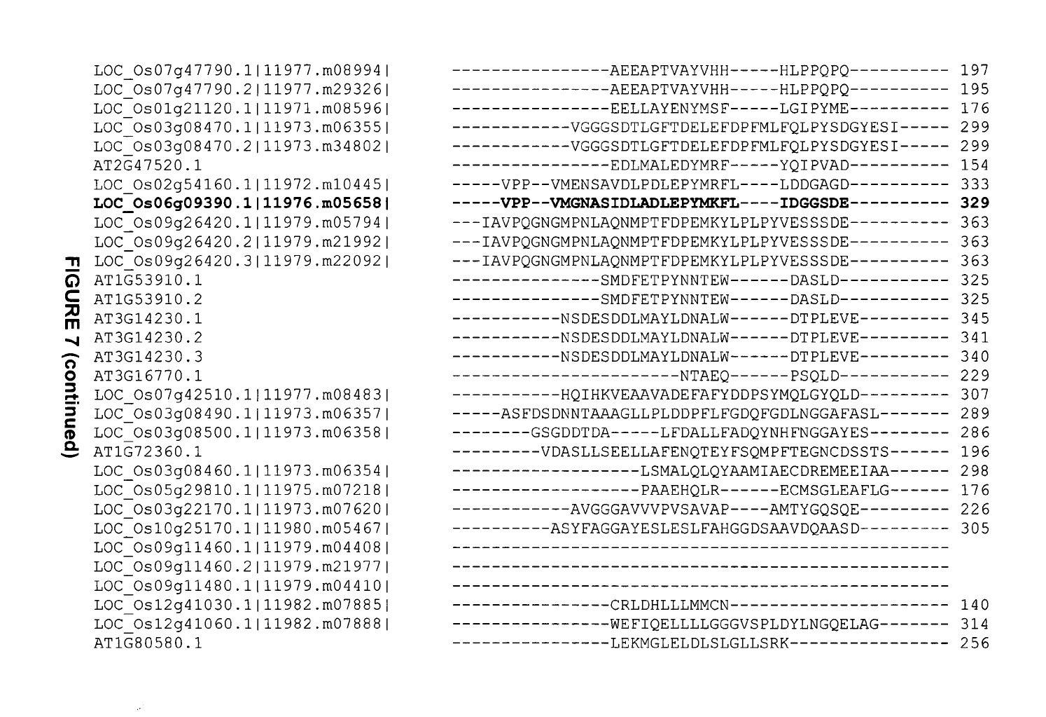

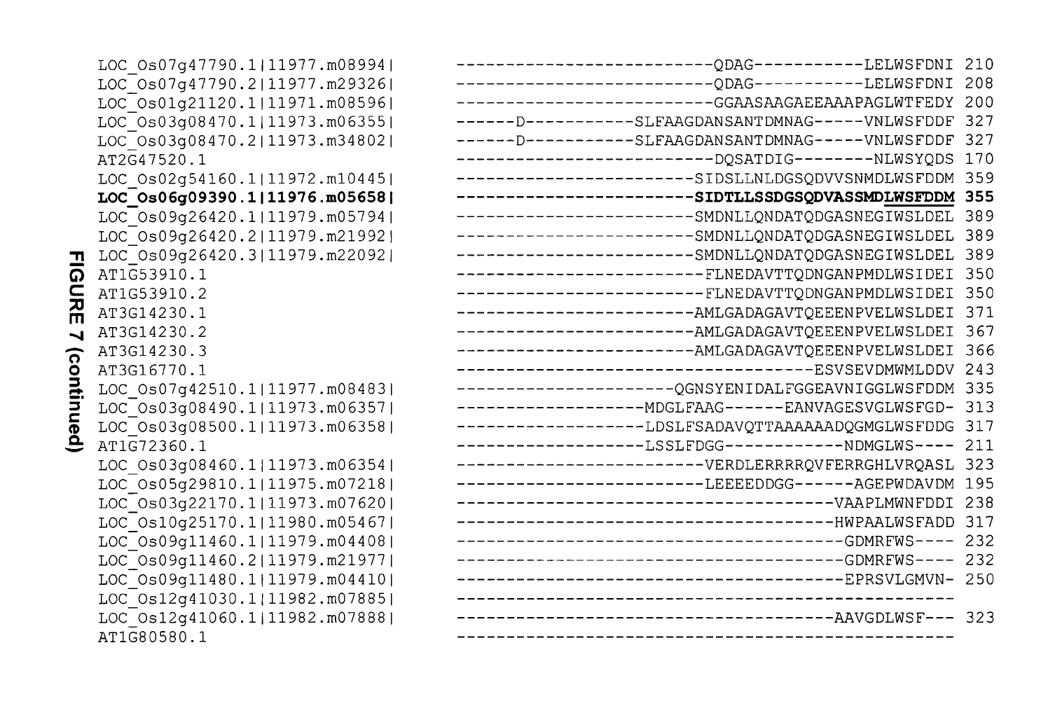

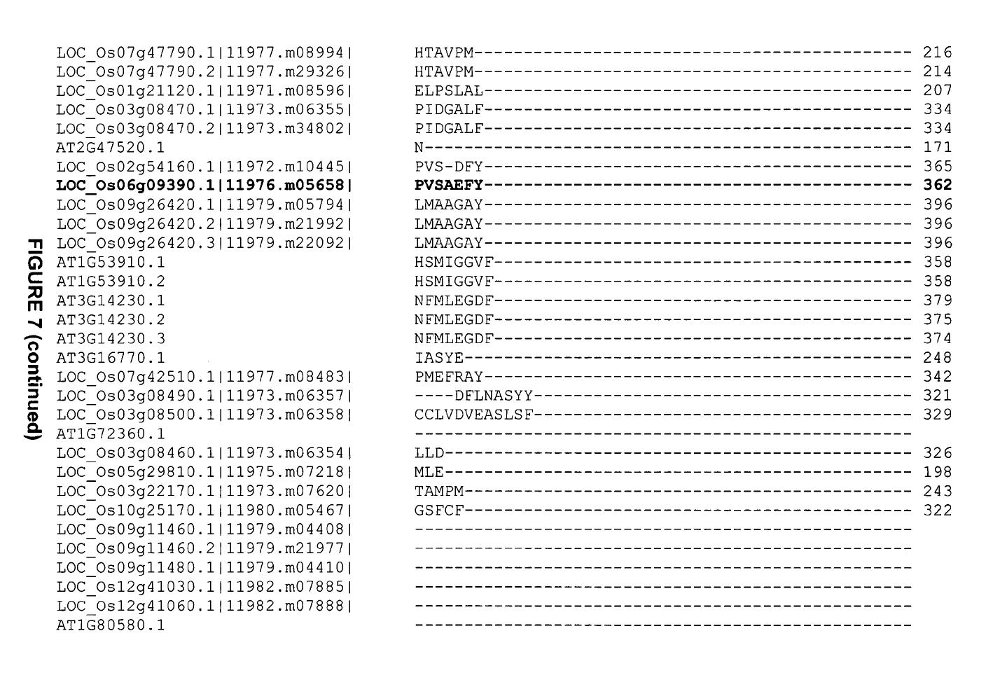

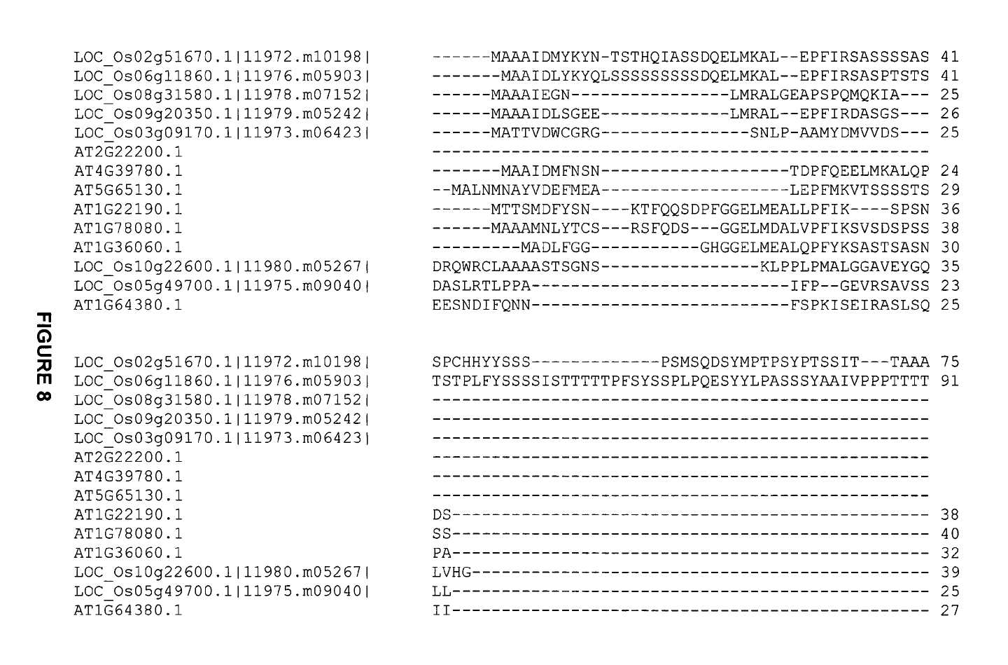

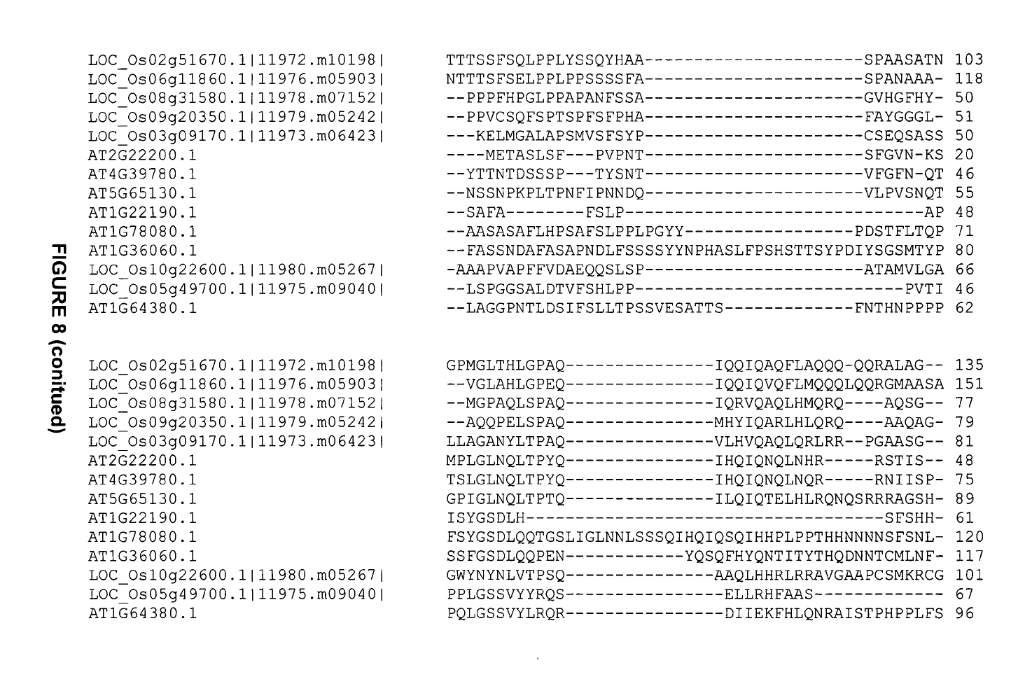

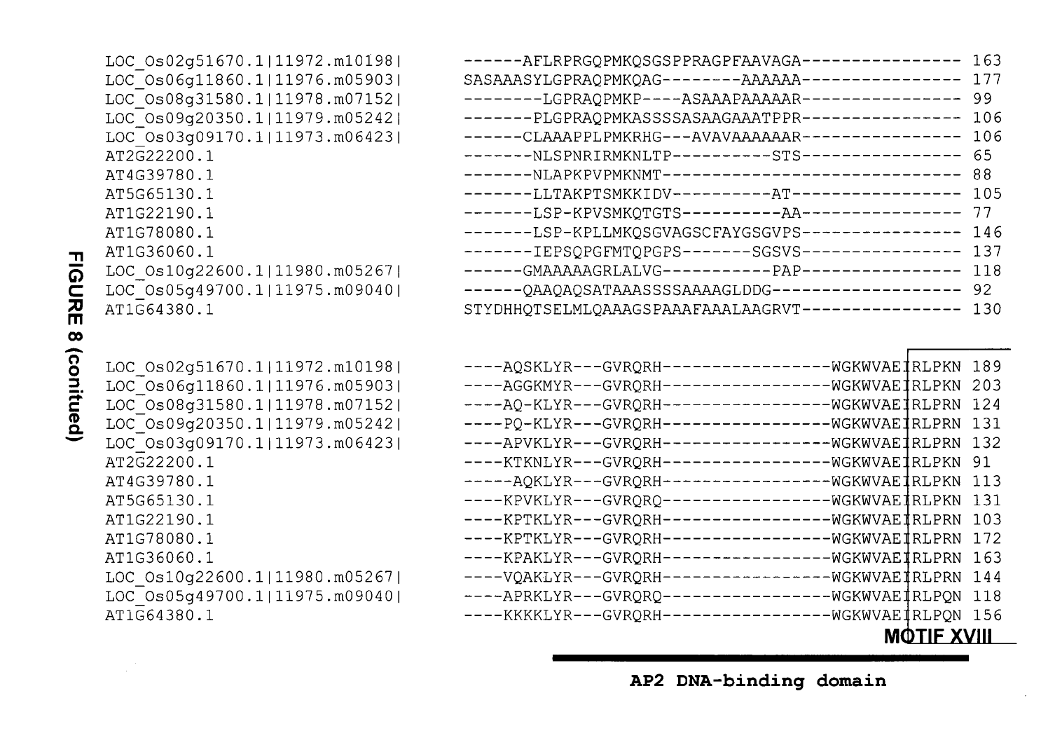

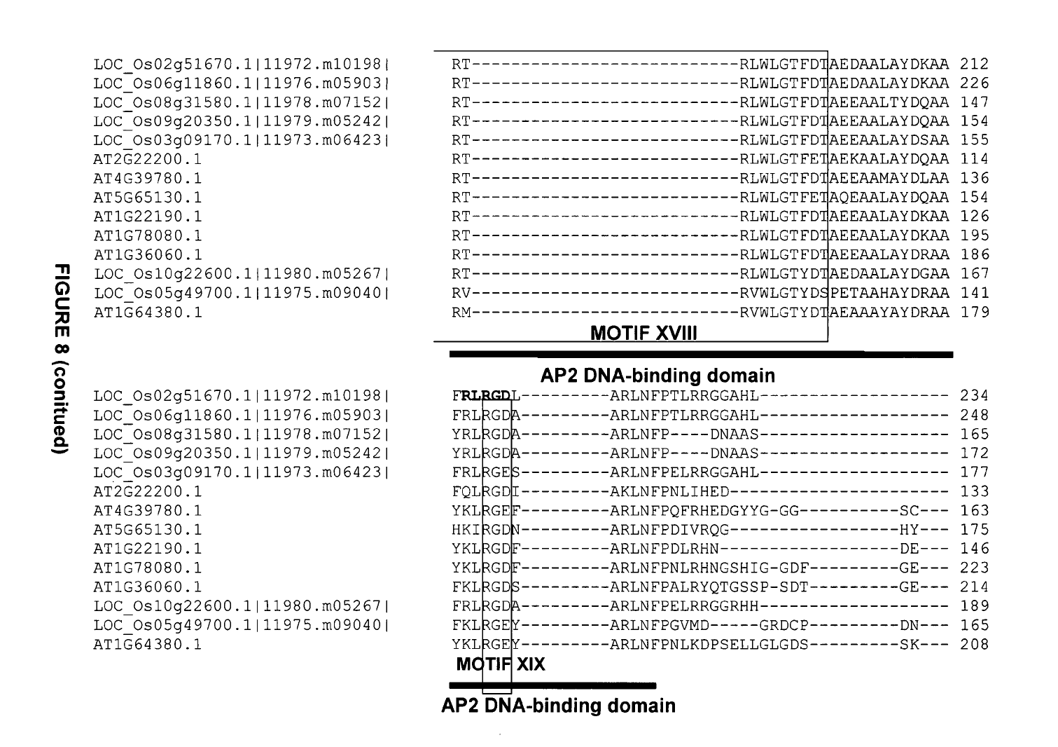

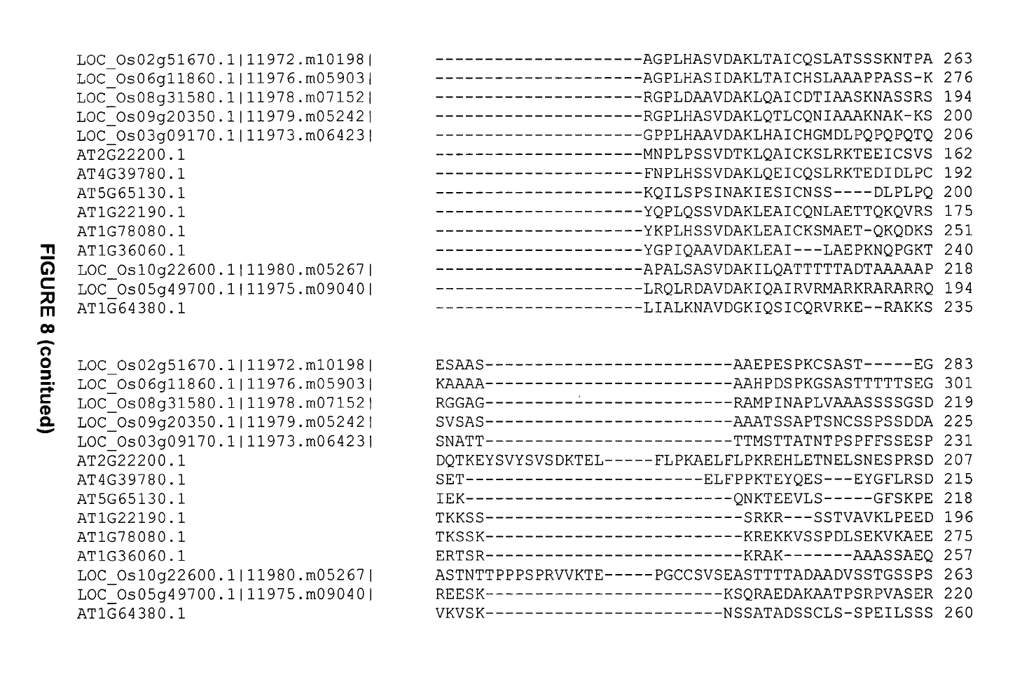

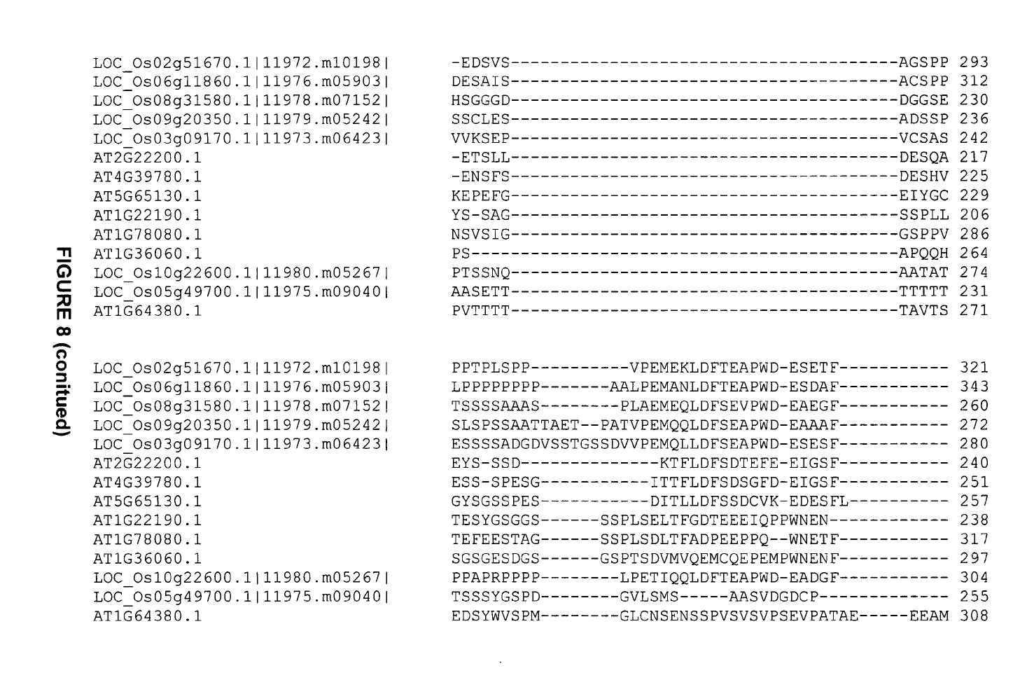

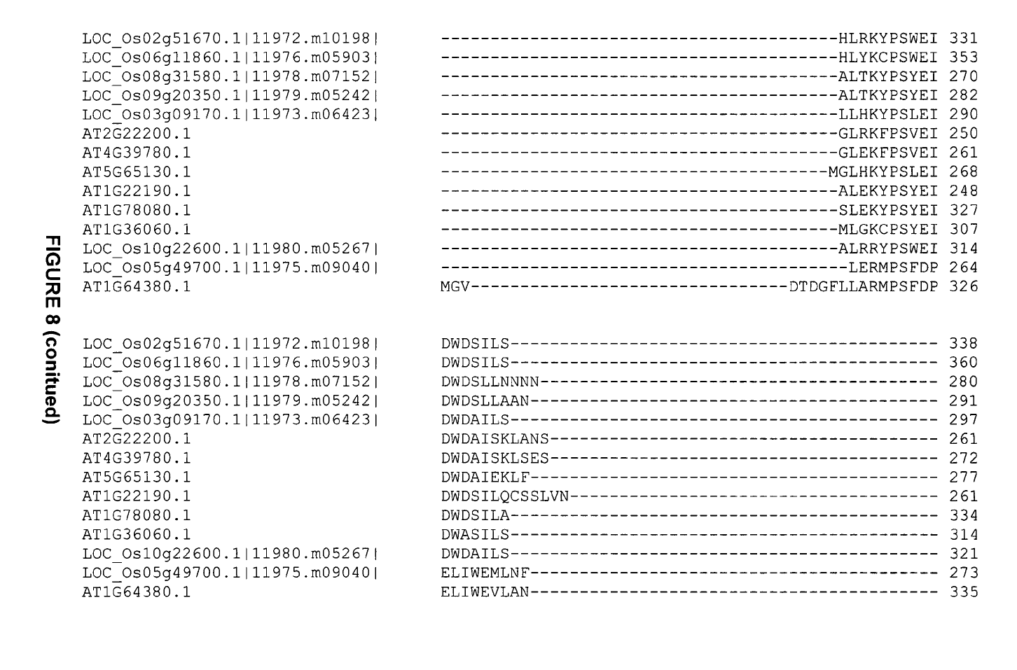

1. A method for enhancing yield-related traits in plants, comprising modulating expression in a plant of a nucleic acid encoding a NAC transcription factor, wherein the amino acid sequence of said NAC transcription factor, when used in the construction of a NAC phylogenetic tree, such as the one depicted in FIG. 1, tends to cluster with the group of NACs comprising the amino acid sequence represented by SEQ ID NO: 2, rather than with any other NAC group.

2-82. (canceled)
